(19) United States
(12) Patent Application Publication
(10) Pub. No.: US 2004/0096451 A1
(43) Pub. Date: May 20, 2004

(54) Methods of treating and preventing RSV, hMPV, and PIV using anti-RSV, anti-hMPV, and anti-PIV antibodies

(76) Inventors: James F. Young, Potomac, MD (US); Peter Kiener, Potomac, MD (US); Albertus Dominicus Marcellinus Erasmus Osterhaus, Bunnik (NL); Ronaldus Adrianus Maria Fouchier, Rotterdam (NL)

Correspondence Address: JONES DAY, 222 EAST 41ST STREET, NEW YORK, NY, 10017, US

(21) Appl. No.: 10/628,088

(22) Filed: Jul. 25, 2003

Related U.S. Application Data
(60) Provisional application No. 60/398,475, filed on Jul. 25, 2002.

(51) Int. Cl. A61K 39/42

(52) U.S. Cl. 424/147.1; 424/159.1

(57) ABSTRACT

The present invention relates to methods for broad spectrum prevention and treatment of viral respiratory infection. In particular, the present invention relates to methods for preventing, treating or ameliorating symptoms associated with respiratory syncytial virus (RSV), parainfluenza virus (PIV), and/or human metapneumovirus (hMPV) infection, the methods comprising administering to a subject an effective amount of one or more anti-RSV-antigen antibodies or antigen-binding fragments thereof, one or more anti-hMPV-antigen antibodies or antigen-binding fragments thereof, and/or one or more anti-PIV-antigen antibodies or antigen-binding fragments thereof. In certain embodiments, a certain serum titer of the anti-RSV-antigen antibodies, anti-PIV-antigen antibodies, and/or anti-hMPV-antigen antibodies or antigen-binding fragments thereof is achieved in said subject. In certain specific embodiments, the subject is human and, preferably, the anti-RSV-antigen antibody, anti-PIV-antigen antibody, and/or anti-hMPV-antigen antibodies are human or humanized. The present invention relates further to compositions comprising the anti-RSV-antigen antibodies, anti-PIV-antigen antibodies, and/or anti-hMPV-antigen antibodies or antigen-binding fragments thereof. The present invention also relates to detectable or diagnostic compositions comprising the one or more anti-RSV-antigen antibodies, anti-PIV-antigen antibodies, and/or anti-hMPV-antigen antibodies or antigen-binding fragments thereof and methods for detecting or diagnosing RSV, PIV and/or hMPV infection utilizing the compositions.

### RELATED APPLICATIONS

This application claims benefit of U.S. provisional application No. 60/398,475, filed Jul. 25, 2002, which is incorporated herein by reference in its entirety.

### 1. INTRODUCTION

The present invention provides methods for broad spectrum prevention and treatment of viral respiratory infection. In particular, the present invention relates to methods for preventing, treating or ameliorating symptoms associated with respiratory syncytial virus (RSV), parainfluenza virus (PIV), and/or human metapneumovirus (hMPV) infection, the methods comprising administering to a subject an effective amount of one or more anti-RSV-antigen antibodies or antigen-binding fragments thereof, one or more anti-hMPV-antigen antibodies or antigen-binding fragments thereof, and/or one or more anti-PIV-antigen antibodies or antigen-binding fragments thereof. In certain embodiments, a certain serum titer of the anti-RSV-antigen antibodies, anti-PIV-antigen antibodies, and/or anti-hMPV-antigen antibodies or antigen-binding fragments thereof is achieved in said subject. In certain specific embodiments, the subject is human and, preferably, the anti-RSV-antigen antibody, anti-PIV-antigen antibody, and/or anti-hMPV-antigen antibodies are human or humanized. The present invention relates further to compositions comprising the anti-RSV-antigen antibodies, anti-PIV-antigen antibodies, and/or anti-hMPV-antigen antibodies or antigen-binding fragments thereof. The present invention also relates to detectable or diagnostic compositions comprising the one or more anti-RSV-antigen antibodies, anti-PIV-antigen antibodies, and/or anti-hMPV-antigen antibodies or antigen-binding fragments thereof and methods for detecting or diagnosing RSV, PIV and/or hMPV infection utilizing the compositions.

### 2. BACKGROUND OF THE INVENTION

2.1. PIV Infections

Parainfluenza viral infection results in serious respiratory tract disease in infants and children. (Tao, et al., 1999, Vaccine 17: 1100-08). Infectious parainfluenza viral infections account for approximately 20% of all hospitalizations of pediatric patients suffering from respiratory tract infections worldwide. Id.

PIV is a member of the paramyxovirus genus of the paramyxovirus family. PIV is made up of two structural modules: (1) an internal ribonucleoprotein core, or nucleocapsid, containing the viral genome, and (2) an outer, roughly spherical lipoprotein envelope. Its genome is a single strand of negative sense RNA, approximately 15,456 nucleotides in length, encoding at least eight polypeptides. These proteins include, but are not limited to, the nucleocapsid structural protein (NP, NC, or N depending on the genera), the phospoprotein (P), the matrix protein (M), the fusion glycoprotein (F), the hemagglutinin-neuraminidase glycoprotein (HN), the large polymerase protein (L), and the C and D proteins of unknown function. Id.

The parainfluenza nucleocapsid protein (NP, NC, or N) consists of two domains within each protein unit including an amino-terminal domain, comprising about two-thirds of the molecule, which interacts directly with the RNA, and a carboxyl-terminal domain, which lies on the surface of the assembled nucleocapsid. A hinge is thought to exist at the junction of these two domains thereby imparting some flexibility to this protein (see Fields et al. (ed.), 1991, Fundamental Virology, Second Edition, Raven Press, New York, incorporated by reference herein in its entirety). The matrix protein (M), is apparently involved with viral assembly and interacts with both the viral membrane as well as the nucleocapsid proteins. The phosphoprotein (P), which is subject to phosphorylation, is thought to play a regulatory role in transcription, and may also be involved in methylation, phosphorylation and polyadenylation. The fusion glycoprotein (F) interacts with the viral membrane and is first produced as an inactive precursor, then cleaved post-translationally to produce two disulfide linked polypeptides. The active F protein is also involved in penetration of the parainfluenza virion into host cells by facilitating fusion of the viral envelope with the host cell plasma membrane. Id. The glycoprotein, hemagglutinin-neuraminidase (HN), protrudes from the envelope allowing the virus to contain both hemagglutinin and neuraminidase activities. HN is strongly hydrophobic at its amino terminal which functions to anchor the HN protein into the lipid bilayer. Id. Finally, the large polymerase protein (L) plays an important role in both transcription and replication. Id.

2.2 RSV Infections

Respiratory syncytial virus (RSV) is the leading cause of serious lower respiratory tract disease in infants and children (Feigen et al., eds., 1987, In: Textbook of Pediatric Infectious Diseases, W B Saunders, Philadelphia at pages 1653-1675; New Vaccine Development, Establishing Priorities, Vol. 1, 1985, National Academy Press, Washington D.C. at pages 397-409; and Ruuskanen et al., 1993, Curr. Probl. Pediatr. 23:50-79). The yearly epidemic nature of RSV infection is evident worldwide, but the incidence and severity of RSV disease in a given season vary by region (Hall, C. B., 1993, Contemp. Pediatr. 10:92-110). In temperate regions of the northern hemisphere, it usually begins in late fall and ends in late spring. Primary RSV infection occurs most often in children from 6 weeks to 2 years of age and uncommonly in the first 4 weeks of life during nosocomial epidemics (Hall et al., 1979, New Engl. J. Med. 300:393-396). Children at increased risk from RSV infection include, but are not limited to, preterm infants (Hall et al., 1979, New Engl. J. Med. 300:393-396) and children with bronchopulmonary dysplasia (Groothuis et al., 1988, Pediatrics 82:199-203), congenital heart disease (MacDonald et al., New Engl. J. Med. 307:397-400), congenital or acquired immunodeficiency (Ogra et al., 1988, Pediatr. Infect. Dis. J. 7:246-249; and Pohl et al., 1992, J. Infect. Dis. 165:166-169), and cystic fibrosis (Abman et al., 1988, J. Pediatr. 113:826-830). The fatality rate in infants with heart or lung disease who are hospitalized with RSV infection is 3%-4% (Navas et al., 1992, J. Pediatr. 121:348-354).

RSV infects adults as well as infants and children. In healthy adults, RSV causes predominantly upper respiratory tract disease. It has recently become evident that some adults, especially the elderly, have symptomatic RSV infections more frequently than had been previously reported (Evans, A. S., eds., 1989, Viral Infections of Humans. Epidemiology and Control, 3^{rd} ed., Plenum Medical Book, New York at pages 525-544). Several epidemics also have been reported among nursing home patients and institutionalized young adults (Falsey, A. R., 1991, Infect. Control Hosp. Epidemiol. 12:602608; and Garvie et al., 1980, Br. Med. J. 281:1253-1254). Finally, RSV may cause serious disease in immunosuppressed persons, particularly bone marrow transplant patients (Hertz et al., 1989, Medicine 68:269281).

Treatment options for established RSV disease are limited. Severe RSV disease of the lower respiratory tract often requires considerable supportive care, including administration of humidified oxygen and respiratory assistance (Fields et al., eds, 1990, Fields Virology, 2^{nd} ed., Vol. 1, Raven Press, New York at pages 1045-1072).

While a vaccine might prevent RSV infection, no vaccine is yet licensed for this indication. A major obstacle to vaccine development is safety. A formalin-inactivated vaccine, though immunogenic, unexpectedly caused a higher and more severe incidence of lower respiratory tract disease due to RSV in immunized infants than in infants immunized with a similarly prepared trivalent parainfluenza vaccine (Kim et al., 1969, Am. J. Epidemiol. 89:422-434; and Kapikian et al., 1969, Am. J. Epidemiol. 89:405-421). Several candidate RSV vaccines have been abandoned and others are under development (Murphy et al., 1994, Virus Res. 32:13-36), but even if safety issues are resolved, vaccine efficacy must also be improved. A number of problems remain to be solved. Immunization would be required in the immediate neonatal period since the peak incidence of lower respiratory tract disease occurs at 2-5 months of age. The immaturity of the neonatal immune response together with high titers of maternally acquired RSV antibody may be expected to reduce vaccine immunogenicity in the neonatal period (Murphy et al., 1988, J. Virol. 62:3907-3910; and Murphy et al., 1991, Vaccine 9:185-189). Finally, primary RSV infection and disease do not protect well against subsequent RSV disease (Henderson et al., 1979, New Engl. J. Med. 300:530-534).

Currently, the only approved approach to prophylaxis of RSV disease is passive immunization. Initial evidence suggesting a protective role for IgG was obtained from observations involving maternal antibody in ferrets (Prince, G. A., Ph.D. diss., University of California, Los Angeles, 1975) and humans (Lambrecht et al, 1976, J. Infect. Dis. 134:211-217; and Glezen et al., 1981, J. Pediatr. 98:708-715). Hemming et al. (Morell et al., eds., 1986, Clinical Use of Intravenous Immunoglobulins, Academic Press, London at pages 285-294) recognized the possible utility of RSV antibody in treatment or prevention of RSV infection during studies involving the pharmacokinetics of an intravenous immune globulin (IVIG) in newborns suspected of having neonatal sepsis. They noted that 1 infant, whose respiratory secretions yielded RSV, recovered rapidly after IVIG infusion. Subsequent analysis of the IVIG lot revealed an unusually high titer of RSV neutralizing antibody. This same group of investigators then examined the ability of hyperimmune serum or immune globulin, enriched for RSV neutralizing antibody, to protect cotton rats and primates against RSV infection (Prince et al., 1985, Virus Res. 3:193-206; Prince et al., 1990, J. Virol. 64:3091-3092; Hemming et al., 1985, J. Infect. Dis. 152:1083-1087; Prince et al., 1983, Infect. Immun. 42:81-87; and Prince et al., 1985, J. Virol. 55:517-520). Results of these studies suggested that RSV neutralizing antibody given prophylactically inhibited respiratory tract replication of RSV in cotton rats. When given therapeutically, RSV antibody reduced pulmonary viral replication both in cotton rats and in a nonhuman primate model. Furthermore, passive infusion of immune serum or immune globulin did not produce enhanced pulmonary pathology in cotton rats subsequently challenged with RSV.

Recent clinical studies have demonstrated the ability of this passively administered RSV hyperimmune globulin (RSV IVIG) to protect at-risk children from severe lower respiratory infection by RSV (Groothius et al., 1993, New Engl. J. Med. 329:1524-1530; and The PREVENT Study Group, 1997, Pediatrics 99:93-99). While this is a major advance in preventing RSV infection, this treatment poses certain limitations in its widespread use. First, RSV IVIG must be infused intravenously over several hours to achieve an effective dose. Second, the concentrations of active material in hyperimmune globulins are insufficient to treat adults at risk or most children with comprised cardiopulmonary function. Third, intravenous infusion necessitates monthly hospital visits during the RSV season.

Finally, it may prove difficult to select sufficient donors to produce a hyperimmune globulin for RSV to meet the demand for this product. Currently, only approximately 8% of normal donors have RSV neutralizing antibody titers high enough to qualify for the production of hyperimmune globulin.

One way to improve the specific activity of the immunoglobulin would be to develop one or more highly potent RSV neutralizing monoclonal antibodies (MAbs). Such MAbs should be human or humanized in order to retain favorable pharmacokinetics and to avoid generating a human anti-mouse antibody response, as repeat dosing would be required throughout the RSV season. Two glycoproteins, F and G, on the surface of RSV have been shown to be targets of neutralizing antibodies (Fields et al., 1990, supra; and Murphy et al., 1994, supra). These two proteins are also primarily responsible for viral recognition and entry into target cells; G protein binds to a specific cellular receptor and the F protein promotes fusion of the virus with the cell. The F protein is also expressed on the surface of infected cells and is responsible for subsequent fusion with other cells leading to syncytia formation. Thus, antibodies to the F protein may directly neutralize virus or block entry of the virus into the cell or prevent syncytia formation. Although antigenic and structural differences between A and B subtypes have been described for both the G and F proteins, the more significant antigenic differences reside on the G glycoprotein, where amino acid sequences are only 53% homologous and antigenic relatedness is 5% (Walsh et al., 1987, J. Infect. Dis. 155:1198-1204; and Johnson et al., 1987, Proc. Natl. Acad. Sci. USA 84:5625-5629). Conversely, antibodies raised to the F protein show a high degree of cross-reactivity among subtype A and B viruses. Beeler and Coelingh (1989, J. Virol. 7:2941-2950) conducted an extensive analysis of 18 different murine MAbs directed to the RSV F protein. Comparison of the biologic and biochemical properties of these MAbs resulted in the identification of three distinct antigenic sites (designated A, B, and C). Neutralization studies were performed against a panel of RSV strains isolated from 1956 to 1985 that demonstrated that epitopes within antigenic sites A and C are highly conserved, while the epitopes of antigenic site B are variable.

A humanized antibody directed to an epitope in the A antigenic site of the F protein of RSV, SYNAGIS®, is approved for intramuscular administration to pediatric patients for prevention of serious lower respiratory tract disease caused by RSV at recommended monthly doses of 15 mg/kg of body weight throughout the RSV season (November through April in the northern hemisphere). SYNAGIS® is a composite of human (95%) and murine (5%) antibody sequences. See, Johnson et al., 1997, J. Infect. Diseases 176:1215-1224 and U.S. Pat. No. 5,824,307, the entire contents of which are incorporated herein by reference. The human heavy chain sequence was derived from the constant domains of human IgG₁ and the variable framework regions of the VH genes of Cor (Press et al., 1970, Biochem. J. 117:641-660) and Cess (Takashi et al., 1984, Proc. Natl. Acad. Sci. USA 81:194-198). The human light chain sequence was derived from the constant domain of Cκ and the variable framework regions of the VL gene K104 with Jκ-4 (Bentley et al., 1980, Nature 288:5194-5198). The murine sequences derived from a murine monoclonal antibody, Mab 1129 (Beeler et al., 1989, J. Virology 63:2941-2950), in a process which involved the grafting of the murine complementarity determining regions into the human antibody frameworks.

2.3 Avian and Human Metapneumovirus

Recently, a new member of the Paramyxoviridae family has been isolated from 28 children with clinical symptoms reminiscent of those caused by hRSV infection, ranging from mild upper respiratory tract disease to severe bronchiolitis and pneumonia (Van Den Hoogen et al., 2001, Nature Medicine 7:719-724). The new virus was named human metapneumovirus (hMPV) based on sequence homology and gene constellation. The study further showed that by the age of five years virtually all children in the Netherlands have been exposed to hMPV and that the virus has ben circulating in humans for at least half a century.

The genomic organization of human metapneumovirus is described in van den Hoogen et al, 2002, Virology 295:119-132. Human metapneumovirus has recently been isolated from patients in North America (Peret et al., 2002, J. Infect. Diseases 185:1660-1663).

Human metapneumovirus is related to avian metapneumovirus. For exampe, the F protein of hMPV is highly homologous to the F protein of APV. Alignment of the human metapneumoviral F protein with the F protein of an avian pneumovirus isolated from Mallard Duck shows 85.6% identity in the ectodomain. Alignment of the human metapneumoviral F protein with the F protein of an avian pneumovirus isolated from Turkey (subgroup B) shows 75% identity in the ectodomain. See, e.g., co-owned and co-pending Provisional Application No. 60/358,934, entitled “Recombinant Parainfluenza Virus Expression Systems and Vaccines Comprising Heterologous Antigens Derived from Metapneumovirus”, filed on Feb. 21, 2002, by Haller and Tang, which is incorporated herein by reference in its entirety.

Respiratory disease caused by an avian pneumovirus (APV) was first described in South Africa in the late 1970s (Buys et al., 1980, Turkey 28:36-46) where it had a devastating effect on the turkey industry. The disease in turkeys was characterized by sinusitis and rhinitis and was called turkey rhinotracheitis (TRT). The European isolates of APV have also been strongly implicated as factors in swollen head syndrome (SHS) in chickens (O'Brien, 1985, Vet. Rec. 117:619-620). Originally, the disease appeared in broiler chicken flocks infected with Newcastle disease virus (NDV) and was assumed to be a secondary problem associated with Newcastle disease (ND). Antibody against European APV was detected in affected chickens after the onset of SHS (Cook et al., 1988, Avian Pathol. 17:403-410), thus implicating APV as the cause.

The avian pneumovirus is a single stranded, non-segmented RNA virus that belongs to the sub-family Pneumovirinae of the family Paramyxoviridae, genus metapneumovirus (Cavanagh and Barrett, 1988, Virus Res. 11:241-256; Ling et al., 1992, J. Gen. Virol. 73:1709-1715; Yu et al., 1992, J. Gen. Virol. 73:1355-1363). The Paramyxoviridae family is divided into two sub-families: the Paramyxovirinae and Pneumovirinae. The subfamily Paramyxovirinae includes, but is not limited to, the genera: Paramyxovirus, Rubulavirus, and Morbillivirus. Recently, the sub-family Pneumovirinae was divided into two genera based on gene order, i.e. pneumovirus and metapneumovirus (Naylor et al., 1998, J. Gen. Virol., 79:1393-1398; Pringle, 1998, Arch. Virol. 143:1449-1159). The pneumovirus genus includes, but is not limited to, human respiratory syncytial virus (hRSV), bovine respiratory syncytial virus (BRSV), ovine respiratory syncytial virus, and mouse pneumovirus. The metapneumovirus genus includes, but is not limited to, European avian pneumovirus (subgroups A and B), which is distinguished from hRSV, the type species for the genus pneumovirus (Naylor et al., 1998, J. Gen. Virol., 79:1393-1398; Pringle, 1998, Arch. Virol. 143:1449-1159). The US isolate of APV represents a third subgroup (subgroup C) within metapneumovirus genus because it has been found to be antigenically and genetically different from European isolates (Seal, 1998, Virus Res. 58:45-52; Senne et al., 1998, In: Proc. 47^{th} WPDC, California, pp. 67-68).

Electron microscopic examination of negatively stained APV reveals pleomorphic, sometimes spherical, virions ranging from 80 to 200 nm in diameter with long filaments ranging from 1000 to 2000 nm in length (Collins and Gough, 1988, J. Gen. Virol. 69:909-916). The envelope is made of a membrane studded with spikes 13 to 15 nm in length. The nucleocapsid is helical, 14 nm in diameter and has 7 nm pitch. The nucleocapsid diameter is smaller than that of the genera Paramyxovirus and Morbillivirus, which usually have diameters of about 18 nm.

Avian pneumovirus infection is an emerging disease in the USA despite its presence elsewhere in the world in poultry for many years. In May 1996, a highly contagious respiratory disease of turkeys appeared in Colorado, and an APV was subsequently isolated at the National Veterinary Services Laboratory (NVSL) in Ames, Iowa (Senne et al., 1997, Proc. 134^{th} Ann. Mtg., AVMA, pp. 190). Prior to this time, the United States and Canada were considered free of avian pneumovirus (Pearson et al., 1993, In: Newly Emerging and Re-emerging Avian Diseases: Applied Research and Practical Applications for Diagnosis and Control, pp. 78-83; Hecker and Myers, 1993, Vet. Rec. 132:172). Early in 1997, the presence of APV was detected serologically in turkeys in Minnesota. By the time the first confirmed diagnosis was made, APV infections had already spread to many farms. The disease is associated with clinical signs in the upper respiratory tract: foamy eyes, nasal discharge and swelling of the sinuses. It is exacerbated by secondary infections. Morbidity in infected birds can be as high as 100%. The mortality can range from 1 to 90% and is highest in six to twelve week old poults.

Avian pneumovirus is transmitted by contact. Nasal discharge, movement of affected birds, contaminated water, contaminated equipment; contaminated feed trucks and load-out activities can contribute to the transmission of the virus. Recovered turkeys are thought to be carriers. Because the virus is shown to infect the epithelium of the oviduct of laying turkeys and because APV has been detected in young poults, egg transmission is considered a possibility.

Based upon the recent work with hMPV, hMPV likewise appears to be a significant factor in human, particularly, juvenile respiratory disease.

Thus, theses three viruses, RSV, hMPV, and PIV, cause a significant portion of human respiratory disease. What is needed is a broad spectrum prophylaxis to reduce the incidence of viral respiratory disease.

Citation or discussion of a reference herein shall not be construed as an admission that such is prior art to the present invention.

2.4 Virus Entry into Host Cell

It is emerging that some of the enveloped viruses, e.g., retrovirus, orthomyxovirus, filovirus, and paramyxovirus, might use a fusion mechanism involving so-called heptad repeats to gain entry into a host cell (Eckert et al., 2001, Annu. Rev. Biochem. 70:777-810; Weissenhom et. al., 1999, Mol. Membr. Biol. 16:3-9; Lamb et. al., 1999, Mol. Membr. Biol. 16:11-19; Skehel et al., 2000, Annu. Rev. Biochem. 69:531-569; Bentz, J., 2000, Biophys J. 78:886-900; Peisajovich et. al., 2002, Trends Biochem. Sci. 27:183-190). According to this model, the fusion peptide located at the N-terminus of the F protein (e.g., of paramyxovirus) is exposed to insert itself into the cell membrane. Further, fusion proteins undergo conformational changes during fusion (Wang et al., 2003, Biochem. Biophys. Res. Comm. 302:469-475). The highly conserved heptad repeat (HR) regions have been implicated in facilitation of the fusion process (Wang et al., 2003, Biochem. Biophys. Res. Comm. 302:469-475). Therefore, the heptad repeats are an attractive target for the prevention of virus infection and/or propagation through the inhibition of fusion with a host cell.

### 3 SUMMARY OF THE INVENTION

The present invention provides methods for broad spectrum prevention and treatment of viral respiratory infections. Viruses are major causes of severe respiratory infections, particularly in infants, prematurely born infants, the elderly, immunocompromised patients, recipients of transplants, etc. Respiratory infections can be effectively prevented and/or treated using the combination therapies/prophylaxes provided by the present invention. The present invention provides broad spectrum combination therapy/prophylaxis comprising administering to a subject (i) one or more anti-RSV-antigen antibodies or antigen-binding fragments thereof; (ii) one or more anti-PIV-antigen antibodies or antigen-binding fragments thereof; and/or (iii) one or more anti-hMPV-antigen antibodies or antigen-binding fragments thereof. By providing to the subject a plurality of antibodies directed to antigens of a variety of viruses, the risk of respiratory viral infection is reduced in the subject. A particular advantage of administering antibodies of different immunospecificities is that different strains of viruses and viruses with naturally occuring modifications do not escape the immunity of the subject but are recognized by at least one of the plurality of antibodies.

In certain embodiments, the invention provides a method of preventing a viral infection in a subject, said method comprising administering to the subject: (i) a prophylactically effective amount of one or more first antibodies or antigen-binding fragments thereof, wherein said one or more first antibodies or antigen-binding fragments thereof bind immunospecifically to a RSV antigen; and (ii) a prophylactically effective amount of one or more second antibodies or antigen-binding fragments thereof, wherein said one or more second antibodies or antigen-binding fragments thereof bind immunospecifically to a hMPV antigen. In certain embodiments, the one or more anti-RSV-antigen antibodies or antigen-binding fragments thereof neutralize RSV. In certain embodiments, the one or more anti-hMPV-antigen antibodies or antigen-binding fragments thereof neutralize hMPV. In certain embodiments, the one or more anti-RSV-antigen antibodies or antigen-binding fragments thereof block RSV infection of cells of the subject. In certain embodiments, the one or more anti-hMPV-antigen antibodies antibodies or antigen-binding fragments thereof block hMPV infection of cells of the subject.

In certain embodiments, the invention provides a method of treating one or more symptoms of a respiratory viral infection in a subject, said method comprising administering to the subject: (i) a therapeutically effective amount of one or more first antibodies or antigen-binding fragments thereof, wherein said one or more first antibodies or antigen-binding fragments thereof bind immunospecifically to a RSV antigen; and (ii) a therapeutically effective amount of one or more second antibodies or antigen-binding fragments thereof, wherein said one or more second antibodies or antigen-binding fragments thereof bind immunospecifically to a hMPV antigen.

In certain embodiments, the invention provides a method of passive immunotherapy, said method comprising administering to a subject: (i) a first dose of one or more first antibodies or antigen-binding fragments thereof, wherein said one or more first antibodies or a fragments thereof bind immunospecifically to a RSV antigen; and (ii) a second dose of one or more second antibodies or antigen-binding fragments thereof, wherein said one or more second antibodies or a fragments thereof bind immunospecifically to a hMPV antigen, wherein the first dose reduces the incidence of RSV infection by at least 25% and wherein the second dose reduces the incidence of hMPV infection by at least 25%. In certain embodiments, the first dose reduces the incidence of RSV infection by at least 50% and wherein the second dose reduces the incidence of hMPV infection by at least 50%. In certain embodiments, the first dose reduces the incidence of RSV infection by at least 75% and wherein the second dose reduces the incidence of hMPV infection by at least 75%. In certain embodiments, the first dose reduces the incidence of RSV infection by at least 90% and wherein the second dose reduces the incidence of hMPV infection by at least 90%.

In certain embodiments, the invention provides a method of passive immunotherapy, said method comprising administering to a subject: (i) a first dose of one or more first antibodies or antigen-binding fragments thereof, wherein said one or more first antibodies or antigen-binding fragments thereof bind immunospecifically to a RSV antigen; and (ii) a second dose of one or more second antibodies or antigen-binding fragments thereof, wherein said one or more second antibodies or antigen-binding fragments thereof bind immunospecifically to a hMPV antigen, wherein the serum titer of said one or more anti-RSV-antigen antibodies or antigen-binding fragments thereof in the subject is at least 10 μg/ml after 15 days of administering said one or more anti-RSV-antigen antibodies or antigen-binding fragments thereof and wherein the serum titer of said one or more anti-hMPV-antigen antibodies or antigen-binding fragments thereof in the subject is at least 10 μg/ml after 15 days of administering said one or more anti-hMPV-antigen antibodies or antigen-binding fragments thereof.

In certain embodiments, the amino acid sequence of the RSV antigen is that of SEQ ID NO:390 to 398, respectively. In certain embodiments, the amino acid sequence of the RSV antigen is 90% identical to the amino acid sequence of RSV nucleoprotein, RSV phosphoprotein, RSV matrix protein, RSV small hydrophobic protein, RSV RNA-dependent RNA polymerase, RSV F protein, or RSV G protein. In certain embodiments, the the RSV antigen is selected from the group consisting of RSV nucleoprotein, RSV phosphoprotein, RSV matrix protein, RSV small hydrophobic protein, RSV RNA-dependent RNA polymerase, RSV F protein, and RSV G protein. In certain embodiments, the one or more anti-RSV-antigen antibodies immunospecifically bind to an antigen of Group A or Group B RSV. In certain embodiments, the RSV antigen is RSV F protein. In certain embodiments, the one or more anti-hMPV-antigen antibodies cross-react with a turkey APV antigen. In certain embodiments, the one or more anti-hMPV-antigen antibodies are (i) human or humanized antibodies and (ii) cross-react with a turkey APV antigen. In certain embodiments, the turkey APV antigen is selected from the group consisting of turkey APV nucleoprotein, turkey APV phosphoprotein, turkey APV matrix protein, turkey APV small hydrophobic protein, turkey APV RNA-dependent RNA polymerase, turkey APV F protein, and turkey APV G protein. In certain embodiments, the turkey APV antigen is an antigen of avian pneumovirus type A, avian pneumovirus type B, or avian pneumovirus type C. In certain embodiments, the amino acid sequence of said turkey APV antigen is that of SEQ ID NO:424 to 429, respectively. In certain embodiments, the amino acid sequence of the hMPV antigen is that of SEQ ID NO:399 to 406, 420, or 421, respectively. In certain embodiments, the hMPV antigen is selected from the group consisting of hMPV nucleoprotein, hMPV phosphoprotein, hMPV matrix protein, hMPV small hydrophobic protein, hMPV RNA-dependent RNA polymerase, hMPV F protein, and hMPV G protein. In certain embodiments, the hMPV antigen is hMPV F protein. In certain embodiments, the anti-RSV-antigen antibody is SYNAGIS™ (Palivizumab); AFFF; P12f2 P12f4; P11d4; Ale9; A12a6; A13c4; A17d4; A4B4; 1X-493L1; FR H3-3F4; M3H9; Y10H6; DG; AFFF(1); 6H8; L1-7E5; L2-15B10; A13a11; A1h5; A4B4(1);A4B4-F52S; or A4B4L1FR-S28R. In certain embodiments, the effective amount of said one or more anti-RSV-antigen antibodies is 100 mg/kg or less. In certain embodiments, the effective amount of said one or more anti-RSV-antigen antibodies is 10 mg/kg or less. In certain embodiments, the effective amount of said one or more anti-RSV-antigen antibodies is 1 mg/kg or less. In certain embodiments, the effective amount of said one or more anti-hMPV-antigen antibodies or antigen-binding fragments thereof is 100 mg/kg or less. In certain embodiments, the effective amount of said one or more anti-hMPV-antigen antibodies or antigen-binding fragments thereof is 10 mg/kg or less. In certain embodiments, the effective amount of said one or more anti-hMPV-antigen antibodies or antigen-binding fragments thereof is 1 mg/kg or less. In certain embodiments, the one or more anti-RSV-antigen antibodies or antigen-binding fragments thereof are administered at a time period prior to administering of said one or more anti-hMPV-antigen antibodies or antigen-binding fragments thereof. In certain embodiments, the one or more anti-hMPV-antigen antibodies or antigen-binding fragments thereof are administered at a time period prior to administering of said one or more anti-RSV-antigen antibodies or antigen-binding fragments thereof. In certain embodiments, the one or more anti-RSV-antigen antibodies or antigen-binding fragments thereof and said one or more anti-hMPV-antigen antibodies or antigen-binding fragments thereof are administered concurrently. In certain embodiments, the one or more anti-RSV-antigen antibodies or antigen-binding fragments thereof are administered in a sequence of two or more administrations, wherein the administrations of said one or more anti-RSV-antigen antibodies or antigen-binding fragments thereof are separated by a time period from each other, and wherein said one or more anti-hMPV-antigen antibodies or antigen-binding fragments thereof are administered before, during, or after the sequence. In certain embodiments, the one or more anti-RSV-antigen antibodies or antigen-binding fragments thereof are administered in a sequence of two or more administrations, wherein the administrations of said one or more anti-hMPV-antigen antibodies or antigen-binding fragments thereof are separated by a time period from each other, and wherein said one or more anti-RSV-antigen antibodies or antigen-binding fragments thereof are administered before, during, or after the sequence. In certain embodiments, the one or more anti-RSV-antigen antibodies or antigen-binding fragments thereof and said one or more anti-hMPV-antigen antibodies or antigen-binding fragments thereof are administered in a sequence of two or more administrations, wherein the administrations are separated by a time period from each other. In certain embodiments, the time period is at least 1 day, 2 days, 3 days, 4 days, 5 days, 6 days, 1 week, 2 weeks, 3 weeks, 1 month, 2 months, or 3 months. In certain embodiments, the one or more anti-RSV-antigen antibodies or antigen-binding fragments thereof and/or said one or more anti-hMPV-antigen antibodies or antigen-binding fragments thereof are administered by a nebulizer or an inhaler. In certain embodiments, the one or more anti-RSV-antigen antibodies or antigen-binding fragments thereof and/or said one or more anti-hMPV-antigen antibodies or antigen-binding fragments thereof are administered intramuscularly, intravenously or subcutaneously. In certain embodiments, the viral infection is an infection with RSV and hMPV. In certain embodiments, the viral infection is an infection with RSV and APV. In certain embodiments, at least one of said antibodies is a monoclonal antibody, a synthetic antibody, an intrabody, a chimeric antibody, a human antibody, a humanized chimeric antibody, a humanized antibody, a glycosylated antibody, a multispecific antibody, a human antibody, a single-chain antibody, or a bispecific antibody. In certain embodiments, at least one of said antibodies is a human antibody. In certain embodiments, at least one of said antibodies is a humanized antibody. In certain embodiments, at least one of said antibodies is a synthetic antibody. In certain embodiments, the subject is a mammal. In certain embodiments, the mammal is a primate. In certain embodiments, the primate is a human. In certain embodiments, the human is an elderly human. In certain embodiments, the human is a transplant recipient. In certain embodiments, the human is an immunocompromised patient. In certain embodiments, the human is an A/DS patient. In certain embodiments, the human is an infant. In certain embodiments, the human has cystic fibrosis, bronchopulmonary dysplasia, congenital heart disease, congenital immunodeficiency, or acquired immunodeficiency or has had a bone marrow transplant. In certain embodiments, infant was born prematurely or is at risk of hospitalization for a RSV infection and/or for a hMPV infection. In certain embodiments, the human infant was born prematurely. In certain embodiments, the infant is less than 32 weeks of gestational age. In certain embodiments, the infant is between 32 and 35 weeks of gestational age. In certain embodiments, the infant is more than 35 weeks of gestational age. In certain embodiments, the infant is more than 38 weeks of gestational age. In certain embodiments, the mammal is not a primate. In certain embodiments, the non-primate mammal is an animal model for RSV infection and/or hMPV infection. In certain embodiments, the non-primate mammal is a cotton rat. In certain embodiments, the antibody is administered once a month just prior to and during the RSV season. In certain embodiments, the antibody is administered every two months just prior to and during the RSV season. In certain embodiments, the antibody is administered once just prior to or during the RSV season. In certain embodiments, at least one of said fragments is a Fab fragment, a F(ab′) fragment, a F(ab′)₂ fragment, a Fd, a single-chain Fv, a disulfide-linked Fv, a fragment comprising a V_{L} domain, or a fragment comprising a V_{H} domain.

In certain embodiments, the invention provides a method of preventing a viral infection in a subject, said method comprising administering to the subject: (i) a dose of one or more antibodies or antigen-binding fragments thereof, wherein said one or more antibodies or antigen-binding fragments thereof (i) are human or humanized, (ii) cross-react with a turkey APV antigen, and (iii) bind immunospecifically to a hMPV antigen.

In certain embodiments, the invention provides method of treating one or more symptoms of a respiratory viral infection in a subject, said method comprising administering to the subject: (i) a dose of one or more antibodies or antigen-binding fragments thereof, wherein said one or more antibodies or antigen-binding fragments thereof (i) are human or humanized, (ii) cross-react with a turkey APV antigen, and (iii) bind immunospecifically to a hMPV antigen.

In certain embodiments, the invention provides a method of passive immunotherapy, said method comprising administering to a subject: (i) a dose of one or more antibodies or antigen-binding fragments thereof, wherein said one or more antibodies or antigen-binding fragments thereof (i) are human or humanized, (ii) cross-react with a turkey APV antigen, and (iii) bind immunospecifically to a hMPV antigen, wherein the dose reduces the incidence of hMPV infection by at least 25%. In certain embodiments, wherein the dose reduces the incidence of hMPV infection by at least 50%. In certain embodiments, wherein the dose reduces the incidence of hMPV infection by at least 75%. In certain embodiments, wherein the dose reduces the incidence of hMPV infection by at least 90%.

In certain embodiments, the invention provides a method of passive immunotherapy, said method comprising administering to a subject: (i) a dose of one or more antibodies or antigen-binding fragments thereof, wherein said one or more antibodies or antigen-binding fragments thereof (i) are human or humanized, (ii) cross-react with a turkey APV antigen, and (iii) bind immunospecifically to a hMPV antigen, wherein the serum titer of said one or more antibodies or antigen-binding fragments thereof in the subject is at least 10 μg/ml after 15 days of administering said one or more antibodies or antigen-binding fragments thereof.

In certain embodiments, the invention provides a pharmaceutical composition, said composition comprising: (i) one or more first antibodies or antigen-binding fragments thereof, wherein said one or more first antibodies or antigen-binding fragments thereof bind immunospecifically to a RSV antigen; and (ii) one or more second antibodies or antigen-binding fragments thereof, wherein said one or more second antibodies or antigen-binding fragments thereof bind immunospecifically to a hMPV antigen. In certain embodiments, the amino acid sequence of the RSV antigen is that of SEQ ID NO:390 to 398, respectively. In certain embodiments, the amino acid sequence of the RSV antigen is 90% identical to the amino acid sequence of RSV nucleoprotein, RSV phosphoprotein, RSV matrix protein, RSV small hydrophobic protein, RSV RNA-dependent RNA polymerase, RSV F protein, or RSV G protein. In certain embodiments, the RSV antigen is selected from the group consisting of RSV nucleoprotein, RSV phosphoprotein, RSV matrix protein, RSV small hydrophobic protein, RSV RNA-dependent RNA polymerase, RSV F protein, and RSV G protein. In certain embodiments, said one or more anti-RSV-antigen antibodies or antigen-binding fragments thereof immunospecifically bind to an antigen of Group A or Group B RSV. In certain embodiments, the RSV antigen is RSV F protein. In certain embodiments, said one or more anti-hMPV-antigen antibodies cross-react with a turkey APV antigen. In certain embodiments, said one or more anti-hMPV-antigen antibodies are (i) human or humanized antibodies and (ii) cross-react with a turkey APV antigen. In certain embodiments, said turkey APV antigen is selected from the group consisting of turkey APV nucleoprotein, turkey APV phosphoprotein, turkey APV matrix protein, turkey APV small hydrophobic protein, turkey APV RNA-dependent RNA polymerase, turkey APV F protein, and turkey APV G protein. In certain embodiments, said turkey APV antigen is an antigen of avian pneumovirus type A, avian pneumovirus type B, or avian pneumovirus type C. In certain embodiments, the amino acid sequence of said turkey APV antigen is that of SEQ ID NO:424 to 429, respectively. In certain embodiments, the amino acid sequence of the hMPV antigen is that of SEQ ID NO:399 to 406, 420, or 421, respectively. In certain embodiments, the hMPV antigen is selected from the group consisting of hMPV nucleoprotein, hMPV phosphoprotein, hMPV matrix protein, hMPV small hydrophobic protein, hMPV RNA-dependent RNA polymerase, hMPV F protein, and hMPV G protein. In certain embodiments, the hMPV antigen is hMPV F protein. In certain embodiments, the anti-RSV-antigen antibody is SYNAGIS™; AFFF; P12f2 P12f4; P11d4; Ale9; A12a6; A13c4; A17d4; A4B4; 1X-493L1; FR H3-3F4; M3H9; Y10H6; DG; AFFF(1); 6H8; L1-7E5; L2-15B10; A13a11; A1h5; A4B4(1);A4B4-F52S; or A4B4L1FR-S28R. In certain embodiments, at least one of said antibodies is a monoclonal antibody, a synthetic antibody, an intrabody, a chimeric antibody, a human antibody, a humanized chimeric antibody, a humanized antibody, a glycosylated antibody, a multispecific antibody, a human antibody, a single-chain antibody, or a bispecific antibody. In certain embodiments, at least one of said antibodies is a human antibody. In certain embodiments, at least one of said antibodies is a humanized antibody. In certain embodiments, at least one of said antibodies is a synthetic antibody. In certain embodiments, at least one of said fragments is a Fab fragment, a F(ab′) fragment, a F(ab′)₂ fragment, a Fd, a single-chain Fv, a disulfide-linked Fv, a fragment comprising a V_{L} domain, or a fragment comprising a V_{H} domain.

In certain embodiments, the application provides a pharmaceutical composition, said composition comprising: one or more antibodies or antigen-binding fragments thereof, wherein said one or more antibodies or antigen-binding fragments thereof (i) are human or humanized, (ii) cross-react with a turkey APV antigen, and (iii) bind immunospecifically to a hMPV antigen.

In certain embodiments, the invention provides a method comprising administering to the subject: (i) a prophylactically effective amount of one or more first antibodies or antigen-binding fragments thereof, wherein said one or more first antibodies or antigen-binding fragments thereof bind immunospecifically to a PIV antigen; and (ii) a prophylactically effective amount of one or more second antibodies or antigen-binding fragments thereof, wherein said one or more second antibodies or antigen-binding fragments thereof bind immunospecifically to a hMPV antigen. In certain embodiments, said one or more anti-PIV-antigen antibodies or antigen-binding fragments thereof neutralize PIV. In certain embodiments, said one or more anti-hMPV-antigen antibodies or antigen-binding fragments thereof neutralize hMPV. In certain embodiments, said one or more anti-PIV-antigen antibodies or antigen-binding fragments thereof block PIV infection of cells of the subject. In certain embodiments, said one or more anti-hMPV-antigen antibodies or antigen-binding fragments thereof block hMPV infection of cells of the subject.

In certain embodiments, the invention provides a method of treating one or more symptoms of a respiratory viral infection in a subject, said method comprising administering to the subject: (i) a therapeutically effective amount of one or more first antibodies or antigen-binding fragments thereof, wherein said one or more first antibodies or antigen-binding fragments thereof bind immunospecifically to a PIV antigen; and (ii) a therapeutically effective amount of one or more second antibodies or antigen-binding fragments thereof, wherein said one or more second antibodies or antigen-binding fragments thereof bind immunospecifically to a hMPV antigen.

In certain embodiments, the invention provides a method of passive immunotherapy, said method comprising administering to a subject: (i) a first dose of one or more first antibodies or antigen-binding fragments thereof, wherein said one or more first antibodies or a fragments thereof bind immunospecifically to a PIV antigen; and (ii) a second dose of one or more second antibodies or antigen-binding fragments thereof, wherein said one or more second antibodies or a fragments thereof bind immunospecifically to a hMPV antigen, wherein the first dose reduces the incidence of PIV infection by at least 25% and wherein the second dose reduces the incidence of hMPV infection by at least 25%. In certain embodiments, the first dose reduces the incidence of PIV infection by at least 50% and wherein the second dose reduces the incidence of hMPV infection by at least 50%. In certain embodiments, the first dose reduces the incidence of PIV infection by at least 75% and wherein the second dose reduces the incidence of hMPV infection by at least 75%. In certain embodiments, the first dose reduces the incidence of PIV infection by at least 90% and wherein the second dose reduces the incidence of hMPV infection by at least 90%.

In certain embodiments, the invention provides a method of passive immunotherapy, said method comprising administering to a subject: (i) a first dose of one or more anti-PIV-antigen antibodies or antigen-binding fragments thereof, wherein said one or more first antibodies or antigen-binding fragments thereof bind immunospecifically to a PIV antigen; and (ii) a second dose of one or more anti-hMPV-antigen antibodies or antigen-binding fragments thereof, wherein said one or more anti-hMPV-antigen antibodies or antigen-binding fragments thereof bind immunospecifically to a hMPV antigen, wherein the serum titer of said one or more anti-PIV-antigen antibodies or antigen-binding fragments thereof in the subject is at least 10 μg/ml after 15 days of administering said one or more anti-PIV-antigen antibodies or antigen-binding fragments thereof and wherein the serum titer of said one or more anti-hMPV-antigen antibodies or antigen-binding fragments thereof in the subject is at least 10 μg/ml after 15 days of administering said one or more anti-hMPV-antigen antibodies or antigen-binding fragments thereof. In certain embodiments, the amino acid sequence of the PIV antigen is that of SEQ ID NO:407 to 419, respectively. In certain embodiments, the amino acid sequence of the PIV antigen is 90% identical to the amino acid sequence of PIV nucleocapsid phosphoprotein, PIV L protein, PIV matrix protein, PIV HN glycoprotein, PIV RNA-dependent RNA polymerase, PIV Y1 protein, PIV D protein, or PIV C protein. In certain embodiments, the PIV antigen is selected from the group consisting of PIV nucleocapsid phosphoprotein, PIV L protein, PIV matrix protein, PIV HN glycoprotein, PIV RNA-dependent RNA polymerase, PIV Y1 protein, PIV D protein, or PIV C protein. In certain embodiments, said one or more anti-hMPV-antigen antibodies immunospecifically bind to an antigen of human PIV type 1, human PIV type 2, human PIV type 3, or human PIV type 4. In certain embodiments, the PIV antigen is PIV F protein. In certain embodiments, said one or more anti-hMPV-antigen antibodies cross-react with a turkey APV antigen. In certain embodiments, said one or more anti-hMPV-antigen antibodies are (i) human or humanized antibodies and (ii) cross-react with a turkey APV antigen. In certain embodiments, said turkey APV antigen is selected from the group consisting of turkey APV nucleoprotein, turkey APV phosphoprotein, turkey APV matrix protein, turkey APV small hydrophobic protein, turkey APV RNA-dependent RNA polymerase, turkey APV F protein, and turkey APV G protein. In certain embodiments, said turkey APV antigen is an antigen of avian pneumovirus type A, avian pneumovirus type B, or avian pneumovirus type C. In certain embodiments, the amino acid sequence of said turkey APV antigen is that of SEQ ID NO:424 to 429, respectively. In certain embodiments, the amino acid sequence of the hMPV antigen is that of SEQ ID NO:399-406, 420, or 421, respectively. In certain embodiments, the hMPV antigen is selected from the group consisting of hMPV nucleoprotein, hMPV phosphoprotein, hMPV matrix protein, hMPV small hydrophobic protein, hMPV RNA-dependent RNA polymerase, hMPV F protein, and hMPV G protein. In certain embodiments, the hMPV antigen is hMPV F protein. In certain embodiments, the effective amount of said one or more anti-PIV-antigen antibodies is 100 mg/kg or less. In certain embodiments, the effective amount of said one or more anti-PIV-antigen antibodies is 10 mg/kg or less. In certain embodiments, the effective amount of said one or more anti-PIV-antigen antibodies is 1 mg/kg or less. In certain embodiments, the effective amount of said one or more anti-hMPV-antigen antibodies or antigen-binding fragments thereof is 100 mg/kg or less. In certain embodiments, the effective amount of said one or more anti-hMPV-antigen antibodies or antigen-binding fragments thereof is 10 mg/kg or less. In certain embodiments, the effective amount of said one or more anti-hMPV-antigen antibodies or antigen-binding fragments thereof is 1 mg/kg or less. In certain embodiments, said one or more anti-PIV-antigen antibodies or antigen-binding fragments thereof are administered at a time period prior to administering of said one or more anti-hMPV-antigen antibodies or antigen-binding fragments thereof. In certain embodiments, said one or more anti-hMPV-antigen antibodies or antigen-binding fragments thereof are administered at a time period prior to administering of said one or more anti-PIV-antigen antibodies or antigen-binding fragments thereof. In certain embodiments, said one or more anti-PIV-antigen antibodies or antigen-binding fragments thereof and said one or more anti-hMPV-antigen antibodies or antigen-binding fragments thereof are administered concurrently. In certain embodiments, said one or more anti-PIV-antigen antibodies or antigen-binding fragments thereof are administered in a sequence of two or more administrations, wherein the administrations of said one or more anti-PIV-antigen antibodies or antigen-binding fragments thereof are separated by a time period from each other, and wherein said one or more anti-hMPV-antigen antibodies or antigen-binding fragments thereof are administered before, during, or after the sequence. In certain embodiments, said one or more anti-PIV-antigen antibodies or antigen-binding fragments thereof are administered in a sequence of two or more administrations, wherein the administrations of said one or more anti-hMPV-antigen antibodies or antigen-binding fragments thereof are separated by a time period from each other, and wherein said one or more anti-PIV-antigen antibodies or antigen-binding fragments thereof are administered before, during, or after the sequence. In certain embodiments, said one or more anti-PIV-antigen antibodies or antigen-binding fragments thereof and said one or more anti-hMPV-antigen antibodies or antigen-binding fragments thereof are administered in a sequence of two or more administrations, wherein the administrations are separated by a time period from each other. In certain embodiments, the time period is at least 1 day, 2 days, 3 days, 4 days, 5 days, 6 days, 1 week, 2 weeks, 3 weeks, 1 month, 2 months, or 3 months. In certain embodiments, said one or more anti-PIV-antigen antibodies or antigen-binding fragments thereof and/or said one or more anti-hMPV-antigen antibodies or antigen-binding fragments thereof are administered by a nebulizer or an inhaler. In certain embodiments, said one or more anti-PIV-antigen antibodies or antigen-binding fragments thereof and/or said one or more anti-hMPV-antigen antibodies or antigen-binding fragments thereof are administered intramuscularly, intravenously or subcutaneously. In certain embodiments, the viral infection is an infection with PIV and hMPV. In certain embodiments, the viral infection is an infection with PIV and APV. In certain embodiments, at least one of said antibodies is a monoclonal antibody, a synthetic antibody, an intrabody, a chimeric antibody, a human antibody, a humanized chimeric antibody, a humanized antibody, a glycosylated antibody, a multispecific antibody, a human antibody, a single-chain antibody, or a bispecific antibody. In certain embodiments, at least one of said antibodies is a human antibody. In certain embodiments, at least one of said antibodies is a humanized antibody. In certain embodiments, at least one of said antibodies is a synthetic antibody. In certain embodiments, the subject is a mammal. In certain embodiments, the mammal is a primate. In certain embodiments, the primate is a human. In certain embodiments, the human is an elderly human. In certain embodiments, the human is a transplant recipient. In certain embodiments, the human is an immunocompromised patient. In certain embodiments, the human is an AIDS patient. In certain embodiments, the human is an infant. In certain embodiments, the human has cystic fibrosis, bronchopulmonary dysplasia, congenital heart disease, congenital immunodeficiency, or acquired immunodeficiency or has had a bone marrow transplant. In certain embodiments, the infant was born prematurely or is at risk of hospitalization for a PUV infection and/or a hMPV infection. In certain embodiments, the infant was born prematurely. In certain embodiments, the infant is less than 32 weeks of gestational age. In certain embodiments, the infant is 32 and 35 weeks of gestational age. In certain embodiments, the infant is 35 weeks of gestational age. In certain embodiments, infant is more than 38 weeks of gestational age. In certain embodiments, the mammal is not a primate. In certain embodiments, the non-primate mammal is an animal model for PIV infection and/or hMPV infection. In certain embodiments, the non-primate mammal is a cotton rat. In certain embodiments, the antibody is administered once a month just prior to and during the PIV season. In certain embodiments, the antibody is administered every two months just prior to and during the PIV season. In certain embodiments, the antibody is administered once just prior to or during the PIV season. In certain embodiments, at least one of said fragments is a Fab fragment, a F(ab′) fragment, a F(ab′)₂ fragment, a Fd, a single-chain Fv, a disulfide-linked Fv, a fragment comprising a V_{L} domain, or a fragment comprising a V_{H} domain.

In certain embodiments, the invention provides a method of preventing a viral infection in a subject, said method comprising administering to the subject: (i) a prophylactically effective amount of one or more first antibodies or antigen-binding fragments thereof, wherein said one or more first antibodies or antigen-binding fragments thereof bind immunospecifically to a RSV antigen; (ii) a prophylactically effective amount of one or more second antibodies or antigen-binding fragments thereof, wherein said one or more second antibodies or antigen-binding fragments thereof bind immunospecifically to a hMPV antigen; and (iii) a prophylactically effective amount of one or more third antibodies or antigen-binding fragments thereof, wherein said one or more third antibodies or antigen-binding fragments thereof bind immunospecifically to a PIV antigen. In certain embodiments, said one or more anti-RSV-antigen antibodies or antigen-binding fragments thereof neutralize RSV. In certain embodiments, said one or more anti-hMPV-antigen antibodies or antigen-binding fragments thereof neutralize hMPV. In certain embodiments, said one or more anti-PIV-antigen antibodies or antigen-binding fragments thereof neutralize PIV. In certain embodiments, said one or more anti-RSV-antigen antibodies or antigen-binding fragments thereof block RSV infection of cells of the subject. In certain embodiments, said one or more anti-hMPV-antigen antibodies or antigen-binding fragments thereof block hMPV infection of cells of the subject. In certain embodiments, said one or more anti-PIV-antigen antibodies or antigen-binding fragments thereof block PIV infection of cells of the subject.

In certain embodiments, the invention provides a method of treating one or more symptoms of a respiratory viral infection in a subject, said method comprising administering to the subject: (i) a therapeutically effective amount of one or more first antibodies or antigen-binding fragments thereof, wherein said one or more first antibodies or antigen-binding fragments thereof bind immunospecifically to a RSV antigen; (ii) a therapeutically effective amount of one or more second antibodies or antigen-binding fragments thereof, wherein said one or more second antibodies or antigen-binding fragments thereof bind immunospecifically to a hMPV antigen; and (iii) a therapeutically effective amount of one or more third antibodies or antigen-binding fragments thereof, wherein said one or more third antibodies or antigen-binding fragments thereof bind immunospecifically to a PIV antigen.

In certain embodiments, the invention provides a method of passive immunotherapy, said method comprising administering to a subject: (i) a first dose of one or more first antibodies or antigen-binding fragments thereof, wherein said one or more first antibodies or a fragments thereof bind immunospecifically to a RSV antigen; (ii) a second dose of one or more second antibodies or antigen-binding fragments thereof, wherein said one or more second antibodies or a fragments thereof bind immunospecifically to a hMPV antigen; and (iii) a third dose of one or more third antibodies or antigen-binding fragments thereof, wherein said one or more third antibodies or antigen-binding fragments thereof bind immunospecifically to a PIV antigen, wherein the first dose reduces the incidence of RSV infection by at least 25%, wherein the second dose reduces the incidence of hMPV infection by at least 25%, and wherein the third dose reduces the incidence of PIV infection by at least 25%. In certain embodiments, the first dose reduces the incidence of RSV infection by at least 50%, the second dose reduces the incidence of hMPV infection by at least 50%, and the third dose reduces the incidence of PIV infection by at least 50%. In certain embodiments, the first dose reduces the incidence of RSV infection by at least 75%, the second dose reduces the incidence of hMPV infection by at least 75%, and the third dose reduces the incidence of PIV infection by at least 75%. In certain embodiments, the first dose reduces the incidence of RSV infection by at least 90%, the second dose reduces the incidence of hMPV infection by at least 90%, and the third antibody reduces the incidence of PIV infection by at least 90%.

In certain embodiments, the invention provides a method of passive immunotherapy, said method comprising administering to a subject: (i) a first dose of one or more first antibodies or antigen-binding fragments thereof, wherein said one or more first antibodies or antigen-binding fragments thereof bind immunospecifically to a RSV antigen; (ii) a second dose of one or more second antibodies or antigen-binding fragments thereof, wherein said one or more second antibodies or antigen-binding fragments thereof bind immunospecifically to a hMPV antigen; and (iii) a third dose of one or more third antibodies or antigen-binding fragments thereof, wherein said one or more third antibodies or antigen-binding fragments thereof bind immunospecifically to a PIV antigen, wherein the serum titer of said one or more anti-RSV-antigen antibodies or antigen-binding fragments thereof in the subject is at least 10 μg/ml after 15 days of administering said one or more anti-RSV-antigen antibodies or antigen-binding fragments thereof, wherein the serum titer of said one or more anti-hMPV-antigen antibodies or antigen-binding fragments thereof in the subject is at least 10 μg/ml after 15 days of administering said one or more anti-hMPV-antigen antibodies or antigen-binding fragments thereof, and wherein the serum titer of said one or more anti-PIV-antigen antibodies or antigen-binding fragments thereof in the subject is at least 10 μg/ml after 15 days of administering said one or more anti-PIV-antigen antibodies or antigen-binding fragments thereof. In certain embodiments, the amino acid sequence of the PIV antigen is that of SEQ ID NO:407 to 419, respectively. In certain embodiments, the amino acid sequence of the PIV antigen is 90% identical to the amino acid sequence of PIV nucleoprotein, PIV phosphoprotein, PIV matrix protein, PIV small hydrophobic protein, PIV RNA-dependent RNA polymerase, PIV F protein, or PIV G protein. In certain embodiments, the PIV antigen is selected from the group consisting of PIV nucleoprotein, PIV phosphoprotein, PIV matrix protein, PIV small hydrophobic protein, PIV RNA-dependent RNA polymerase, PIV F protein, and PIV G protein.

In certain embodiments, the invention provides a method of preventing a viral infection in a subject, said method comprising administering to the subject: (i) a prophylactically effective amount of one or more first antibodies or antigen-binding fragments thereof, wherein said one or more first antibodies or antigen-binding fragments thereof bind immunospecifically to a RSV antigen; and (ii) a prophylactically effective amount of one or more second antibodies or antigen-binding fragments thereof, wherein said one or more second antibodies or antigen-binding fragments thereof bind immunospecifically to a PIV antigen. In certain embodiments, said one or more anti-RSV-antigen antibodies or antigen-binding fragments thereof neutralize RSV. In certain embodiments, said one or more anti-PIV-antigen antibodies or antigen-binding fragments thereof neutralize PIV. In certain embodiments, said one or more anti-RSV-antigen antibodies or antigen-binding fragments thereof block RSV infection of cells of the subject. In certain embodiments, said one or more anti-PIV-antigen antibodies or antigen-binding fragments thereof block PIV infection of cells of the subject.

In certain embodiments, the invention provides a method of treating one or more symptoms of a respiratory viral infection in a subject, said method comprising administering to the subject: (i) a therapeutically effective amount of one or more first antibodies or antigen-binding fragments thereof, wherein said one or more first antibodies or antigen-binding fragments thereof bind immunospecifically to a RSV antigen; and (ii) a therapeutically effective amount of one or more second antibodies or antigen-binding fragments thereof, wherein said one or more second antibodies or antigen-binding fragments thereof bind immunospecifically to a PIV antigen.

In certain embodiments, the invention provides a method of passive immunotherapy, said method comprising administering to a subject: (i) a first dose of one or more first antibodies or antigen-binding fragments thereof, wherein said one or more first antibodies or a fragments thereof bind immunospecifically to a RSV antigen; and (ii) a second dose of one or more second antibodies or antigen-binding fragments thereof, wherein said one or more second antibodies or a fragments thereof bind immunospecifically to a PIV antigen, wherein the first dose reduces the incidence of RSV infection by at least 25% and wherein the second dose reduces the incidence of PIV infection by at least 25%. In certain embodiments, the first dose reduces the incidence of RSV infection by at least 50% and wherein the second dose reduces the incidence of hMPV infection by at least 50%. In certain embodiments, the first dose reduces the incidence of RSV infection by at least 75% and wherein the second dose reduces the incidence of hMPV infection by at least 75%. In certain embodiments, the first dose reduces the incidence of RSV infection by at least 90% and wherein the second dose reduces the incidence of hMPV infection by at least 90%.

In certain embodiments, the invention provides a method of passive immunotherapy, said method comprising administering to a subject: (i) a first dose of one or more first antibodies or antigen-binding fragments thereof, wherein said one or more first antibodies or antigen-binding fragments thereof bind immunospecifically to a RSV antigen; and (ii) a second dose of one or more second antibodies or antigen-binding fragments thereof, wherein said one or more second antibodies or antigen-binding fragments thereof bind immunospecifically to a PIV antigen, wherein the serum titer of said one or more anti-RSV-antigen antibodies or antigen-binding fragments thereof in the subject is at least 10 μg/ml after 15 days of administering said one or more anti-RSV-antigen antibodies or antigen-binding fragments thereof and wherein the serum titer of said one or more anti-PIV-antigen antibodies or antigen-binding fragments thereof in the subject is at least 10 μg/ml after 15 days of administering said one or more anti-PIV-antigen antibodies or antigen-binding fragments thereof.

3.1 Preferred Embodiments

In certain embodiments, the invention provides a method of preventing a viral infection in a subject, said method comprising administering to the subject: (i) a prophylactically effective amount of one or more first antibodies or antigen-binding fragments thereof, wherein one or more of said first antibodies or antigen-binding fragments thereof bind immunospecifically to a RSV antigen; and (ii) a prophylactically effective amount of one or more second antibodies or antigen-binding fragments thereof, wherein one or more of said second antibodies or antigen-binding fragments thereof bind immunospecifically to a hMPV antigen.

In certain embodiments, the invention provides a method wherein one or more of said first antibodies or antigen-binding fragments thereof neutralize RSV.

In certain embodiments, the invention provides a method wherein one or more of said second antibodies or antigen-binding fragments thereof neutralize hMPV.

In certain embodiments, the invention provides a method wherein one or more of said first antibodies or antigen-binding fragments thereof block RSV infection of cells of the subject.

In certain embodiments, the invention provides a method wherein one or more of said second antibodies or antigen-binding fragments thereof block hMPV infection of cells of the subject.

In certain embodiments, the invention provides a method of treating one or more symptoms of a respiratory viral infection in a subject, said method comprising administering to the subject: (i) a therapeutically effective amount of one or more first antibodies or antigen-binding fragments thereof, wherein one or more of said first antibodies or antigen-binding fragments thereof bind immunospecifically to a RSV antigen; and (ii) a therapeutically effective amount of one or more second antibodies or antigen-binding fragments thereof, wherein one or more of said second antibodies or antigen-binding fragments thereof bind immunospecifically to a hMPV antigen.

In certain embodiments, the invention provides a method of passive immunotherapy, said method comprising administering to a subject: (i) a first dose of one or more first antibodies or antigen-binding fragments thereof, wherein one or more of said first antibodies or a fragments thereof bind immunospecifically to a RSV antigen; and (ii) a second dose of one or more second antibodies or antigen-binding fragments thereof, wherein one or more of said second antibodies or a fragments thereof bind immunospecifically to a hMPV antigen, wherein the first dose reduces the incidence of RSV infection by at least 25% and wherein the second dose reduces the incidence of hMPV infection by at least 25%.

In certain embodiments, the invention provides a method wherein the first dose reduces the incidence of RSV infection by at least 50% and wherein the second dose reduces the incidence of hMPV infection by at least 50%.

In certain embodiments, the invention provides a method wherein the first dose reduces the incidence of RSV infection by at least 75% and wherein the second dose reduces the incidence of hMPV infection by at least 75%.

In certain embodiments, the invention provides a method wherein the first dose reduces the incidence of RSV infection by at least 90% and wherein the second dose reduces the incidence of hMPV infection by at least 90%.

In certain embodiments, the invention provides a method of passive immunotherapy, said method comprising administering to a subject: (i) a first dose of one or more first antibodies or antigen-binding fragments thereof, wherein one or more of said first antibodies or antigen-binding fragments thereof bind immunospecifically to a RSV antigen; and (ii) a second dose of one or more second antibodies or antigen-binding fragments thereof, wherein one or more of said second antibodies or antigen-binding fragments thereof bind immunospecifically to a hMPV antigen, wherein the serum titer of one or more of said first antibodies or antigen-binding fragments thereof in the subject is at least 10 μg/ml after 15 days of administering one or more of said first antibodies or antigen-binding fragments thereof and wherein the serum titer of one or more of said second antibodies or antigen-binding fragments thereof in the subject is at least 10 μg/ml after 15 days of administering one or more of said second antibodies or antigen-binding fragments thereof.

In certain embodiments, the invention provides a method wherein the amino acid sequence of the RSV antigen is that of SEQ ID NO:390 to 398, respectively.

In certain embodiments, the invention provides a method wherein the amino acid sequence of the RSV antigen is 90% identical to the amino acid sequence of RSV nucleoprotein, RSV phosphoprotein, RSV matrix protein, RSV small hydrophobic protein, RSV RNA-dependent RNA polymerase, RSV F protein, or RSV G protein.

In certain embodiments, the invention provides a method wherein the RSV antigen is selected from the group consisting of RSV nucleoprotein, RSV phosphoprotein, RSV matrix protein, RSV small hydrophobic protein, RSV RNA-dependent RNA polymerase, RSV F protein, and RSV G protein.

In certain embodiments, the invention provides a method wherein one or more of said first antibodies immunospecifically bind to an antigen of Group A or Group B RSV.

In certain embodiments, the invention provides a method wherein the RSV antigen is RSV F protein.

In certain embodiments, the invention provides a method wherein one or more of said second antibodies cross-react with a turkey APV antigen.

In certain embodiments, the invention provides a method wherein one or more of said second antibodies are (i) human or humanized antibodies and (ii) cross-react with a turkey APV antigen.

In certain embodiments, the invention provides a method wherein said turkey APV antigen is selected from the group consisting of turkey APV nucleoprotein, turkey APV phosphoprotein, turkey APV matrix protein, turkey APV small hydrophobic protein, turkey APV RNA-dependent RNA polymerase, turkey APV F protein, and turkey APV G protein.

In certain embodiments, the invention provides a method wherein said turkey APV antigen is an antigen of avian pneumovirus type A, avian pneumovirus type B, or avian pneumovirus type C.

In certain embodiments, the invention provides a method wherein the amino acid sequence of said turkey APV antigen is that of SEQ ID NO:424 to 429, respectively.

In certain embodiments, the invention provides a method wherein the amino acid sequence of the hMPV antigen is that of SEQ ID NO: 399-406, 420, or 421, respectively.

In certain embodiments, the invention provides a method wherein the hMPV antigen is selected from the group consisting of hMPV nucleoprotein, hMPV phosphoprotein, hMPV matrix protein, hMPV small hydrophobic protein, hMPV RNA-dependent RNA polymerase, hMPV F protein, and hMPV G protein.

In certain embodiments, the invention provides a method wherein the hMPV antigen is hMPV F protein.

In certain embodiments, the invention provides a method wherein the first antibody is Palivizumab; AFFF; P12f2 P12f4; P11d4; Ale9; A12a6; A13c4; A17d4; A4B4; 1X-493L1; FR H3-3F4; M3H9; Y10H6; DG; AFFF(1); 6H8; L1-7E5; L2-15B10; A13a11; A1h5; A4B4(1);A4B4-F52S; or A4B4L1FR-S28R.

In certain embodiments, the invention provides a method wherein the effective amount of one or more of said first antibodies is 15 mg/kg or less.

In certain embodiments, the invention provides a method wherein the effective amount of one or more of said first antibodies is 10 mg/kg or less.

In certain embodiments, the invention provides a method wherein the effective amount of one or more of said first antibodies is 1 mg/kg or less.

In certain embodiments, the invention provides a method wherein the effective amount of one or more of said second antibodies or antigen-binding fragments thereof is 15 mg/kg or less.

In certain embodiments, the invention provides a method wherein the effective amount of one or more of said second antibodies or antigen-binding fragments thereof is 10 mg/kg or less.

In certain embodiments, the invention provides a method wherein the effective amount of one or more of said second antibodies or antigen-binding fragments thereof is 1 mg/kg or less.

In certain embodiments, the invention provides a method wherein one or more of said first antibodies or antigen-binding fragments thereof are administered at a time period prior to administering of one or more of said second antibodies or antigen-binding fragments thereof.

In certain embodiments, the invention provides a method wherein one or more of said second antibodies or antigen-binding fragments thereof are administered at a time period prior to administering of one or more of said first antibodies or antigen-binding fragments thereof.

In certain embodiments, the invention provides a method wherein one or more of said first antibodies or antigen-binding fragments thereof and one or more of said second antibodies or antigen-binding fragments thereof are administered concurrently.

In certain embodiments, the invention provides a method wherein one or more of said first antibodies or antigen-binding fragments thereof are administered in a sequence of two or more administrations, wherein the administrations of one or more of said first antibodies or antigen-binding fragments thereof are separated by a time period from each other, and wherein one or more of said second antibodies or antigen-binding fragments thereof are administered before, during, or after the sequence.

In certain embodiments, the invention provides a method wherein one or more of said first antibodies or antigen-binding fragments thereof are administered in a sequence of two or more administrations, wherein the administrations of one or more of said second antibodies or antigen-binding fragments thereof are separated by a time period from each other, and wherein one or more of said first antibodies or antigen-binding fragments thereof are administered before, during, or after the sequence.

In certain embodiments, the invention provides a method wherein one or more of said first antibodies or antigen-binding fragments thereof and one or more of said second antibodies or antigen-binding fragments thereof are administered in a sequence of two or more administrations, wherein the administrations are separated by a time period from each other.

In certain embodiments, the invention provides a method wherein the time period is at least 1 day, 2 days, 3 days, 4 days, 5 days, 6 days, 1 week, 2 weeks, 3 weeks, 1 month, 2 months, or 3 months.

In certain embodiments, the invention provides a method wherein the time period is at least 1 day, 2 days, 3 days, 4 days, 5 days, 6 days, 1 week, 2 weeks, 3 weeks, 1 month, 2 months, or 3 months.

In certain embodiments, the invention provides a method wherein the time period is at least 1 day, 2 days, 3 days, 4 days, 5 days, 6 days, 1 week, 2 weeks, 3 weeks, 1 month, 2 months, or 3 months.

In certain embodiments, the invention provides a method wherein the time period is at least 1 day, 2 days, 3 days, 4 days, 5 days, 6 days, 1 week, 2 weeks, 3 weeks, 1 month, 2 months, or 3 months.

In certain embodiments, the invention provides a method wherein the time period is at least 1 day, 2 days, 3 days, 4 days, 5 days, 6 days, 1 week, 2 weeks, 3 weeks, 1 month, 2 months, or 3 months.

In certain embodiments, the invention provides a method wherein the time period is at least 1 day, 2 days, 3 days, 4 days, 5 days, 6 days, 1 week, 2 weeks, 3 weeks, 1 month, 2 months, or 3 months.

In certain embodiments, the invention provides a method wherein one or more of said first antibodies or antigen-binding fragments thereof and/or one or more of said second antibodies or antigen-binding fragments thereof are administered by a nebulizer or an inhaler.

In certain embodiments, the invention provides a method wherein one or more of said first antibodies or antigen-binding fragments thereof and/or one or more of said second antibodies or antigen-binding fragments thereof are administered intramuscularly, intravenously or subcutaneously.

In certain embodiments, the invention provides a method wherein the viral infection is an infection with RSV and hMPV.

In certain embodiments, the invention provides a method wherein the viral infection is an infection with RSV and APV.

In certain embodiments, the invention provides a method wherein at least one of said antibodies is a monoclonal antibody, a synthetic antibody, an intrabody, a chimeric antibody, a human antibody, a humanized chimeric antibody, a humanized antibody, a glycosylated antibody, a multispecific antibody, a human antibody, a single-chain antibody, or a bispecific antibody.

In certain embodiments, the invention provides a method wherein at least one of said antibodies is a human antibody.

In certain embodiments, the invention provides a method wherein at least one of said antibodies is a humanized antibody.

In certain embodiments, the invention provides a method wherein at least one of said antibodies is a synthetic antibody.

In certain embodiments, the invention provides a method wherein the subject is a mammal.

In certain embodiments, the invention provides a method wherein the mammal is a primate.

In certain embodiments, the invention provides a method wherein the primate is a human.

In certain embodiments, the invention provides a method wherein the human is an elderly human.

In certain embodiments, the invention provides a method wherein the human is a transplant recipient.

In certain embodiments, the invention provides a method wherein the human is an immunocompromised patient.

In certain embodiments, the invention provides a method wherein the human is an AIDS patient.

In certain embodiments, the invention provides a method wherein the human is an infant.

In certain embodiments, the invention provides a method wherein the human has cystic fibrosis, bronchopulmonary dysplasia, congenital heart disease, congenital immunodeficiency, or acquired immunodeficiency or has had a bone marrow transplant.

In certain embodiments, the invention provides a method wherein the infant was born prematurely or is at risk of hospitalization for a RSV infection and/or for a hMPV infection.

In certain embodiments, the invention provides a method wherein the human infant was born prematurely.

In certain embodiments, the invention provides a method wherein the infant was born at 32 weeks of gestational age.

In certain embodiments, the invention provides a method wherein the infant was born at between 32 and 35 weeks of gestational age.

In certain embodiments, the invention provides a method wherein the infant was born at more than 35 weeks of gestational age.

In certain embodiments, the invention provides a method wherein the infant is more than 38 weeks of gestational age.

In certain embodiments, the invention provides a method wherein the mammal is not a primate.

In certain embodiments, the invention provides a method wherein the non-primate mammal is an animal model for RSV infection and/or hMPV infection.

In certain embodiments, the invention provides a method wherein the non-primate mammal is a cotton rat.

In certain embodiments, the invention provides a method wherein the antibody is administered once a month just prior to and during the RSV season.

In certain embodiments, the invention provides a method wherein the antibody is administered every two months just prior to and during the RSV season.

In certain embodiments, the invention provides a method wherein the antibody is administered once just prior to or during the RSV season.

In certain embodiments, the invention provides a method wherein at least one of said fragments is a Fab fragment, a F(ab′) fragment, a F(ab′)₂ fragment, a Fd, a single-chain Fv, a disulfide-linked Fv, a fragment comprising a V_{L} domain, or a fragment comprising a V_{H} domain.

In certain embodiments, the invention provides a method of preventing a viral infection in a subject, said method comprising administering to the subject: (i) a dose of one or more antibodies or antigen-binding fragments thereof, wherein one or more of said antibodies or antigen-binding fragments thereof (i) are human or humanized, (ii) cross-react with a turkey APV antigen, and (iii) bind immunospecifically to a hMPV antigen.

In certain embodiments, the invention provides a method of treating one or more symptoms of a respiratory viral infection in a subject, said method comprising administering to the subject: (i) a dose of one or more antibodies or antigen-binding fragments thereof, wherein one or more of said antibodies or antigen-binding fragments thereof (i) are human or humanized, (ii) cross-react with a turkey APV antigen, and (iii) bind immunospecifically to a hMPV antigen.

In certain embodiments, the invention provides a method of passive immunotherapy, said method comprising administering to a subject: (i) a dose of one or more antibodies or antigen-binding fragments thereof, wherein one or more of said antibodies or antigen-binding fragments thereof (i) are human or humanized, (ii) cross-react with a turkey APV antigen, and (iii) bind immunospecifically to a hMPV antigen, wherein the dose reduces the incidence of hMPV infection by at least 25%.

In certain embodiments, the invention provides a method wherein the dose reduces the incidence of hMPV infection by at least 50%.

In certain embodiments, the invention provides a method wherein the dose reduces the incidence of hMPV infection by at least 75%.

In certain embodiments, the invention provides a method wherein the dose reduces the incidence of hMPV infection by at least 90%.

In certain embodiments, the invention provides a method of passive immunotherapy, said method comprising administering to a subject: (i) a dose of one or more antibodies or antigen-binding fragments thereof, wherein one or more of said antibodies or antigen-binding fragments thereof (i) are human or humanized, (ii) cross-react with a turkey APV antigen, and (iii) bind immunospecifically to a hMPV antigen, wherein the serum titer of one or more of said antibodies or antigen-binding fragments thereof in the subject is at least 10 μg/ml after 15 days of administering one or more of said antibodies or antigen-binding fragments thereof.

In certain embodiments, the invention provides a pharmaceutical composition, said composition comprising: (i) one or more first antibodies or antigen-binding fragments thereof, wherein one or more of said first antibodies or antigen-binding fragments thereof bind immunospecifically to a RSV antigen; and (ii) one or more second antibodies or antigen-binding fragments thereof, wherein one or more of said second antibodies or antigen-binding fragments thereof bind immunospecifically to a hMPV antigen.

In certain embodiments, the invention provides a pharmaceutical composition wherein the amino acid sequence of the RSV antigen is that of SEQ ID NO:390 to 398, respectively.

In certain embodiments, the invention provides a pharmaceutical composition wherein the amino acid sequence of the RSV antigen is 90% identical to the amino acid sequence of RSV nucleoprotein, RSV phosphoprotein, RSV matrix protein, RSV small hydrophobic protein, RSV RNA-dependent RNA polymerase, RSV F protein, or RSV G protein.

In certain embodiments, the invention provides a pharmaceutical composition wherein the RSV antigen is selected from the group consisting of RSV nucleoprotein, RSV phosphoprotein, RSV matrix protein, RSV small hydrophobic protein, RSV RNA-dependent RNA polymerase, RSV F protein, and RSV G protein.

In certain embodiments, the invention provides a pharmaceutical composition wherein one or more of said first antibodies or antigen-binding fragments thereof immunospecifically bind to an antigen of Group A or Group B RSV.

In certain embodiments, the invention provides a pharmaceutical composition wherein the RSV antigen is RSV F protein.

In certain embodiments, the invention provides a pharmaceutical composition wherein one or more of said second antibodies cross-react with a turkey APV antigen.

In certain embodiments, the invention provides a pharmaceutical composition wherein one or more of said second antibodies are (i) human or humanized antibodies and (ii) cross-react with a turkey APV antigen.

In certain embodiments, the invention provides a pharmaceutical composition wherein said turkey APV antigen is selected from the group consisting of turkey APV nucleoprotein, turkey APV phosphoprotein, turkey APV matrix protein, turkey APV small hydrophobic protein, turkey APV RNA-dependent RNA polymerase, turkey APV F protein, and turkey APV G. protein.

In certain embodiments, the invention provides a pharmaceutical composition wherein said turkey APV antigen is an antigen of avian pneumovirus type A, avian pneumovirus type B, or avian pneumovirus type C.

In certain embodiments, the invention provides In certain embodiments, the invention provides a pharmaceutical composition wherein the amino acid sequence of said turkey APV antigen is that of SEQ ID NO:424 to 429, respectively.

In certain embodiments, the invention provides a pharmaceutical composition wherein the amino acid sequence of the hMPV antigen is that of SEQ ID NO: 399-406, 420, or 421, respectively.

In certain embodiments, the invention provides a pharmaceutical composition wherein the hMPV antigen is selected from the group consisting of hMPV nucleoprotein, hMPV phosphoprotein, hMPV matrix protein, hMPV small hydrophobic protein, hMPV RNA-dependent RNA polymerase, hMPV F protein, and hMPV G protein.

In certain embodiments, the invention provides a pharmaceutical composition wherein the hMPV antigen is hMPV F protein.

In certain embodiments, the invention provides a pharmaceutical composition wherein the first antibody is Palivizumab; AFFF; P12f2 P12f4; P11d4; Ale9; A12a6; A13c4; A17d4; A4B4; 1X-493 μl; FR H3-3F4; M3H9; Y10H6; DG; AFFF(1); 6H8; L1-7E5; L2-15B10; A13a11; A1h5; A4B4(1);A4B4-F52S; or A4B4L1FR-S28R.

In certain embodiments, the invention provides a pharmaceutical composition wherein at least one of said antibodies is a monoclonal antibody, a synthetic antibody, an intrabody, a chimeric antibody, a human antibody, a humanized chimeric antibody, a humanized antibody, a glycosylated antibody, a multispecific antibody, a human antibody, a single-chain antibody, or a bispecific antibody.

In certain embodiments, the invention provides a pharmaceutical composition wherein at least one of said antibodies is a human antibody.

In certain embodiments, the invention provides a pharmaceutical composition wherein at least one of said antibodies is a humanized antibody.

In certain embodiments, the invention provides a pharmaceutical composition wherein at least one of said antibodies is a synthetic antibody.

In certain embodiments, the invention provides a pharmaceutical composition wherein at least one of said fragments is a Fab fragment, a F(ab′) fragment, a F(ab′)₂ fragment, a Fd, a single-chain Fv, a disulfide-linked Fv, a fragment comprising a V_{L} domain, or a fragment comprising a V_{H} domain.

In certain embodiments, the invention provides a pharmaceutical composition, said composition comprising: one or more antibodies or antigen-binding fragments thereof, wherein one or more of said antibodies or antigen-binding fragments thereof (i) are human or humanized, (ii) cross-react with a turkey APV antigen, and (iii) bind immunospecifically to a hMPV antigen.

In certain embodiments, the invention provides a method of preventing a viral infection in a subject, said method comprising administering to the subject: (i) a prophylactically effective amount of one or more first antibodies or antigen-binding fragments thereof, wherein one or more of said first antibodies or antigen-binding fragments thereof bind immunospecifically to a PIV antigen; and (ii) a prophylactically effective amount of one or more second antibodies or antigen-binding fragments thereof, wherein one or more of said second antibodies or antigen-binding fragments thereof bind immunospecifically to a hMPV antigen.

In certain embodiments, the invention provides a method wherein one or more of said first antibodies or antigen-binding fragments thereof neutralize PIV.

In certain embodiments, the invention provides a method wherein one or more of said second antibodies or antigen-binding fragments thereof neutralize hMPV.

In certain embodiments, the invention provides a method wherein one or more of said first antibodies or antigen-binding fragments thereof block PIV infection of cells of the subject.

In certain embodiments, the invention provides a method wherein one or more of said second antibodies or antigen-binding fragments thereof block hMPV infection of cells of the subject.

In certain embodiments, the invention provides a method of treating one or more symptoms of a respiratory viral infection in a subject, said method comprising administering to the subject: (i) a therapeutically effective amount of one or more first antibodies or antigen-binding fragments thereof, wherein one or more of said first antibodies or antigen-binding fragments thereof bind immunospecifically to a PIV antigen; and (ii) a therapeutically effective amount of one or more second antibodies or antigen-binding fragments thereof, wherein one or more of said second antibodies or antigen-binding fragments thereof bind immunospecifically to a hMPV antigen.

In certain embodiments, the invention provides a method of passive immunotherapy, said method comprising administering to a subject: (i) a first dose of one or more first antibodies or antigen-binding fragments thereof, wherein one or more of said first antibodies or a fragments thereof bind immunospecifically to a PIV antigen; and (ii) a second dose of one or more second antibodies or antigen-binding fragments thereof, wherein one or more of said second antibodies or a fragments thereof bind immunospecifically to a hMPV antigen, wherein the first dose reduces the incidence of PIV infection by at least 25% and wherein the second dose reduces the incidence of hMPV infection by at least 25%.

In certain embodiments, the invention provides a method wherein the first dose reduces the incidence of PIV infection by at least 50% and wherein the second dose reduces the incidence of hMPV infection by at least 50%.

In certain embodiments, the invention provides a method wherein the first dose reduces the incidence of PIV infection by at least 75% and wherein the second dose reduces the incidence of hMPV infection by at least 75%.

In certain embodiments, the invention provides a method wherein the first dose reduces the incidence of PIV infection by at least 90% and wherein the second dose reduces the incidence of hMPV infection by at least 90%.

In certain embodiments, the invention provides a method of passive immunotherapy, said method comprising administering to a subject: (i) a first dose of one or more first antibodies or antigen-binding fragments thereof, wherein one or more of said first antibodies or antigen-binding fragments thereof bind immunospecifically to a PIV antigen; and (ii) a second dose of one or more second antibodies or antigen-binding fragments thereof, wherein one or more of said second antibodies or antigen-binding fragments thereof bind immunospecifically to a hMPV antigen, wherein the serum titer of one or more of said first antibodies or antigen-binding fragments thereof in the subject is at least 10 μg/ml after 15 days of administering one or more of said first antibodies or antigen-binding fragments thereof and wherein the serum titer of one or more of said second antibodies or antigen-binding fragments thereof in the subject is at least 10 μg/ml after 15 days of administering one or more of said second antibodies or antigen-binding fragments thereof.

In certain embodiments, the invention provides a method wherein the amino acid sequence of the PIV antigen is that of SEQ ID NO:407 to 419, respectively.

In certain embodiments, the invention provides a method wherein the amino acid sequence of the PIV antigen is 90% identical to the amino acid sequence of PIV nucleocapsid phosphoprotein, PIV L protein, PIV matrix protein, PIV HN glycoprotein, PIV RNA-dependent RNA polymerase, PIV Y1 protein, PIV D protein, or PIV C protein.

In certain embodiments, the invention provides a method wherein the PIV antigen is selected from the group consisting of PIV nucleocapsid phosphoprotein, PIV L protein, PIV matrix protein, PIV HN glycoprotein, PUV RNA-dependent RNA polymerase, PIV Y1 protein, PIV D protein, or PIV C protein.

In certain embodiments, the invention provides a method wherein one or more of said first antibodies immunospecifically bind to an antigen of human PIV type 1, human PIV type 2, human PIV type 3, or human PIV type 4.

In certain embodiments, the invention provides a method wherein the PIV antigen is PIV F protein.

In certain embodiments, the invention provides a method wherein one or more of said second antibodies cross-react with a turkey APV antigen.

In certain embodiments, the invention provides a method wherein one or more of said second antibodies are (i) human or humanized antibodies and (ii) cross-react with a turkey APV antigen.

In certain embodiments, the invention provides a method wherein said turkey APV antigen is selected from the group consisting of turkey APV nucleoprotein, turkey APV phosphoprotein, turkey APV matrix protein, turkey APV small hydrophobic protein, turkey APV RNA-dependent RNA polymerase, turkey APV F protein, and turkey APV G protein.

In certain embodiments, the invention provides a method wherein said turkey APV antigen is an antigen of avian pneumovirus type A, avian pneumovirus type B, or avian pneumovirus type C.

In certain embodiments, the invention provides a method wherein the amino acid sequence of said turkey APV antigen is that of SEQ ID NO:424 to 429, respectively.

In certain embodiments, the invention provides a method wherein the amino acid sequence of the hMPV antigen is that of SEQ ID NO: 399-406, 420, or 421, respectively.

In certain embodiments, the invention provides a method wherein the hMPV antigen is selected from the group consisting of hMPV nucleoprotein, hMPV phosphoprotein, hMPV matrix protein, hMPV small hydrophobic protein, hMPV RNA-dependent RNA polymerase, hMPV F protein, and hMPV G protein.

In certain embodiments, the invention provides a method wherein the hMPV antigen is hMPV F protein.

In certain embodiments, the invention provides a method wherein the effective amount of one or more of said first antibodies is 100 mg/kg or less.

In certain embodiments, the invention provides a method wherein the effective amount of one or more of said first antibodies is 10 mg/kg or less.

In certain embodiments, the invention provides a method wherein the effective amount of one or more of said first antibodies is 1 mg/kg or less.

In certain embodiments, the invention provides a method wherein the effective amount of one or more of said second antibodies or antigen-binding fragments thereof is 100 mg/kg or less.

In certain embodiments, the invention provides a method wherein the effective amount of one or more of said second antibodies or antigen-binding fragments thereof is 10 mg/kg or less.

In certain embodiments, the invention provides a method wherein the effective amount of one or more of said second antibodies or antigen-binding fragments thereof is 1 mg/kg or less.

In certain embodiments, the invention provides a method wherein one or more of said first antibodies or antigen-binding fragments thereof are administered at a time period prior to administering of one or more of said second antibodies or antigen-binding fragments thereof.

In certain embodiments, the invention provides a method wherein one or more of said second antibodies or antigen-binding fragments thereof are administered at a time period prior to administering of one or more of said first antibodies or antigen-binding fragments thereof.

In certain embodiments, the invention provides a method wherein one or more of said first antibodies or antigen-binding fragments thereof and one or more of said second antibodies or antigen-binding fragments thereof are administered concurrently.

In certain embodiments, the invention provides a method wherein one or more of said first antibodies or antigen-binding fragments thereof are administered in a sequence of two or more administrations, wherein the administrations of one or more of said first antibodies or antigen-binding fragments thereof are separated by a time period from each other, and wherein one or more of said second antibodies or antigen-binding fragments thereof are administered before, during, or after the sequence.

In certain embodiments, the invention provides a method wherein one or more of said first antibodies or antigen-binding fragments thereof are administered in a sequence of two or more administrations, wherein the administrations of one or more of said second antibodies or antigen-binding fragments thereof are separated by a time period from each other, and wherein one or more of said first antibodies or antigen-binding fragments thereof are administered before, during, or after the sequence.

In certain embodiments, the invention provides a method wherein one or more of said first antibodies or antigen-binding fragments thereof and one or more of said second antibodies or antigen-binding fragments thereof are administered in a sequence of two or more administrations, wherein the administrations are separated by a time period from each other.

In certain embodiments, the invention provides a method wherein the time period is at least 1 day, 2 days, 3 days, 4 days, 5 days, 6 days, 1 week, 2 weeks, 3 weeks, 1 month, 2 months, or 3 months.

In certain embodiments, the invention provides a method wherein the time period is at least 1 day, 2 days, 3 days, 4 days, 5 days, 6 days, 1 week, 2 weeks, 3 weeks, 1 month, 2 months, or 3 months.

In certain embodiments, the invention provides a method wherein the time period is at least 1 day, 2 days, 3 days, 4 days, 5 days, 6 days, 1 week, 2 weeks, 3 weeks, 1 month, 2 months, or 3 months.

In certain embodiments, the invention provides a method wherein the time period is at least 1 day, 2 days, 3 days, 4 days, 5 days, 6 days, 1 week, 2 weeks, 3 weeks, 1 month, 2 months, or 3 months.

In certain embodiments, the invention provides a method wherein the time period is at least 1 day, 2 days, 3 days, 4 days, 5 days, 6 days, 1 week, 2 weeks, 3 weeks, 1 month, 2 months, or 3 months.

In certain embodiments, the invention provides a method wherein one or more of said first antibodies or antigen-binding fragments thereof and/or one or more of said second antibodies or antigen-binding fragments thereof are administered by a nebulizer or an inhaler.

In certain embodiments, the invention provides a method wherein one or more of said first antibodies or antigen-binding fragments thereof and/or one or more of said second antibodies or antigen-binding fragments thereof are administered intramuscularly, intravenously or subcutaneously.

In certain embodiments, the invention provides a method wherein the viral infection is an infection with PIV and hMPV.

In certain embodiments, the invention provides a method wherein the viral infection is an infection with PIV and APV.

In certain embodiments, the invention provides a method wherein at least one of said antibodies is a monoclonal antibody, a synthetic antibody, an intrabody, a chimeric antibody, a human antibody, a humanized chimeric antibody, a humanized antibody, a glycosylated antibody, a multispecific antibody, a human antibody, a single-chain antibody, or a bispecific antibody.

In certain embodiments, the invention provides a method wherein at least one of said antibodies is a human antibody.

In certain embodiments, the invention provides a method wherein at least one of said antibodies is a humanized antibody.

In certain embodiments, the invention provides a method wherein at least one of said antibodies is a synthetic antibody.

In certain embodiments, the invention provides a method wherein the subject is a mammal.

In certain embodiments, the invention provides a method wherein the mammal is a primate.

In certain embodiments, the invention provides a method wherein the primate is a human.

In certain embodiments, the invention provides a method wherein the human is an elderly human.

In certain embodiments, the invention provides a method wherein the human is a transplant recipient.

In certain embodiments, the invention provides a method wherein the human is an immunocompromised patient.

In certain embodiments, the invention provides a method wherein the human is an AIDS patient.

In certain embodiments, the invention provides a method wherein the human is an infant.

In certain embodiments, the invention provides a method wherein the human has cystic fibrosis, bronchopulmonary dysplasia, congenital heart disease, congenital immunodeficiency, or acquired immunodeficiency or has had a bone marrow transplant.

In certain embodiments, the invention provides a method wherein the infant was born prematurely or is at risk of hospitalization for a PIV infection and/or a hMPV infection.

In certain embodiments, the invention provides a method wherein the infant was born prematurely.

In certain embodiments, the invention provides a method wherein the infant was born at less than 32 weeks of gestational age.

In certain embodiments, the invention provides a method wherein the infant was born at 32 and 35 weeks of gestational age.

In certain embodiments, the invention provides a method wherein the infant was born at 35 weeks of gestational age.

In certain embodiments, the invention provides a method wherein the infant is more than 38 weeks of gestational age.

In certain embodiments, the invention provides a method wherein the mammal is not a primate.

In certain embodiments, the invention provides a method wherein the non-primate mammal is an animal model for PIV infection and/or hMPV infection.

In certain embodiments, the invention provides a method wherein the non-primate mammal is a cotton rat.

In certain embodiments, the invention provides a method wherein the antibody is administered once a month just prior to and during the PIV season.

In certain embodiments, the invention provides a method wherein the antibody is administered every two months just prior to and during the PIV season.

In certain embodiments, the invention provides a method wherein the antibody is administered once just prior to or during the PIV season.

In certain embodiments, the invention provides a method wherein at least one of said fragments is a Fab fragment, a F(ab′) fragment, a F(ab′)₂ fragment, a Fd, a single-chain Fv, a disulfide-linked Fv, a fragment comprising a VL domain, or a fragment comprising a VH domain.

In certain embodiments, the invention provides a method of preventing a viral infection in a subject, said method comprising administering to the subject: (i) a prophylactically effective amount of one or more first antibodies or antigen-binding fragments thereof, wherein one or more of said first antibodies or antigen-binding fragments thereof bind immunospecifically to a RSV antigen; (ii) a prophylactically effective amount of one or more second antibodies or antigen-binding fragments thereof, wherein one or more of said second antibodies or antigen-binding fragments thereof bind immunospecifically to a hMPV antigen; and (iii) a prophylactically effective amount of one or more third antibodies or antigen-binding fragments thereof, wherein one or more of said third antibodies or antigen-binding fragments thereof bind immunospecifically to a PUV antigen.

In certain embodiments, the invention provides a method wherein one or more of said first antibodies or antigen-binding fragments thereof neutralize RSV.

In certain embodiments, the invention provides a method wherein one or more of said second antibodies or antigen-binding fragments thereof neutralize hMPV.

In certain embodiments, the invention provides a method wherein one or more of said third antibodies or antigen-binding fragments thereof neutralize PIV.

In certain embodiments, the invention provides a method wherein one or more of said first antibodies or antigen-binding fragments thereof block RSV infection of cells of the subject.

In certain embodiments, the invention provides a method wherein one or more of said second antibodies or antigen-binding fragments thereof block hMPV infection of cells of the subject.

In certain embodiments, the invention provides a method wherein one or more of said third antibodies or antigen-binding fragments thereof block PIV infection of cells of the subject.

In certain embodiments, the invention provides a method of treating one or more symptoms of a respiratory viral infection in a subject, said method comprising administering to the subject: (i) a therapeutically effective amount of one or more first antibodies or antigen-binding fragments thereof, wherein one or more of said first antibodies or antigen-binding fragments thereof bind immunospecifically to a RSV antigen; (ii) a therapeutically effective amount of one or more second antibodies or antigen-binding fragments thereof, wherein one or more of said second antibodies or antigen-binding fragments thereof bind immunospecifically to a hMPV antigen; and (iii) a therapeutically effective amount of one or more third antibodies or antigen-binding fragments thereof, wherein one or more of said third antibodies or antigen-binding fragments thereof bind immunospecifically to a PIV antigen.

In certain embodiments, the invention provides a method of passive immunotherapy, said method comprising administering to a subject: (i) a first dose of one or more first antibodies or antigen-binding fragments thereof, wherein one or more of said first antibodies or a fragments thereof bind immunospecifically to a RSV antigen; (ii) a second dose of one or more second antibodies or antigen-binding fragments thereof, wherein one or more of said second antibodies or a fragments thereof bind immunospecifically to a hMPV antigen; and (iii) a third dose of one or more third antibodies or antigen-binding fragments thereof, wherein one or more of said third antibodies or antigen-binding fragments thereof bind immunospecifically to a PIV antigen wherein the first dose reduces the incidence of RSV infection by at least 25%, wherein the second dose reduces the incidence of hMPV infection by at least 25%, and wherein the third dose reduces the incidence of PIV infection by at least 25%.

In certain embodiments, the invention provides a method wherein the first dose reduces the incidence of RSV infection by at least 50%, wherein the second dose reduces the incidence of hMPV infection by at least 50%, and wherein the third dose reduces the incidence of PIV infection by at least 50%.

In certain embodiments, the invention provides a method wherein the first dose reduces the incidence of RSV infection by at least 75%, wherein the second dose reduces the incidence of hMPV infection by at least 75%, and wherein the third dose reduces the incidence of PIV infection by at least 75%.

In certain embodiments, the invention provides a method wherein the first dose reduces the incidence of RSV infection by at least 90%, wherein the second dose reduces the incidence of hMPV infection by at least 90%, and wherein the third antibody reduces the incidence of PIV infection by at least 90%.

In certain embodiments, the invention provides a method of passive immunotherapy, said method comprising administering to a subject: (i) a first dose of one or more first antibodies or antigen-binding fragments thereof, wherein one or more of said first antibodies or antigen-binding fragments thereof bind immunospecifically to a RSV antigen; (ii) a second dose of one or more second antibodies or antigen-binding fragments thereof, wherein one or more of said second antibodies or antigen-binding fragments thereof bind immunospecifically to a hMPV antigen; and (iii) a third dose of one or more third antibodies or antigen-binding fragments thereof, wherein one or more of said third antibodies or antigen-binding fragments thereof bind immunospecifically to a PIV antigen, wherein the serum titer of one or more of said first antibodies or antigen-binding fragments thereof in the subject is at least 10 μg/ml after 15 days of administering one or more of said first antibodies or antigen-binding fragments thereof, wherein the serum titer of one or more of said second antibodies or antigen-binding fragments thereof in the subject is at least 10 μg/ml after 15 days of administering one or more of said second antibodies or antigen-binding fragments thereof, and wherein the serum titer of one or more of said third antibodies or antigen-binding fragments thereof in the subject is at least 10 μg/ml after 15 days of administering one or more of said third antibodies or antigen-binding fragments thereof.

In certain embodiments, the invention provides a method wherein the amino acid sequence of the PIV antigen is that of SEQ ID NO:407 to 419, respectively.

In certain embodiments, the invention provides a method wherein the amino acid sequence of the PIV antigen is 90% identical to the amino acid sequence of PIV nucleoprotein, PIV phosphoprotein, PIV matrix protein, PIV small hydrophobic protein, PIV RNA-dependent RNA polymerase, PIV F protein, or PIV G protein.

In certain embodiments, the invention provides a method wherein the PIV antigen is selected from the group consisting of PIV nucleoprotein, PIV phosphoprotein, PIV matrix protein, PIV small hydrophobic protein, PIV RNA-dependent RNA polymerase, PIV F protein, and PIV G protein.

In certain embodiments, the invention provides a method of preventing a viral infection in a subject, said method comprising administering to the subject: (i) a prophylactically effective amount of one or more first antibodies or antigen-binding fragments thereof, wherein one or more of said first antibodies or antigen-binding fragments thereof bind immunospecifically to a RSV antigen; and (ii) a prophylactically effective amount of one or more second antibodies or antigen-binding fragments thereof, wherein one or more of said second antibodies or antigen-binding fragments thereof bind immunospecifically to a PIV antigen.

In certain embodiments, the invention provides a method wherein one or more of said first antibodies or antigen-binding fragments thereof neutralize RSV.

In certain embodiments, the invention provides a method wherein one or more of said second antibodies or antigen-binding fragments thereof neutralize PIV.

In certain embodiments, the invention provides a method wherein one or more of said first antibodies or antigen-binding fragments thereof block RSV infection of cells of the subject.

In certain embodiments, the invention provides a method wherein one or more of said second antibodies or antigen-binding fragments thereof block PIV infection of cells of the subject.

In certain embodiments, the invention provides a method of treating one or more symptoms of a respiratory viral infection in a subject, said method comprising administering to the subject: (i) a therapeutically effective amount of one or more first antibodies or antigen-binding fragments thereof, wherein one or more of said first antibodies or antigen-binding fragments thereof bind immunospecifically to a RSV antigen; and (ii) a therapeutically effective amount of one or more second antibodies or antigen-binding fragments thereof, wherein one or more of said second antibodies or antigen-binding fragments thereof bind immunospecifically to a PIV antigen.

In certain embodiments, the invention provides a method of passive immunotherapy, said method comprising administering to a subject: (i) a first dose of one or more first antibodies or antigen-binding fragments thereof, wherein one or more of said first antibodies or a fragments thereof bind immunospecifically to a RSV antigen; and (ii) a second dose of one or more second antibodies or antigen-binding fragments thereof, wherein one or more of said second antibodies or a fragments thereof bind immunospecifically to a PIV antigen, wherein the first dose reduces the incidence of RSV infection by at least 25% and wherein the second dose reduces the incidence of PIV infection by at least 25%.

In certain embodiments, the invention provides a method wherein the first dose reduces the incidence of RSV infection by at least 50% and wherein the second dose reduces the incidence of hMPV infection by at least 50%.

In certain embodiments, the invention provides a method wherein the first dose reduces the incidence of RSV infection by at least 75% and wherein the second dose reduces the incidence of hMPV infection by at least 75%.

In certain embodiments, the invention provides a method wherein the first dose reduces the incidence of RSV infection by at least 90% and wherein the second dose reduces the incidence of hMPV infection by at least 90%.

In certain embodiments, the invention provides a method of passive immunotherapy, said method comprising administering to a subject: (i) a first dose of one or more first antibodies or antigen-binding fragments thereof, wherein one or more of said first antibodies or antigen-binding fragments thereof bind immunospecifically to a RSV antigen; and (ii) a second dose of one or more second antibodies or antigen-binding fragments thereof, wherein one or more of said second antibodies or antigen-binding fragments thereof bind immunospecifically to a PIV antigen, wherein the serum titer of one or more of said first antibodies or antigen-binding fragments thereof in the subject is at least 10 μg/ml after 15 days of administering one or more of said first antibodies or antigen-binding fragments thereof and wherein the serum titer of one or more of said second antibodies or antigen-binding fragments thereof in the subject is at least 10 μg/ml after 15 days of administering one or more of said second antibodies or antigen-binding fragments thereof.

### 3.2. BRIEF DESCRIPTION OF THE FIGURES

FIG. 1. Expression constructs for the expression of the hMPV F protein.

3.3. Definitions

The term “analog” of a certain polypeptide as used herein refers to a polypeptide that possesses a similar or identical function as the certain polypeptide or a fragment of the certain polypeptide, the certain polypeptide can be, e.g., an antibody or an antigen-binding fragment thereof, but does not necessarily comprise a similar or identical amino acid sequence to the certain polypeptide or fragment thereof, or possess a similar or identical structure to the certain polypeptide.

A polypeptide that has a similar amino acid sequence to a certain polypeptide refers to a polypeptide that satisfies at least one of the following: (a) a polypeptide having an amino acid sequence that is at least 30%, at least 35%, at least 40%, at least 45%, at least 50%, at least 55%, at least 60%, at least 65%, at least 70%, at least 75%, at least 80%, at least 85%, at least 90%, at least 95% or at least 99% identical to the amino acid sequence the certain polypeptide; (b) a polypeptide encoded by a nucleotide sequence that hybridizes under stringent conditions to a nucleotide sequence encoding the certain polypeptide of at least 5 amino acid residues, at least 10 amino acid residues, at least 15 amino acid residues, at least 20 amino acid residues, at least 25 amino acid residues, at least 40 amino acid residues, at least 50 amino acid residues, at least 60 amino residues, at least 70 amino acid residues, at least 80 amino acid residues, at least 90 amino acid residues, at least 100 amino acid residues, at least 125 amino acid residues, or at least 150 amino acid residues; and (c) a polypeptide encoded by a nucleotide sequence that is at least 30%, at least 35%, at least 40%, at least 45%, at least 50%, at least 55%, at least 60%, at least 65%, at least 70%, at least 75%, at least 80%, at least 85%, at least 90%, at least 95% or at least 99% identical to the nucleotide sequence encoding the certain polypeptide. A polypeptide with similar structure to a certain polypeptide refers to a polypeptide that has a similar secondary, tertiary or quaternary structure to a certain polypeptide. The structure of a polypeptide can be determined by methods known to those skilled in the art, including but not limited to, X-ray crystallography, nuclear magnetic resonance, and crystallographic electron microscopy. A certain polypeptide in the context of the present invention can be RSV polypeptide, an APV polypeptide, a hMPV polypeptide, a PIV polypeptide, a fragment of a RSV polypeptide, a fragment of an APV polypeptide, a fragment of a hMPV polypeptide, a fragment of a PIV polypeptide, an antibody that immunospecifically binds to a RSV polypeptide, an antibody that immunospecifically binds to an APV polypeptide, an antibody that immunospecifically binds to a PIV polypeptide, an antibody that immunospecifically binds to a hMPV polypeptide, an antibody fragment that immunospecifically binds to a RSV polypeptide, an antibody fragment that immunospecifically binds to an APV polypeptide, an antibody fragment that immunospecifically binds to a PIV polypeptide, or an antibody fragment that immunospecifically binds to a hMPV polypeptide.

As used herein, the terms “antibody” and “antibodies” refer to monoclonal antibodies, multispecific antibodies (e.g., bi-specific), human antibodies, humanized antibodies, camelised antibodies, chimeric antibodies, single-chain Fvs (scFv), single chain antibodies, synthetic antibodies, single domain antibodies, Fab fragments, F(ab) fragments, disulfide-linked Fvs (sdFv), and anti-idiotypic (anti-Id) antibodies, and epitope-binding fragments of any of the above. In particular, antibodies include immunoglobulin molecules and immunologically active fragments of immunoglobulin molecules, i.e., molecules that contain an antigen binding site. Immunoglobulin molecules can be of any type (e.g., IgG, IgE, IgM, IgD, IgA and IgY), class (e.g., IgG₁, IgG₂, IgG₃, IgG₄, IgA₁ and IgA₂) or subclass.

As used herein, the term “in combination” refers to the use of more than one prophylactic and/or therapeutic agents. The use of the term “in combination” does not restrict the order in which prophylactic and/or therapeutic agents are administered to a subject with a respiratory viral infection. A first prophylactic or therapeutic agent can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second prophylactic or therapeutic agent to a subject which was or is susceptible to a respiratory viral infection. Any additional prophylactic or therapeutic agent can be administered in any order with the other additional prophylactic or therapeutic agents.

As used herein, the term “synergistic” refers to a combination of prophylactic or therapeutic agents which is more effective than the additive effects of any two or more single agents. A synergistic effect of a combination of prophylactic or therapeutic agents permits the use of lower dosages of one or more of the agents and/or less frequent administration of said agents to a subject with a respiratory viral infection. The ability to utilize lower dosages of prophylactic or therapeutic agents and/or to administer said agents less frequently reduces the toxicity associated with the administration of said agents to a subjectd without reducing the efficacy of said agents in the prevention or treatment of respiratory viral infections. In addition, a synergistic effect can result in improved efficacy of agents in the prevention or treatment of respiratory viral infections. Finally, synergistic effect of a combination of prophylactic or therapeutic agents may avoid or reduce adverse or unwanted side effects associated with the use of any single therapy.

The term “derivative” as used herein refers to a polypeptide that has been altered by the introduction of amino acid residue substitutions, deletions or additions. The term “derivative” refers also to a polypeptide that has been modified, i.e, by the covalent attachment of any type of molecule to the polypeptide. Further modifications are, inter alia, glycosylation, acetylation, pegylation, phosphorylation, amidation, derivatization by known protecting/blocking groups, proteolytic cleavage, linkage to a cellular ligand or other protein. Modifications include, inter alia, chemical modifications by techniques known to those of skill in the art, e.g., chemical cleavage, acetylation, formylation, synthesis in the presence of tunicamycin, etc. Further, a derivative if a certain polypeptide can be generated by introducing one or more non-classical amino acids into the certain polypeptide. A polypeptide derivative possesses a similar or identical function as the certain polypeptide from which it is derived.

The term “effective neutralizing titer” as used herein refers to the amount of antibody which corresponds to the amount present in the serum of animals (human or cotton rat) that has been shown to be either clinically efficacious (in humans) or to reduce virus by 99% in, for example, cotton rats. The 99% reduction is defined by a specific challenge of, e.g., 10³ pfu, 10⁴ pfu, 10⁵ pfu, 10⁶ pfu, 10⁷ pfu, 10⁸ pfu, or 10⁹ pfu of RSV, PIV, and/or hMPV.

The term “epitopes” as used herein refers to a portion of a protein or polypeptide having antigenic and/or immunogenic activity in an animal, preferably a mammal, and most preferably in a human. An epitope having immunogenic activity is a portion of a protein or polypeptide that elicits an antibody response in an animal. An epitope having antigenic activity is a portion of a protein or polypeptide to which an antibody immunospecifically binds as determined by any method well known in the art, for example, by the immunoassays described herein. Antigenic epitopes need not necessarily be immunogenic.

The term “fragment” as used herein refers to a peptide or polypeptide comprising an amino acid sequence of at least 5 contiguous amino acid residues, at least 10 contiguous amino acid residues, at least 15 contiguous amino acid residues, at least 20 contiguous amino acid residues, at least 25 contiguous amino acid residues, at least 40 contiguous amino acid residues, at least 50 contiguous amino acid residues, at least 60 contiguous amino residues, at least 70 contiguous amino acid residues, at least contiguous 80 amino acid residues, at least contiguous 90 amino acid residues, at least contiguous 100 amino acid residues, at least contiguous 125 amino acid residues, at least 150 contiguous amino acid residues, at least contiguous 175 amino acid residues, at least contiguous 200 amino acid residues, or at least contiguous 250 amino acid residues of the amino acid sequence of a polypeptide, protein, or antibody. Preferably, a fragment has the reactive activity of the polypeptide, protein, or antibody.

The term “human infant” as used herein refers to a human less than 24 months, preferably less than 16 months, less than 12 months, less than 6 months, less than 3 months, less than 2 months, or less than 1 month of age. In certain embodiments, the human infant is born at more than 38 weeks of gestational age.

The term “human infant born prematurely” as used herein refers to a human born at less than 40 weeks gestational age, less than 35 weeks gestational age. In specific embodiments, the prematurely born human infant is of between 30-35 weeks of gestational age. In specific embodiments, the prematurely born human infant is of between 35-38 weeks of gestational age. In certain embodiments, the prematurely born infant is of 38 weeks gestational age, preferably, the infant is of less than 38 weeks gestational age.

An “isolated” or “purified” antibody or fragment thereof is substantially free of cellular material or other contaminating proteins from the cell or tissue source from which the protein is derived, or substantially free of chemical precursors or other chemicals when chemically synthesized. The language “substantially free of cellular material” includes preparations of an antibody or antibody fragment in which the antibody or antibody fragment is separated from cellular components of the cells from which it is isolated or recombinantly produced. Thus, an antibody or antibody fragment that is substantially free of cellular material includes preparations of antibody or antibody fragment having less than about 30%, 20%, 10%, or 5% (by dry weight) of heterologous protein (also referred to herein as a “contaminating protein”). When the antibody or antibody fragment is recombinantly produced, it is also preferably substantially free of culture medium, i.e., culture medium represents less than about 20%, 10%, or 5% of the volume of the protein preparation. When the antibody or antibody fragment is produced by chemical synthesis, it is preferably substantially free of chemical precursors or other chemicals, i.e., it is separated from chemical precursors or other chemicals which are involved in the synthesis of the protein. Accordingly such preparations of the antibody or antibody fragment have less than about 30%, 20%, 10%, 5% (by dry weight) of chemical precursors or compounds other than the antibody or antibody fragment of interest. In a preferred embodiment, antibodies of the invention or fragments thereof are isolated or purified.

An “isolated” nucleic acid molecule is one which is separated from other nucleic acid molecules which are present in the natural source of the nucleic acid molecule. Moreover, an “isolated” nucleic acid molecule, such as a cDNA molecule, can be substantially free of other cellular material, or culture medium when produced by recombinant techniques, or substantially free of chemical precursors or other chemicals when chemically synthesized. In a preferred embodiment, nucleic acid molecules encoding antibodies of the invention or fragments thereof are isolated or purified.

The term “fusion protein” as used herein refers to a polypeptide that comprises an amino acid sequence of an antibody or fragment thereof and an amino acid sequence of a heterologous polypeptide (e.g., a non-anti-RSV antibody, a non-anti-PIV antibody, a non-anti-APV antibody and/or a non-anti-hMPV antibody).

The term “high potency” as used herein refers to antibodies or antigen-binding fragments thereof that exhibit high potency as determined in various assays for biological activity (e.g., neutralization of RSV, APV, hMPV, PIV) such as those described herein. For example, high potency antibodies of the present invention or fragments thereof have an EC₅₀ value less than 0.01 nM, less than 0.025 nM, less than 0.05 nM, less than 0.1 nM, less than 0.25 nM, less than 0.5 nM, less than 0.75 nM, less than 1 nM, less than 1.25 nM, less than 1.5 nM, less than 1.75 nM, or less than 2 nM as measured by a microneutralization assay described herein. Further, high potency antibodies of the present invention or fragments thereof result in at least a 30%, 40%, 50%, 60%, 75%, preferably at least a 95% and more preferably a 99% lower RSV titer, PIV titer, APV titer, and/or hMPV titer in a subject, such as a cotton rat 5 days after challenge with 105 pfu relative to a subject, such as a cotton rat, not administered with said antibodies or antibody fragments. In certain embodiments of the invention, high potency antibodies of the present invention or fragments thereof exhibit a high affinity and/or high avidity for one or more RSV antigens, one or more PIV antigens, one or more hMPV antigens, and/or one or more APV antigens (e.g., antibodies or antibody fragments having an affinity of at least 2×10⁸ M⁻¹, at least 2.5×10⁸ M⁻¹, at least 5×10⁸ M⁻¹, at least 10⁹ M⁻¹, at least 5×10⁹ M⁻¹, at least 10¹⁰ M⁻¹, at least 5×10¹⁰ M⁻¹, at least 10¹¹ M⁻¹, at least 5×10¹¹ M⁻¹, at least 10¹² M⁻¹, or at least 5×10¹² M⁻¹ for one or more RSV antigens, one or more PIV antigens, one or more hMPV antigens, and/or one or more APV antigens).

The term “host” as used herein refers to a mammal, preferably a human.

The term “host cell” as used herein refers to the particular subject cell transfected with a nucleic acid molecule and the progeny or potential progeny of such a cell. Progeny of such a cell may not be identical to the parent cell transfected with the nucleic acid molecule due to mutations or environmental influences that may occur in succeeding generations or integration of the nucleic acid molecule into the host cell genome.

In certain embodiments of the invention, a “prophylactically effective serum titer” is the serum titer in a mammal, preferably a human, that reduces the incidence of a respiratory viral infection, particularly a RSV infection, a hMPV infection, a PIV infection, and/or a APV infection in a subject. Preferably, the prophylactically effective serum titer reduces the incidence of RSV infections, hMPV infections, PIV infections, and/or APV infections in a subject with the greatest probability of complications resulting from RSV infection, hMPV infection, PIV infection, and/or APV infection, respectively (e.g., a subject with cystic fibrosis, bronchopulmonary dysplasia, congenital heart disease, congenital immunodeficiency or acquired immunodeficiency, a subject who has had a bone marrow transplant, a human infant, or an elderly human). In certain other embodiments of the invention, a “prophylactically effective serum titer” is the serum titer in a cotton rat that results in a RSV titer, hMPV titer, PIV titer, and/or APV titer 5 days after challenge with 10⁵ pfu that is 90%, i.e., 1 log, lower than the RSV titer, hMPV titer, PIV titer, and/or APV titer 5 days after challenge with 10⁵ pfu of RSV, hMPV, APV, and/or PIV, respectively, in a cotton rat not administered an antibody or antibody fragment that immunospecifically binds to a RSV antigen, hMPV antigen, PIV antigen, and/or APV antigen, respectively. A prophylactically effective amount includes an amount that is prophylactically effective in combination with other agents, even if it is not prophylactically effective by itself.

In certain embodiments of the invention, a “therapeutically effective serum titer” is the serum titer in a mammal, preferably a human, that reduces the severity, the duration and/or the symptoms associated with a respiratory viral infection, particularly with a RSV infection, a hMPV infection, an APV infection, and/or a PIV infection in said mammal. Preferably, the therapeutically effective serum titer reduces the severity, the duration and/or the number symptoms associated with RSV infections, hMPV infections, APV infections, and/or PIV infections in humans with the greatest probability of complications resulting from a RSV, APV, hMPV, and/or PIV infection (e.g., a human with cystic fibrosis, bronchopulmonary dysplasia, congenital heart disease, congenital immunodeficiency or acquired immunodeficiency, a human who has had a bone marrow transplant, a human infant, or an elderly human). In certain other embodiments of the invention, a “therapeutically effective serum titer” is the serum titer in a cotton rat that results in a RSV, APV, hMPV, and/or PIV titer 5 days after challenge with 10⁵ pfu that is 90%, i.e., 1 log, lower than the RSV, APV, hMPV, and/or PIV titer 5 days after challenge with 10⁵ pfu of RSV APV, hMPV, and/or PIV, respectively, in a cotton rat not administered an antibody or antibody fragment that immunospecifically binds to a RSV, APV, hMPV, and/or PIV antigen, respectively. A therapeutically effective amount includes an amount that is therapeutically effective in combination with other agents, even if it is not therapeutically effective by itself.

The term “anti-PIV-antigen antibody” refers to an antibody or antibody fragment thereof that binds immunospecifically to a PIV antigen. A PIV antigen refers to a PIV polypeptide or fragment thereof such as of PIV nucleocapsid structural protein, PIV phosphoprotein, PIV fusion glycoprotein, PIV L protein, PIV matrix protein, PIV HN glycoprotein, PIV RNA-dependent RNA polymerase, PIV Y1 protein, PIV D protein, or PIV C protein. A PIV antigen also refers to a polypeptide that has a similar amino acid sequence compared to a PIV nucleocapsid structural protein, PIV phosphoprotein, PIV fusion glycoprotein, PIV L protein, PIV matrix protein, PIV HN glycoprotein, PIV RNA-dependent RNA polymerase, PIV Y1 protein, PIV D protein, or PIV C protein.

The term “anti-RSV-antigen antibody” refers to an antibody or antibody fragment thereof that binds immunospecifically to a RSV antigen. A RSV antigen refers to a RSV polypeptide or fragment thereof such as of RSV nucleoprotein, RSV phosphoprotein, RSV matrix protein, RSV small hydrophobic protein, RSV RNA-dependent RSV polymerase, RSV F protein, and RSV G protein. A RSV antigen also refers to a polypeptide that has a similar amino acid sequence compared to a RSV polypeptide or fragment thereof such as of RSV nucleoprotein, RSV phosphoprotein, RSV matrix protein, RSV small hydrophobic protein, RSV RNA-dependent RSV polymerase, RSV F protein, and RSV G protein.

The term “anti-hMPV-antigen antibody” refers to an antibody or antibody fragment thereof that binds immunospecifically to a hMPV antigen. A hMPV antigen refers to a hMPV polypeptide or fragment thereof such as of hMPV nucleoprotein, hMPV phosphoprotein, hMPV matrix protein, hMPV small hydrophobic protein, hMPV RNA-dependent hMPV polymerase, hMPV F protein, and hMPV G protein. A hMPV antigen also refers to a polypeptide that has a similar amino acid sequence compared to a hMPV polypeptide or fragment thereof such as of hMPV nucleoprotein, hMPV phosphoprotein, hMPV matrix protein, hMPV small hydrophobic protein, hMPV RNA-dependent hMPV polymerase, hMPV F protein, and hMPV G protein.

The term “serum titer” as used herein refers to an average serum titer in a population of least 10, preferably at least 20, and most preferably at least 40 subjects.

The term “subject” as used herein refers to vertebrate, preferably to a mammal. A subject can be a primate, a rat, a mouse, or a cotton rat. Most preferably, the subject is a human.

As used herein, the terms “immunospecifically binds” and “anti-RSV, anti-hMPV, or anti-PIV antibodies” and analogous terms refer to antibodies or fragments thereof that specifically bind to a RSV antigen, a hMPV antigen, or a PIV antigen in an ELISA assay or any other immuno-assay well-known to the skilled artisan (e.g., as described in section 4.8, infra). In certain embodiments, an antibody or fragment thereof that immunospecifically binds to a RSV antigen, a hMPV antigen, or a PIV antigen may bind to other peptides or polypeptides with lower or equal affinity as determined by, e.g., immunoassays, BIAcore, or other assays known in the art. In certain other embodiments, an antibody or fragment thereof that immunospecifically binds to a RSV antigen, a hMPV antigen, or a PIV antigen does not bind to other peptides or polypeptides as determined by, e.g., immunoassays, BIAcore, or other assays known in the art. Antibodies or fragments that immunospecifically bind to a RSV antigen, a hMPV antigen, or a PIV antigen may be cross-reactive with related antigens. Preferably, antibodies or fragments that immunospecifically bind to a RSV antigen, a hMPV antigen, or a PIV antigen do not cross-react with other antigens. Antibodies or fragments that immunospecifically bind to a RSV antigen, a hMPV antigen, or a PIV antigen can be identified, for example, by immunoassays, BIAcore, or other techniques known to those of skill in the art. In certain embodiments, an antibody or fragment thereof binds specifically to a RSV antigen, a hMPV antigen, or a PIV antigen when it binds to a RSV antigen, a hMPV antigen, or a PIV antigen with higher affinity than to any cross-reactive antigen as determined using experimental techniques, such as, but not limited to, radioimmunoassays (RIA), enzyme-linked immunosorbent assays (ELISAs), BIAcore, or other techniques known to those of skill in the art. See, e.g., Paul, ed., 1989, Fundamental Immunology Second Edition, Raven Press, New York at pages 332-336 for a discussion regarding antibody specificity. In certain embodiments, an antibody or fragment thereof binds specifically to a RSV antigen, a hMPV antigen, or a PIV antigen with equal affinity as to any cross-reactive antigen as determined using experimental techniques, such as radioimmunoassays (RIA) and enzyme-linked immunosorbent assays (ELISAs).

To determine the percent identity of two amino acid sequences or of two nucleic acid sequences, the sequences are aligned for optimal comparison purposes (e.g., gaps can be introduced in the sequence of a first amino acid or nucleic acid sequence for optimal alignment with a second amino acid or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=number of identical overlapping positions/total number of positions×100%). In one embodiment, the two sequences are the same length.

The determination of percent identity between two sequences can also be accomplished using a mathematical algorithm. A preferred, non-limiting example of a mathematical algorithm utilized for the comparison of two sequences is the algorithm of Karlin and Altschul, 1990, Proc. Natl. Acad. Sci. U.S.A. 87:2264-2268, modified as in Karlin and Altschul, 1993, Proc. Natl. Acad. Sci. U.S.A. 90:5873-5877. Such an algorithm is incorporated into the NBLAST and XBLAST programs of Altschul et al., 1990, J. Mol. Biol. 215:403. BLAST nucleotide searches can be performed with the NBLAST nucleotide program parameters set, e.g., for score=100, wordlength=12 to obtain nucleotide sequences homologous to a nucleic acid molecules of the present invention. BLAST protein searches can be performed with the XBLAST program parameters set, e.g., to score-50, wordlength=3 to obtain amino acid sequences homologous to a protein molecule of the present invention. To obtain gapped alignments for comparison purposes, Gapped BLAST can be utilized as described in Altschul et al., 1997, Nucleic Acids Res. 25:3389-3402. Alternatively, PSI-BLAST can be used to perform an iterated search which detects distant relationships between molecules (Id.). When utilizing BLAST, Gapped BLAST, and PSI-Blast programs, the default parameters of the respective programs (e.g., of XBLAST and NBLAST) can be used (see, e.g., http://www.ncbi.nlm.nih.gov). Another preferred, non-limiting example of a mathematical algorithm utilized for the comparison of sequences is the algorithm of Myers and Miller, 1988, CABIOS 4:11-17. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

The percent identity between two sequences can be determined using techniques similar to those described above, with or without allowing gaps. In calculating percent identity, typically only exact matches are counted.

References to RSV, PIV, hMPV, and APV include all groups, subgroups, isolates, types and strains of the respective virus. In a specific embodiment, RSV, PIV, and hMPV refer to all groups, subgroups, isolates, types and strains of human RSV, PIV, and hMPV, respectively.

### Abbreviations

| | |
|---|---|
| cDNA | complementary DNA |
| L | large protein |
| M | matrix protein (lines inside of envelope) |
| F | fusion glycoprotein |
| HN | hemagglutinin-neuraminidase glycoprotein |
| N, NP or NC | nucleoprotein (associated with RNA and |
| | required for polymerase activity) |
| P | phosphoprotein |
| MOI | multiplicity of infection |
| NA | neuraminidase (envelope glycoprotein) |
| PIV | parainfluenza virus |
| nt | nucleotide |
| hMPV | human metapneumovirus |
| APV | avian pneumovirus |

### 4. DETAILED DESCRIPTION OF THE INVENTION

4.1 Antibodies

The invention provides methods of passive immunotherapy for broad-spectrum prevention and, in certain embodiments, treatment of viral respiratory infection. The antibodies to be used with the methods of the invention include antibodies or antigen-binding fragments thereof that bind immunospecifically to a RSV antigen, antibodies or antigen-binding fragments thereof that bind immunospecifically to a hMPV antigen, antibodies or antigen-binding fragments thereof that bind immunospecifically to a PIV antigen, and, in a specific embodiment, human or humanized antibodies that bind immunospecifically to a hMPV antigen and that cross-react with an APV antigen. In a specific embodiment, the antibody to be used with the methods of the invention is an antibody that binds immunospecifically to a hMPV antigen and that cross-reacts with a turkey APV antigen. In a specific embodiment, the antibody to be used with the methods of the invention is a human or humanized antibody that binds immunospecifically to a hMPV antigen and that cross-reacts with a turkey APV antigen. In other specific embodiments, the anti-hMPV antibody does not react with a turkey APV antigen or an APV antigen from any other species of APV.

In certain embodiments, fragments of viral antigens are used as immunogen to produce antibodies to be used with the methods of the invention. In certain embodiments, fragments preferably contain a sequence of at least 4, at least 5, at least 6, at least 7, more preferably at least 8, at least 9, at least 10, at least 11, at least 12, at least 13, at least 14, at least 15, at least 20, at least 25, at least 30, at least 40, at least 50, at least 75 or at least 100 amino acids. In certain, more specific embodiments, a fragment is about 15 to about 30 amino acids long. Preferred polypeptides comprising immunogenic or antigenic epitopes are at least 10, 15, 20, 25, 30, 35, 40, 45, 50, 55, 60, 65, 70, 75, 80, 85, 90, 95, or 100 amino acid residues in length. Additional non-exclusive preferred antigenic epitopes include the antigenic epitopes disclosed herein, as well as portions thereof.

In certain embodiments, the anti-PIV-antigen antibody, the anti-RSV-antigen antibody, and/or the anti-hMPV-antigen antibody inhibit the binding of a virus that causes respiratory infection to a cell. In certain embodiments, the anti-PIV-antigen antibody, the anti-RSV-antigen antibody, and/or the anti-hMPV-antigen antibody inhibit in a subject the binding of a virus that causes respiratory infection to a cell of the subject. In certain embodiments, the anti-PIV-antigen antibody, the anti-RSV-antigen antibody, and/or the anti-hMPV-antigen antibody inhibit the infection of a subject with a virus that causes respiratory infections. In certain embodiments, the anti-PIV-antigen antibody, the anti-RSV-antigen antibody, and/or the anti-hMPV-antigen antibody cause neutralization of the virus that causes respiratory infections.

The antibodies to be used with the methods of the invention bind immunospecifically to a variety of viral antigens as discussed in sections 4.1.5, 4.1.6, and 4.1.7 below. In certain embodiments, at least one antibody to be used with the methods of the invention binds immunospecifically to an epitope of an antigen of PIV, hMPV, or RSV, and cross-reacts with another epitope on the same antigen of PIV, hMPV, or RSV, respectively. In certain embodiments, at least one antibody to be used with the methods of the invention binds immunospecifically to an epitope of an antigen of PIV, hMPV, or RSV, and cross-reacts with the analogous antigen of a different virus. For example, an antibody that binds immunospecifically to the F protein of RSV cross reacts with the F protein of hMPV. In a specific embodiment, the anti-RSV-antigen antibody is SYNAGIS®. SYNAGIS® is also known as Palivizumab. The amino acid sequence of SYNAGIS® (Palivizumab) is disclosed in International Application Publication WO 02/43660, entitled “Methods of Administering/Dosing Anti-RSV Antibodies for Prophylaxis and Treatment”, by Young et al., which is incorporated herein by reference in its entirety. In another specific embodiment, the anti-RSV-antigen antibody is not SYNAGIS®. In certain specific embodiments, the anti-RSV-antigen antibody is AFFF; P12f2 P12f4; P11d4; Ale9; A12a6; A13c4; A17d4; A4B4; 1X-493L1; FR H3-3F4; M3H9; Y10H6; DG; AFFF(1); 6H8; L1-7E5; L2-15B10; A13a11; A1h5; A4B4(1);A4B4-F52S; or A4B4L1FR-S28R. These antibodies are disclosed in International Application Publication No.: WO 02/43660, entitled “Methods of Administering/Dosing Anti-RSV Antibodies for Prophylaxis and Treatment”, by Young et al., which is incorporated herein by reference in its entirety.

In certain embodiments, at least one antibody to be used with the methods of the invention binds immunospecifically to an antigen of one subgroup (type, subtype, group, isolate etc.) of PIV, hMPV, or RSV and to the analogous antigen of another subgroup (type, subtype, group, isolate etc.) of PIV, hMPV, or RSV, respectively (see sections 4.1.5, 4.1.6, and 4.1.7, respectively).

Antibodies of the invention include, but are not limited to, monoclonal antibodies, multispecific antibodies, synthetic antibodies, human antibodies, humanized antibodies, chimeric antibodies, single-chain Fvs (scFv), single chain antibodies, Fab fragments, F(ab′) fragments, disulfide-linked Fvs (sdFv), and anti-idiotypic (anti-Id) antibodies (including, e.g., anti-Id antibodies to antibodies of the invention), and epitope-binding fragments of any of the above. In particular, antibodies of the present invention include immunoglobulin molecules and immunologically active portions of immunoglobulin molecules, i.e., molecules that contain an antigen binding site that immunospecifically binds to a RSV, PIV, APV, and/or hMPV antigen. The immunoglobulin molecules of the invention can be of any type (e.g., IgG, IgE, IgM, IgD, IgA and IgY), class (e.g., IgG₁, IgG₂, IgG₃, IgG₄, IgA₁ and IgA₂) or subclass of immunoglobulin molecule.

The antibodies of the invention may be from any animal origin including birds and mammals (e.g., human, murine, donkey, sheep, rabbit, goat, guinea pig, camel, horse, or chicken). Preferably, the antibodies of the invention are human or humanized monoclonal antibodies. As used herein, “human” antibodies include antibodies having the amino acid sequence of a human immunoglobulin and include antibodies isolated from human immunoglobulin libraries (including, but not limited to, synthetic libraries of immunoglobulin sequences homologous to human immunoglobulin sequences) or from mice that express antibodies from human genes.

The antibodies of the present invention may be monospecific, bispecific, trispecific or of greater multispecificity. Multispecific antibodies may be specific for different epitopes of one antigen of RSV, PIV, or hMPV. In certain embodiments, multispecific antibodies are specific for more than one antigen of RSV, PIV, or hMPV. In certain embodiments, multispecific antibodies are specific for an antigen of RSV and an antigen of hMPV. In certain embodiments, multispecific antibodies are specific for an antigen of PIV and an antigen of hMPV. In certain embodiments, multispecific antibodies are specific for an antigen of PIV and an antigen of RSV. In certain embodiments, multispecific antibodies are specific for an antigen of RSV, an antigen of PIV, and an antigen of hMPV. For multispecific antibodies see, e.g., PCT publications WO 93/17715, WO 92/08802, WO 91/00360, and WO 92/05793; Tutt, et al., J. Immunol. 147:60-69(1991); U.S. Pat. Nos. 4,474,893, 4,714,681, 4,925,648, 5,573,920, and 5,601,819; and Kostelny et al., J. Immunol. 148:1547-1553 (1992).

In certain embodiments, high potency antibodies can be used in the methods of the invention. For example, high potency antibodies can be produced by genetically engineering appropriate antibody gene sequences and expressing the antibody sequences in a suitable host. The antibodies produced can be screened to identify antibodies with, e.g., high kₒₙ values in a BIAcore assay (see section 4.8.3).

In certain embodiments, an antibody to be used with the methods of the present invention or fragment thereof has an affinity constant or Kₐ (kₒₙ/k_{off}) of at least 10² M⁻¹, at least 5×10² M⁻¹, at least 10³ M⁻¹, at least 5×10³ M⁻¹, at least 10⁴ M⁻¹, at least 5×10⁴ M⁻¹, at least 10⁵ M⁻¹, at least 5×10⁵ M⁻¹, at least 10⁶ M⁻¹, at least 5×10⁶ M⁻¹, at least 10⁷ M⁻¹, at least 5×10⁷ M⁻¹, at least 10⁸ M⁻¹, at least 5×10⁸ M⁻¹, at least 10⁹ M⁻¹, at least 5×10⁹ M⁻¹, at least 10¹⁰ M⁻¹, at least 5×10¹⁰ M⁻¹, at least 10¹¹ M⁻¹, at least 5×10¹¹ M⁻¹, at least 10¹² M⁻¹, at least 5×10¹² M⁻¹, at least 10¹³ M⁻¹, at least 5×10¹³ M⁻¹, at least 10¹⁴ M⁻¹, at least 5×10¹⁴ M⁻¹, at least 10¹⁵ M⁻¹, or at least 5×10¹⁵ M⁻¹. In yet another embodiment, an antibody to be used with the methods of the invention or fragment thereof has a dissociation constant or K_{d} (k_{off}/kₒₙ) of less than 10⁻² M, less than 5×10⁻² M, less than 10⁻³ M, less than 5×10⁻³ M, less than 10⁻⁴ M, less than 5×10⁻⁴ M, less than 10⁻⁵ M, less than 5×10⁻⁵ M, less than 10⁻⁶ M, less than 5×10⁻⁶ M, less than 10⁻⁷ M, less than 5×10⁻⁷ M, less than 10⁻⁸ M, less than 5×10⁻⁸ M, less than 10⁻⁹ M, less than 5×10⁻⁹ M, less than 10⁻¹⁰ M, less than 5×10⁻¹⁰ M, less than 10⁻¹¹ M, less than 5×10⁻¹¹ M, less than 10⁻¹² M, less than 5×10⁻¹² M, less than 10⁻¹³ M, less than 5×10⁻¹³ M, less than 10⁻¹⁴ M, less than 5×10⁻¹⁴ M, less than 10⁻¹⁵ M, or less than 5×10⁻¹⁵ M.

In certain embodiments, an antibody to be used with the methods of the invention or fragment thereof that has a median effective concentration (EC₅₀) of less than 0.01 nM, less than 0.025 nM, less than 0.05 nM, less than 0.1 nM, less than 0.25 nM, less than 0.5 nM, less than 0.75 nM, less than 1 nM, less than 1.25 nM, less than 1.5 nM, less than 1.75 nM, or less than 2 nM, in an in vitro microneutralization assay. The median effective concentration is the concentration of antibody or antibody fragments that neutralizes 50% of the RSV in an in vitro microneutralization assay. In a preferred embodiment, an antibody to be used with the methods of the invention or fragment thereof has an EC₅₀ of less than 0.01 nM, less than 0.025 nM, less than 0.05 nM, less than 0.1 nM, less than 0.25 nM, less than 0.5 nM, less than 0.75 nM, less than 1 nM, less than 1.25 nM, less than 1.5 nM, less than 1.75 nM, or less than 2 nM, in an in vitro microneutralization assay.

In certain embodiments, the antibodies to be used with the methods of the invention are derivatives of anti-RSV antigen, anti-PIV antigen, and/or anti-hMPV antigen antibodies. Standard techniques known to those of skill in the art can be used to introduce mutations in the nucleotide sequence encoding an antibody to be used with the methods of the invention, including, for example, site-directed mutagenesis and PCR-mediated mutagenesis which result in amino acid substitutions. Preferably, the derivatives include less than 25 amino acid substitutions, less than 20 amino acid substitutions, less than 15 amino acid substitutions, less than 10 amino acid substitutions, less than 5 amino acid substitutions, less than 4 amino acid substitutions, less than 3 amino acid substitutions, or less than 2 amino acid substitutions relative to the original molecule. In a preferred embodiment, the derivatives have conservative amino acid substitutions are made at one or more predicted non-essential amino acid residues. A “conservative amino acid substitution” is one in which the amino acid residue is replaced with an amino acid residue having a side chain with a similar charge. Families of amino acid residues having side chains with similar charges have been defined in the art. These families include amino acids with basic side chains (e.g. lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Alternatively, mutations can be introduced randomly along all or part of the coding sequence, such as by saturation mutagenesis, and the resultant mutants can be screened for biological activity to identify mutants that retain activity. Following mutagenesis, the encoded protein can be expressed and the activity of the protein can be determined.

The antibodies to be used with the methods of the invention include derivatives that are modified, i.e, by the covalent attachment of any type of molecule to the antibody such that covalent attachment. For example, but not by way of limitation, the antibody derivatives include antibodies that have been modified, e.g., by glycosylation, acetylation, pegylation, phosphorylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, synthesis in the presence of tunicamycin, etc. Additionally, the derivative may contain one or more non-classical amino acids.

The present invention also provides antibodies of the invention or fragments thereof that comprise a framework region known to those of skill in the art. In certain embodiments, one or more framework regions, preferably, all of the framework regions, of an antibody to be used in the methods of the invention or fragment thereof are human. In certain other embodiments of the invention, the fragment region of an antibody of the invention or fragment thereof is humanized. In certain embodiments, the antibody to be used with the methods of the invention is a synthetic antibody, a monoclonal antibody, an intrabody, a chimeric antibody, a human antibody, a humanized chimeric antibody, a humanized antibody, a glycosylated antibody, a multispecific antibody, a human antibody, a single-chain antibody, or a bispecific antibody.

In certain embodiments of the invention, the antibodies to be used with the invention have half-lives in a mammal, preferably a human, of greater than 12 hours, greater than 1 day, greater than 3 days, greater than 6 days, greater than 10 days, greater than 15 days, greater than 20 days, greater than 25 days, greater than 30 days, greater than 35 days, greater than 40 days, greater than 45 days, greater than 2 months, greater than 3 months, greater than 4 months, or greater than 5 months. Antibodies or antigen-binding fragments thereof having increased in vivo half-lives can be generated by techniques known to those of skill in the art. For example, antibodies or antigen-binding fragments thereof with increased in vivo half-lives can be generated by modifying (e.g., substituting, deleting or adding) amino acid residues identified as involved in the interaction between the Fc domain and the FcRn receptor (see, e.g., PCT Publication No. WO 97/34631 and U.S. patent application No.: Ser. No. 10/020,354, entitled “Molecules with Extended Half-Lives, Compositions and Uses Thereof”, filed Dec. 12, 2001, by Johnson et al., which are incorporated herein by reference in their entireties). Such antibodies or antigen-binding fragments thereof can be tested for binding activity to RSV antigens as well as for in vivo efficacy using methods known to those skilled in the art, for example, by immunoassays described herein.

Further, antibodies or antigen-binding fragments thereof with increased in vivo half-lives can be generated by attaching to said antibodies or antibody fragments polymer molecules such as high molecular weight polyethyleneglycol (PEG). PEG can be attached to said antibodies or antibody fragments with or without a multifunctional linker either through site-specific conjugation of the PEG to the N- or C-terminus of said antibodies or antibody fragments or via epsilon-amino groups present on lysine residues. Linear or branched polymer derivatization that results in minimal loss of biological activity will be used. The degree of conjugation will be closely monitored by SDS-PAGE and mass spectrometry to ensure proper conjugation of PEG molecules to the antibodies. Unreacted PEG can be separated from antibody-PEG conjugates by, e.g., size exclusion or ion-exchange chromatography. PEG-derivatizated antibodies or antigen-binding fragments thereof can be tested for binding activity to RSV antigens as well as for in vivo efficacy using methods known to those skilled in the art, for example, by immunoassays described herein.

In certain embodiments, the antibodies to be used with the methods of the invention are fusion proteins comprising an antibody or fragment thereof that immunospecifically binds to a RSV, PIV, and/or hMPV antigen and a heterologous polypeptide. Preferably, the heterologous polypeptide that the antibody or antibody fragment is fused to is useful for targeting the antibody to respiratory epithelial cells.

In certain embodiments, antibodies to be used with the methods of the invention or fragments thereof disrupt or prevent the interaction between a RSV antigen, a PIV antigen, and/or a hMPV antigen and its host cell receptor.

In certain embodiments, antibodies to be used with the methods of the invention are single-chain antibodies. The design and construction of a single-chain antibody is described in Marasco et al, 1993, Proc Natl Acad Sci 90:7889-7893, which is incorporated herein by reference in its entirety.

In certain embodiments, the antibodies to be used with the invention binds to an intracellular epitope, i.e., are intrabodies. An intrabody comprises at least a portion of an antibody that is capable of immunospecifically binding an antigen and preferably does not contain sequences coding for its secretion. Such antibodies will bind its antigen intracellularly. In one embodiment, the intrabody comprises a single-chain Fv (“sFv”). sFv are antibody fragments comprising the V_{H} and V_{L} domains of antibody, wherein these domains are present in a single polypeptide chain. Generally, the Fv polypeptide further comprises a polypeptide linker between the V_{H} and V_{L} domains which enables the sFv to form the desired structure for antigen binding. For a review of sFv see Pluckthun in The Pharmacology of Monoclonal Antibodies, vol. 113, Rosenburg and Moore eds. Springer-Verlag, New York, pp. 269-315 (1994). In a further embodiment, the intrabody preferably does not encode an operable secretory sequence and thus remains within the cell (see generally Marasco, WA, 1998, “Intrabodies: Basic Research and Clinical Gene Therapy Applications” Springer:New York).

Generation of intrabodies is well-known to the skilled artisan and is described for example in U.S. Pat. Nos. 6,004,940; 6,072,036; 5,965,371, which are incorporated by reference in their entireties herein. Further, the construction of intrabodies is discussed in Ohage and Steipe, 1999, J. Mol. Biol. 291:1119-1128; Ohage et al., 1999, J. Mol. Biol. 291:1129-1134; and Wirtz and Steipe, 1999, Protein Science 8:2245-2250, which references are incorporated herein by reference in their entireties. Recombinant molecular biological techniques such as those described for recombinant production of antibodies (e.g., Section 4.1.2 and 4.1.3) may also be used in the generation of intrabodies. A discussion of intrabodies as antiviral agents can also be found in Marasco, 2001, Curr. Top. Microbiol. Immunol. 260:247-270, which is incorporated by reference herein in its entirety.

In particular, the invention provides methods for treating, preventing, and/or ameliorating one or more symptoms of a respiratory infection by administering either: (i) one or more anti-RSV-antigen intrabodies or fragments thereof and one or more anti-PIV-antigen intrabodies or fragments thereof; (ii) one or more anti-PIV-antigen intrabodies or fragments thereof and one or more anti-hMPV-antigen intrabodies or fragments thereof; or (iii) one or more anti-RSV-antigen intrabodies or fragments thereof, one or more anti-PIV-antigen intrabodies or fragments thereof, and one or more anti-hMPV-antigen intrabodies or fragments thereof. The invention also encompasses administering combinations of intrabodies and antibodies or antigen-binding fragments thereof. For example, but not by way of limitation, a method of the invention comprises administering one or more anti-RSV-antigen antibodies or antigen-binding fragments thereof and one or more anti-hMPV-antigen intrabodies or fragments thereof.

In one embodiment, intrabodies of the invention retain at least about 75% of the binding effectiveness of the complete antibody (i.e., having constant as well as variable regions) to the antigen. More preferably, the intrabody retains at least 85% of the binding effectiveness of the complete antibody. Still more preferably, the intrabody retains at least 90% of the binding effectiveness of the complete antibody. Even more preferably, the intrabody retains at least 95% of the binding effectiveness of the complete antibody.

In producing intrabodies, polynucleotides encoding variable region for both the V_{H} and V_{L} chains of interest can be cloned by using, for example, hybridoma mRNA or splenic mRNA as a template for PCR amplification of such domains (Huse et al., 1989, Science 246:1276). In one preferred embodiment, the polynucleotides encoding the V_{H} and V_{L} domains are joined by a polynucleotide sequence encoding a linker to make a single chain antibody (sFv). The sFv typically comprises a single peptide with the sequence V_{H}-linker-V_{L} or V_{L}-linker-V_{H}. The linker is chosen to permit the heavy chain and light chain to bind together in their proper conformational orientation (see for example, Huston, et al., 1991, Methods in Enzym. 203:46-121, which is incorporated herein by reference). In a further embodiment, the linker can span the distance between its points of fusion to each of the variable domains (e.g., 3.5 nm) to minimize distortion of the native Fv conformation. In such an embodiment, the linker is a polypeptide of at least 5 amino acid residues, at least 10 amino acid residues, at least 15 amino acid residues, or greater. In a further embodiment, the linker should not cause a steric interference with the V_{H} and V_{L} domains of the combining site. In such an embodiment, the linker is 35 amino acids or less, 30 amino acids or less, or 25 amino acids or less. Thus, in a most preferred embodiment, the linker is between 15-25 amino acid residues in length. In a further embodiment, the linker is hydrophilic and sufficiently flexible such that the V_{H} and V_{L} domains can adopt the conformation necessary to detect antigen. Intrabodies can be generated with different linker sequences inserted between identical V_{H} and V_{L} domains. A linker with the appropriate properties for a particular pair of V_{H} and V_{L} domains can be determined empirically by assess the degree of antigen binding for each. Examples of linkers include, but are not limited to, those sequences disclosed in Table 1.

TABLE 1
Sequence

(Gly Gly Gly Gly Ser)₃
Glu Ser Gly Arg Ser Gly Gly Gly Gly Ser Gly Gly Gly Gly Ser
Glu Gly Lys Ser Ser Gly Ser Gly Ser Glu Ser Lys Ser Thr
Glu Gly Lys Ser Ser Gly Ser Gly Ser Glu Ser Lys Ser Thr Gln
Glu Gly Lys Ser Ser Gly Ser Gly Ser Glu Ser Lys Val Asp
Gly Ser Thr Ser Gly Ser Gly Lys Ser Ser Glu Gly Lys Gly
Lys Glu Ser Gly Ser Val Ser Ser Glu Gln Leu Ala Gln Phe Arg Ser
Leu Asp
Glu Ser Gly Ser Val Ser Ser Glu Glu Leu Ala Phe Arg Ser Leu Asp

In one embodiment, intrabodies are expressed in the cytoplasm. In other embodiments, the intrabodies are localized to various intracellular locations. In such embodiments, specific localization sequences can be atached to the intranucleotide polypepetide to direct the intrabody to a specific location. Intrabodies can be localized, for example, to the folowing intracellular locations: endoplasmic reticulum (Munro et al., 1987, Cell 48:899-907; Hangejorden et al., 1991, J. Biol. Chem. 266:6015); nucleus (Lanford et al., 1986, Cell 46:575; Stanton et al., 1986, PNAS 83:1772; Harlow et al., 1985, Mol. Cell Biol. 5:1605); nucleolar region (Seomi et al., 1990, J. Virology 64:1803; Kubota et al., 1989, Biochem. Biophys. Res. Comm. 162:963; Siomi et al., 1998, Cell 55:197); endosomal compartment (Bakke et al., 1990, Cell 63:707-716); mitochondrial matrix (Pugsley, A. P., 1989, “Protein Targeting”, Academic Press, Inc.); Golgi apparatus (Tang et al., 1992, J. Bio. Chem. 267:10122-6); liposomes (Letourneur et al., 1992, Cell 69:1183); and plasma membrane (Marchildon et al., 1984, PNAS 81:7679-82; Henderson et al., 1987, PNAS 89:339-43; Rhee et al., 1987, J. Virol. 61:1045-53; Schultz et al., 1984, J. Virol. 133:431-7; Ootsuyama et al., 1985, Jpn. J Can. Res. 76:1132-5; Ratner et al., 1985, Nature 313:277-84). Examples of localization signals include, but are not limited to, those sequences disclosed in Table 2.

TABLE 2

| Localization | Sequence |
|---|---|
| endoplasmic reticulum | Lys Asp Glu Leu |
| endoplasmic reticulum | Asp Asp Glu Leu |
| endoplasmic reticulum | Asp Glu Glu Leu |
| endoplasmic reticulum | Gln Glu Asp Leu |
| endoplasmic reticulum | Arg Asp Glu Leu |
| nucleus | Pro Lys Lys Lys Arg Lys Val |
| nucleus | Pro Gln Lys Lys Ile Lys Ser |
| nucleus | Gln Pro Lys Lys Pro |
| nucleus | Arg Lys Lys Arg |
| nucleolar region | Arg Lys Lys Arg Arg Gln Arg Arg Arg |
| | Ala His Gln |
| nucleolar region | Arg Gln Ala Arg Arg Asn Arg Arg Arg |
| | Arg Trp Arg Glu Arg Gln Arg |
| nucleolar region | Met Pro Leu Thr Arg Arg Arg Pro Ala Ala |
| | Ser Gln Ala Leu Ala Pro Pro Thr Pro |
| endosomal compartment | Met Asp Asp Gln Arg Asp Leu Ile Ser |
| | Asn Asn Glu Gln Leu Pro |
| mitochondrial matrix | Met Leu Phe Asn Leu Arg Xaa Xaa Leu |
| | Asn Asn Ala Ala Phe Arg His Gly His |
| | Asn Phe Met Val Arg Asn Phe Arg Cys |
| | Gly Gln Pro Leu Xaa |
| plasma membrane | GCVCSSNP |
| plasma membrane | GQTVTTPL |
| plasma membrane | GQELSQHE |
| plasma membrane | GNSPSYNP |
| plasma membrane | GVSGSKGQ |
| plasma membrane | GQTITTPL |
| plasma membrane | GQTLTTPL |
| plasma membrane | GQIFSRSA |
| plasma membrane | GQIHGLSP |
| plasma membrane | GARASVLS |
| plasma membrane | GCTLSAEE |

V_{H} and V_{L} domains are made up of the immunoglobulin domains that generally have a conserved structural disulfide bond. In embodiments where the intrabodies are expressed in a reducing environment (e.g., the cytoplasm), such a structural feature cannot exist. Mutations can be made to the intrabody polypeptide sequence to compensate for the decreased stability of the immunoglobulin structure resulting from the absence of disulfide bond formation. In one embodiment, the V_{H} and/or V_{L} domains of the intrabodies contain one or more point mutations such that their expression is stabilized in reducing environments (see Steipe et al., 1994, J. Mol. Biol. 240:188-92; Wirtz and Steipe, 1999, Protein Science 8:2245-50; Ohage and Steipe, 1999, J. Mol. Biol. 291:1119-28; Ohage et al., 1999, J. Mol. Biol. 291:1129-34).

4.1.1 Methods for Producing Antibodies

The antibodies to be used with the methods of the invention or fragments thereof can is be produced by any method known in the art for the synthesis of antibodies, in particular, by chemical synthesis or preferably, by recombinant expression techniques.

Polyclonal antibodies to a RSV, PIV, and/or hMPV antigen can be produced by various procedures well known in the art. For example, a RSV, PIV, and/or hMPV antigen can be administered to various host animals including, but not limited to, rabbits, mice, rats, etc. to induce the production of sera containing polyclonal antibodies specific for the RSV, PIV, and/or hMPV antigen. Various adjuvants may be used to increase the immunological response, depending on the host species, and include but are not limited to, Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and Corynebacterium parvum. Such adjuvants are also well known in the art.

Monoclonal antibodies can be prepared using a wide variety of techniques known in the art including the use of hybridoma, recombinant, and phage display technologies, or a combination thereof. For example, monoclonal antibodies can be produced using hybridoma techniques including those known in the art and taught, for example, in Harlow et al., Antibodies: A Laboratory Manual, (Cold Spring Harbor Laboratory Press, 2nd ed. 1988); Hammerling, et al., in: Monoclonal Antibodies and T-Cell Hybridomas 563-681 (Elsevier, N.Y., 1981) (said references incorporated by reference in their entireties). The term “monoclonal antibody” as used herein is not limited to antibodies produced through hybridoma technology. The term “monoclonal antibody” refers to an antibody that is derived from a single clone, including any eukaryotic, prokaryotic, or phage clone, and not the method by which it is produced.

Methods for producing and screening for specific antibodies using hybridoma technology are routine and well known in the art. Briefly, mice can be immunized with a RSV, PIV, and/or hMPV antigen and once an immune response is detected, e.g., antibodies specific for the RSV, PIV, and/or hMPV antigen are detected in the mouse serum, the mouse spleen is harvested and splenocytes isolated. The splenocytes are then fused by well known techniques to any suitable myeloma cells, for example cells from cell line SP20 available from the ATCC. Hybridomas are selected and cloned by limited dilution. The hybridoma clones are then assayed by methods known in the art for cells that secrete antibodies capable of binding a polypeptide of the invention. Ascites fluid, which generally contains high levels of antibodies, can be generated by immunizing mice with positive hybridoma clones.

In a specific embodiment, an antigen of APV is used to generate antibodies agains hMPV.

In certain embodiments, a method of generating monoclonal antibodies comprises culturing a hybridoma cell secreting an antibody of the invention wherein, preferably, the hybridoma is generated by fusing splenocytes isolated from a mouse immunized with a RSV, PIV, and/or hMPV antigen with myeloma cells and then screening the hybridomas resulting from the fusion for hybridoma clones that secrete an antibody able to bind a RSV, PIV, and/or hMPV antigen.

Antibody fragments which recognize specific RSV, PIV, and/or hMPV epitopes may be generated by any technique known to those of skill in the art. For example, Fab and F(ab′)2 fragments of the invention may be produced by proteolytic cleavage of immunoglobulin molecules, using enzymes such as papain (to produce Fab fragments) or pepsin (to produce F(ab′)2 fragments). F(ab′)2 fragments contain the variable region, the light chain constant region and the CH1 domain of the heavy chain. Further, the antibodies to be used with the present invention can also be generated using various phage display methods known in the art.

In phage display methods, functional antibody domains are displayed on the surface of phage particles which carry the polynucleotide sequences encoding them. In particular, DNA sequences encoding VH and VL domains are amplified from animal cDNA libraries (e.g., human or murine cDNA libraries of lymphoid tissues). The DNA encoding the VH and VL domains are recombined together with an scFv linker by PCR and cloned into a phagemid vector (e.g., p CANTAB 6 or pComb 3 HSS). The vector is electroporated in E. coli and the E. coli is infected with helper phage. Phage used in these methods are typically filamentous phage including fd and M13 and the VH and VL domains are usually recombinantly fused to either the phage gene III or gene VIII. Phage expressing an antigen binding domain that binds to a RSV, PIV, and/or hMPV antigen of interest can be selected or identified with antigen, e.g., using labeled antigen or antigen bound or captured to a solid surface or bead. Examples of phage display methods that can be used to make the antibodies of the present invention include those disclosed in Brinkman et al., 1995, J. Immunol. Methods 182:41-50; Ames et al., 1995, J. Immunol. Methods 184:177-186; Kettleborough et al., 1994, Eur. J. Immunol. 24:952-958; Persic et al., 1997, Gene 187:9-18; Burton et al., 1994, Advances in Immunology 57:191-280; PCT application No. PCT/GB91/O1 134; PCT publication Nos. WO 90/02809, WO 91/10737, WO 92/01047, WO 92/18619, WO 93/1 1236, WO 95/15982, WO 95/20401, and WO97/13844; and U.S. Pat. Nos. 5,698,426, 5,223,409, 5,403,484, 5,580,717, 5,427,908, 5,750,753, 5,821,047, 5,571,698, 5,427,908, 5,516,637, 5,780,225, 5,658,727, 5,733,743 and 5,969,108; each of which is incorporated herein by reference in its entirety.

As described in the above references, after phage selection, the antibody coding regions from the phage can be isolated and used to generate whole antibodies, including human antibodies, or any other desired antigen binding fragment, and expressed in any desired host, including mammalian cells, insect cells, plant cells, yeast, and bacteria, e.g., as described below. Techniques to recombinantly produce Fab, Fab′ and F(ab′)2 fragments can also be employed using methods known in the art such as those disclosed in PCT publication No. WO 92/22324; Mullinax et al., 1992, BioTechniques 12(6):864-869; Sawai et al., 1995, AJRI 34:26-34; and Better et al., 1988, Science 240:1041-1043 (said references incorporated by reference in their entireties).

To generate whole antibodies, PCR primers including VH or VL nucleotide sequences, a restriction site, and a flanking sequence to protect the restriction site can be used to amplify the VH or VL sequences in scFv clones. Utilizing cloning techniques known to those of skill in the art, the PCR amplified VH domains can be cloned into vectors expressing a VH constant region, e.g., the human gamma 4 constant region, and the PCR amplified VL domains can be cloned into vectors expressing a VL constant region, e.g., human kappa or lamba constant regions. Preferably, the vectors for expressing the VH or VL domains comprise an EF-1α promoter, a secretion signal, a cloning site for the variable domain, constant domains, and a selection marker such as neomycin. The VH and VL domains may also cloned into one vector expressing the necessary constant regions. The heavy chain conversion vectors and light chain conversion vectors are then co-transfected into cell lines to generate stable or transient cell lines that express full-length antibodies, e.g., IgG, using techniques known to those of skill in the art.

For some uses, including in vivo use of antibodies in humans and in vitro detection assays, it may be preferable to use human or chimeric antibodies. Completely human antibodies are particularly desirable for therapeutic treatment of human subjects. Human antibodies can be made by a variety of methods known in the art including phage display methods described above using antibody libraries derived from human immunoglobulin sequences or synthetic sequences homologous to human immunoglobulin sequences. See also U.S. Pat. Nos. 4,444,887 and 4,716,111; and PCT publications WO 98/46645, WO 98/50433, WO 98/24893, WO98/16654, WO 96/34096, WO 96/33735, and WO 91/10741; each of which is incorporated herein by reference in its entirety.

Human antibodies can also be produced using transgenic mice which are incapable of expressing functional endogenous immunoglobulins, but which can express human immunoglobulin genes. For example, the human heavy and light chain immunoglobulin gene complexes may be introduced randomly or by homologous recombination into mouse embryonic stem cells. Alternatively, the human variable region, constant region, and diversity region may be introduced into mouse embryonic stem cells in addition to the human heavy and light chain genes. The mouse heavy and light chain immunoglobulin genes may be rendered non-functional separately or simultaneously with the introduction of human immunoglobulin loci by homologous recombination. In particular, homozygous deletion of the JH region prevents endogenous antibody production. The modified embryonic stem cells are expanded and microinjected into blastocysts to produce chimeric mice. The chimeric mice are then be bred to produce homozygous offspring which express human antibodies. The transgenic mice are immunized in the normal fashion with a selected antigen, e.g., all or a portion of a polypeptide of the invention. Monoclonal antibodies directed against the antigen can be obtained from the immunized, transgenic mice using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA, IgM and IgE antibodies. For an overview of this technology for producing human antibodies, see Lonberg and Huszar (1995, Int. Rev. Immunol. 13:65-93). For a detailed discussion of this technology for producing human antibodies and human monoclonal antibodies and protocols for producing such antibodies, see, e.g., PCT publication Nos. WO 98/24893, WO 96/34096, and WO 96/33735; and U.S. Pat. Nos. 5,413,923, 5,625,126, 5,633,425, 5,569,825, 5,661,016, 5,545,806, 5,814,318, and 5,939,598, which are incorporated by reference herein in their entireties. In addition, companies such as Medarex, Inc. (Princeton, N.J.), Abgenix, Inc. (Freemont, Calif.) and Genpharm (San Jose, Calif.) can be engaged to provide human antibodies directed against a selected antigen using technology similar to that described above.

A chimeric antibody is a molecule in which different portions of the antibody are derived from different immunoglobulin molecules such as antibodies having a variable region derived from a non-human (e.g., murine) antibody and a human immunoglobulin constant region. Methods for producing chimeric antibodies are known in the art. See e.g., Morrison, 1985, Science 229:1202; Oi et al., 1986, BioTechniques 4:214; Gillies et al., 1989, J. Immunol. Methods 125:191-202; and U.S. Pat. Nos. 5,807,715, 4,816,567, and 4,816,397, which are incorporated herein by reference in their entireties. Chimeric antibodies comprising one or more CDRs from human species and framework regions from a non-human immunoglobulin molecule can be produced using a variety of techniques known in the art including, for example, CDR-grafting (EP 239,400; PCT publication No. WO 91/09967; and U.S. Pat. Nos. 5,225,539, 5,530,101, and 5,585,089), veneering or resurfacing (EP 592,106; EP 519,596; Padlan, 1991, Molecular Immunology 28(4/5):489-498; Studnicka et al., 1994, Protein Engineering 7(6):805-814; and Roguska et al., 1994, PNAS 91:969-973), and chain shuffling (U.S. Pat. No. 5,565,332). In a preferred embodiment, antibodies comprise one or more CDRs listed in Table 3 (preferably all CDRs) and human framework regions. Often, framework residues in the framework regions will be substituted with the corresponding residue from the CDR donor antibody to alter, preferably improve, antigen binding. These framework substitutions are identified by methods well known in the art, e.g., by modeling of the interactions of the CDR and framework residues to identify framework is residues important for antigen binding and sequence comparison to identify unusual framework residues at particular positions. (See, e.g., Queen et al., U.S. Pat. No. 5,585,089; and Riechmann et al., 1988, Nature 332:323, which are incorporated herein by reference in their entireties.)

Further, the antibodies to be used with the methods of the invention can, in turn, be utilized to generate anti-idiotype antibodies that “mimic” RSV, PIV, and/or hMPV antigens using techniques well known to those skilled in the art. (See, e.g., Greenspan & Bona, 1989, FASEB J. 7(5):437-444; and Nissinoff, 1991, J. Immunol. 147(8):2429-2438). For example, antibodies of the invention which bind to and competitively inhibit the binding of RSV, PIV, and/or hMPV (as determined by assays well known in the art) to its host cell receptor can be used to generate anti-idiotypes that “mimic” a RSV, PUV, and/or hMPV antigen and bind to the RSV, PIV, and/or hMPV receptors, i.e., compete with the virus for binding to the host cell, therefore decreasing the infection rate of host cells with virus.

In certain other embodiments, anti-anti-idiotypes, generated by techniques well-known to the skilled artisan, are used in the methods of the invention. Such anti-anti-idiotypes mimic the binding domain of the anti-RSV, -PIV, and/or -hMPV antibody and, as a consequence, bind to and neutralize RSV, PIV, and/or hMPV. Such neutralizing anti-anti-idiotypes or Fab fragments of such anti-anti-idiotypes can be used in therapeutic regimens to neutralize RSV, PIV, and/or hMPV. For example, such anti-anti-idiotypic antibodies can be used to bind RSV, PIV, and/or hMPV and thereby prevent infection.

In certain embodiments, a fragment of a protein of RSV, PIV, or hMPV is used as an immunogen for the generation of antibodies to be used with the methods of the invention. A fragment of a protein of RSV, PIV, or hMPV to be used as an immunogen can be at least 10, 20, 30, 40, 50, 75, 100, 250, 500, 750, or at least 1000 amino acids in length. In certain embodiments a synthetic peptide of a protein of RSV, PIV, or hMPV is used as an immunogen.

In certain embodiments, fragments of viral antigens are used as immunogen to produce antibodies to be used with the methods of the invention. In certain embodiments, fragments preferably contain a sequence of at least 4, at least 5, at least 6, at least 7, more preferably at least 8, at least 9, at least 10, at least 11, at least 12, at least 13, at least 14, at least 15, at least 20, at least 25, at least 30, at least 40, at least 50, at least 75 or at least 100 amino acids. In certain, more specific embodiments, a fragment is about 15 to about 30 amino acids long. Preferred polypeptides comprising immunogenic or antigenic epitopes are at least 10, 15, 20, 25, 30, 35, 40, 45, 50, 55, 60, 65, 70, 75, 80, 85, 90, 95, or 100 amino acid residues in length. Additional non-exclusive preferred antigenic epitopes include the antigenic epitopes disclosed herein, as well as portions thereof.

4.1.2 Polynucleotides Encoding an Antibody

Polynucleotides encoding antibodies to be used with the invention may be obtained, and the nucleotide sequence of the polynucleotides determined, by any method known in the art. Since amino acid sequences of some antibodies are known (as described in Table 2), nucleotide sequences encoding these antibodies can be determined using methods well known in the art, i.e., nucleotide codons known to encode particular amino acids are assembled in such a way to generate a nucleic acid that encodes the antibody or fragment thereof of the invention. Such a polynucleotide encoding the antibody may be assembled from chemically synthesized oligonucleotides (e.g., as described in Kutmeier et al., 1994, BioTechniques 17:242), which, briefly, involves the synthesis of overlapping oligonucleotides containing portions of the sequence encoding the antibody, annealing and ligating of those oligonucleotides, and then amplification of the ligated oligonucleotides by PCR.

Alternatively, a polynucleotide encoding an antibody may be generated from nucleic acid from a suitable source. If a clone containing a nucleic acid encoding a particular antibody is not available, but the sequence of the antibody molecule is known, a nucleic acid encoding the immunoglobulin may be chemically synthesized or obtained from a suitable source (e.g., an antibody cDNA library, or a cDNA library generated from, or nucleic acid, preferably poly A+ RNA, isolated from, any tissue or cells expressing the antibody, such as hybridoma cells selected to express an antibody of the invention) by PCR amplification using synthetic primers hybridizable to the 3′ and 5′ ends of the sequence or by cloning using an oligonucleotide probe specific for the particular gene sequence to identify, e.g., a cDNA clone from a cDNA library that encodes the antibody. Amplified nucleic acids generated by PCR may then be cloned into replicable cloning vectors using any method well known in the art.

Once the nucleotide sequence of the antibody is determined, the nucleotide sequence of the antibody may be manipulated using methods well known in the art for the manipulation of nucleotide sequences, e.g., recombinant DNA techniques, site directed mutagenesis, PCR, etc. (see, for example, the techniques described in Sambrook et al., 1990, Molecular Cloning, A Laboratory Manual, 2d Ed., Cold Spring Harbor Laboratory, Cold Spring Harbor, N.Y. and Ausubel et al., eds., 1998, Current Protocols in Molecular Biology, John Wiley & Sons, NY, which are both incorporated by reference herein in their entireties), to generate antibodies having a different amino acid sequence, for example to create amino acid substitutions, deletions, and/or insertions.

In a specific embodiment, one or more of the CDRs is inserted within framework regions using routine recombinant DNA techniques. The framework regions may be naturally occurring or consensus framework regions, and preferably human framework regions (see, e.g., Chothia et al., 1998, J. Mol. Biol. 278: 457-479 for a listing of human framework regions). Preferably, the polynucleotide generated by the combination of the framework regions and CDRs encodes an antibody that specifically binds to a RSV, PIV, and/or hMPV antigen. In certain embodiments, one or more amino acid substitutions may be made within the framework regions, and, preferably, the amino acid substitutions improve binding of the antibody to its antigen. Additionally, such methods may be used to make amino acid substitutions or deletions of one or more variable region cysteine residues participating in an intrachain disulfide bond to generate antibody molecules lacking one or more intrachain disulfide bonds. Other alterations to the polynucleotide are encompassed by the present invention and within the skill of the art.

4.1.3 Recombinant Expression of an Antibody

Recombinant expression of an antibody to be used with the methods of the invention, derivative or analog thereof, (e.g., a heavy or light chain of an antibody of the invention or a portion thereof or a single chain antibody of the invention), requires construction of an expression vector containing a polynucleotide that encodes the antibody. Once a polynucleotide encoding an antibody molecule or a heavy or light chain of an antibody, or portion thereof (preferably, but not necessarily, containing the heavy or light chain variable domain), of the invention has been obtained, the vector for the production of the antibody molecule may be produced by recombinant DNA technology using techniques well known in the art. Thus, methods for preparing a protein by expressing a polynucleotide containing an antibody encoding nucleotide sequence are described herein. Methods which are well known to those skilled in the art can be used to construct expression vectors containing antibody coding sequences and appropriate transcriptional and translational control signals. These methods include, for example, in vitro recombinant DNA techniques, synthetic techniques, and in vivo genetic recombination. The invention, thus, provides replicable vectors comprising a nucleotide sequence encoding an antibody molecule of the invention, a heavy or light chain of an antibody, a heavy or light chain variable domain of an antibody or a portion thereof, or a heavy or light chain CDR, operably linked to a promoter. Such vectors may include the nucleotide sequence encoding the constant region of the antibody molecule (see, e.g., PCT Publication WO 86/05807; PCT Publication WO 89/01036; and U.S. Pat. No. 5,122,464) and the variable domain of the antibody may be cloned into such a vector for expression of the entire heavy, the entire light chain, or both the entire heavy and light chains.

The expression vector is transferred to a host cell by conventional techniques and the transfected cells are then cultured by conventional techniques to produce an antibody of the invention. Thus, the invention includes host cells containing a polynucleotide encoding an antibody of the invention or fragments thereof, or a heavy or light chain thereof, or portion thereof, or a single chain antibody of the invention, operably linked to a heterologous promoter. In preferred embodiments for the expression of double-chained antibodies, vectors encoding both the heavy and light chains may be co-expressed in the host cell for expression of the entire immunoglobulin molecule, as detailed below.

A variety of host-expression vector systems may be utilized to express the antibody molecules of the invention (see, e.g., U.S. Pat. No. 5,807,715). Such host-expression systems represent vehicles by which the coding sequences of interest may be produced and subsequently purified, but also represent cells which may, when transformed or transfected with the appropriate nucleotide coding sequences, express an antibody molecule of the invention in situ. These include but are not limited to microorganisms such as bacteria (e.g., E. coli and B. subtilis) transformed with recombinant bacteriophage DNA, plasmid DNA or cosmid DNA expression vectors containing antibody coding sequences; yeast (e.g., Saccharomyces Pichia) transformed with recombinant yeast expression vectors containing antibody coding sequences; insect cell systems infected with recombinant virus expression vectors (e.g., baculovirus) containing antibody coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing antibody coding sequences; or mammalian cell systems (e.g., COS, CHO, BHK, 293, NSO, and 3T3 cells) harboring recombinant expression constructs containing promoters derived from the genome of mammalian cells (e.g., metallothionein promoter) or from mammalian viruses (e.g., the adenovirus late promoter; the vaccinia virus 7.5K promoter). Preferably, bacterial cells such as Escherichia coli, and more preferably, eukaryotic cells, especially for the expression of whole recombinant antibody molecule, are used for the expression of a recombinant antibody molecule. For example, mammalian cells such as Chinese hamster ovary cells (CHO), in conjunction with a vector such as the major intermediate early gene promoter element from human cytomegalovirus is an effective expression system for antibodies (Foecking et al., 1986, Gene 45:101; and Cockett et al., 1990, Bio/Technology 8:2). In a specific embodiment, the expression of nucleotide sequences encoding antibodies or antigen-binding fragments thereof which immunospecifically bind to one or more RSV antigens is regulated by a constitutive promoter, inducible promoter or tissue specific promoter.

In bacterial systems, a number of expression vectors may be advantageously selected depending upon the use intended for the antibody molecule being expressed. For example, when a large quantity of such a protein is to be produced, for the generation of pharmaceutical compositions of an antibody molecule, vectors which direct the expression of high levels of fusion protein products that are readily purified may be desirable. Such vectors include, but are not limited to, the E. coli expression vector pUR278 (Ruther et al., 1983, EMBO 12:1791), in which the antibody coding sequence may be ligated individually into the vector in frame with the lac Z coding region so that a fusion protein is produced; pIN vectors (Inouye & Inouye, 1985, Nucleic Acids Res. 13:3101-3109; Van Heeke & Schuster, 1989, J. Biol. Chem. 24:5503-5509); and the like. pGEX vectors may also be used to express foreign polypeptides as fusion proteins with glutathione 5-transferase (GST). In general, such fusion proteins are soluble and can easily be purified from lysed cells by adsorption and binding to matrix glutathione agarose beads followed by elution in the presence of free glutathione. The pGEX vectors are designed to include thrombin or factor Xa protease cleavage sites so that the cloned target gene product can be released from the GST moiety.

In an insect system, Autographa californica nuclear polyhedrosis virus (AcNPV) is used as a vector to express foreign genes. The virus grows in Spodoptera frugiperda cells. The antibody coding sequence may be cloned individually into non-essential regions (for example, the polyhedrin gene) of the virus and placed under control of an AcNPV promoter (for example, the polyhedrin promoter).

In mammalian host cells, a number of viral-based expression systems may be utilized.

In cases where an adenovirus is used as an expression vector, the antibody coding sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing the antibody molecule in infected hosts (e.g., see Logan & Shenk, 1984, Proc. Natl. Acad. Sci. USA 81:355-359). Specific initiation signals may also be required for efficient translation of inserted antibody coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see, e.g., Bittner et al., 1987, Methods in Enzymol. 153:516-544).

In addition, a host cell strain may be chosen which modulates the expression of the inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product may be used. Such mammalian host cells include but are not limited to CHO, VERY, BHK, Hela, COS, MDCK, 293, 3T3, W138, BT483, Hs578T, HTB2, BT20 and T47D, NS0 (a murine myeloma cell line that does not endogenously produce any immunoglobulin chains), CRL7O3O and HsS78Bst cells.

For long-term, high-yield production of recombinant proteins, stable expression is preferred. For example, cell lines which stably express the antibody molecule may be engineered. Rather than using expression vectors which contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines which express the antibody molecule. Such engineered cell lines may be particularly useful in screening and evaluation of compositions that interact directly or indirectly with the antibody molecule.

A number of selection systems may be used, including but not limited to, the herpes simplex virus thymidine kinase (Wigler et al., 1977, Cell 11:223), hypoxanthineguanine phosphoribosyltransferase (Szybalska & Szybalski, 1992, Proc. Natl. Acad. Sci. USA 48:202), and adenine phosphoribosyltransferase (Lowy et al., 1980, Cell 22:8-17) genes can be employed in tk-, hgprt- or aprt-cells, respectively. Also, antimetabolite resistance can be used as the basis of selection for the following genes: dhfr, which confers resistance to methotrexate (Wigler et al., 1980, Natl. Acad. Sci. USA 77:357; O'Hare et al., 1981, Proc. Natl. Acad. Sci. USA 78:1527); gpt, which confers resistance to mycophenolic acid (Mulligan & Berg, 1981, Proc. Natl. Acad. Sci. USA 78:2072); neo, which confers resistance to the aminoglycoside G-418 (Wu and Wu, 1991, Biotherapy 3:87-95; Tolstoshev, 1993, Ann. Rev. Pharmacol. Toxicol. 32:573-596; Mulligan, 1993, Science 260:926-932; and Morgan and Anderson, 1993, Ann. Rev. Biochem. 62: 191-217; May, 1993, TIB TECH 11(5):155-215); and hygro, which confers resistance to hygromycin (Santerre et al., 1984, Gene 30:147). Methods commonly known in the art of recombinant DNA technology may be routinely applied to select the desired recombinant clone, and such methods are described, for example, in Ausubel et al. (eds.), Current Protocols in Molecular Biology, John Wiley & Sons, NY (1993); Kriegler, Gene Transfer and Expression, A Laboratory Manual, Stockton Press, NY (1990); and in Chapters 12 and 13, Dracopoli et al. (eds), Current Protocols in Human Genetics, John Wiley & Sons, NY (1994); Colberre-Garapin et al., 1981, J. Mol. Biol. 150:1, which are incorporated by reference herein in their entireties.

The expression levels of an antibody molecule can be increased by vector amplification (for a review, see Bebbington and Hentschel, The use of vectors based on gene amplification for the expression of cloned genes in mammalian cells in DNA cloning, Vol.3. (Academic Press, New York, 1987)). When a marker in the vector system expressing antibody is amplifiable, increase in the level of inhibitor present in culture of host cell will increase the number of copies of the marker gene. Since the amplified region is associated with the antibody gene, production of the antibody will also increase (Crouse et al., 1983, Mol. Cell. Biol. 3:257).

The host cell may be co-transfected with two expression vectors of the invention, the first vector encoding a heavy chain derived polypeptide and the second vector encoding a light chain derived polypeptide. The two vectors may contain identical selectable markers which enable equal expression of heavy and light chain polypeptides. Alternatively, a single vector may be used which encodes, and is capable of expressing, both heavy and light chain polypeptides. In such situations, the light chain should be placed before the heavy chain to avoid an excess of toxic free heavy chain (Proudfoot, 1986, Nature 322:52; and Kohler, 1980, Proc. Natl. Acad. Sci. USA 77:2 197). The coding sequences for the heavy and light chains may comprise cDNA or genomic DNA.

Once an antibody molecule to be used with the methods of the invention has been produced by recombinant expression, it may be purified by any method known in the art for purification of an immunoglobulin molecule, for example, by chromatography (e.g., ion exchange, affinity, particularly by affinity for the specific antigen after Protein A, and sizing column chromatography), centrifugation, differential solubility, or by any other standard technique for the purification of proteins. Further, the antibodies of the present invention or fragments thereof may be fused to heterologous polypeptide sequences described herein or otherwise known in the art to facilitate purification.

4.1.4 BiTE Technology

In certain embodiments, antibodies to be used with the methods of the invention and antibodies of the pharmaceutical compositions of the invention are bispecific T cell engagers (BiTEs). Bispecific T cell engagers (BiTE) are bispecific antibodies that can redirect T cells for antigen-specific elimination of targets. A BiTE molecule has an antigen-binding domain that binds to a T cell antigen (e.g. CD3) at one end of the molecule and an antigen binding domain that will bind to an antigen on the target cell. A BiTE molecule was recently described in WO 99/54440, which is herein incorporated by reference. This publication describes a novel single-chain multifunctional polypeptide that comprises binding sites for the CD19 and CD3 antigens (CD19×CD3). This molecule was derived from two antibodies, one that binds to CD19 on the B cell and an antibody that binds to CD3 on the T cells. The variable regions of these different antibodies are linked by a polypeptide sequence, thus creating a single molecule. Also described, is the linking of the variable heavy chain (VH) and light chain (VL) of a specific binding domain with a flexible linker to create a single chain, bispecific antibody.

In an embodiment of this invention, an antibody or a fragment thereof that immunospecifically binds a polypeptide of interest (e.g., an antigen of MPV, RSV and/or PIV) will comprise a portion of the BiTE molecule. For example, the VH and/or VL (preferably a scFV) of an antibody that binds a polypeptide of interest (e.g., an antigen of MPV, RSV and/or PIV) can be fused to an anti-CD3 binding portion such as that of the molecule described above, thus creating a BiTE molecule that targets the polypeptide of interest (e.g., an antigen of MPV, RSV and/or PIV). In addition to the variable heavy and or light chain of antibody against a polypeptide of interest (e.g., an antigen of MPV, RSV and/or PIV), other molecules that bind the polypeptide of interest (e.g., an antigen of MPV, RSV and/or PIV) can comprise the BiTE molecule, for example antiviral compounds. In another embodiment, the BiTE molecule can comprise a molecule that binds to other T cell antigens (other than CD3). For example, ligands and/or antibodies that immunospecifically bind to T-cell antigens like CD2, CD4, CD8, CD11a, TCR, and CD28 are contemplated to be part of this invention. This list is not meant to be exhaustive but only to illustrate that other molecules that can immunospecifically bind to a T cell antigen can be used as part of a BiTE molecule. These molecules can include the VH and/or VL portions of the antibody or natural ligands (for example LFA3 whose natural ligand is CD3). A BiTE molecule can be an antagonist.

The “binding domain” as used in accordance with the present invention denotes a domain comprising a three-dimensional structure capable of specifically binding to an epitope like native antibodies, free scFv fragments or one of their corresponding immunoglobulin chains, preferably the VH chain. Thus, said domain can comprise the VH and/or VL domain of an antibody or an immunoglobulin chain, preferably at least the VH domain or more preferably the VH and VL domain linked by a flexible polypeptide linker (scFv). On the other hand, said binding domain contained in the polypeptide of interest may comprise at least one complementarity determining region (CDR) of an antibody or immunoglobulin chain recognizing an antigen on the T cell or a cellular antigen. In this respect, it is noted that the binding domain present in the polypeptide of interest may not only be derived from antibodies but also from other T cell or cellular antigen binding protein, such as naturally occurring surface receptors or ligands. It is further contemplated that in an embodiment of the invention, said first and or second domain of the above-described polypeptide mimic or correspond to a VH and VL region from a natural antibody. The antibody providing the binding site for the polypeptide of interest can be, e.g., a monoclonal antibody, polyclonal antibody, chimeric antibody, humanized antibody, bispecific antibody, synthetic antibody, antibody fragment, such as Fab, Fv or scFv fragments etc., or a chemically modified derivative of any of these.

4.1.5 Antibody Conjugates

In certain embodiments, the antibodies to be used with the methods of the invention or fragments thereof are recombinantly fused or chemically conjugated (including both covalently and non-covalently conjugations) to a heterologous polypeptide (or portion thereof, preferably at least 10, at least 20, at least 30, at least 40, at least 50, at least 60, at least 70, at least 80, at least 90 or at least 100 amino acids of the polypeptide) to generate fusion proteins. The fusion does not necessarily need to be direct, but may occur through linker sequences. For example, antibodies may be used to target heterologous polypeptides to particular cell types (e.g., respiratory epithelial cells), either in vitro or in vivo, by fusing or conjugating the antibodies to antibodies specific for particular cell surface receptors. Antibodies fused or conjugated to heterologous polypeptides may also be used in in vitro immunoassays and purification methods using methods known in the art. See e.g., PCT publication WO 93/21232; EP 439,095; Naramura et al., Immunol. Lett. 39:91-99 (1994); U.S. Pat. No. 5,474,981; Gillies et al., PNAS 89:1428-1432 (1992); and Fell et al., J. Immunol. 146:2446-2452(1991), which are incorporated by reference in their entireties.

In certain embodiments, the anti-RSV-antigen antibody is an antibody conjugate. In other embodiments, the anti-PIV-antigen antibody is an antibody conjugate. In other embodiments, the anti-hMPV-antigen antibody is an antibody conjugate.

Additional fusion proteins of the antibodies to be used with the methods of the invention or fragments thereof may be generated through the techniques of gene-shuffling, motif-shuffling, exon-shuffling, and/or codon-shuffling (collectively referred to as “DNA shuffling”). DNA shuffling may be employed to alter the activities of antibodies of the invention or fragments thereof (e.g., antibodies or antigen-binding fragments thereof with higher affinities and lower dissociation rates). See, generally, U.S. Pat. Nos. 5,605,793; 5,811,238; 5,830,721; 5,834,252; and 5,837,458, and Patten et al., Curr. Opinion Biotechnol. 8:724-33 (1997); Harayama, Trends Biotechnol. 16(2):76-82 (1998); Hansson, et al., J. Mol. Biol. 287:265-76 (1999); and Lorenzo and Blasco, Biotechniques 24(2):308-13 (1998) (each of these patents and publications are hereby incorporated by reference in its entirety). In one embodiment, antibodies or antigen-binding fragments thereof, or the encoded antibodies or antigen-binding fragments thereof, may be altered by being subjected to random mutagenesis by error-prone PCR, random nucleotide insertion or other methods prior to recombination. In another embodiment, one or more portions of a polynucleotide encoding an antibody or antibody fragment, which portions immunospecifically bind to a RSV, PIV, and/or hMPV antigen may be recombined with one or more components, motifs, sections, parts, domains, fragments, etc. of one or more heterologous molecules.

Moreover, the antibodies to be used with the methods of the present invention or fragments thereof can be fused to marker sequences, such as a peptide to facilitate purification. In preferred embodiments, the marker amino acid sequence is a hexa-histidine peptide, such as the tag provided in a pQE vector (QIAGEN, Inc., 9259 Eton Avenue, Chatsworth, Calif., 91311), among others, many of which are commercially available. As described in Gentz et al., 1989, Proc. Natl. Acad. Sci. USA 86:821-824, for instance, hexa-histidine provides for convenient purification of the fusion protein. Other peptide tags useful for purification include, but are not limited to, the hemagglutinin “HA” tag, which corresponds to an epitope derived from the influenza hemagglutinin protein (Wilson et al., 1984, Cell 37:767) and the “flag” tag.

An antibody or fragment thereof may be conjugated to a therapeutic moiety such as, but not limited to, a cytotoxin, e.g., a cytostatic or cytocidal agent, a therapeutic agent or a radioactive metal ion, e.g., alpha-emitters. A cytotoxin or cytotoxic agent includes, but is not limited to, any agent that is detrimental to cells. Examples include, but are not limited to, paclitaxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), anti-mitotic agents (e.g., vincristine and vinblastine), and antivirals, such as, but not limited to: nucleoside analogs, such as zidovudine, acyclovir, gangcyclovir, vidarabine, idoxuridine, trifluridine, and ribavirin, as well as foscamet, amantadine, rimantadine, saquinavir, indinavir, ritonavir, and the alpha-interferons.

Further, an antibody to be used with the methods of the invention or fragment thereof may be conjugated to a therapeutic agent or drug moiety that modifies a given biological response. Therapeutic agents or drug moieties are not to be construed as limited to classical chemical therapeutic agents. For example, the drug moiety may be a protein or polypeptide possessing a desired biological activity. Such proteins may include, but are not limited to, a toxin such as abrin, ricin A, pseudomonas exotoxin, or diphtheria toxin; a protein such as tumor necrosis factor, α-interferon, β-interferon, nerve growth factor, platelet derived growth factor, tissue plasminogen activator, an apoptotic agent, e.g., TNF-α, TNF-β, AIM I (see, International Publication No. WO 97/33899), AIM II (see, International Publication No. WO 97/34911), Fas Ligand (Takahashi et al., 1994, J. Iminunol., 6:1567-1574), and VEGI (see, International Publication No. WO 99/23105), a thrombotic agent or an anti-angiogenic agent, e.g., angiostatin or endostatin; or, a biological response modifier such as, for example, a lymphokine (e.g., interleukin-1 (“IL-1”), interleukin-2 (“IL-2”), interleukin-6 (“IL-6”), granulocyte macrophage colony stimulating factor (“GM-CSF”), and granulocyte colony stimulating factor (“G-CSF”)), or a growth factor (e.g., growth hormone (“GH”)).

Techniques for conjugating such therapeutic moieties to antibodies are well known, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., 1982, Immunol. Rev. 62:119-58.

An antibody or fragment thereof, with or without a therapeutic moiety conjugated to it, administered alone or in combination with cytotoxic factor(s) and/or cytokine(s) can be used as a therapeutic.

Alternatively, an antibody can be conjugated to a second antibody to form an antibody heteroconjugate as described by Segal in U.S. Pat. No. 4,676,980, which is incorporated herein by reference in its entirety.

Antibodies may also be attached to solid supports, which are particularly useful for immunoassays or purification of the target antigen. Such solid supports include, but are not limited to, glass, cellulose, polyacrylamide, nylon, polystyrene, polyvinyl chloride or polypropylene.

4.1.6 Anti-RSV-Antigen Antibodies

Anti-RSV-antigen antibodies that can be used with the methods of the invention bind immunospecifically to an antigen of RSV. In certain embodiments, the anti-RSV-antigen antibody binds immunospecifically to an RSV antigen of the Group A of RSV. In certain embodiments, the anti-RSV-antigen antibody binds immunospecifically to an RSV antigen of the Group B of RSV. In certain embodiments, an antibody binds to an antigen of RSV of one Group and cross reacts with the analogous antigen of the other Group.

In certain embodiments, an anti-RSV-antigen antibody binds immunospecifically to a RSV nucleoprotein, RSV phosphoprotein, RSV matrix protein, RSV small hydrophobic protein, RSV RNA-dependent RNA polymerase, RSV F protein, and/or RSV G protein.

In certain embodiments, an anti-RSV-antigen antibody binds to allelic variants of a RSV nucleoprotein, a RSV phosphoprotein, a RSV matrix protein, a RSV small hydrophobic protein, a RSV RNA-dependent RNA polymerase, a RSV F protein, and/or a RSV G protein.

In certain embodiments, the anti-RSV-antigen antibody binds immunospecifically to, inter alia, an RSV attachment glycoprotein, e.g., having an amino acid sequence of SEQ ID NO:390; a RSV fusion glycoprotein, e.g., having an amino acid sequence of SEQ ID NO:391; a RSV small hydrophobic protein, e.g., having an amino acid sequence of SEQ ID NO:392; a RSV RNA polymerase beta subunit (Large structural protein) (L protein), e.g., having an amino acid sequence of SEQ ID NO:393; a RSV phosphoprotein P, e.g., having an amino acid sequence of SEQ ID NO:394; an RSV attachment glycoprotein G, e.g., having an amino acid sequence of SEQ ID NO:395; a RSV nucleocapsid protein, e.g. having an amino acid sequence of SEQ ID NO:396; a RSV nucleoprotein (N), e.g., having an amino acid sequence of SEQ ID NO:397; and/or a RSV matrix protein, e.g., having an amino acid sequence of SEQ ID NO:398.

In certain embodiments, the anti-RSV-antigen antibody binds immunospecifically to a protein/polypeptide that consists of an amino acid sequence that is at least 60%, 70%, 80%, 90%, 95%, or at least 98% identical to the amino acid sequence of the attachment glycoprotein of SEQ ID NO:390; the fusion glycoprotein of SEQ ID NO:391; the small hydrophobic protein of SEQ ID NO:392; the RNA polymerase beta subunit (Large structural protein) (L protein) of SEQ ID NO:393; the phosphoprotein P of SEQ ID NO:394; the attachment glycoprotein G of SEQ ID NO:395; the nucleocapsid protein of SEQ ID NO:396; the nucleoprotein (N)of SEQ ID NO:397; and/or the matrix protein of SEQ ID NO:398. In certain embodiments, the anti-RSV-antigen antibody binds immunospecifically to a protein/polypeptide that consists of an amino acid sequence that is at most 70%, 80%, 90%, 95%, 98% or at most 100% identical to the amino acid sequence of the attachment glycoprotein of SEQ ID NO:390; the fusion glycoprotein of SEQ ID NO:391; the small hydrophobic protein of SEQ ID NO:392; the RNA polymerase beta subunit (Large structural protein) (L protein) of SEQ ID NO:393; the phosphoprotein P of SEQ ID NO:394; the attachment glycoprotein G of SEQ ID NO:395; the nucleocapsid protein of SEQ ID NO:396; the nucleoprotein (N) of SEQ ID NO:397; and/or the matrix protein of SEQ ID NO:398.

In certain embodiments, the anti-RSV-antigen antibodies are the anti-RSV-antigen antibodies of or are prepared by the methods of U.S. application Ser. No. 09/724,531, filed Nov. 28, 2000; Ser. No. 09/996,288, filed Nov. 28, 2001; and Ser. No. 09/996,265, filed Nov. 28, 2001, all entitled “Methods of Administering/Dosing Anti-RSV Antibodies for Prophylaxis and Treatment”, by Young et al., which are incorporated by reference herein in their entireties. Methods and composition for stabilized antibody formulations that can be used in the methods of the present invention are disclosed in U.S. Provisional Application Nos.: 60/388,921, filed Jun. 14, 2002, and 60/388,920, filed Jun. 14, 2002, which are incorporated by reference herein in their entireties.

In certain embodiments, the one or more antibodies or antigen-binding fragments thereof that bind immunospecifically to a RSV antigen comprise a Fc domain with a higher affinity for the FcRn receptor than the Fc domain of SYNAGIS® (Palivizumab). Such antibodies are described in U.S. patent application Ser. No. 10/020,354, filed Dec. 12, 2001, which is incorporated herein by reference in its entireties.

In certain embodiments, the one or more anti-RSV-antigen antibodies include, but are not limited to, SYNAGIS® (Palivizumab). In certain embodiments, the one or more anti-RSV-antigen antibodies include, but are not limited to, A4B4 (see section 4.1.5). In certain specific embodiments, the anti-RSV-antigen antibody is AFFF; P12f2 P12f4; P11d4; Ale9; A12a6; A13c4; A17d4; A4B4; 1X-493L1; FR H3-3F4; M3H9; Y10H6; DG; AFFF(1); 6H8; L1-7E5; L2-15B10; A13a11; A1h5; A4B4(1);A4B4-F52S; or A4B4L1FR-S28R. These antibodies are disclosed in International Application Publication No.: WO 02/43660, entitled “Methods of Administering/Dosing Anti-RSV Antibodies for Prophylaxis and Treatment”, by Young et al., which is incorporated herein by reference in its entirety.

In certain embodiments, the one or more antibodies that bind to a RSV antigen has a higher avidity and/or affinity for a RSV antigen than SYNAGIS® has for the RSV F glycoprotein. In certain embodiments, the one or more antibodies that bind immunospecifically to a RSV antigen has a higher affinity and/or avidity for a RSV antigen than any previously known anti-RSV-antigen specific antibodies or antigen-binding fragments thereof. In certain embodiments, anti-RSV-antigen antibody is not SYNAGIS®.

For the methods of the present invention, antibodies or antigen-binding fragments thereof which immunospecifically bind to a RSV antigen with an affinity constant of at least 2×10⁸ M⁻¹, at least 2.5×10⁸ M⁻¹, at least 5×10⁸ M⁻¹, at least 10⁹ M⁻¹, at least 5×10⁹ M⁻¹, at least 10¹⁰ M⁻¹, at least 5×10¹⁰ M⁻¹, at least 10¹¹ M⁻¹, at least 5×10 ¹¹ M⁻¹, at least 10¹² M⁻¹, at least 5×10¹² M⁻¹, at least 10¹³ M⁻¹, at least 5×10¹³ M⁻¹, at least 10¹⁴ M⁻¹, at least 5×10¹⁴ M⁻¹, at least 10¹⁵ M⁻¹, or at least 5×10¹⁵ M⁻¹ can be used. In a specific embodiment, the antibody that binds immunospecifically to a RSV antigen is SYNAGIS®, which binds to the RSV F glycoprotein. The present invention also provides pharmaceutical compositions comprising (i) one or more antibodies which immunospecifically bind to a RSV antigen with an affinity constant of at least 2×10⁸ M⁻¹, at least 2.5×10⁸ M⁻¹, at least 5×10⁸ M⁻¹, at least 10⁹ M⁻¹, at least 5×10⁹ M⁻¹, at least 10¹⁰ M⁻¹, at least 5×10¹⁰ M⁻¹, at least 10¹¹ M⁻¹, at least 5×10¹¹ M⁻¹, at least 10¹² M⁻¹, at least 5×10¹² M⁻¹, at least 10¹³ M⁻¹, at least 5×10¹³ M⁻¹, at least 10¹⁴ M⁻¹, at least 5×10¹⁴ M⁻¹, at least 10¹⁵ M⁻¹, or at least 5×10¹⁵ M⁻¹ and (ii) one or more antibodies which immunospecifically bind to a RSV antigen with an affinity constant of at least 2×10⁸ M⁻¹, at least 2.5×10⁸ M⁻¹, at least 5×10⁸ M⁻¹, at least 10⁹ M⁻¹, at least 5×10⁹ M⁻¹, at least 10¹⁰ M⁻¹, at least 5×10¹⁰ M⁻¹, at least 10¹¹ M⁻¹, at least 5×10¹¹ M⁻¹, at least 10¹² M⁻¹, at least 5×10¹² M⁻¹, at least 10¹³ M⁻¹, at least 5×10¹³ M⁻¹, at least 10¹⁴ M⁻¹, at least 5×10¹⁴M⁻¹, at least 10¹⁵ M⁻¹, or at least 5×10¹⁵ M⁻¹.

It should be recognized that antibodies that immunospecifically bind to a RSV antigen are known in the art. For example, SYNAGIS® is a humanized monoclonal antibody presently used for the prevention of RSV infection in pediatric patients. In a specific embodiment, an antibody to be used with the methods of the present invention is SYNAGIS® or an antibody-binding fragment thereof (e.g., contains one or more complementarity determining regions (CDRs) and preferably, the variable domain of SYNAGIS®). The amino acid sequence of SYNAGIS® is disclosed, e.g., in Johnson et al., 1997, J. Infectious Disease 176:1215-1224, and U.S. Pat. No. 5,824,307 and International Application Publication No.: WO 02/43660, entitled “Methods of Administering/Dosing Anti-RSV Antibodies for Prophylaxis and Treatment”, by Young et al., which are incorporated herein by reference in their entireties.

In certain embodiments, the antibodies to be used with the methods and compositions of the invention or fragments thereof bind immunospecifically to one or more RSV antigens regardless of the strain of RSV. In particular, the anti-RSV-antigen antibodies bind to an antigen of human RSV A and human RSV B. In certain embodiments, the anti-RSV-antigen antibodies bind to RSV antigens from one strain of RSV versus another RSV strain. In particular, the anti-RSV-antigen antibody binds to an antigen of human RSV A and not to human RSV B or vice versa. In a specific embodiment, the antibodies or antigen-binding fragments thereof immunospecifically bind to the RSV F glycoprotein, G glycoprotein or SH protein. In certain embodiments, the anti-RSV-antigen antibodies bind immunospecifically to the RSV F glycoprotein. In another preferred embodiment, the anti-RSV-antigen antibodies or antigen-binding fragments thereof bind to the A, B, C, I, II, IV, V, or VI antigenic sites of the RSV F glycoprotein (see, e.g., Lopez et al., 1998, J. Virol. 72:6922-6928, which is incorporated herein by reference in its entirety). In certain embodiments, the anti-RSV-antigen antibodies bind to a RSV nucleoprotein, a RSV phosphoprotein, a RSV matrix protein, a RSV small hydrophobic protein, a RSV RNA-dependent RNA polymerase, a RSV F protein, or a RSV G protein.

In certain embodiments, the anti-RSV-antigen antibodies or antigen-binding fragments thereof have a high binding affinity for one or more RSV antigens. In a specific embodiment, an anti-RSV antibody or an antigen-binding fragment thereof has an association rate constant or kₒₙ rate (antibody (Ab)+antigen (Ag)^{k}^{ₒₙ}→Ab−Ag)<|<BOX1>|> of at least 10⁵ M⁻¹s⁻¹, at least 5×10⁵ M⁻¹s⁻¹, at least 10⁶M⁻¹s⁻¹, at least 5×10⁶M⁻¹s⁻¹, at least 10⁷ M⁻¹s⁻¹, at least 5×10⁷ M⁻¹s⁻¹, or at least 10⁸ M⁻¹s⁻¹. In a preferred embodiment, an antibody of the present invention or fragment thereof has a kₒₙ of at least 2×10⁵ M⁻¹s⁻¹, at least 5×10⁵ M⁻¹s⁻¹, at least 10⁶ M⁻¹s⁻¹, at least 5×10⁶ M⁻¹s⁻¹, at least 10⁷ M⁻¹s⁻¹, at least 5×10⁷ M⁻¹s⁻¹, or at least 10⁸ M⁻¹s⁻¹.

In another embodiment, anti-RSV-antigen antibodies or fragment thereof has a k_{off} rate (antibody (Ab)+antigen) of less than 10⁻¹ s⁻¹, less than 5×10⁻¹ s⁻¹, less than 10⁻² s⁻¹, less than 5×10⁻² s⁻¹, less than 10⁻³ s⁻¹, less than 5×10⁻³ s⁻¹, less than 10⁻⁴ s⁻¹, less than 5×10⁻⁴ s⁻¹, less than 10⁻⁵ s⁻¹, less than 5×10⁻⁵ s⁻¹, less than 10⁻⁶ s⁻¹, less than 5×10⁻⁶ s⁻¹, less than 10⁻⁷ s⁻¹, less than 5×10⁻⁷ s⁻¹, less than 10⁻⁸ s⁻¹, less than 5×10⁻⁸ s⁻¹, less than 10⁻⁹ s⁻¹, less than 5×10⁻⁹ s⁻¹, or less than 10⁻¹⁰ s⁻¹. In a preferred embodiment, an anti-RSV-antigen antibodies or fragment thereof has a kₒₙ of less than 5×10⁻⁴ s⁻¹ less than 10⁻⁵ s⁻¹, less than 5×10⁻⁵ s⁻¹, less than 10⁻⁶ s⁻¹, less than 5×10⁻⁶ s⁻¹, less than 10⁻⁷ s⁻¹, less than 5×10⁻⁷ s⁻¹, less than 10⁻⁸ s⁻¹, less than 5×10⁻⁸ s⁻¹, less than 10⁻⁹ s⁻¹, less than 5×10⁻⁹ s⁻¹, or less than 10⁻¹⁰ s⁻¹.

In certain embodiments, the antibodies to be used with the methods of the invention or fragments thereof comprise the amino acid sequence of SYNAGIS® with one or more amino acid residue substitutions in one or more VL CDRs and/or one or more VH CDRs. In a specific embodiment, an antibody to be used with the methods of the invention comprises the amino acid sequence of SYNAGIS® with one or more amino acid residue substitutions of the amino acid residues indicated in bold face and underlining in Table 3. In accordance with this embodiment, the amino acid residue substitutions can be conservative or non-conservative. The antibody or antibody fragment generated by introducing substitutions in the VH domain, VH CDRs, VL domain and/or VL CDRs of SYNAGIS® can be tested in vitro and in vivo, for example, for its ability to bind to RSV F antigen, for its ability to neutralize RSV, or for its ability to prevent, treat or ameliorate one or more symptoms associated with a RSV infection.

TABLE 3
CDR Sequences Of SYNAGIS ®

| CDR | Sequence |
|---|---|
| VH1 | TSGMSVG |
| VH2 | DIWWDDKKDYNPSLKS |
| VH3 | SMITNWYFDV |
| VL1 | KCQLSVGYMH |
| VL2 | DTSKLAS |
| VL3 | FQGSGYPFT |

Bold faced & underlined amino acid residues are preferred residues which should be substituted.

In certain specific embodiments, the amino acid sequences of the different domains of one or more anti-RSV-antigen antibodies are as follows: VH Domain: SEQ ID NO:422; VH CDR1: TAGMSVG; VH CDR2: DIWWDDKKHYNPSLKD; VH CDR3: DMIFNFYFDV; VL Domain: SEQ ID NO:423; VL CDR1: SASSRVGYMH; VL CDR2: DTLLLDS; VL CDR3: FQGSGYPFT. This antibody has been disclosed as A4B4(1) in International Application Publication No.: WO 02/43660, entitled “Methods of Administering/Dosing Anti-RSV Antibodies for Prophylaxis and Treatment”, by Young et al., which is incorporated by reference herein in its entirety.

In certain specific embodiments, the anti-RSV-antigen antibody is AFFF; P12f2 P12f4; P11d4; Ale9; A12a6; A13c4; A17d4; A4B4; 1X-493L1; FR H3-3F4; M3H9; Y10H6; DG; AFFF(1); 6H8; L1-7E5; L2-15B10; A13al 1; A1h5; A4B4(1);A4B4-F52S; or A4B4L1FR-S28R. These antibodies are disclosed in International Application Publication No.: WO 02/43660, entitled “Methods of Administering/Dosing Anti-RSV Antibodies for Prophylaxis and Treatment”, by Young et al., which is incorporated herein by reference in its entirety.

4.1.7 Anti-hMPV-Antigen Antibodies

Any antibody that immunospecifically binds to an hMPV or to a protein of hMPV or a fragment, an analog, a derivative or a homolog thereof can be used with the methods of the invention. Mammalian MPV and proteins of mammalian MPV and homologs thereof are described in section 4.1.7.1.

4.1.7.1 hMPV

Structural Characteristics of a Mammalian Metapneumovirus

A Mammalian MPV is a negative-sense single stranded RNA virus belonging to the sub-family Pneumovirinae of the family Paramyxoviridae. Moreover, the mammalian MPV is identifiable as phylogenetically corresponding to the genus Metapneumovirus, wherein the mammalian MPV is phylogenetically more closely related to a virus isolate deposited as I-2614 with CNCM, Paris (SEQ ID NO: 19) than to turkey rhinotracheitis virus, the etiological agent of avian rhinotracheitis. A virus is identifiable as phylogenetically corresponding to the genus Metapneumovirus by, e.g., obtaining nucleic acid sequence information of the virus and testing it in phylogenetic analyses. Any technique known to the skilled artisan can be used to determine phylogenetic relationships between strains of viruses. Other techniques are disclosed in International Patent Application PCT/NL02/00040, published as WO 02/057302, which is incorporated by reference in its entirety herein. In particular, PCT/NL02/00040 discloses nucleic acid sequences that are suitable for phylogenetic analysis at page 12, line 27 to page 19, line 29, which are incorporated by reference herein. A virus can further be identified as a mammalian MPV on the basis of sequence similarity as described in more detail below.

In a specific embodiment, the mammalian MPV is a human MPV.

In addition to phylogenetic relatedness and sequence similarity of a virus to a mammalian MPV as disclosed herein, the similarity of the genomic organization of a virus to the genomic organization of a mammalian MPV disclosed herein can also be used to identify the virus as a mammalian MPV. In certain embodiments, the genomic organization of a mammalian MPV is different from the genomic organization of pneumoviruses within the sub-family Pneumovirinae of the family Paramyxoviridae. The classification of the two genera, metapneumovirus and pneumovirus, is based primarily on their gene constellation; metapneumoviruses generally lack non-structural proteins such as NS1 or NS2 (see also Randhawa et al., 1997, J. Virol. 71:9849-9854) and the gene order is different from that of pneumoviruses (RSV: ‘3-NS1-NS2-N-P-M-SH-G-F-M2-L-5’, APV: ‘3-N-P-M-F-M2-SH-G-L-5’) (Lung, et al., 1992, J. Gen. Virol. 73:1709-17 15; Yu, et al., 1992, Virology 186:426-434; Randhawa, et al., 1997, J. Virol. 71:9849-9854).

Further, a mammalian MPV of the invention can be identified by its immunological properties. In certain embodiments, specific anti-sera can be raised against mammalian MPV that can neutralize mammalian MPV. Monoclonal and polyclonal antibodies can be raised against MPV that can also neutralize mammalian MPV. (See, WO 02/057302, which is incorporated by reference herein.

The mammalian MPV of the invention is further characterized by its ability to infect a mammalian host, i.e., a mammalian cultured cell or a mammal. Unlike APV, mammalian MPV does not replicate or replicates only at low levels in chickens and turkeys. Mammalian MPV replicates, however, in mammalian hosts, such as cynomolgous macaques. In certain, more specific, embodiments, a mammalian MPV is further characterized by its ability to replicate in a mammalian host. In certain, more specific embodiments, a mammalian MPV is further characterized by its ability to cause the mammalian host to express proteins encoded by the genome of the mammalian MPV. In even more specific embodiments, the viral proteins expressed by the mammalian MPV are inserted into the cytoplasmic membranes of the mammalian host. In certain embodiments, the mammalian MPV of the invention can infect a mammalian host and cause the mammalian host to produce new infectious viral particles of the mammalian MPV. For a more detailed description of the functional characteristics of the mammalian MPV of the invention, see below.

In certain embodiments, the appearance of a virus in an electron microscope or its sensitivity to chloroform can be used to identify the virus as a mammalian MPV. The mammalian MPV of the invention appears in an electron microscope as paramyxovirus-like particle. Consistently, a mammalian MPV is sensitive to treatment with chloroform; a mammalian MPV is cultured optimally on tMK cells or cells functionally equivalent thereto and it is essentially trypsine dependent in most cell cultures. Furthermore, a mammalian MPV has a typical cytopathic effects (CPE) and lacks haemagglutinating activity against species of red blood cells. The CPE induced by MPV isolates are similar to the CPE induced by hRSV, with characteristic syncytia formation followed by rapid internal disruption of the cells and subsequent detachment from the culture plates. Although most paramyxoviruses have haemagglutinating activity, most of the pneumoviruses do not (Pringle, C. R. In: The Paramyxoviruses; (ed. D. W. Kingsbury) 1-39 (Plenum Press, New York, 1991)). A mammalian MPV contains a second overlapping ORF (M2-2) in the nucleic acid fragment encoding the M2 protein. The occurrence of this second overlapping ORF occurs in other pneumoviruses as shown in Ahmadian et al., 1999, J. Gen. Vir. 80:2011-2016.

In certain embodiments, a viral isolate can be identified as a mammalian MPV by the following method. A test sample can, e.g., be obtained from an animal or human. The sample is then tested for the presence of a virus of the sub-family Pneumovirinae. If a virus of the sub-family Pneumovirinae is present, the virus can be tested for any of the characteristics of a mammalian MPV as discussed herein, such as, but not limited to, phylogenetic relatedness to a mammalian MPV, nucleotide sequence identity to a nucleotide sequence of a mammalian MPV, amino acid sequence identity/homology to a amino acid sequence of a mammalian MPV, and genomic organization. Furthermore, the virus can be identified as a mammalian MPV by cross-hybridization experiments using nucleic acid sequences from a MPV isolate, RT-PCR using primers specific to mammalian MPV, or in classical cross-serology experiments using antibodies directed against a mammalian MPV isolate. In certain other embodiments, a mammalian MPV can be identified on the basis of its immunological distinctiveness, as determined by quantitative neutralization with animal antisera. The antisera can be obtained from, e.g., ferrets, pigs or macaques that are infected with a mammalian MPV.

In certain embodiments, the serotype does not cross-react with viruses other than mammalian MPV. In other embodiments, the serotype shows a homologous-to-heterologous titer ratio >16 in both directions If neutralization shows a certain degree of cross-reaction between two viruses in either or both directions (homologous-to-heterologous titer ration of eight or sixteen), distinctiveness of serotype is assumed if substantial biophysical/biochemical differences of DNA sequences exist. If neutralization shows a distinct degree of cross-reaction between two viruses in either or both directions (homologous-to-heterologous titer ratio of smaller than eight), identity of serotype of the isolates under study is assumed. Isolate I-2614, herein also known as MPV isolate 00-1 (as deposited with CNCM, Paris (SEQ ID NO:19)), can be used as prototype.

In certain embodiments, a virus can be identified as a mammalian MPV by means of sequence homology/identity of the viral proteins or nucleic acids in comparison with the amino acid sequence and nucleotide sequences of the viral isolates disclosed herein by sequence or deposit. In particular, a virus is identified as a mammalian MPV when the genome of the virus contains a nucleic acid sequence that has a percentage nucleic acid identity to a virus isolate deposited as I-2614 with CNCM, Paris which is higher than the percentages identified herein for the nucleic acids encoding the L protein, the M protein, the N protein, the P protein, or the F protein as identified herein below in comparison with APV-C (see Table 4). (See, PCT WO 02/05302, at pp. 12 to 19, which is incorporated by reference herein. Without being bound by theory, it is generally known that viral species, especially RNA virus species, often constitute a quasi species wherein the members of a cluster of the viruses display sequence heterogeneity. Thus, it is expected that each individual isolate may have a somewhat different percentage of sequence identity when compared to APV-C.

The highest amino sequence identity between the proteins of MPV and any of the known other viruses of the same family to date is the identity between APV-C and human MPV. Between human MPV and APV-C, the amino acid sequence identity for the matrix protein is 87%, 88% for the nucleoprotein, 68% for the phosphoprotein, 81% for the fusion protein and 56-64% for parts of the polymerase protein, as can be deduced when comparing the sequences given in FIG. 30, see also Table 4. Viral isolates that contain ORFs that encode proteins with higher homology compared to these maximum values are considered mammalian MPVs. It should be noted that, similar to other viruses, a certain degree of variation is found between different isolated of mammalian MPVs.

TABLE 4
Amino acid sequence identity between the ORFs
of MPV and those of other paramyxoviruses.

| | N | P | M | F | M2-1 | M2-2 | L |
|---|---|---|---|---|---|---|---|
| APV A | 69 | 55 | 78 | 67 | 72 | 26 | 64 |
| APV B | 69 | 51 | 76 | 67 | 71 | 27 | ⁻² |
| APV C | 88 | 68 | 87 | 81 | 84 | 56 | ⁻² |
| hRSVA | 42 | 24 | 38 | 34 | 36 | 18 | 42 |
| hRSV B | 41 | 23 | 37 | 33 | 35 | 19 | 44 |
| bRSV | 42 | 22 | 38 | 34 | 35 | 13 | 44 |
| PVM | 45 | 26 | 37 | 39 | 33 | 12 | ⁻² |
| others³ | 7-11 | 4-9 | 7-10 | 10-18 | ⁻⁴ | ⁻⁴ | 13-14 |

Footnotes:
¹No sequence homologies were found with known G and SH proteins and were thus excluded
²Sequences not available.
³others: human parainfluenza virus type 2 and 3, Sendai virus, measles virus, nipah virus, phocine distemper virus, and New Castle Disease virus.
⁴ORF absent in viral genome.

Any protein of a mammalian MPV can be used as an immunogen to generate antibodies to be used with the methods of the invention. In certain embodiments, an antibody to be used with the methods of treatment of the present invention bind immunospecifically to a protein of mammlian MPV as set forth below.

In certain embodiments, the amino acid sequence of the SH protein of the mammalian MPV is at least 30%, at least 35%, at least 40%, at least 45%, at least 50%, at least 55%, at least 60%, at least 65%, at least 70%, at least 75%, at least 80%, at least 85%, at least 90%, at least 95%, at least 98%, at least 99%, or at least 99.5% identical to the amino acid sequence of SEQ ID NO:382 (SH protein of isolate NL/1/00; see Table 5). The isolated negative-sense single stranded RNA metapneumovirus that comprises the SH protein that is at least 30% identical to SEQ ID NO:382 (SH protein of isolate NL/1/00; see Table 5) is capable of infecting a mammalian host. In certain embodiments, the isolated negative-sense single stranded RNA metapneumovirus that comprises the SH protein that is at least 30% identical to SEQ ID NO:382 (SH protein of isolate NL/1/00; see Table 5) is capable of replicating in a mammalian host. In certain embodiments, a mammalian MPV contains a nucleotide sequence that encodes a SH protein that is at least 30% identical to SEQ ID NO:382 (SH protein of isolate NL/1/00; see Table 5).

In certain embodiments, the amino acid sequence of the G protein of the mammalian MPV is at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 98%, at least 99%, or at least 99.5% identical to the amino acid sequence of SEQ ID NO:322 (G protein of isolate NL/1/00; see Table 5). The isolated negative-sense single stranded RNA metapneumovirus that comprises the G protein that is at least 20% identical to SEQ ID NO:322 (G protein of isolate NL/1/00; see Table 5) is capable of infecting a mammalian host. In certain embodiments, the isolated negative-sense single stranded RNA metapneumovirus that comprises the G protein that is at least 20% identical to SEQ ID NO:322 (G protein of isolate NL/1/00; see Table 5) is capable of replicating in a mammalian host. In certain embodiments, a mammalian MPV contains a nucleotide sequence that encodes a G protein that is at least 20% identical to SEQ ID NO:322 (G protein of isolate NL/1/00; see Table 5).

In certain embodiments, the amino acid sequence of the L protein of the mammalian MPV is at least 85%, at least 90%, at least 95%, at least 98%, at least 99%, or at least 99.5% identical to the amino acid sequence of SEQ ID NO:330 (L protein of isolate NL/1/00; see Table 5). The isolated negative-sense single stranded RNA metapneumovirus that comprises the L protein that is at least 85% identical to SEQ ID NO:330 (L protein of isolate NL/1/00; see Table 5) is capable of infecting a mammalian host. In certain embodiments, the isolated negative-sense single stranded RNA metapneumovirus that comprises the L protein that is at least 85% identical to SEQ ID NO:330 (L protein of isolate NL/1/00; see Table 5) is capable of replicating in a mammalian host. In certain embodiments, a mammalian MPV contains a nucleotide sequence that encodes a L protein that is at least 20% identical to SEQ ID NO:330 (L protein of isolate NL/1/00; see Table 5).

In certain embodiments, the amino acid sequence of the N protein of the mammalian MPV is at least 90%, at least 95%, or at least 98% identical to the amino acid sequence of SEQ ID NO:366. The isolated negative-sense single stranded RNA metapneumovirus that comprises the N protein that is at least 90% identical in amino acid sequence to SEQ ID NO:366 is capable of infecting mammalian host. In certain embodiments, the isolated negative-sense single stranded RNA metapneumovirus that comprises the N protein that is 90% identical in amino acid sequence to SEQ ID NO:366 is capable of replicating in a mammalian host. The amino acid identity is calculated over the entire length of the N protein. In certain embodiments, a mammalian MPV contains a nucleotide sequence that encodes a N protein that is at least 90%, at least 95%, or at least 98% identical to the amino acid sequence of SEQ ID NO:366.

The amino acid sequence of the P protein of the mammalian MPV is at least 70%, at least 80%, at least 90%, at least 95% or at least 98% identical to the amino acid sequence of SEQ ID NO:374. The mammalian MPV that comprises the P protein that is at least 70% identical in amino acid sequence to SEQ ID NO:374 is capable of infecting a mammalian host. In certain embodiments, the mammalian MPV that comprises the P protein that is at least 70% identical in amino acid sequence to SEQ ID NO:374 is capable of replicating in a mammalian host. The amino acid identity is calculated over the entire length of the P protein. In certain embodiments, a mammalian MPV contains a nucleotide sequence that encodes a P protein that is at least 70%, at least 80%, at least 90%, at least 95% or at least 98% identical to the amino acid sequence of SEQ ID NO:374.

The amino acid sequence of the M protein of the mammalian MPV is at least 90%, at least 95% or at least 98% identical to the amino acid sequence of SEQ ID NO:358. The mammalian MPV that comprises the M protein that is at least 90% identical in amino acid sequence to SEQ ID NO:358 is capable of infecting mammalian host. In certain embodiments, the isolated negative-sense single stranded RNA metapneumovirus that comprises the M protein that is 90% identical in amino acid sequence to SEQ ID NO:358 is capable of replicating in a mammalian host. The amino acid identity is calculated over the entire length of the M protein. In certain embodiments, a mammalian MPV contains a nucleotide sequence that encodes a M protein that is at least 90%, at least 95% or at least 98% identical to the amino acid sequence of SEQ ID NO:358.

The amino acid sequence of the F protein of the mammalian MPV is at least 85%, at least 90%, at least 95% or at least 98% identical to the amino acid sequence of SEQ ID NO:314. The mammalian MPV that comprises the F protein that is at least 85% identical in amino acid sequence to SEQ ID NO:314 is capable of infecting a mammalian host. In certain embodiments, the isolated negative-sense single stranded RNA metapneumovirus that comprises the F protein that is 85% identical in amino acid sequence to SEQ ID NO:314 is capable of replicating in mammalian host. The amino acid identity is calculated over the entire length of the F protein. In certain embodiments, a mammalian MPV contains a nucleotide sequence that encodes a F protein that is at least 85%, at least 90%, at least 95% or at least 98% identical to the amino acid sequence of SEQ ID NO:314.

The amino acid sequence of the M2-1 protein of the mammalian MPV is at least 85%, at least 90%, at least 95% or at least 98% identical to the amino acid sequence of SEQ ID NO:338. The mammalian MPV that comprises the M2-1 protein that is at least 85% identical in amino acid sequence to SEQ ID NO:338 is capable of infecting a mammalian host. In certain embodiments, the isolated negative-sense single stranded RNA metapneumovirus that comprises the M2-1 protein that is 85% identical in amino acid sequence to SEQ ID NO:338 is capable of replicating in a mammalian host. The amino acid identity is calculated over the entire length of the M2-1 protein. In certain embodiments, a mammalian MPV contains a nucleotide sequence that encodes a M2-1 protein that is at least 85%, at least 90%, at least 95% or at least 98% identical to the amino acid sequence of SEQ ID NO:338.

The amino acid sequence of the M2-2 protein of the mammalian MPV is at least 60%, at least 70%, at least 80%, at least 90%, at least 95% or at least 98% identical to the amino acid sequence of SEQ ID NO:346 The isolated mammalian MPV that comprises the M2-2 protein that is at least 60% identical in amino acid sequence to SEQ ID NO:346 is capable of infecting mammalian host. In certain embodiments, the isolated negative-sense single stranded RNA metapneumovirus that comprises the M2-2 protein that is 60% identical in amino acid sequence to SEQ ID NO:346 is capable of replicating in a mammalian host. The amino acid identity is calculated over the entire length of the M2-2 protein. In certain embodiments, a mammalian MPV contains a nucleotide sequence that encodes a M2-1 protein that is is at least 60%, at least 70%, at least 80%, at least 90%, at least 95% or at least 98% identical to the amino acid sequence of SEQ ID NO:346.

In certain embodiments, the negative-sense single stranded RNA metapneumovirus encodes at least two proteins, at least three proteins, at least four proteins, at least five proteins, or six proteins selected from the group consisting of (i) a N protein with at least 90% amino acid sequence identity to SEQ ID NO:366; (ii) a P protein with at least 70% amino acid sequence identity to SEQ ID NO:374 (iii) a M protein with at least 90% amino acid sequence identity to SEQ ID NO:358 (iv) a F protein with at least 85% amino acid sequence identity to SEQ ID NO:314 (v) a M2-1 protein with at least 85% amino acid sequence identity to SEQ ID NO:338; and (vi) a M2-2 protein with at least 60% amino acid sequence identity to SEQ ID NO:346.

Mammalian MPV, can be divided into two subgroups, subgroup A and subgroup B, and the two subgroups can each be devided into two variants, A1 and A2, and B1 and B2. A mammalian MPV can be identified as a member of subgroup A if it is phylogenetically closer related to the isolate 00-1 (SEQ ID NO:19) than to the isolate 99-1 (SEQ ID NO:18). A mammalian MPV can be identified as a member of subgroup B if it is phylogenetically closer related to the isolate 99-1 (SEQ ID NO:18) than to the isolate 00-1 (SEQ ID NO:19). In other embodiments, nucleotide or amino acid sequence homologies of individual ORFs can be used to classify a mammalian MPV as belonging to subgroup A or B.

The different isolates of mammalian MPV can be divided into four different variants, variant A1, variant A2, variant B1 and variant B2 (see FIGS. 21 and 22). The isolate 00-1 (SEQ ID NO: 19) is an example of the variant A1 of mammalian MPV. The isolate 99-1 (SEQ ID NO:18) is an example of the variant B1 of mammalian MPV. A mammalian MPV can be grouped into one of the four variants using a phylogenetic analysis. Thus, a mammalian MPV belongs to a specific variant if it is phylogenetically closer related to a known member of that variant than it is phylogenetically related to a member of another variant of mammalian MPV. The sequence of any ORF and the encoded polypeptide may be used to type a MPV isolate as belonging to a particular subgroup or variant, including N, P, L, M, SH, G, M2 or F polypeptides. In a specific embodiment, the classification of a mammalian MPV into a variant is based on the sequence of the G protein. Without being bound by theory, the G protein sequence is well suited for phylogenetic analysis because of the high degree of variation among G proteins of the different variants of mammalian MPV.

In certain embodiments of the invention, sequence homology may be determined by the ability of two sequences to hybridize under certain conditions, as set forth below. A nucleic acid which is hybridizable to a nucleic acid of a mammalian MPV, or to its reverse complement, or to its complement can be used in the methods of the invention to determine their sequence homology and identities to each other. In certain embodiments, the nucleic acids are hybridized under conditions of high stringency.

It is well-known to the skilled artisan that hybridization conditions, such as, but not limited to, temperature, salt concentration, pH, formamide concentration (see, e.g., Sambrook et al., 1989, Chapters 9 to 11, Molecular Cloning, A Laboratory Manual, 2d Ed., Cold Spring Harbor Laboratory Press, Cold Spring Harbor, N.Y., incorporated herein by reference in its entirety). In certain embodiments, hybridization is performed in aqueous solution and the ionic strength of the solution is kept constant while the hybridization temperature is varied dependent on the degree of sequence homology between the sequences that are to be hybridized. For DNA sequences that 100% identical to each other and are longer than 200 basebairs, hybridization is carried out at approximately 15-25° C. below the melting temperature (Tm) of the perfect hybrid. The melting temperature (Tm) can be calculated using the following equation (Bolton and McCarthy, 1962, Proc. Natl. Acad. Sci. USA 84:1390):

Tm=81.5° C.−16.6(log10[Na+])+(% G+C)−0.63(% formamide)−(600/l)

Wherein (Tm) is the melting temperature, [Na+] is the sodium concentration, G+C is the Guanine and Cytosine content, and l is the length of the hybrid in basepairs. The effect of mismatches between the sequences can be calculated using the formula by Bonner et al. (Bonner et al., 1973, J. Mol. Biol. 81:123-135): for every 1% of mismatching of bases in the hybrid, the melting temperature is reduced by 1-1.5° C.

Thus, by determining the temperature at which two sequences hybridize, one of skill in the art can estimate how similar a sequence is to a known sequence. This can be done, e.g., by comparison of the empirically determined hybridization temperature with the hybridization temperature calculated for the know sequence to hybridize with its perfect match. Through the use of the formula by Bonner et al., the relationship between hybridization temperature and percent mismatch can be exploited to provide information about sequence similarity.

By way of example and not limitation, procedures using such conditions of high stringency are as follows. Prehybridization of filters containing DNA is carried out for 8 h to overnight at 65 C in buffer composed of 6×SSC, 50 mM Tris-HCl (pH 7.5), 1 mM EDTA, 0.02% PVP, 0.02% Ficoll, 0.02% BSA, and 500 μg/ml denatured salmon sperm DNA. Filters are hybridized for 48 h at 65 C in prehybridization mixture containing 100 μg/ml denatured salmon sperm DNA and 5-20×106 cpm of ³²P-labeled probe. Washing of filters is done at 37 C for 1 h in a solution containing 2×SSC, 0.01% PVP, 0.01% Ficoll, and 0.01% BSA. This is followed by a wash in 0.1×SSC at 50 C for 45 min before autoradiography. Other conditions of high stringency which may be used are well known in the art. In other embodiments of the invention, hybridization is performed under moderate of low stringency conditions, such conditions are well-known to the skilled artisan (see e.g., Sambrook et al., 1989, Molecular Cloning, A Laboratory Manual, 2d Ed., Cold Spring Harbor Laboratory Press, Cold Spring Harbor, N.Y.; see also, Ausubel et al., eds., in the Current Protocols in Molecular Biology series of laboratory technique manuals, 1987-1997 Current Protocols,© 1994-1997 John Wiley and Sons, Inc., each of which is incorporated by reference herein in their entirety). An illustrative low stringency condition is provided by the following system of buffers: hybridization in a buffer comprising 35% formamide, 5×SSC, 50 mM Tris-HCl (pH 7.5), 5 mM EDTA, 0.02% PVP, 0.02% Ficoll, 0.2% BSA, 100 μg/ml denatured salmon sperm DNA, and 10% (wt/vol) dextran sulfate for 18-20 hours at 40 □C, washing in a buffer consisting of 2×SSC, 25 mM Tris-HCl (pH 7.4), 5 mM EDTA, and 0.1% SDS for 1.5 hours at 55 □C, and washing in a buffer consisting of 2×SSC, 25 mM Tris-HCl (pH 7.4), 5 mM EDTA, and 0.1% SDS for 1.5 hours at 60 □C.

In certain embodiments, a mammalian MPV can be classified into one of the variant using probes that are specific for a specific variant of mammalian MPV. Such probes include primers for RT-PCR (Table 5) and antibodies.

In certain embodiments of the invention, the different variants of mammalian MPV can be distinguished from each other by way of the amino acid sequences of the different viral proteins. In other embodiments, the different variants of mammalian MPV can be distinguished from each other by way of the nucleotide sequences of the different ORFs encoded by the viral genome. A variant of mammalian MPV can be, but is not limited to, A1, A2, B1 or B2.

An isolate of mammalian MPV is classified as a variant B1 if it is phylogenetically closer related to the viral isolate NL/1/99 (SEQ ID NO:18) than it is related to any of the following other viral isolates: NL/1/00 (SEQ ID NO:19), NL/17/00 (SEQ ID NO:20) and NL/1/94 (SEQ ID NO:21). One or more of the ORFs of a mammalian MPV can be used to classify the mammalian MPV into a variant. A mammalian MPV can be classified as an MPV variant B1, if the amino acid sequence of its G protein is at least 66%, at least 70%, at least 75%, at least 80%, at least 85%, at least 90%, at least 95%, at least 98%, or at least 99% or at least 99.5% identical to the G protein of a mammalian MPV variant B1 as represented by the prototype NL/1/99 (SEQ ID NO:324); if the amino acid sequence of its N proteint is at least 98.5% or at least 99% or at least 99.5% identical to the N protein of a mammalian MPV variant B1 as represented by the prototype NL/1/99 (SEQ ID NO:368); if the amino acid sequence of its P protein is at least 96%, at least 98%, or at least 99% or at least 99.5% identical to the P protein of a mammalian MPV variant B1 as represented by the prototype NL/1/99 (SEQ ID NO:376); if the amino acid sequence of its M protein is identical to the M protein of a mammalian MPV variant B1 as represented by the prototype NL/1/99 (SEQ ID NO:360); if the amino acid sequence of its F protein is at least 99% identical to the F protein of a mammalian MPV variant B1 as represented by the prototype NL/1/99 (SEQ ID NO:316); if the amino acid sequence of its M2-1 protein is at least 98% or at least 99% or at least 99.5% identical to the M2-1 protein of a mammalian MPV variant B1 as represented by the prototype NL/1/99 (SEQ ID NO:340); if the amino acid sequence of its M2-2 protein is at least 99% or at least 99.5% identical to the M2-2 protein of a mammalian MPV variant B1 as represented by the prototype NL/1/99 (SEQ ID NO:348); if the amino acid sequence of its SH protein is at least 83%, at least 85%, at least 90%, at least 95%, at least 98%, or at least 99% or at least 99.5% identical to the SH protein of a mammalian MPV variant B1 as represented by the prototype NL/1/99 (SEQ ID NO:384); and/or if the amino acid sequence of its L protein is at least 99% or at least 99.5% identical to the L protein a mammalian MPV variant B1 as represented by the prototype NL/1/99 (SEQ ID NO:332).

An isolate of mammalian MPV is classified as a variant A1 if it is phylogenetically closer related to the viral isolate NL/1/00 (SEQ ID NO:19) than it is related to any of the following other viral isolates: NL/1/99 (SEQ ID NO:18), NL/17/00 (SEQ ID NO:20) and NL/1/94 (SEQ ID NO:21). One or more of the ORFs of a mammalian MPV can be used to classify the mammalian MPV into a variant. A mammalian MPV can be classified as an MPV variant A1, if the amino acid sequence of its G protein is at least 66%, at least 70%, at least 75%, at least 80%, at least 85%, at least 90%, at least 95%, at least 98%, or at least 99% or at least 99.5% identical to the G protein of a mammalian MPV variant A1 as represented by the prototype NL/1/00 (SEQ ID NO:322); if the amino acid sequence of its N protein is at least 99.5% identical to the N protein of a mammalian MPV variant A1 as represented by the prototype NL/1/00 (SEQ ID NO:366); if the amino acid sequence of its P protein is at least 96%, at least 98%, or at least 99% or at least 99.5% identical to the P protein of a mammalian MPV variant A1 as represented by the prototype NL/1/00 (SEQ ID NO:374); if the amino acid sequence of its M protein is at least 99% or at least 99.5% identical to the M protein of a mammalian MPV variant A1 as represented by the prototype NL/1/00 (SEQ ID NO:358); if the amino acid sequence of its F protein is at least 98% or at least 99% or at least 99.5% identical to the F protein of a mammalian MPV variant A1 as represented by the prototype NL/1/00 (SEQ ID NO:314); if the amino acid sequence of its M2-1 protein is at least 99% or at least 99.5% identical to the M2-1 protein of a mammalian MPV variant A1 as represented by the prototype NL/1/00 (SEQ ID NO:338); if the amino acid sequence of its M2-2 protein is at least 96% or at least 99% or at least 99.5% identical to the M2-2 protein of a mammalian MPV variant A1 as represented by the prototype NL/1/00 (SEQ ID NO:346); if the amino acid sequence of its SH protein is at least 84%, at least 90%, at least 95%, at least 98%, or at least 99% or at least 99.5% identical to the SH protein of a mammalian MPV variant A1 as represented by the prototype NL/1/00 (SEQ ID NO:382); and/or if the amino acid sequence of its L protein is at least 99% or at least 99.5% identical to the L protein of a virus of a mammalian MPV variant A1 as represented by the prototype NL/1/00 (SEQ ID NO:330).

An isolate of mammalian MPV is classified as a variant A2 if it is phylogenetically closer related to the viral isolate NL/17/00 (SEQ ID NO:20) than it is related to any of the following other viral isolates: NL/1/99 (SEQ ID NO:18), NL/1/00 (SEQ ID NO:19) and NL/1/94 (SEQ ID NO:21). One or more of the ORFs of a mammalian MPV can be used to classify the mammalian MPV into a variant. A mammalian MPV can be classified as an MPV variant A2, if the amino acid sequence of its G protein is at least 66%, at least 70%, at least 75%, at least 80%, at least 85%, at least 90%, at least 95%, at least 98%, at least 99% or at least 99.5% identical to the G protein of a mammalian MPV variant A2 as represented by the prototype NL/17/00 (SEQ ID NO:332); if the amino acid sequence of its N protein is at least 99.5% identical to the N protein of a mammalian MPV variant A2 as represented by the prototype NL/17/00 (SEQ ID NO:367); if the amino acid sequence of its P protein is at least 96%, at least 98%, at least 99% or at least 99.5% identical to the P protein of a mammalian MPV variant A2 as represented by the prototype NL/17/00 (SEQ ID NO:375); if the amino acid sequence of its M protein is at least 99%, or at least 99.5% identical to the M protein of a mammalian MPV variant A2 as represented by the prototype NL/17/00 (SEQ ID NO:359); if the amino acid sequence of its F protein is at least 98%, at least 99% or at least 99.5% identical to the F protein of a mammalian MPV variant A2 as represented by the prototype NL/17/00 (SEQ ID NO:315); if the amino acid sequence of its M2-1 protein is at least 99%, or at least 99.5% identical to the M2-1 protein of a mammalian MPV variant A2 as represented by the prototype NL/17/00 (SEQ ID NO: 339); if the amino acid sequence of its M2-2 protein is at least 96%, at least 98%, at least 99% or at least 99.5% identical to the M22 protein of a mammalian MPV variant A2 as represented by the prototype NL/17/00 (SEQ ID NO:347); if the amino acid sequence of its SH protein is at least 84%, at least 85%, at least 90%, at least 95%, at least 98%, at least 99% or at least 99.5% identical to the SH protein of a mammalian MPV variant A2 as represented by the prototype NL/17/00 (SEQ ID NO:383); if the amino acid sequence of its L protein is at least 99% or at least 99.5% identical to the L protein of a mammalian MPV variant A2 as represented by the prototype NL/17/00 (SEQ ID NO:331).

An isolate of mammalian MPV is classified as a variant B2 if it is phylogenetically closer related to the viral isolate NL/1/94 (SEQ ID NO:21) than it is related to any of the following other viral isolates: NL/1/99 (SEQ ID NO:18), NL/1/00 (SEQ ID NO:19) and NL/17/00 (SEQ ID NO:20). One or more of the ORFs of a mammalian MPV can be used to classify the mammalian MPV into a variant. A mammalian MPV can be classified as an MPV variant B2, if the amino acid sequence of its G protein is at least 66%, at least 70%, at least 75%, at least 80%, at least 85%, at least 90%, at least 95%, at least 98%, or at least 99% or at least 99.5% identical to the G protein of a mammalian MPV variant B2 as represented by the prototype NL/1/94 (SEQ ID NO:325); if the amino acid sequence of its N protein is at least 99% or at least 99.5% identical to the N protein of a mammalian MPV variant B2 as represented by the prototype NL/1/94 (SEQ ID NO:369); if the amino acid sequence of its P protein is at least 96%, at least 98%, or at least 99% or at least 99.5% identical to the P protein of a mammalian MPV variant B2 as represented by the prototype NL/1/94 (SEQ ID NO:377); if the amino acid sequence of its M protein is identical to the M protein of a mammalian MPV variant B2 as represented by the prototype NL/1/94 (SEQ ID NO:361); if the amino acid sequence of its F protein is at least 99% or at least 99.5% identical to the F protein of a mammalian MPV variant B2 as represented by the prototype NL/1/94 (SEQ ID NO:317); if the amino acid sequence of the M2-1 protein is at least 98% or at least 99% or at least 99.5% identical to the M2-1 protein of a mammalian MPV variant B2 as represented by the prototype NL/1/94 (SEQ ID NO:341); if the amino acid sequence that is at least 99% or at least 99.5% identical to the M2-2 protein of a mammalian MPV variant B2 as represented by the prototype NL/1/94 (SEQ ID NO:349); if the amino acid sequence of its SH protein is at least 84%, at least 85%, at least 90%, at least 95%, at least 98%, or at least 99% or at least 99.5% identical to the SH protein of a mammalian MPV variant B2 as represented by the prototype NL/1/94 (SEQ ID NO:385); and/or if the amino acid sequence of its L protein is at least 99% or at least 99.5% identical to the L protein of a mammalian MPV variant B2 as represented by the prototype NL/1/94 (SEQ ID NO:333).

In certain embodiments, the percentage of sequence identity is based on an alignment of the full length proteins. In other embodiments, the percentage of sequence identity is based on an alignment of contiguous amino acid sequences of the proteins, wherein the amino acid sequences can be 25 amino acids, 50 amino acids, 75 amino acids, 100 amino acids, 125 amino acids, 150 amino acids, 175 amino acids, 200 amino acids, 225 amino acids, 250 amino acids, 275 amino acids, 300 amino acids, 325 amino acids, 350 amino acids, 375 amino acids, 400 amino acids, 425 amino acids, 450 amino acids, 475 amino acids, 500 amino acids, 750 amino acids, 1000 amino acids, 1250 amino acids, 1500 amino acids, 1750 amino acids, 2000 amino acids or 2250 amino acids in length.

Functional Characteristics of a Mammalian MPV

In addition to the structural definitions of the mammalian MPV, a mammalian MPV can also be defined by its functional characteristics. In certain embodiments, a mammalian MPV is capable of infecting a mammalian host. The mammalian host can be a mammalian cell, tissue, organ or a mammal. In a specific embodiment, the mammalian host is a human or a human cell, tissue or organ. Any method known to the skilled artisan can be used to test whether the mammalian host has been infected with the mammalian MPV. In certain embodiments, the virus is tested for its ability to attach to a mammalian cell. In certain other embodiments, the virus is tested for its ability to transfer its genome into the mammalian cell.

In an illustrative embodiment, the genome of the virus is detectably labeled, e.g., radioactively labeled. The virus is then incubated with a mammalian cell for at least 1 minute, at least 5 minutes at least 15 minutes, at least 30 minutes, at least 1 hour, at least 2 hours, at least 5 hours, at least 12 hours, or at least 1 day. The cells are subsequently washed to remove any viral particles from the cells and the cells are then tested for the presence of the viral genome by virtue of the detectable label. In another embodiment, the presence of the viral genome in the cells is detected using RT-PCR using mammalian MPV specific primers. (See, PCT WO 02/057302 at pp.37 to 44, which is incorporated by reference herein).

In certain embodiments, a mammalian virus is capable to infect a mammalian host and to cause proteins of the mammalian MPV to be inserted into the cytoplasmic membrane of the mammalian host. The mammalian host can be a cultured mammalian cell, organ, tissue or mammal. In an illustrative embodiment, a mammalian cell is incubated with the mammalian virus. The cells are subsequently washed under conditions that remove the virus from the surface of the cell. Any technique known to the skilled artisan can be used to detect the newly expressed viral protein inserted in the cytoplasmic membrane of the mammalian cell. For example, after infection of the cell with the virus, the cells are maintained in medium comprising a detectably labeled amino acid. The cells are subsequently harvested, lysed, and the cytoplasmic fraction is separated from the membrane fraction. The proteins of the membrane fraction are then solubilized and then subjected to an immunoprecipitation using antibodies specific to a protein of the mammalian MPV, such as, but not limited to, the F protein or the G protein. The immunoprecipitated proteins are then subjected to SDS PAGE. The presence of viral protein can then be detected by autoradiography. In another embodiment, the presence of viral proteins in the cytoplasmic membrane of the host cell can be detected by immunocytochemistry using one or more antibodies specific to proteins of the mammalian MPV.

In even other embodiments, a mammalian MPV is capable of infecting a mammalian host and of replicating in the mammalian host. The mammalian host can be a cultured mammalian cell, organ, tissue or mammal. Any technique known to the skilled artisan can be used to determine whether a virus is capable of infecting a mammalian cell and of replicating within the mammalian host. In a specific embodiment, mammalian cells are infected with the virus. The cells are subsequently maintained for at least 30 minutes, at least 1 hour, at least 2 hours, at least 5 hours, at least 12 hours, at least 1 day, or at least 2 days. The level of viral genomic RNA in the cells can be monitored using Northern blot analysis, RT-PCR or in situ hybridization using probes that are specific to the viral genome. An increase in viral genomic RNA demonstrates that the virus can infect a mammalian cell and can replicate within a mammalian cell.

In even other embodiments, a mammalian MPV is capable of infecting a mammalian host, wherein the infection causes the mammalian host to produce new infectious mammalian MPV. The mammalian host can be a cultured mammalian cell or a mammal. Any technique known to the skilled artisan can be used to determine whether a virus is capable of infecting a mammalian host and cause the mammalian host to produce new infectious viral particles. In an illustrative example, mammalian cells are infected with a mammalian virus. The cells are subsequently washed and incubated for at least 30 minutes, at least 1 hour, at least 2 hours, at is least 5 hours, at least 12 hours, at least 1 day, at least 2 days, at least one week, or at least twelve days. The titer of virus can be monitored by any method known to the skilled artisan. For exemplary methods see section 5.8.

In certain, specific embodiments, a mammalian MPV is a human MPV. The tests described in this section can also be performed with a human MPV. In certain embodiments, the human MPV is capable of infecting a mammalian host, such as a mammal or a mammalian cultured cell.

In certain embodiments, a human MPV is capable to infect a mammalian host and to cause proteins of the human MPV to be inserted into the cytoplasmic membrane of the mammalian host.

In even other embodiments, a human MPV is capable of infecting a mammalian host and of replicating in the mammalian host.

In even other embodiments, the human MPV of the invention is capable of infecting a mammalian host and of replicating in the mammalian host, wherein the infection and replication causes the mammalian host to produce and package new infectious human MPV.

In certain embodiments, a mammalian MPV, even though it is capable of infecting a mammalian host, is also capable of infecting an avian host, such as a bird or an avian cultured cell. In certain embodiments, the mammalian MPV is capable to infect an avian host and to cause proteins of the mammalian MPV to be inserted into the cytoplasmic membrane of the avian host. In even other embodiments, the mammalian MPV of the invention is capable of infecting an avian host and of replicating in the avian host. In even other embodiments, the mammalian MPV of the invention is capable of infecting an avian host and of replicating in the avian host, wherein the infection and replication causes the avian host to produce and package new infectious mammalian MPV.

A description of mammalian MPV can also be found in co-owned and co-pending U.S. application Nos.: Ser. No. 10/371,099 and Ser. No. 10/371,122; both filed on Feb. 21, 2003; both of which are incorporated herein by reference in their entireties.

4.1.7.2 Anti-hMPV Antibodies

An anti-hMPV-antigen antibody to be used with the methods of the invention can be an antibody that immunospecifically binds to hMPV nucleoprotein, hMPV phosphoprotein, hMPV matrix protein, hMPV small hydrophobic protein, hMPV RNA-dependent RNA polymerase, hMPV F protein, and hMPV G protein.

In certain embodiments, the anti-hMPV-antigen antibody binds immunospecifically to a hMPV antigen of a hMPV isolate from Canadian, to a hMPV isolate from The Netherlands, and/or to a hMPV antigen from a hMPV isolate from Australia. The different isolates are described in Peret et al, 2002, J Infect Dis 185:1660-1663, which is incorporated herein by reference in its entirety.

In certain embodiments, an anti-hMPV-antigen antibody binds to allelic variants of a hMPV nucleoprotein, hMPV phosphoprotein, hMPV matrix protein, hMPV small hydrophobic protein, hMPV RNA-dependent RNA polymerase, hMPV F protein, and/or hMPV G protein.

In certain embodiments, an antibody to be used with the methods of treatment of the invention is an antibody that immunospecifically binds to a mammalian MPV, or a protein of a mammalian MPV as described in section 4.1.7.1. In certain embodiments, an antibody to be used with the methods of treatment of the invention is an antibody that immunospecifically binds to a human MPV.

In certain embodiments, the anti-hMPV-antigen antibody binds immunospecifically to a protein/polypeptide that consists, e.g., of an amino acid sequence of SEQ ID NOs: 399-406, 420, or 421, respectively.

In certain embodiments, the anti-hMPV-antigen antibody binds immunospecifically to a protein/polypeptide that consists of an amino acid sequence that is at least 60%, 70%, 80%, 90%, 95%, or at least 98% identical to the amino acid sequence of SEQ ID NOs: 399-406, 420, or 421, respectively. In certain embodiments, the anti-hMPV-antigen antibody binds immunospecifically to a protein/polypeptide that consists of an amino acid sequence that is at most 70%, 80%, 90%, 95%, 98% or at most 100% identical to the amino acid sequence of SEQ ID NOs: 399-406, 420, or 421, respectively.

In certain embodiments, the anti-hMPV-antigen antibody cross reacts with an APV antigen from APV associated with any avian, particularly turkey, duck, or chicken. In certain, more specific embodiments, the anti-hMPV-antibody cross-reacts with an antigen of APV-A, APV-B, APV-C, and/or APV-D, or any combination thereof, particularly turkey APV. In certain more specific embodiments, the anti-hMPV-antigen antibody cross-reacts with an antigen from a European APV isolate. In certain other embodiments, the anti-hMPV-antigen antibody cross-reacts with an antigen from a North American APV isolate. In certain embodiments, the anti-hMPV-antigen antibody cross-reacts with a APV nucleoprotein, APV phosphoprotein, APV matrix protein, APV small hydrophobic protein, APV RNA-dependent RNA polymerase, APV F protein, and/or APV G protein. In certain embodiments, the anti-hMPV-antigen antibody does not cross-react with an APV antigen. In certain embodiments, the anti-hMPV-antigen antibody cross reacts with an APV antigen of an amino acid sequence of, e.g., SEQ ID NO:424 to 429, respectively.

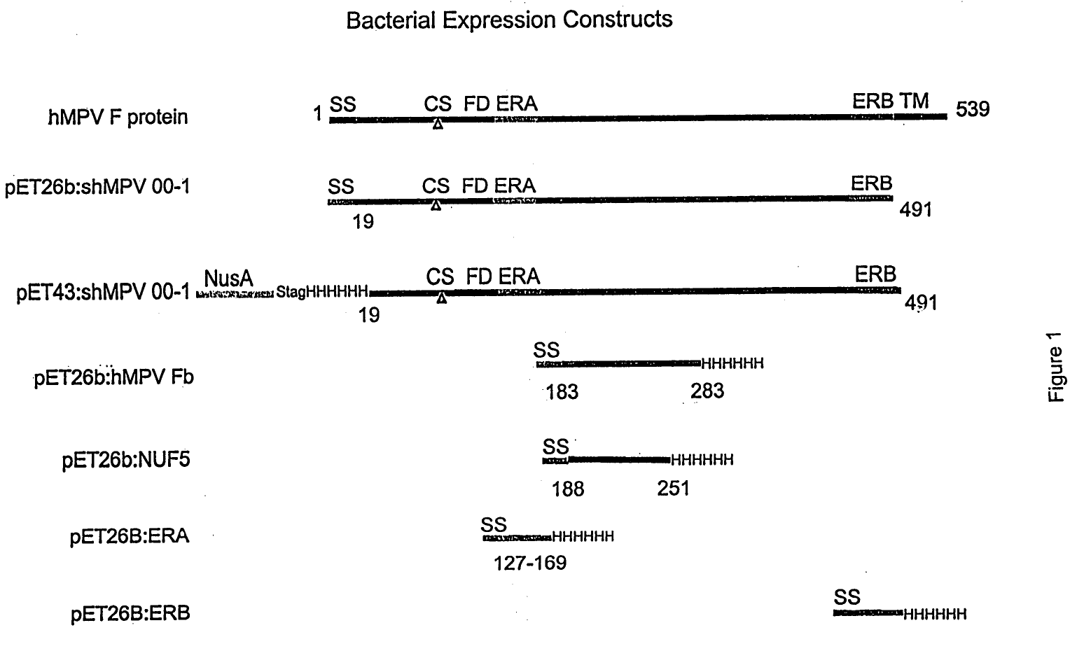

In a specific embodiment, a monoclonal antibody against the F protein of hMPV is generated. In a more specific embodiment, the F protein of hMPV is produced using a baculovirus expression system (e.g., the BD BaculoGold™ Baculovirus Expression Vector System can be used from BD Biosciences, NJ). In certain embodiments, the F protein is expressed without the transmembrane domain to induce secretion of the F protein from the cell in which the protein is expressed. Exemplary expression constructs that can be used for the expression of F protein for the generation of antibodies against the F protein are shown in FIG. 1.

In certain embodiments, peptides that contain the following amino acid sequences are used for the generation of antibodies for use with the methods of the invention: amino acid 19 to 28; amino acid 94 to 106; amino acid 476 to 409, and/or amino acid 223 to 236 of SEQ ID NO:234 or SEQ ID NO:279. In certain embodiments, peptides that contain the amino acid sequences of SEQ ID NOs:430-437 are used as immunogens for the generation of antibodies for use with the methods of the invention. Without being bound by theory the sequences of SEQ ID NOs:430-437 contain the heptad repeats of the F proteins of different strains of human metapneumoviruses.

In certain embodiments, an antibody to be used with the methods of the invention binds to a heptad repeat. In certain, more specific embodiments, an antibody to be used with the methods of the invention binds to a heptad repeat of the F protein of a mammalian metapneumovirus (e.g., hMPV). In certain, even more specific embodiments, an antibody to be used with the methods of the invention binds to heptad repeat 1 or heptad repeat 2 of the F protein of a mammalian metapneumovirus (e.g., hMPV). In certain embodiments, an antibody to be used with the methods of the invention binds to a heptad repeat of the F protein of APV.

Alignment of the human metapneumoviral F protein with the F protein of an avian pneumovirus isolated from Mallard Duck shows 85.6% identity in the ectodomain.

Alignment of the human metapneumoviral F protein with the F protein of an avian pneumovirus isolated from Turkey (subgroup B) shows 75% identity in the ectodomain. See, e.g., co-owned and co-pending Provisional Application No. 60/358,934, entitled “Recombinant Parainfluenza Virus Expression Systems and Vaccines Comprising Heterologous Antigens Derived from Metapneumovirus”, filed on Feb. 21, 2002, by Haller and Tang, which is incorporated herein by reference in its entirety. Therefore, an antigen from avian metapneumovirus, and in particular the F protein from turkey metapneumovirus is a useful antigen for generating antibodies against human metapneumovirus.

In certain embodiments, the anti-hMPV-antigen antibody is a bispecific antibody. In certain embodiments, the bispecific antibody binds to two different epitopes of the same hMPV antigen. In certain other embodiments, the bispecific antibody binds to epitopes on two different hMPV antigens. In certain embodiments, the bispecific antibody binds immunospecifically to (i) a hMPV antigen and (ii) to an APV, a PIV, and/or a RSV antigen.

In certain embodiments, an antibody to be used with the methods of the invention is a bispecific antibody that binds to the F protein of RSV and to the F protein of hMPV. The bispecific antibody can be generated by chemical procedure or a recombinant approach. The antibody can be diabody, F(ab′)₂, F(ab′)₂ fused with lucine zippers, single chain diabodies, etc. The antibody can also be a multivalent antibody, such as quadruplebody. In certain embodiments, a bispecific antibody is constructed using Numax or Synagis for the part of the antibody that binds the RSV F protein in combination with an antibody that binds the hMPV F protein.

4.1.7.3 Multiple Protein Monoclonal Antibodies

To generate multiple protein monoclonal antibodies, Balb/c or SJL mice (mice can be obtained, e.g., from The Jackson Laboratory, Maine) are immunized first with live hMPV and later with adjuvanted hMPV, bovine PIV or purified F protein of hMPV. In a more specific embodiment, mice are immunized intranasally one to two times with hMPV followed by intraperitoneal injections with either hMPV (to produce all types of neutralizing antibodies, e.g., F or G protein) or with intranasal immunization with bPIV/hMPV F or intraperitoneal immunization of purified F protein. bPIV/hMPV F is a chimeric virus wherein the coding sequence for the hMPV F protein is inserted into bovine PIV. A more detailed description of PIV vectors and their use as expression systems can be found in co-owned and co-pending U.S. application Nos.: Ser. No. 10/371,264 and Ser. No. 10/373,567, both filed on Feb. 21, 2003, both of which are incorporated herein by reference in their entireties. In certain specific embodiments, for each immunization 100 microliter of virus at 10⁶-10⁷ pfu/ml per mouse are used.

4.1.8 Anti-PIV-Antigen Antibodies

In certain embodiments, an anti-PIV-antigen antibody binds immunospecifically to a PIV nucleocapsid structural protein, a PIV fusion glycoprotein, a PIV phosphoprotein, a PIV L protein, a PIV matrix protein, a PIV HN glycoprotein, a PIV RNA-dependent RNA polymerase, a PIV Y1 protein, a PIV D protein, a F glycoprotein, a PIV hemagglutinin-neuraminidase, or a PIV C protein.

In certain embodiments, the anti-PIV-antigen antibody binds to an antigen of PIV type 1, PIV type 2, and/or PIV type 3, or any combination thereof.

In certain embodiments, an anti-PIV-antigen antibody binds to allelic variants of a PIV nucleocapsid structural protein, a PIV fusion glycoprotein, a PIV phosphoprotein, a PIV L protein, a PIV matrix protein, a PIV HN glycoprotein, a PIV RNA-dependent RNA polymerase, a PIV Y1 protein, a PIV D protein, a F glycoprotein, a PIV hemagglutinin-neuraminidase, or a PIV C protein.

In certain embodiments, the anti-PIV-antigen antibody binds immunospecifically to a PIV RNA polymerase alpha subunit (Nucleocapsid phosphoprotein), e.g., having an amino acid sequence of SEQ ID NO:407; a PIV L polymerase protein, e.g., having an amino acid sequence of SEQ ID NO:408; a PIV HN glycoprotein, e.g. having an amino acid sequence of SEQ ID NO:409; a PIV matrix protein, e.g., having an amino acid sequence of SEQ ID NO:410; a PIV Y1 protein, e.g., having an amino acid sequence of SEQ ID NO:411; a PIV C protein, e.g., having an amino acid sequence of SEQ ID NO:412; a PIV phosphoprotein, e.g., having an amino acid sequence of SEQ ID NO:413; a PIV nucleoprotein, e.g., having an amino acid sequence of SEQ ID NO:414; a PIV F glycoprotein, e.g., having an amino acid sequence of SEQ ID NO:415; a PIV D protein, e.g., having an amino acid sequence of SEQ ID NO:416; a PIV hemagglutinin-neuraminidase, e.g., having an amino acid sequence of SEQ ID NO:417; a PIV nucleocapsid protein, e.g., having an amino acid sequence of SEQ ID NO:418; a PIV P protein, e.g., having an amino acid sequence of SEQ ID NO:419.

In certain embodiments, the anti-PIV-antigen antibody binds immunospecifically to a protein/polypeptide that consists of an amino acid sequence that is at least 60%, 70%, 80%, 90%, 95%, or at least 98% identical to the amino acid sequence of an RNA polymerase alpha subunit (Nucleocapsid phosphoprotein) SEQ ID NO:407; L polymerase protein SEQ ID NO:408; HN glycoprotein SEQ ID NO:409; matrix protein SEQ ID NO:410; Y1 protein SEQ ID NO:411; C protein SEQ ID NO:412; phosphoprotein SEQ ID NO:413; nucleoprotein SEQ ID NO:414; F glycoprotein SEQ ID NO:415; D protein SEQ ID NO:416; hemagglutinin-neuraminidase SEQ ID NO:417; nucleocapsid protein SEQ ID NO:418; P protein SEQ ID NO:419. In certain embodiments, the anti-PIV-antigen antibody binds immunospecifically to a protein/polypeptide that consists of an amino acid sequence that is at most 70%, 80%, 90%, 95%, 98% or at most 100% identical to the amino acid sequence of an RNA polymerase alpha subunit (Nucleocapsid phosphoprotein) SEQ ID NO:407; L polymerase protein SEQ ID NO:408; HN glycoprotein SEQ ID NO:409; matrix protein SEQ ID NO:410; Y1 protein SEQ ID NO:411; C protein SEQ ID NO:412; phosphoprotein SEQ ID NO:413; nucleoprotein SEQ ID NO:414; F glycoprotein SEQ ID NO:415; D protein SEQ ID NO:416; hemagglutinin-neuraminidase SEQ ID NO:417; nucleocapsid protein SEQ ID NO:418; P protein SEQ ID NO:419.

4.2 Prophylaxis and Therapy of Respiratory Viral Infections

The invention provides methods for broad-spectrum treatment and prevention of respiratory viral infections. To obtain broad-spectrum protection against respiratory viral infection in a subject, a plurality of antibodies, each of which can bind immunospecifically to an epitope on a different virus that causes respiratory infections, is administered to the subject. In certain embodiments, a plurality of antibodies that bind immunospecifically to antigens of different viruses that cause respiratory infections is administered. In certain embodiments, a plurality of antibodies that bind immunospecifically to different antigens of hMPV, PIV, and/or RSV, is administered. In certain embodiments, antibodies that cross-react with antigens from different respiratory viruses are administered. In specific embodiments, an antibody that immunospecifically binds to an antigen of hMPV cross reacts with an antigen of APV, particularly turkey APV. More specifically, an antibody that binds immunospecifically to the F protein of hMPV cross-reacts with the F protein of APV.

In certain embodiments, at least one of the antibodies to be administered to a subject is an antibody-conjugate.

Administering different antibodies with different immunospecificities ensures that the prophylaxis/therapy is effective against respiratory viruses even if some antigens of the viruses have modified amino acid sequences. In general there are two approaches to ensure that at least one of the administered plurality of antibodies binds immunospecifically to one or more of the infectious respiratory viral particles. First, antibodies against different epitopes of one or more viruses may be included in the plurality of antibodies. Thus, even if one of the epitopes of the infectious respiratory viral particle is different from the corresponding epitope against which one of the antibodies was raised, another antibody of the plurality of antibodies binds immunospecifically to an epitope of the infectious respiratory viral particle. In certain embodiments, even if one of the antigens of the infectious respiratory viral particle is different from the corresponding antigen against which one of the antibodies of the plurality of antibodies was raised, another antibody of the plurality of antibodies binds immunospecifically to an antigen of the infectious respiratory viral particle. Secondly, antibodies that cross-react with different antigens from different viruses, such as the F protein from RSV and the F protein from hMPV can be included in the plurality of antibodies to broaden the spectrum of viruses, subtypes of viruses, subgroups of viruses, mutated viruses, groups of viruses, and types of viruses against which the plurality of antibodies is effective.

In certain embodiment of the invention, the antibodies that are administered to the subject have a synergistic effect in treating and/or preventing an respiratory viral infection. In certain embodiments, the combination of a variety of antibodies is effective in treating or preventing a respiratory viral infection while the individual administration of only one antibody is not effective in treating or preventing a respiratory viral infection.

In certain embodiments, the methods of the invention include administering (i) one or more anti-RSV-antigen antibodies or antigen-binding fragments thereof; (ii) one or more anti-hMPV-antigen antibodies or antigen-binding fragments thereof; and/or (iii) one or more anti-PIV-antigen antibodies or antigen-binding fragmens thereof; and (iv) and one or more vaccines directed against viruses that cause respiratory infections. In a specific embodiment, the vaccine is directed against hMPV. Such vaccines are described in U.S. Provisional Application No. 60/358,934, entitled “Recombinant Parainfluenza Virus Expression Systems and Vaccines Comprising Heterologous Antigens Derived from Metapneumovirus”, filed Feb. 21, 2002, which is incorporated by reference in its entirety herein.

In certain other embodiments, the methods further include administering an anti-viral agent. Anti-viral agents include, but are not limited to, nucleoside analogs, such as zidovudine, acyclovir, gangcyclovir, vidarabine, idoxuridine, trifluridine, and ribavirin, as well as foscamet, amantadine, rimantadine, saquinavir, indinavir, ritonavir, and the alpha-interferons.

4.2.1 Combination Prophylaxis and Therapy with Anti-RSV-Antigen Antibodies, Anti-hMPV-Antigen Antibodies, and Anti-PIV-Antigen Antibodies

In certain embodiments, the invention provides methods for preventing, treating and/or ameliorating one or more symptoms of a respiratory viral infection in a subject, the method comprising administering to the subject one or more anti-RSV-antigen antibodies or antigen-binding fragments thereof, one or more anti-PIV-antigen antibodies or antigen-binding fragments thereof, and one or more anti-hMPV-antigen antibodies or antigen-binding fragments thereof. In specific embodiments, the invention provides administering to a subject a prophylactically effective amount of one or more anti-RSV-antigen antibodies or antigen-binding fragments thereof, a prophylactically effective amount of one or more anti-PIV-antigen antibodies or antigen-binding fragments thereof, and a prophylactically effective amount of one or more anti-hMPV-antigen antibodies or antigen-binding fragments thereof to prevent a respiratory viral infection in a subject. In specific embodiments, the invention provides administering to a subject a therapeutically effective amount of one or more anti-RSV-antigen antibodies or antigen-binding fragments thereof, a therapeutically effective amount of one or more anti-PIV-antigen antibodies or antigen-binding fragments thereof, and a therapeutically effective amount of one or more anti-hMPV-antigen antibodies or antigen-binding fragments thereof to treat a respiratory viral infection in a subject. In specific emodiments of the invention, the respiratory viral infection is an infection with RSV, PIV, and/or hMPV. In certain embodiments, the subject is exposed to a risk of infection with RSV, PIV, and/or hMPV.

In certain embodiments, the invention provides methods of passive immunotherapy, wherein the methods comprises administering a first dose of one or more anti-RSV-antigen antibodies or antigen-binding fragments thereof, a second dose of one or more anti-PIV-antigen antibodies or antigen-binding fragments thereof, and a third dose of one or more anti-hMPV-antigen antibodies or antigen-binding fragments thereof and wherein the first dose reduces the incidence of a RSV infection by at least 25%, wherein the second dose reduces the incidence of a PIV infection by at least 25%, and wherein the third dose reduces the incidence of a hMPV infection by at least 25%. In certain embodiments, the first dose reduces the incidence of a RSV infection by at least 10%, 15%, 20%, 30%, 40%, 50%, 60%, 70%, 75%, 80%, 90%, 95%, or by at least 98%, wherein the second dose reduces the incidence of a PIV infection by at least 10%, 15%, 20%, 30%, 40%, 50%, 60%, 70%, 75%, 80%, 90%, 95%, or by at least 98%, and wherein the third dose reduces the incidence of a hMPV infection by at least 10%, 15%, 20%, 30%, 40%, 50%, 60%, 70%, 75%, 80%, 90%, 95%, or by at least 98%.

In certain embodiments, the invention provides a method of passive immunotherapy wherein the method comprises administering to a subject: (i) a first dose of one or more first antibodies or antigen-binding fragments thereof, wherein said one or more first antibodies or antigen-binding fragments thereof bind immunospecifically to a RSV antigen; (ii) a second dose of one or more second antibodies or antigen-binding fragments thereof, wherein said one or more second antibodies or antigen-binding fragments thereof bind immunospecifically to a hMPV antigen, and (iii) a third dose of one or more third antibodies wherein the one or more third antibodies or antigen-binding fragments thereof bind immunospecifically to a PIV antigen, wherein the serum titer of said one or more first antibodies or antigen-binding fragments thereof in the subject is at least 10 μg/ml after 15 days of administering said one or more first antibodies or antigen-binding fragments thereof, wherein the serum titer of said one or more second antibodies or antigen-binding fragments thereof in the subject is at least 10 μg/ml after 15 days of administering said one or more second antibodies or antigen-binding fragments thereof, and wherein the serum titer of said one or more third antibodies or antigen-binding fragments thereof in the subject is at least 10 μg/ml after 15 days of administering said one or more second antibodies or antigen-binding fragments thereof. In certain embodiments, the serum titer of said one or more first antibodies or antigen-binding fragments thereof in the subject is at least 0.1 μg/ml, 0.5 μg/ml, 1 μg/ml, 5 μg/ml, 10 μg/ml, 20 μg/ml, 30 μg/ml, 40 μg/ml, 50 μg/ml, 75 μg/ml, 100 μg/ml, 150 μg/ml, 250 μg/ml, or at least 500 μg/ml after 15 days of administering said one or more first antibodies or antigen-binding fragments thereof, wherein the serum titer of said one or more second antibodies or antigen-binding fragments thereof in the subject is at least 0.1 μg/ml, 0.5 μg/ml, 1 μg/ml, 5 μg/ml, 10 μg/ml, 20 μg/ml, 30 μg/ml, 40 μg/ml, 50 μg/ml, 75 μg/ml, 100 μg/ml, 150 μg/ml, 250 μg/ml, or at least 500 μg/ml after 15 days of administering said one or more second antibodies or antigen-binding fragments thereof, and wherein the serum titer of said one or more third antibodies or antigen-binding fragments thereof in the subject is at least 0.1 μg/ml, 0.5 μg/ml, 1 μg/ml, 5 μg/ml, 10 μg/ml, 20 μg/ml, 30 μg/ml, 40 μg/ml, 50 μg/ml, 75 μg/ml, 100 μg/ml, 150 μg/ml, 250 μg/ml, or at least 500 μg/ml after 15 days of administering said one or more second antibodies or antigen-binding fragments thereof.

In certain embodiments, the one or more anti-RSV-antigen antibodies, the one or more anti-PIV-antigen antibodies, and the one or more anti-hMPV-antigen antibodies, or any combination of these antibodies, are administered concurrently. In certain, more specific embodiments, the antibodies are administered concurrently via the same route, e.g., but not limited to, intravenous or intramuscular. In certain other embodiments, the antibodies are administered concurrently via different routes.

In other embodiments, the one or more anti-RSV-antigen antibodies, the one or more anti-PIV-antigen antibodies, and the one or more anti-hMPV-antigen antibodies are administered subsequent to each other separated by a time period. In certain embodiments, the time period is 1 day, 2 days, 3 days, 4 days, 5 days, 6 days, 1 week, 2 weeks, 3 weeks, 1 month, 2 months, or 3 months. In a specific embodiment of the invention, the one or more anti-RSV-antigen antibodies are administered first, the one or more anti-PIV-antigen antibodies are administered second, and the one or more anti-hMPV-antigen antibodies are administered third. In a specific embodiment of the invention, the one or more anti-hMPV-antigen antibodies are administered first, the one or more anti-RSV-antigen antibodies are administered second, and the one or more anti-PIV-antigen antibodies are administered third.

In a specific embodiment of the invention, the one or more anti-PIV-antigen antibodies are administered first, the one or more anti-hMPV-antigen antibodies are administered second, and the one or more anti-RSV-antigen antibodies are administered third. In certain embodiments, at least one of the antibodies is administered in a sequence of several administrations separated by a time period. Any other order of administration is also encompassed by the methods of the present invention.

The one or more anti-PIV-antigen antibodies, the one or more anti-hMPV-antigen antibodies, and the one or more anti-RSV-antigen antibodies can also be cyclically administered. Cycling therapy involves the administration of a first prophylactic or therapeutic agent for a period of time, followed by the administration of a second prophylactic or therapeutic agent for a period of time, followed by the administration of a third prophylactic or therapeutic agent for a period of time and so forth, and repeating this sequential administration, i.e., the cycle, in order to reduce the development of resistance to one of the agents, to avoid or reduce the side effects of one of the agents, and/or to improve the efficacy of the treatment.

In certain embodiments, administration of the same antibody may be repeated and the administrations may be separated by at least 10 days, 15 days, 30 days, 2 months, 3 months, or at least 6 months. In certain embodiments, administration of the same antibody may be repeated and the administrations may be separated by at most 10 days, 15 days, 30 days, 2 months, 3 months, or at least 6 months.

4.2.2 Combination Prophylaxis and Therapy with Anti-RSV-Antigen Antibodies and Anti-hMPV-Antigen Antibodies

The present invention provides methods of preventing and/or treating and ameliorating one or more symptoms associated with a respiratory viral infection in a subject comprising administering to said subject (i) one or more first antibodies or antigen-binding fragments thereof which immunospecifically bind to one or more RSV antigens; and (ii) one or more second antibodies or antigen-binding fragments thereof which immunospecifically bind to one or more hMPV antigens. In a specific embodiment, the subject is a human. In a specific embodiment, the subject has a viral respiratory infection, in particular, is infected with RSV and/or hMPV. In a specific embodiment, the method prevents a subject from infection with RSV and/or hMPV. In a specific embodiment, the subject is susceptible to RSV and/or hMPV infection. In a specific embodiment, the subject is exposed to the risk of infection with RSV and/or hMPV infection.

In certain embodiments, the one or more first antibodies neutralize RSV. In certain embodiments, the one or more first antibodies neutralize at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 98% or at least 99% of the RSV in an in vitro microneutralization assay (see below). In certain embodiments, the one or more first antibodies neutralize at least 25%, at most 30%, at most 35%, at most 40%, at most 45%, at most 50%, at most 55%, at most 60%, at most 65%, at most 70%, at most 75%, at most 80%, at most 85%, at most 90%, at most 95%, at most 98% or at most 99% of the RSV in an in vitro microneutralization assay (as described in section 4.8.4).

In certain embodiments, the one or more second antibodies neutralize hMPV. In certain embodiments, the one or more second antibodies neutralize at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 98% or at least 99% of the hMPV in an in vitro microneutralization assay (see below). In certain embodiments, the one or more first antibodies neutralize at least 25%, at most 30%, at most 35%, at most 40%, at most 45%, at most 50%, at most 55%, at most 60%, at most 65%, at most 70%, at most 75%, at most 80%, at most 85%, at most 90%, at most 95%, at most 98% or at most 99% of the hMPV in an in vitro microneutralization assay.

In certain embodiments, at least one of the one or more antibodies that bind immunospecifically to a RSV antigen is a high affinity and/or high avidity antibody and/or has a longer serum half-life. In certain embodiments, at least one of the one or more antibodies that bind immunospecifically to a hMPV antigen is a high affinity and/or high avidity antibody and/or has a longer serum half-life.

The high affinity and/or high avidity of the antibodies of the invention enable the use of lower doses of the antibodies compared to non-high affinity or non-high avidity for the amelioration of symptoms associated with RSV infection and/or hMPV infection. The use of lower doses of antibodies which immunospecifically bind to one or more RSV antigens and the use of lower doses of antibodies which immunospecifically bind to one or more hMPV antigens reduces the likelihood of adverse effects, as well as providing a more effective prophylaxis. Further, high affinity and/or high avidity of the antibodies enable less frequent administration of said antibodies than previously thought to be necessary for the prevention, neutralization, treatment and the amelioration of symptoms associated with RSV infection and hMPV infection, respecively.

In certain embodiments, the one or more antibodies that bind immunospecifically to a RSV antigen and/or the one or more antibodies that bind immunospecifically to a hMPV antigen can be administered directly to the site of RSV infection. In particular, at least one of the antibodies can be administered by pulmonary delivery. Such a mode of administration can reduce the dosage and frequency of administration of the antibodies to a subject.

In certain embodiments, the serum titer of at least one of the administered antibodies is 1 μg/ml or less, 2 μg/ml or less, 5 μg/ml or less, 6 μg/ml or less, 10 μg/ml or less, 15 μg/ml or less, 20 μg/ml or less, or 25 μg/ml or less. In certain embodiments, the serum titer of at least one of the administered antibodies is at least 1 μg/ml, at least 2 μg/ml, at least 5 μg/ml, at least 6 μg/ml, at least 10 μg/ml, at least 15 μg/ml, at least 20 μg/ml, at least 25 μg/ml, at least 50 μg/ml, at least 100 μg/ml, at least 125 μg/ml, at least 150 μg/ml, at least 175 μg/ml, at least 200 μg/ml, at least 225 μg/ml, at least 250 μg/ml, at least 275 μg/ml, at least 300 μg/ml, at least 325 μg/ml, at least 350 μg/ml, at least 375 μg/ml, or at least 400 μg/ml.

Preferably a serum titer or serum titer of 1 μg/ml or less, 2 μg/ml or less, 5 μg/ml or less, 6 μg/ml or less, 10 μg/ml or less, 15 μg/ml or less, 20 μg/ml or less, or 25 μg/ml or less is achieved approximately 20 days (preferably 25, 30, 35 or 40 days) after administration of a first dose of antibodies or antigen-binding fragments thereof which immunospecifically bind to a RSV antigen and/or to a hMPV antigen and without administration of any other doses of said antibodies or antigen-binding fragments thereof. Preferably a serum titer or serum titer of at least 1 μg/ml, at least 2 μg/ml, at least 5 μg/ml, at least 6 μg/ml, at least 10 μg/ml, at least 15 μg/ml, at least 20 μg/ml, at least 25 μg/ml, at least 50 μg/ml, at least 100 μg/ml, at least 125 μg/ml, at least 150 μg/ml, at least 175 μg/ml, at least 200 μg/ml, at least 225 μg/ml, at least 250 μg/ml, at least 275 μg/ml, at least 300 μg/ml, at least 325 μg/ml, at least 350 μg/ml, at least 375 μg/ml, or at least 400 μg/ml is achieved approximately 20 days (preferably 25, 30, 35 or 40 days) after administration of a first dose of antibodies or antigen-binding fragments thereof which immunospecifically bind to a RSV antigen and/or to a hMPV antigen and without administration of any other doses of said antibodies or antigen-binding fragments thereof.

In specific embodiments, a serum titer in a non-primate mammal of at least 0.4 μg/ml, 1 μg/ml, 4 μg/ml, 10 μg/ml, 40 μg/ml, at least 80 μg/ml, at least 100 μg/ml, at least 120 μg/ml, at least 150 μg/ml, at least 200 μg/ml, at least 250 μg/ml, or at least 300 μg/ml, of one or more antibodies or antigen-binding fragments thereof that immunospecifically bind to a RSV antigen and/or of one or more antibodies or antigen-binding fragments thereof that bind immunospecifically to a hMPV antigen is achieved at least 1 day after administering a dose of less than 20 mg/kg, 15 mg/kg, 10 mg/kg, less than 2.5 mg/kg, less than 1 mg/kg, or less than 0.5 mg/kg of the antibodies or antibody fragments to the non-primate mammal. In another embodiment, a serum titer in a non-primate mammal of at least 150 μg/ml, at least 200 μg/ml, at least 250 μg/ml, at least 300 μg/ml, at least 350 μg/ml, or at least 400 μg/ml of one or more antibodies or antigen-binding fragments thereof that immunospecifically bind to one or more RSV antigens and/or that bind immunospecifically to a hMPV antigen is achieved at least 1 day after administering a dose of approximately 5 mg/kg, 10 mg/kg, 15 mg/kg, 20 mg/kg, 25 mg/kg, or 30 mg/kg of the antibodies or antibody fragments to the non-primate mammal.

In another embodiment, a serum titer in a primate of at least 0.4 μg/ml, 11 g/ml, 10 μg/ml, 40 μg/ml, preferably at least 80 μg/ml, at least 100 μg/ml, at least 120 μg/ml, at least 150 μg/ml, at least 200 μg/ml, at least 250 μg/ml, or at least 300 μg/ml of one or more antibodies or antigen-binding fragments thereof that immunospecifically bind to one or more RSV antigens and/or to one or more hMPV antigens is achieved at least 30 days after administering a first dose of less than 5 mg/kg, 10 mg/kg, 15 mg/kg, 20 mg/kg, 25 mg/kg, or 30 mg/kg, preferably less than 3 mg/kg, less than 1 mg/kg, or less than 0.5 mg/kg of the antibodies or antigen-binding fragments thereof to the primate. In yet another embodiment, a serum titer in a primate of at least 200 μg/ml, at least 250 μg/ml, at least 300 μg/ml, at least 350 μg/ml, or at least 400 μg/ml of one or more antibodies or antigen-binding fragments thereof that immunospecifically bind to one or more RSV antigens and/or one or more hMPV antigens is achieved at least 30 days after administering a first dose of approximately 5 mg/kg, 10 mg/kg, 15 mg/kg, 20 mg/kg, 25 mg/kg, or 30 mg/kg of the antibodies or antigen-binding fragments thereof to the primate. In accordance with these embodiments, the primate is preferably a human.

The present invention provides methods for preventing, treating, or ameliorating one or more symptoms associated with a respiratory viral infection in a mammal, preferably a human, said methods comprising administering a first dose to said mammal of (i) a prophylactically or therapeutically effective amount of one or more antibodies or antigen-binding fragments thereof that immunospecifically bind to one or more RSV antigens, and (ii) a prophylactically or therapeutically effective amount of one or more antibodies or antigen-binding fragments thereof that immunospecifically bind to one or more hMPV antigens, wherein said effective amount is less than 1.5 mg/kg, 8 mg/kg, 15 mg/kg, 50 mg/kg, or less than 100 mg/kg or approximately this amount of said antibodies or antigen-binding fragments thereof and which results in a serum titer of greater than 40 μg/ml 30 days after the first administration and prior to any subsequent administration. In one embodiment, the respiratory viral infection in a human subject is prevented or treated, or one or more symptoms associated with the respiratory viral infection is ameliorated by administering (i) a first dose of less than 20 mg/kg, 15 mg/kg, 10 mg/kg, preferably less than 5 mg/kg, less than 3 mg/kg, or less than 1 mg/kg or approximately this amount of one or more antibodies or antigen-binding fragments thereof that immunospecifically bind to one or more RSV antigens; and (ii) a second dose of less than 20 mg/kg, 15 mg/kg, 10 mg/kg, less than 5 mg/kg, less than 3 mg/kg, or less than 1 mg/kg or approximately this amount of one or more antibodies or antigen-binding fragments thereof that immunospecifically bind to one or more hMPV antigens so that a serum antibody titer of at least 40 μg/ml, at least 80 μg/ml, or at least 120 μg/ml, at least 150 μg/ml, at least 200 μg/ml, at least 250 μg/ml, or at least 300 μg/ml is achieved 30 days after the administration of the first dose of the antibodies or antibody fragments and prior to the administration of a subsequent dose. In another embodiment, a respiratory infection in a human subject is prevented or treated, or one or more symptoms associated with a respiratory viral infection is ameliorated by administering a first dose of approximately 15 mg/kg of (i) one or more antibodies or antigen-binding fragments thereof that immunospecifically bind to one or more RSV antigens; and (ii) one or more antibodies or antigen-binding fragments thereof that immunospecifically bind to one or more RSV antigens so that a serum antibody titer of at least 10 μg/ml, 25 μg/ml, 50 μg/ml, 75 μg/ml, or at least 100 μg/ml, at least 200 μg/ml, at least 250 μg/ml, at least 300 μg/ml, at least 350 μg/ml, or at least 400 μg/ml is achieved 30 days after the administration of the first dose of the antibodies or antibody fragments and prior to the administration of a subsequent dose.

In certain embodiments, the respiratory viral infection is an infection with RSV and/or hMPV.

In certain embodiments of the invention, the fragments of the antibodies, i.e., the one or more antibodies that bind immunospecifically to a RSV antigen and/or the one or more antibodies that bind immunospecifically to a hMPV antigen comprise a variable heavy (“VH”) domain.

In certain embodiments of the invention, the fragments of the one or more antibodies that bind immunospecifically to a RSV antigen and/or the fragments of the one or more antibodies that bind immunospecifically to a hMPV antigen comprise a variable light (“VL”).

In certain embodiments, at least one of the fragments or the antibodies comprises a VH domain and a VL domain.

In certain embodiments of the invention, the antibodies are administered via sustained release formulations.

In certain embodiments the one or more antibodies or antigen-binding fragments thereof that bind immunospecifically to one or more RSV antigens (hereafter “anti-RSV-antigen antibodies or antigen-binding fragments thereof”) and the one or more antibodies that bind immunospecifically to one or more hMPV antigens (hereafter “anti-hMPV-antigen antibodies or antigen-binding fragments thereof”) are administered concurrently. In certain, more specific embodiments, the antibodies are administered concurrently via the same route, e.g., but not limited to, intravenous or intramuscular. In certain other embodiments, the antibodies are administered concurrently via different routes.

In certain other embodiments, the anti-RSV-antigen antibodies or antigen-binding fragments thereof are administered prior to the administration of the anti-hMPV-antigen antibodies or antigen-binding fragments thereof. In certain other embodiments, the anti-hMPV-antigen antibodies or antigen-binding fragments thereof are administered prior to the administration of the anti-RSV-antigen antibodies or antigen-binding fragments thereof.

In certain embodiments, the anti-RSV-antigen antibodies or antigen-binding fragments thereof are administered in a sequence of individual administrations separated by a time period and the anti-hMPV-antigen antibodies or antigen-binding fragments thereof are administered prior to, concurrently with, or subsequent to the sequence of administering the anti-RSV-antigen antibodies. In certain embodiments, the anti-hMPV-antigen antibodies or antigen-binding fragments thereof are administered in a sequence of individual administrations separated by a time period and the anti-RSV-antigen antibodies or antigen-binding fragments thereof are administered prior to, concurrently with, or subsequent to the sequence of administering the anti-hMPV-antigen antibodies. In certain embodiments, the time period is 1 day, 2 days, 3 days, 4 days, 5 days, 6 days, 1 week, 2 weeks, 3 weeks, 1 month, 2 months, or 3 months.

In certain embodiments, both the anti-RSV-antigen antibodies or antigen-binding fragments thereof and the anti-hMPV-antigen antibodies or antigen-binding fragments thereof are administered in a sequence of individual administrations separated by a time period. In certain more specific embodiments, the two sequences of administrations are in phase with each other. In other embodiments, the two sequences are out-of-phase with each other.

The present invention provides compositions comprising (i) one or more antibodies or antigen-binding fragments thereof that immunospecifically bind to one or more RSV antigens, and (ii) one or more antibodies or antigen-binding fragments thereof that bind immunospecifically to one or more hMPV antigen. In certain embodiments, the pharmaceutical composition further comprises a pharmaceutically acceptable carrier.

In certain embodiments, administration of the same antibody may be repeated and the administrations may be separated by at least 10 days, 15 days, 30 days, 2 months, 3 months, or at least 6 months. In certain embodiments, administration of the same antibody may be repeated and the administrations may be separated by at most 10 days, 15 days, 30 days, 2 months, 3 months, or at least 6 months.

4.2.3 Combination Prophylaxis and Therapy of Anti-PIV-Antigen Antibodies and Anti-hMPV-Antigen Antibodies

The present invention provides methods of preventing and/or treating and ameliorating one or more symptoms associated with a respiratory viral infection in a subject comprising administering to said subject (i) one or more first antibodies or antigen-binding fragments thereof which immunospecifically bind to one or more PIV antigens; and (ii) one or more second antibodies or antigen-binding fragments thereof which immunospecifically bind to one or more hMPV antigens. In a specific embodiment, the subject is a human infected with PIV and hMPV. In a specific embodiment, the method prevents a subject from infection with PIV and hMPV. In a specific embodiment, the subject is susceptible to PIV and hMPV infection.

In certain embodiments, the one or more first antibodies neutralize PIV. In certain embodiments, the one or more first antibodies neutralize at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 98% or at least 99% of the PIV in an in vitro microneutralization assay (see below). In certain embodiments, the one or more first antibodies neutralize at least 25%, at most 30%, at most 35%, at most 40%, at most 45%, at most 50%, at most 55%, at most 60%, at most 65%, at most 70%, at most 75%, at most 80%, at most 85%, at most 90%, at most 95%, at most 98% or at most 99% of the PIV in an in vitro microneutralization assay (as described in section 4.8.4).

In certain embodiments, the one or more second antibodies neutralize hMPV. In certain embodiments, the one or more second antibodies neutralize at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 98% or at least 99% of the hMPV in an in vitro microneutralization assay (see below). In certain embodiments, the one or more first antibodies neutralize at least 25%, at most 30%, at most 35%, at most 40%, at most 45%, at most 50%, at most 55%, at most 60%, at most 65%, at most 70%, at most 75%, at most 80%, at most 85%, at most 90%, at most 95%, at most 98% or at most 99% of the hMPV in an in vitro microneutralization assay.

In certain embodiments, at least one of the one or more antibodies that bind immunospecifically to a PIV antigen is a high affinity and/or high avidity antibody and/or has a longer serum half-life. In certain embodiments, at least one of the one or more antibodies that bind immunospecifically to a hMPV antigen is a high affinity and/or high avidity antibody and/or has a longer serum half-life.

The high affinity and/or high avidity of the antibodies of the invention enable the use of lower doses of the antibodies compared to non-high affinity or non-high avidity for the amelioration of symptoms associated with PIV infection and/or hMPV infection. The use of lower doses of antibodies which immunospecifically bind to one or more PIV antigens and the use of lower doses of antibodies which immunospecifically bind to one or more hMPV antigens reduces the likelihood of adverse effects, as well as providing a more effective prophylaxis. Further, high affinity and/or high avidity of the antibodies enable less frequent administration of said antibodies than previously thought to be necessary for the prevention, neutralization, treatment and the amelioration of symptoms associated with PIV infection and hMPV infection, respectively.

In certain embodiments, the one or more antibodies that bind immunospecifically to a PIV antigen and/or the one or more antibodies that bind immunospecifically to a hMPV antigen can be administered directly to the site of PIV infection. In particular, at least one of the antibodies can be administered by pulmonary delivery. Such a mode of administration can reduce the dosage and frequency of administration of the antibodies to a subject.

In certain embodiments, the serum titer of at least one of the administered antibodies is 1 μg/ml or less, 2 μg/ml or less, 5 μg/ml or less, 6 μg/ml or less, 10 μg/ml or less, 15 μg/ml or less, 20 μg/ml or less, 25 μg/ml or less, 100 μg/ml or less, or 250 μg/ml or less. In certain embodiments, the serum titer of at least one of the administered antibodies is at least 1 μg/ml, at least 2 μg/ml, at least 5 μg/ml, at least 6 μg/ml, at least 10 μg/ml, at least 15 μg/ml, at least 20 μg/ml, at least 25 μg/ml, at least 50 μg/ml, at least 100 μg/ml, at least 125 μg/ml, at least 150 g/ml, at least 175 μg/ml, at least 200 μg/ml, at least 225 μg/ml, at least 250 μg/ml, at least 275 μg/ml, at least 300 μg/ml, at least 325 μg/ml, at least 350 μg/ml, at least 375 μg/ml, or at least 400 μg/ml. Preferably a serum titer or serum titer of 1 μg/ml or less, 2 μg/ml or less, 5 μg/ml or less, 6 μg/ml or less, 10 μg/ml or less, 15 μg/ml or less, 20 μg/ml or less, or 25 μg/ml or less is achieved approximately 20 days (preferably 25, 30, 35 or 40 days) after administration of a first dose of antibodies or antigen-binding fragments thereof which immunospecifically bind to a PIV antigen and/or to a hMPV antigen and without administration of any other doses of said antibodies or antigen-binding fragments thereof. Preferably a serum titer or serum titer of at least 1 μg/ml, at least 2 μg/ml, at least 5 μg/ml, at least 6 μg/ml, at least 10 μg/ml, at least 15 μg/ml, at least 20 μg/ml, at least 25 μg/ml, at least 50 μg/ml, at least 100 μg/ml, at least 125 μg/ml, at least 150 μg/ml, at least 175 μg/ml, at least 200 μg/ml, at least 225 μg/ml, at least 250 μg/ml, at least 275 μg/ml, at least 300 μg/ml, at least 325 μg/ml, at least 350 μg/ml, at least 375 μg/ml, or at least 400 μg/ml is achieved approximately 20 days (preferably 25, 30, 35 or 40 days) after administration of a first dose of antibodies or antigen-binding fragments thereof which immunospecifically bind to a PIV antigen and/or to a hMPV antigen and without administration of any other doses of said antibodies or antigen-binding fragments thereof.

In specific embodiments, a serum titer in a non-primate mammal of at least 0.4 μg/ml, 1 μg/ml, 4 μg/ml, 10 μg/ml, 40 μg/ml, at least 80 μg/ml, at least 100 μg/ml, at least 120 μg/ml, at least 150 μg/ml, at least 200 μg/ml, at least 250 μg/ml, or at least 300 μg/ml, of one or more antibodies or antigen-binding fragments thereof that immunospecifically bind to a PIV antigen and/or of one or more antibodies or antigen-binding fragments thereof that bind immunospecifically to a hMPV antigen is achieved at least 1 day after administering a dose of less than 100 mg/kg, 50 mg/kg, 10 mg/kg, less than 2.5 mg/kg, less than 1 mg/kg, or less than 0.5 mg/kg of the antibodies or antibody fragments to the non-primate mammal. In another embodiment, a serum titer in a non-primate mammal of at least 150 μg/ml, at least 200 μg/ml, at least 250 μg/ml, at least 300 μg/ml, at least 350 μg/ml, or at least 400 μg/ml of one or more antibodies or antigen-binding fragments thereof that immunospecifically bind to one or more PIV antigens and/or that bind immunospecifically to a hMPV antigen is achieved at least 1 day after administering a dose of approximately 5 mg/kg of the antibodies or antibody fragments to the non-primate mammal.

In another embodiment, a serum titer in a primate of at least 0.4 μg/ml, 1 μg/ml, 4 μg/ml, 10 μg/ml, 40 μg/ml, preferably at least 80 μg/ml, at least 100 μg/ml, at least 120 μg/ml, at least 150 μg/ml, at least 200 μg/ml, at least 250 μg/ml, or at least 300 μg/ml of one or more antibodies or antigen-binding fragments thereof that immunospecifically bind to one or more PIV antigens and/or to one or more hMPV antigens is achieved at least 30 days after administering a first dose of less than 5 mg/kg, preferably less than 3 mg/kg, less than 1 mg/kg, or less than 0.5 mg/kg of the antibodies or antigen-binding fragments thereof to the primate. In yet another embodiment, a serum titer in a primate of at least 200 μg/ml, at least 250 μg/ml, at least 300 μg/ml, at least 350 μg/ml, or at least 400 μg/ml of one or more antibodies or antigen-binding fragments thereof that immunospecifically bind to one or more PIV antigens and/or one or more hMPV antigens is achieved at least 30 days after administering a first dose of approximately 15 mg/kg of the antibodies or antigen-binding fragments thereof to the primate. In accordance with these embodiments, the primate is preferably a human.

The present invention provides methods for preventing, treating, or ameliorating one or more symptoms associated with a respiratory viral infection in a mammal, preferably a human, said methods comprising administering a first dose to said mammal of (i) a prophylactically or therapeutically effective amount of one or more antibodies or antigen-binding fragments thereof that immunospecifically bind to one or more PIV antigens, and (ii) a prophylactically or therapeutically effective amount of one or more antibodies or antigen-binding fragments thereof that immunospecifically bind to one or more hMPV antigens, wherein said effective amount is less than 1.5 mg/kg, 15 mg/kg, 50 mg/kg, or 100 mg/kg or approximately this amount of said antibodies or antigen-binding fragments thereof and which results in a serum titer of greater than 0.4 μg/ml, 1 μg/ml, 4 μg/ml, 10 μg/ml, 40 μg/ml 30 days after the first administration and prior to any subsequent administration. In one embodiment, the respiratory viral infection in a human subject is prevented or treated, or one or more symptoms associated with the respiratory viral infection is ameliorated by administering (i) a first dose of less than 100 mg/kg or less than 10 mg/kg, about 15 mg/kg less than 5 mg/kg, less than 3 mg/kg, or less than 1 mg/kg or approximately this amount of one or more antibodies or antigen-binding fragments thereof that immunospecifically bind to one or more PIV antigens; and (ii) a first dose of less than 10 mg/kg, about 15 mg/kg less than 5 mg/kg, less than 3 mg/kg, or less than 1 mg/kg or approximately this amount of one or more antibodies or antigen-binding fragments thereof that immunospecifically bind to one or more hMPV antigens so that a serum antibody titer of at least 0.4 μg/ml, 1 μg/ml, 4 μg/ml, 10 μg/ml, 40 μg/ml, preferably at least 80 μg/ml, or at least 120 μg/ml, at least 150 μg/ml, at least 200 μg/ml, at least 250 μg/ml, or at least 300 μg/ml is achieved 30 days after the administration of the first dose of the antibodies or antibody fragments and prior to the administration of a subsequent dose. In another embodiment, a respiratory infection in a human subject is prevented or treated, or one or more symptoms associated with a respiratory viral infection is ameliorated by administering a first dose of approximately 15 mg/kg of (i) one or more antibodies or antigen-binding fragments thereof that immunospecifically bind to one or more PIV antigens; and (ii) one or more antibodies or antigen-binding fragments thereof that immunospecifically bind to one or more PIV antigens so that a serum antibody titer of at least 1 μg/ml, 5 μg/ml, 10 μg/ml, 50 μg/ml, 75 μg/ml, or at least 100 μg/ml, at least 200 μg/ml, at least 250 μg/ml, at least 300 μg/ml, at least 350 μg/ml, or at least 400 μg/ml is achieved 30 days after the administration of the first dose of the antibodies or antibody fragments and prior to the administration of a subsequent dose.

In certain embodiments, the respiratory viral infection is an infection with PIV and hMPV.

In certain embodiments of the invention, the fragments of the antibodies, i.e., the one or more antibodies that bind immunospecifically to a PIV antigen and/or the one or more antibodies that bind immunospecifically to a hMPV antigen comprise a variable heavy (“VH”) domain.

In certain embodiments of the invention, the fragments of the one or more antibodies that bind immunospecifically to a PIV antigen and/or the fragments of the one or more antibodies that bind immunospecifically to a hMPV antigen comprise a variable light (“VL”).

In certain embodiments, at least one of the fragments or the antibodies comprises a VH domain and a VL domain.

In certain embodiments of the invention, the antibodies are administered via sustained release formulations.

In certain embodiments the one or more antibodies or antigen-binding fragments thereof that bind immunospecifically to one or more PIV antigens (hereafter “anti-PIV-antigen antibodies or antigen-binding fragments thereof”) and the one or more antibodies that bind immunospecifically to one or more hMPV antigens (hereafter “anti-hMPV-antigen antibodies or antigen-binding fragments thereof”) are administered concurrently. In certain, more specific embodiments, the antibodies are administered concurrently via the same route, e.g., but not limited to, intravenous or intramuscular. In certain other embodiments, the antibodies are administered concurrently via different routes.

In certain other embodiments, the anti-PIV-antigen antibodies or antigen-binding fragments thereof are administered prior to the administration of the anti-hMPV-antigen antibodies or antigen-binding fragments thereof. In certain other embodiments, the anti-hMPV-antigen antibodies or antigen-binding fragments thereof are administered prior to the administration of the anti-PIV-antigen antibodies or antigen-binding fragments thereof.

In certain embodiments, the anti-PIV-antigen antibodies or antigen-binding fragments thereof are administered in a sequence of individual administrations separated by a time period and the anti-hMPV-antigen antibodies or antigen-binding fragments thereof are administered prior to, concurrently with, or subsequent to the sequence of administering the anti-PIV-antigen antibodies. In certain embodiments, the anti-hMPV-antigen antibodies or antigen-binding fragments thereof are administered in a sequence of individual administrations separated by a time period and the anti-PIV-antigen antibodies or antigen-binding fragments thereof are administered prior to, concurrently with, or subsequent to the sequence of administering the anti-hMPV-antigen antibodies. In certain embodiments, the time period is 1 day, 2 days, 3 days, 4 days, 5 days, 6 days, 1 week, 2 weeks, 3 weeks, 1 month, 2 months, or 3 months.

In certain embodiments, both the anti-PIV-antigen antibodies or antigen-binding fragments thereof and the anti-hMPV-antigen antibodies or antigen-binding fragments thereof are administered in a sequence of individual administrations separated by a time period. In certain more specific embodiments, the two sequences of administrations are in phase with each other. In other embodiments, the two sequences are out-of-phase with each other.

The present invention provides compositions comprising (i) one or more antibodies or antigen-binding fragments thereof that immunospecifically bind to one or more PIV antigens, and (ii) one or more antibodies or antigen-binding fragments thereof that bind immunospecifically to one or more hMPV antigen. In certain embodiments, the pharmaceutical compositions further comprise a pharmaceutically acceptable carrier.

In certain embodiments, administration of the same antibody may be repeated and the administrations may be separated by at least 10 days, 15 days, 30 days, 2 months, 3 months, or at least 6 months. In certain embodiments, administration of the same antibody may be repeated and the administrations may be separated by at most 10 days, 15 days, 30 days, 2 months, 3 months, or at least 6 months.

4.3 Prophylactic and Therapeutic Uses of Antibodies

Antibodies to be used with the methods of the invention are anti-RSV-antigen antibodies, anti-PIV-antigen antibodies, and/or anti-hMPV-antigen antibodies.

The present invention is directed to antibody-based therapies which involve administering antibodies or antigen-binding fragments thereof to a mammal, preferably a human, for preventing, treating, or ameliorating one or more symptoms associated with a RSV, PIV, and/or hMPV infection. In particular, the methods of the invention comprise (i) administering one or more anti-RSV-antigen antibodies or antigen-binding fragments thereof and one or more anti-PIV-antigen antibodies or antigen-binding fragments thereof; (ii) administering one or more anti-PIV-antigen antibodies or antigen-binding fragments thereof and one or more anti-hMPV-antigen antibodies or antigen-binding fragments thereof; or (iii) administering one or more anti-RSV-antigen antibodies or antigen-binding fragments thereof, one or more anti-PIV-antigen antibodies or antigen-binding fragments thereof, and one or more anti-hMPV-antigen antibodies or antigen-binding fragments thereof. Prophylactic and therapeutic compositions of the invention include, but are not limited to, (i) one or more anti-RSV-antigen antibodies or antigen-binding fragments thereof and one or more anti-PIV-antigen antibodies or antigen-binding fragments thereof; (ii) one or more anti-PIV-antigen antibodies or antigen-binding fragments thereof and one or more anti-hMPV-antigen antibodies or antigen-binding fragments thereof; or (iii) one or more anti-RSV-antigen antibodies or antigen-binding fragments thereof, one or more anti-PIV-antigen antibodies or antigen-binding fragments thereof, and one or more anti-hMPV-antigen antibodies or antigen-binding fragments thereof. Antibodies to be used with the methods of the invention or fragments thereof may be provided in pharmaceutically acceptable compositions as known in the art or as described herein.

Antibodies or antigen-binding fragments thereof which do not prevent RSV, PIV, and/or hMPV from binding its host cell receptor but inhibit or downregulate RSV, PIV, and/or hMPV replication can also be administered to a mammal to treat, prevent or ameliorate one or more symptoms associated with a respiratory infection. The ability of an antibody or fragment thereof to inhibit or downregulate RSV, PIV, and/or hMPV replication may be determined by techniques described herein or otherwise known in the art. For example, the inhibition or downregulation of RSV, PIV, and/or hMPV replication can be determined by detecting the RSV titer in the lungs of a mammal, preferably a human.

In a specific embodiment, an antibody to be used with the methods of the invention or fragments thereof inhibit or downregulates RSV, PIV, and/or hMPV replication by at least 99%, at least 95%, at least 90%, at least 85%, at least 80%, at least 75%, at least 70%, at least 60%, at least 50%, at least 45%, at least 40%, at least 45%, at least 35%, at least 30%, at least 25%, at least 20%, or at least 10% relative to RSV, PIV, and/or hMPV replication, respectively, in absence of said antibodies or antibody fragments. In another embodiment, a combination of antibodies, a combination of antibody fragments, or a combination of antibodies and antibody fragments inhibit or downregulate a RSV, PIV, and/or hMPV replication, respectively, by at least 99%, at least 95%, at least 90%, at least 85%, at least 80%, at least 75%, at least 70%, at least 60%, at least 50%, at least 45%, at least 40%, at least 45%, at least 35%, at least 30%, at least 25%, at least 20%, or at least 10% relative to RSV replication in absence of said antibodies and/or antibody fragments.

One or more antibodies of the present invention or fragments thereof that immunospecifically bind to one or more RSV antigens, one or more PIV antigens, and/or one or more hMPV antigens may be used locally or systemically in the body as a therapeutic. The antibodies to be used with the methods of this invention or fragments thereof may also be advantageously utilized in combination with other monoclonal or chimeric antibodies, or with lymphokines or hematopoietic growth factors (such as, e.g., IL-2, IL-3 and IL-7), which, for example, serve to increase the number or activity of effector cells which interact with the antibodies. The antibodies to be used with the methods of this invention or fragments thereof may also be advantageously utilized in combination with other monoclonal or chimeric antibodies, or with lymphokines or hematopoietic growth factors (such as, e.g., IL-2, IL-3 and IL-7), which, for example, serve to increase the immune response. The antibodies to be used with the methods of this invention or fragments thereof may also be advantageously utilized in combination with one or more drugs used to treat RSV infection such as, for example anti-viral agents. Antibodies to be used with the methods of the invention or fragments may be used in combination with one or more of the following drugs: NIH-351 (Gemini Technologies), RSVf-2 (Intracel), F-50042 (Pierre Fabre), T-786 (Trimeris), VP-36676 (ViroPharma), RFI-641 (American Home Products), VP-14637 (ViroPharma), PFP-1 and antiviral PFP-2 (American Home Products), RSV vaccine (Avant Immunotherapeutics), and F-50077 (Pierre Fabre). In certain embodiments, antibodies to be used with the methods of the invention or fragments may be used in combination with the high affinity human monoclonal antibodies specific to RSV F-protein as disclosed in U.S. Pat. No. 5,811,524, by Brams et al., issued Sep. 22, 1998, which is incorporated herein by reference in its entirety.

The antibodies to be used with the methods of the invention may be administered alone or in combination with other types of treatments (e.g., hormonal therapy, immunotherapy, and anti-inflammatory agents). Generally, administration of products of a species origin or species reactivity (in the case of antibodies) that is the same species as that of the patient is preferred. Thus, in a preferred embodiment, human or humanized antibodies, fragments derivatives, analogs, or nucleic acids, are administered to a human patient for therapy or prophylaxis.

In certain embodiments, high affinity and/or potent in vivo inhibiting antibodies and/or neutralizing antibodies that immunospecifically bind to a RSV, PIV, and/or hMPV antigen, for both immunoassays directed to RSV, PIV, and/or hMPV, prevention of RSV, PIV, and/or hMPV infection and therapy for RSV, PIV, and/or hMPV infection are used.

In certain embodiments, the therapeutic and/or prophylactic methods of the invention are used to treat, prevent or ameliorate one or more symptoms associated with a respiratory viral infection in a human with cystic fibrosis, bronchopulmonary dysplasia, congenital heart disease, congenital immunodeficiency or acquired immunodeficiency, or to a human who has had a bone marrow transplant. In certain embodiments, the respiratory viral infection is an infection with RSV, PIV, and/or hMPV. In certain embodiments, the therapeutic and/or prophylactic methods of the invention are used to treat, prevent or ameliorate one or more symptoms associated with a respiratory viral infection in a human infant, preferably a human infant born prematurely or a human infant at risk of hospitalization for RSV infection to treat, prevent or ameliorate one or more symptoms associated with RSV infection. In certain embodiments, the therapeutic and/or prophylactic methods of the invention are used to treat, prevent or ameliorate one or more symptoms associated with a respiratory viral infection in the elderly or people in group homes (e.g., nursing homes or rehabilitation centers).

In certain embodiments of the invention, the target population for the therapeutic methods of the invention is defined by age. In certain embodiments, the target population for the therapeutic methods of the invention is characterized by a disease or disorder in addition to a respiratory tract infection.

In a specific embodiment, the target population encompasses young children, below 2 years of age. In a more specific embodiment, the children below the age of 2 years do not suffer from illnesses other than respiratory tract infection.

In other embodiments, the target population encompasses patients above 5 years of age. In a more specific embodiment, the patients above the age of 5 years suffer from an additional disease or disorder including cystic fibrosis, leukaemia, and non-Hodgkin lymphoma, or recently received bone marrow or kidney transplantation.

In a specific embodiment of the invention, the target population encompasses subjects in which the hMPV infection is associated with immunosuppression of the hosts. In a specific embodiment, the subject is an immunocompromised individual. In a specific embodiment, a subject to be treated with the methods of the invention is also infected with HIV.

In a specific embodiments, the subject to be treated with the methods of the invention has been diagnosed with severe respiratory syncytial virus bronchilitis. Without being bound by theory, an individual diagnosed with severe respiratory syncytial virus is also likely to be infected with hMPV. In a specific embodiments, the subject to be treated with the methods of the invention has been diagnosed with acute respiratory tract illness.

In certain embodiments, the target population for the methods of the invention encompasses the elderly.

In a specific embodiment, the subject to be treated or diagnosed with the methods of the invention was infected with hMPV in the winter months.

In certain embodiments, an effective amount of the anti-RSV-antigen antibodies, anti-PIV-antigen antibodies, and/or anti-hMPV-antigen antibodies or antibody fragments thereof reduces the RSV, PIV, and/or hMPV titers in the lung as measured, for example, by the concentration of RSV, PIV, and/or hMPV in sputum samples or a lavage from the lungs from a mammal. In certain embodiments, an effective amount of an antibody to be used with the invention is sufficient to induce an immune response in the mammal.

In certain embodiments, the antibodies to be used with the methods of the invention are administered via sustained release formulations.

In certain embodiments, an antibody to be used with the methods of the invention binds to a heptad repeat. In certain embodiments, an antibody to be used with the methods of the invention binds to a heptad repeat of RSV, PIV, or hMPV. In certain embodiments, an antibody to be used with the methods of the invention binds to a heptad repeat of the F protein of RSV, PIV, or hMPV. In certain, more specific embodiments, an antibody to be used with the methods of the invention binds to a heptad repeat of the F protein of a mammalian metapneumovirus (e.g., hMPV). In certain, even more specific embodiments, an antibody to be used with the methods of the invention binds to heptad repeat 1 or heptad repeat 2 of the F protein of a mammalian metapneumovirus (e.g., hMPV).

In certain embodiments of the invention, an antibody that immunospecifically binds to an antigen of hMPV of subgroup A or subgroup B can be used with the methods of the invention. In certain embodiments of the invention, an antibody that immunospecifically binds to an antigen of hMPV of variant A1, A2, B1 or B2.

4.3.1 Methods of Administration of Antibodies

The invention provides methods of treatment, prophylaxis, and amelioration of one or more symptoms associated with respiratory viral infection by administrating to a subject of an effective amount of one or more antibodies or fragment thereof, or pharmaceutical is composition comprising one or more antibodies of the invention or fragment thereof. In particular, the antibodies to be used with the methods of the invention are administered as a mixture, e.g., a composition comprising anti-RSV-antigen antibodies, anti-PIV-antigen antibodies, and/or anti-hMPV-antigen antibodies, or any combination thereof. In a preferred aspect, an antibody or fragment thereof is substantially purified (i.e., substantially free from substances that limit its effect or produce undesired side-effects). The subject is preferably a mammal such as non-primate (e.g., cows, pigs, horses, cats, dogs, rats etc.) and a primate (e.g., monkey such as a cynomolgous monkey and a human). In a preferred embodiment, the subject is a human. In another preferred embodiment, the subject is a human infant or a human infant born prematurely. In more specific embodiments, the prematurely born infant was born between 30-35 weeks gestational age or between 35-40 weeks of gestational age. In a preferred embodiment, the prematurely born infant was born between 32 and 35 weeks of gestational age. In certain other embodiments, the prematurely born infant was born at less than 32 weeks gestational age. In certain other embodiments, the prematurely born infant was born at 35-38 weeks gestational age. In other embodiments, the subject is an infant born at 38-40 weeks gestational age or greater than 40 weeks gestational age. In another embodiment, the subject is a human with cystic fibrosis, bronchopulmonary dysplasia, congenital heart disease, congenital immunodeficiency or acquired immunodeficiency, a human who has had a bone marrow transplant, or an elderly human.

Various delivery systems are known and can be used to administer an antibody or an antigen-binding fragment thereof, e.g., encapsulation in liposomes, microparticles, microcapsules, recombinant cells capable of expressing the antibody or antibody fragment, receptor-mediated endocytosis (see, e.g., Wu and Wu, J. Biol. Chem. 262:4429-4432 (1987)), construction of a nucleic acid as part of a retroviral or other vector, etc. Methods of administering an antibody or fragment thereof, or pharmaceutical composition include, but are not limited to, parenteral administration (e.g., intradermal, intramuscular, intraperitoneal, intravenous and subcutaneous), epidural, and mucosal (e.g., intranasal and oral routes). In a specific embodiment, antibodies or antigen-binding fragments thereof, or pharmaceutical compositions are administered intramuscularly, intravenously, or subcutaneously. The compositions may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. In addition, pulmonary administration can also be employed, e.g., by use of an inhaler or nebulizer, and formulation with an aerosolizing agent. See, e.g., U.S. Pat. Nos. 6,019,968, 5,985, 320, 5,985,309, 5,934,272, 5,874,064, 5,855,913, 5,290,540, and 4,880,078; and PCT Publication Nos. WO 92/19244, WO 97/32572, WO 97/44013, WO 98/31346, and WO 99/66903, each of which is incorporated herein by reference their entirety. In a preferred embodiment, an antibody or fragment thereof, or composition comprising the antibodies to be used with the methods of the invention using Alkermes AIR™ pulmonary drug delivery technology (Alkermes, Inc., Cambridge, Mass.).

In certain embodiments, an antibody or fragment thereof is packaged in a hermetically sealed container such as an ampoule or sachette indicating the quantity of antibody or antibody fragment. In one embodiment, each antibody or antibody fragment or combination thereof is supplied as a dry sterilized lyophilized powder or water free concentrate in a hermetically sealed container and can be reconstituted, e.g., with water or saline to the appropriate concentration for administration to a subject. For stabilized liquid antibody formulations, see U.S. Provisional Patent Application Nos.: 60/388,920, filed on Jun. 14, 2002, and 60/388,921, filed Jun. 14, 2002, which are incorporated by reference herein in their entireties. Preferably, each antibody or antibody fragment or combination thereof is supplied as a dry sterile lyophilized powder in a hermetically sealed container at a unit dosage for each antibody of at least 5 mg, more preferably at least 10 mg, at least 15 mg, at least 25 mg, at least 35 mg, at least 45 mg, at least 50 mg, or at least 75 mg. Each lyophilized antibody or antibody fragment or combination thereof should be stored at between 2 and 8° C. in its original container and the antibody or antibody fragment should be administered within 12 hours, preferably within 6 hours, within 5 hours, within 3 hours, or within 1 hour after being reconstituted. In an alternative embodiment, an antibody or fragment thereof is supplied in liquid form in a hermetically sealed container indicating the quantity and concentration of the antibody or antibody fragment. Preferably, the liquid form of the antibody or fragment thereof or combination thereof is supplied in a hermetically sealed container at a concentration for each antibody least 1 mg/ml, more preferably at least 2.5 mg/ml, at least 5 mg/ml, at least 8 mg/ml, at least 10 mg/ml, at least 15 mg/ml, at least 25 mg/ml, at least 50 mg/ml, at least 100 mg/ml, at least 125 mg/ml, at least 150 mg/ml, at least 200 mg/ml, or at least 250 mg/ml, or approximately 2.5 mg/ml, 5 mg/ml, 8 mg/ml, 10 mg/ml, 15 mg/ml, 25 mg/ml, 50 mg/ml, 100 mg/ml, 125 mg/ml, 150 mg/ml, 200 mg/ml, or 250 mg/ml.

In a specific embodiment, it may be desirable to administer the antibodies locally to the area in need of treatment; this may be achieved by, for example, and not by way of limitation, local infusion, by injection, or by means of an implant, said implant being of a porous, non-porous, or gelatinous material, including membranes, such as sialastic membranes, or fibers. Preferably, when administering a an antibody or fragment thereof, care must be taken to use materials to which the antibody or antibody fragment does not absorb. In a specific embodiment, the antibodies may be administered by pulmonary delivery.

In another embodiment, an antibody can be delivered in a vesicle, in particular a liposome (see Langer, Science 249:1527-1533 (1990); Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid.).

In yet another embodiment, an antibody can be delivered in a controlled release or sustained release system. In one embodiment, a pump may be used to achieve controlled or sustained release (see Langer, supra; Sefton, 1987, CRC Crit. Ref. Biomed. Eng. 14:20; Buchwald et al., 1980, Surgery 88:507; Saudek et al., 1989, N. Engl. J. Med. 321:574). In another embodiment, polymeric materials can be used to achieve controlled or sustained release of the antibodies of the invention or fragments thereof (see e.g., Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, 1983, J., Macromol. Sci. Rev. Macromol. Chem. 23:61; see also Levy et al., 1985, Science 228:190; During et al., 1989, Ann. Neurol. 25:351; Howard et al., 1989, J. Neurosurg. 7 1:105); U.S. Pat. No. 5,679,377; U.S. Pat. No. 5,916,597; U.S. Pat. No. 5,912,015; U.S. Pat. No. 5,989,463; U.S. Pat. No. 5,128,326; PCT Publication No. WO 99/15154; and PCT Publication No. WO 99/20253. Examples of polymers used in sustained release formulations include, but are not limited to, poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. In a preferred embodiment, the polymer used in a sustained release formulation is inert, free of leachable impurities, stable on storage, sterile, and biodegradable. In yet another embodiment, a controlled or sustained release system can be placed in proximity of the therapeutic target, i.e., the lungs, thus requiring only a fraction of the systemic dose (see, e.g., Goodson, in Medical Applications of Controlled Release, supra, vol. 2, pp. 115-138(1984)).

Controlled release systems are discussed in the review by Langer (1990, Science 249:1527-1533). Any technique known to one of skill in the art can be used to produce sustained release formulations comprising one or more antibodies or antigen-binding fragments thereof. See, e.g., U.S. Pat. No. 4,526,938, PCT publication WO 91/05548, PCT publication WO 96/20698, Ning et al., 1996, “Intratumoral Radioimmunotheraphy of a Human Colon Cancer Xenograft Using a Sustained-Release Gel,” Radiotherapy & Oncology 39:179-189, Song et al., 1995, “Antibody Mediated Lung Targeting of Long-Circulating Emulsions,” PDA Journal of Pharmaceutical Science & Technology 50:372-397, Cleek et al., 1997, “Biodegradable Polymeric Carriers for a bFGF Antibody for Cardiovascular Application,” Pro. Int'l. Symp. Control. Rel. Bioact. Mater. 24:853-854, and Lam et al., 1997, “Microencapsulation of Recombinant Humanized Monoclonal Antibody for Local Delivery,” Proc. Int'l. Symp. Control Rel. Bioact. Mater. 24:759-760, each of which is incorporated herein by reference in their entireties.

In certain embodiments the antibodies are administered repeatedly, wherein the administrations are separated by at least 10 days, 15 days, 30 days, 2 months, 3 months or at least 6 months. In certain embodiments the antibodies are administered repeatedly, wherein the administrations are separated by at most 10 days, 15 days, 30 days, 2 months, 3 months or at most 6 months.

In certain embodiments, the antibodies are administered during the season of increased risk of pulmonary infections. In specific embodiments, the antibodies are administered during the RSV season.

4.4 Pharmaceutical Compositions

The present invention also provides pharmaceutical compositions. Such compositions comprise one or more of the following: (i) one or more anti-RSV-antigen antibodies or antigen-binding fragments thereof and one or more anti-PIV-antigen antibodies or antigen-binding fragments thereof; (ii) one or more anti-PIV-antigen antibodies or antigen-binding fragments thereof and one or more anti-hMPV-antigen antibodies or antigen-binding fragments thereof; or (iii) one or more anti-RSV-antigen antibodies or antigen-binding fragments thereof, one or more anti-PIV-antigen antibodies or antigen-binding fragments thereof. In certain embodiments, the pharmaceutical composition further comprises a pharmaceutically acceptable carrier. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant (e.g., Freund's adjuvant (complete and incomplete)), excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a prophylactically or therapeutically effective amount of the antibody or fragment thereof, preferably in purified form, together with a suitable amount of carrier so as to provide the form for proper administration to the patient. The formulation should suit the mode of administration.

In a specific embodiment, the compositions of the invention may be those disclosed in U.S. Provisional Patent Application No. 60/388,920, filed on Jun. 14, 2002 or 60/388,921, filed on Jun. 14, 2002, which are incorporated be reference herein in their entireties.

In a preferred embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lignocamne to ease pain at the site of the injection.

Generally, the ingredients of compositions of the invention are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The compositions of the invention can be formulated as neutral or salt forms.

Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

The amount of the composition of the invention which will be effective in the treatment, prevention or amelioration of one or more symptoms associated with a respiratory viral infection can be determined by standard clinical techniques. For example, the dosage of the composition which will be effective in the treatment, prevention or amelioration of one or more symptoms associated with a respiratory viral infection can be determined by administering the composition to a cotton rat, measuring the RSV, PIV, and/or hMPV titer after challenging the cotton rat with 10⁵ pfu of RSV, PUV, and/or hMPV, respectively, and comparing the RSV, PIV, and/or hMPV titer, respectively, to that obtain for a cotton rat not administered the composition. Accordingly, a dosage that results in a 1 log decrease or a 90% reduction in RSV, PIV, and/or hMPV titer in the cotton rat challenged with 10⁵ pfu of RSV, PIV, and/or hMPV, respectively, relative to the cotton rat challenged with 10⁵ pfu of RSV, PIV, and/or hMPV, respectively, but not administered the composition is the dosage of the composition that can be administered to a human for the treatment, prevention or amelioration of symptoms associated with RSV infection. The dosage of the composition which will be effective in the treatment, prevention or amelioration of one or more symptoms associated with a respiratory, viral infection can be determined by administering the composition to an animal model (e.g., a cotton rat or monkey) and measuring the serum titer of antibodies or antigen-binding fragments thereof that immunospecifically bind to a RSV, PIV, and/or hMPV antigen. Accordingly, a dosage of the composition that results in a serum titer of at least 1 μg/ml, preferably 2 μg/ml, 5 μg/ml, 10 μg/ml, 20 μg/ml, 25 μg/ml, at least 35 μg/ml, at least 40 μg/ml, at least 50 μg/ml, at least 75 μg/ml, at least 100 μg/ml, at least 125 μg/ml, at least 150 μg/ml, at least 200 μg/ml, at least 250 μg/ml, at least 300 μg/ml, at least 350 μg/ml, at least 400 μg/ml, or at least 450 μg/ml for one or all of the antibodies in the composition can be administered to a human for the treatment, prevention or amelioration of one or more symptoms associated with respiratory viral infection. In addition, in vitro assays may optionally be employed to help identify optimal dosage ranges.

The precise dose to be employed in the formulation will also depend on the route of administration, and the seriousness of the respiratory viral infection, and should be decided according to the judgment of the practitioner and each patient's circumstances. Effective doses may be extrapolated from dose-response curves derived from in vitro or animal model (e.g., the cotton rat or Cynomolgous monkey) test systems.

For antibodies, the dosage administered to a patient is typically 0.1 mg/kg to 100 mg/kg of each antibody per the patient's body weight. Preferably, the dosage administered to a patient is between 0.1 mg/kg and 20 mg/kg of each antibody per patient's body weight, more preferably 1 mg/kg to 10 mg/kg of each antibody per the patient's body weight. Generally, human antibodies have a longer half-life within the human body than antibodies from other species due to the immune response to the foreign polypeptides. Thus, lower dosages of human antibodies and less frequent administration is often possible. Further, the dosage and frequency of administration of antibodies of the invention or fragments thereof may be reduced by enhancing uptake and tissue penetration (e.g., into the lung) of the antibodies by modifications such as, for example, lipidation.

In a specific embodiment, antibodies of the invention or fragments thereof, or compositions comprising antibodies of the invention or fragments thereof are administered once a month, once every 6 weeks, or once every 2 months just prior to or during the RSV season. In a specific embodiment, antibodies of the invention or fragments thereof, or compositions comprising antibodies of the invention or fragments thereof are administered once a month, once every 6 weeks, or once every 2 months just prior to or during the PIV season. In a specific embodiment, antibodies of the invention or fragments thereof, or compositions comprising antibodies of the invention or fragments thereof are administered once a month, once every 6 weeks, or once every 2 months just prior to or during the hMPV season. In another embodiment, antibodies or antigen-binding fragments thereof, or compositions comprising antibodies or antigen-binding fragments thereof are administered every two months just prior to or during the RSV, PIV, or hMPV season. In yet another is embodiment, antibodies or antigen-binding fragments thereof, or compositions comprising antibodies or antigen-binding fragments thereof are administered once just prior to or during the RSV, PIV, or hMPV season. The term “RSV season” refers to the season when RSV infection is most likely to occur. Typically, the RSV season in the northern hemisphere commences in November and lasts through April.

In certain embodiments, the antibodies are administered at least 1 time, 2 times, 3 times, 4 times, 5 times, 6 times, 7 times, 8 times, 9 times, 10 times, 15 times or at least 20 times per RSV season. In certain embodiments, the antibodies are administered at most 1 time, 2 times, 3 times, 4 times, 5 times, 6 times, 7 times, 8 times, 9 times, 10 times, 15 times or at most 20 times per RSV season. In certain embodiments, the antibodies are administered at least 1 time, 2 times, 3 times, 4 times, 5 times, 6 times, 7 times, 8 times, 9 times, 10 times, 15 times or at least 20 times per PIV season. In certain embodiments, the antibodies are administered at most 1 time, 2 times, 3 times, 4 times, 5 times, 6 times, 7 times, 8 times, 9 times, 10 times, 15 times or at most 20 times per PIV season. In certain embodiments, the antibodies are administered at least 1 time, 2 times, 3 times, 4 times, 5 times, 6 times, 7 times, 8 times, 9 times, 10 times, 15 times or at least 20 times per hMPV season. In certain embodiments, the antibodies are administered at most 1 time, 2 times, 3 times, 4 times, 5 times, 6 times, 7 times, 8 times, 9 times, 10 times, 15 times or at most 20 times per hMPV season.

4.5 Gene Therapy

In a specific embodiment, nucleic acids comprising sequences encoding antibodies that immunospecifically bind to an RSV antigen, a PIV antigen, and/or a hMPV antigen or functional derivatives thereof, are administered to treat, prevent or ameliorate one or more symptoms associated with RSV infection, by way of gene therapy. Gene therapy refers to therapy performed by the administration to a subject of an expressed or expressible nucleic acid. In this embodiment of the invention, the nucleic acids produce their encoded antibody or antibody fragment that mediates a prophylactic or therapeutic effect. In a specific embodiment, intrabodies are delivered to a subject via gene therapy (see section 4.1).

Any of the methods for gene therapy available in the art can be used according to the present invention. Exemplary methods are described below.

For general reviews of the methods of gene therapy, see Goldspiel et al., 1993, Clinical Pharmacy 12:488-505; Wu and Wu, 1991, Biotherapy 3:87-95; Tolstoshev, 1993, Ann. Rev. Pharmacol. Toxicol. 32:573-596; Mulligan, Science 260:926-932 (1993); and Morgan and Anderson, 1993, Ann. Rev. Biochem. 62:191-217; May, 1993, TIBTECH 11(5):155-215. Methods commonly known in the art of recombinant DNA technology which can be used are described in Ausubel et al. (eds.), Current Protocols in Molecular Biology, John Wiley & Sons, NY (1993); and Kriegler, Gene Transfer and Expression, A Laboratory Manual, Stockton Press, NY (1990).

In a preferred aspect, a composition of the invention comprises nucleic acids encoding an antibody, said nucleic acids being part of an expression vector that expresses the antibody or fragments or chimeric proteins or heavy or light chains thereof in a suitable host. In particular, such nucleic acids have promoters, preferably heterologous promoters, operably linked to the antibody coding region, said promoter being inducible or constitutive, and, optionally, tissue-specific. In another particular embodiment, nucleic acid molecules are used in which the antibody coding sequences and any other desired sequences are flanked by regions that promote homologous recombination at a desired site in the genome, thus providing for intrachromosomal expression of the antibody encoding nucleic acids (Koller and Smithies, 1989, Proc. Natl. Acad. Sci. USA 86:8932-8935; Zijlstra et al., 1989, Nature 342:435-438). In specific embodiments, the expressed antibody molecule is a single chain antibody; alternatively, the nucleic acid sequences include sequences encoding both the heavy and light chains, or fragments thereof, of the antibody.

Delivery of the nucleic acids into a subject may be either direct, in which case the subject is directly exposed to the nucleic acid or nucleic acid-carrying vectors, or indirect, in which case, cells are first transformed with the nucleic acids in vitro, then transplanted into the subject. These two approaches are known, respectively, as in vivo or ex vivo gene therapy.

In a specific embodiment, the nucleic acid sequences are directly administered in vivo, where it is expressed to produce the encoded product. This can be accomplished by any of numerous methods known in the art, e.g., by constructing them as part of an appropriate nucleic acid expression vector and administering it so that they become intracellular, e.g., by infection using defective or attenuated retrovirals or other viral vectors (see U.S. Pat. No. 4,980,286), or by direct injection of naked DNA, or by use of microparticle bombardment (e.g., a gene gun; Biolistic, Dupont), or coating with lipids or cell-surface receptors or transfecting agents, encapsulation in liposomes, microparticles, or microcapsules, or by administering them in linkage to a peptide which is known to enter the nucleus, by administering it in linkage to a ligand subject to receptor-mediated endocytosis (see, e.g., Wu and Wu, 1987, J. Biol. Chem. 262:4429-4432) (which can be used to target cell types specifically expressing the receptors), etc. In another embodiment, nucleic acid-ligand complexes can be formed in which the ligand comprises a fusogenic viral peptide to disrupt endosomes, allowing the nucleic acid to avoid lysosomal degradation. In yet another embodiment, the nucleic acid can be targeted in vivo for cell specific uptake and expression, by targeting a specific receptor (see, e.g., PCT Publications WO 92/06180; WO 92/22635; WO92/203 16; WO93/14188, WO 93/20221). Alternatively, the nucleic acid can be introduced intracellularly and incorporated within host cell DNA for expression, by homologous recombination (Koller and Smithies, 1989, Proc. Natl. Acad. Sci. USA 86:8932-8935; and Zijlstra et al., 1989, Nature 342:435-438).

In a specific embodiment, viral vectors that contains nucleic acid sequences encoding an antibody of the invention or fragments thereof are used. For example, a retroviral vector can be used (see Miller et al., 1993, Meth. Enzymol. 217:581-599). These retroviral vectors contain the components necessary for the correct packaging of the viral genome and integration into the host cell DNA. The nucleic acid sequences encoding the antibody to be used in gene therapy are cloned into one or more vectors, which facilitates delivery of the gene into a subject. More detail about retroviral vectors can be found in Boesen et al., 1994, Biotherapy 6:291-302, which describes the use of a retroviral vector to deliver the mdr 1 gene to hematopoietic stem cells in order to make the stem cells more resistant to chemotherapy.

Other references illustrating the use of retroviral vectors in gene therapy are: Clowes et al., 1994, J. Clin. Invest. 93:644-651; Klein et al., 1994, Blood 83:1467-1473; Salmons and Gunzberg, 1993, Human Gene Therapy 4:129-141; and Grossman and Wilson, 1993, Curr. Opin. in Genetics and Devel. 3:110-114.

Adenoviruses are other viral vectors that can be used in gene therapy. Adenoviruses are especially attractive vehicles for delivering genes to respiratory epithelia. Adenoviruses naturally infect respiratory epithelia where they cause a mild disease. Other targets for adenovirus-based delivery systems are liver, the central nervous system, endothelial cells, and muscle. Adenoviruses have the advantage of being capable of infecting non-dividing cells. Kozarsky and Wilson, 1993, Current Opinion in Genetics and Development 3:499-503 present a review of adenovirus-based gene therapy. Bout et al., 1994, Human Gene Therapy 5:3-10 demonstrated the use of adenovirus vectors to transfer genes to the respiratory epithelia of rhesus monkeys. Other instances of the use of adenoviruses in gene therapy can is be found in Rosenfeld et al., 1991, Science 252:431-434; Rosenfeld et al., 1992, Cell 68:143-155; Mastrangeli et al., 1993, J. Clin. Invest. 91:225-234; PCT Publication WO94/12649; and Wang et al., 1995, Gene Therapy 2:775-783. In a preferred embodiment, adenovirus vectors are used.

Adeno-associated virus (AAV) has also been proposed for use in gene therapy (Walsh et al., 1993, Proc. Soc. Exp. Biol. Med. 204:289-300; and U.S. Pat. No. 5,436,146).

Another approach to gene therapy involves transferring a gene to cells in tissue culture by such methods as electroporation, lipofection, calcium phosphate mediated transfection, or viral infection. Usually, the method of transfer includes the transfer of a selectable marker to the cells. The cells are then placed under selection to isolate those cells that have taken up and are expressing the transferred gene. Those cells are then delivered to a subject.

In this embodiment, the nucleic acid is introduced into a cell prior to administration in vivo of the resulting recombinant cell. Such introduction can be carried out by any method known in the art, including but not limited to transfection, electroporation, microinjection, infection with a viral or bacteriophage vector containing the nucleic acid sequences, cell fusion, chromosome-mediated gene transfer, microcell-mediated gene transfer, spheroplast fusion, etc. Numerous techniques are known in the art for the introduction of foreign genes into cells (see, e.g., Loeffler and Behr, 1993, Meth. Enzymol. 217:599-618; Cohen et al., 1993, Meth. Enzymol. 217:618-644; Clin. Pharma. Ther. 29:69-92 (1985)) and may be used in accordance with the present invention, provided that the necessary developmental and physiological functions of the recipient cells are not disrupted. The technique should provide for the stable transfer of the nucleic acid to the cell, so that the nucleic acid is expressible by the cell and preferably heritable and expressible by its cell progeny.

The resulting recombinant cells can be delivered to a subject by various methods known in the art. Recombinant blood cells (e.g., hematopoietic stem or progenitor cells) are preferably administered intravenously. The amount of cells envisioned for use depends on the desired effect, patient state, etc., and can be determined by one skilled in the art.

Cells into which a nucleic acid can be introduced for purposes of gene therapy encompass any desired, available cell type, and include but are not limited to epithelial cells, endothelial cells, keratinocytes, fibroblasts, muscle cells, hepatocytes; blood cells such as T lymphocytes, B lymphocytes, monocytes, macrophages, neutrophils, eosinophils, megakaryocytes, granulocytes; various stem or progenitor cells, in particular hematopoietic stem or progenitor cells, e.g., as obtained from bone marrow, umbilical cord blood, peripheral blood, fetal liver, etc.

In a preferred embodiment, the cell used for gene therapy is autologous to the subject.

In an embodiment in which recombinant cells are used in gene therapy, nucleic acid sequences encoding an antibody or fragment thereof are introduced into the cells such that they are expressible by the cells or their progeny, and the recombinant cells are then administered in vivo for therapeutic effect. In a specific embodiment, stem or progenitor cells are used. Any stem and/or progenitor cells which can be isolated and maintained in vitro can potentially be used in accordance with this embodiment of the present invention (see e.g., PCT Publication WO 94/08598; Stemple and Anderson, 1992, Cell 7 1:973-985; Rheinwald, 1980, Meth. Cell Bio. 21A:229; and Pittelkow and Scott, 1986, Mayo Clinic Proc. 61:771).

In a specific embodiment, the nucleic acid to be introduced for purposes of gene therapy comprises an inducible promoter operably linked to the coding region, such that expression of the nucleic acid is controllable by controlling the presence or absence of the appropriate inducer of transcription.

4.6 Antibody Characterization and Demonstration of Therapeutic or Prophylactic Utility

Antibodies may be characterized in a variety of ways. In particular, antibodies may be assayed for the ability to immunospecifically bind to a RSV antigen, a PIV antigen, and/or a hMPV antigen. Such an assay may be performed in solution (e.g., Houghten, 1992, Bio/Techniques 13:412-421), on beads (Lam, 1991, Nature 354:82-84), on chips (Fodor, 1993, Nature 364:555-556), on bacteria (U.S. Pat. No. 5,223,409), on spores (U.S. Pat. Nos. 5,571,698; 5,403,484; and 5,223,409), on plasmids (Cull et al., 1992, Proc. Natl. Acad. Sci. USA 89:1865-1869) or on phage (Scott and Smith, 1990, Science 249:386-390; Devlin, 1990, Science 249:404-406; Cwirla et al., 1990, Proc. Natl. Acad. Sci. USA 87:6378-6382; and Felici, 1991, J. Mol. Biol. 222:301-310) (each of these references is incorporated herein in its entirety by reference). Antibodies or antigen-binding fragments thereof that have been identified to immunospecifically bind to a RSV antigen, a PIV antigen, and/or a hMPV antigen or a fragment thereof can then be assayed for their avidity and affinity for a RSV antigen, a PIV antigen, and/or a hMPV antigen.

Immunospecific binding and cross-reactivity with other antigens of an antibody may be determined by any method known in the art. Immunoassays which can be used to analyze immunospecific binding and cross-reactivity include, but are not limited to, competitive and non-competitive assay systems using techniques such as western blots, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, protein A immunoassays, to name but a few. Such assays are routine and well known in the art (see, e.g., Ausubel et al, eds, 1994, Current Protocols in Molecular Biology, Vol. 1, John Wiley & Sons, Inc., New York, which is incorporated by reference herein in its entirety). Exemplary immunoassays are described briefly below (but are not intended by way of limitation).

Immunoprecipitation protocols generally comprise lysing a population of cells in a lysis buffer such as RIPA buffer (1% NP-40 or Triton X-100, 1% sodium deoxycholate, 0.1% SDS, 0.15 M NaCl, 0.01 M sodium phosphate at pH 7.2, 1% Trasylol) supplemented with protein phosphatase and/or protease inhibitors (e.g., EDTA, PMSF, aprotinin, sodium vanadate), adding the antibody of interest to the cell lysate, incubating for a period of time (e.g., 1 to 4 hours) at 40° C., adding protein A and/or protein G sepharose beads to the cell lysate, incubating for about an hour or more at 40° C., washing the beads in lysis buffer and resuspending the beads in SDS/sample buffer. The ability of the antibody of interest to immunoprecipitate a particular antigen can be assessed by, e.g., western blot analysis. One of skill in the art would be knowledgeable as to the parameters that can be modified to increase the binding of the antibody to an antigen and decrease the background (e.g. pre-clearing the cell lysate with sepharose beads). For further discussion regarding immunoprecipitation protocols see, e.g., Ausubel et al, eds, 1994, Current Protocols in Molecular Biology, Vol. 1, John Wiley & Sons, Inc., New York at 10.16.1.

Western blot analysis generally comprises preparing protein samples, electrophoresis of the protein samples in a polyacrylamide gel (e.g., 8%-20% SDS-PAGE depending on the molecular weight of the antigen), transferring the protein sample from the polyacrylamide gel to a membrane such as nitrocellulose, PVDF or nylon, blocking the membrane in blocking solution (e.g., PBS with 3% BSA or non-fat milk), washing the membrane in washing buffer (e.g., PBS-Tween 20), blocking the membrane with primary antibody (the antibody of interest) diluted in blocking buffer, washing the membrane in washing buffer, blocking the membrane with a secondary antibody (which recognizes the primary antibody, e.g., an anti-human antibody) conjugated to an enzymatic substrate (e.g., horseradish peroxidase or alkaline phosphatase) or radioactive molecule (e.g., ³²P or ¹²⁵I) diluted in blocking buffer, washing the membrane in wash buffer, and detecting the presence of the antigen. One of skill in the art would be knowledgeable as to the parameters that can be modified to increase the signal detected and to reduce the background noise. For further discussion regarding western blot protocols see, e.g., Ausubel et al, eds, 1994, Current Protocols in Molecular Biology, Vol. 1, John Wiley & Sons, Inc., New York at 10.8.1.

ELISAs comprise preparing antigen, coating the well of a 96 well microtiter plate with the antigen, adding the antibody of interest conjugated to a detectable compound such as an enzymatic substrate (e.g., horseradish peroxidase or alkaline phosphatase) to the well and incubating for a period of time, and detecting the presence of the antigen. In ELISAs the antibody of interest does not have to be conjugated to a detectable compound; instead, a second antibody (which recognizes the antibody of interest) conjugated to a detectable compound may be added to the well. Further, instead of coating the well with the antigen, the antibody may be coated to the well. In this case, a second antibody conjugated to a detectable compound may be added following the addition of the antigen of interest to the coated well. One of skill in the art would be knowledgeable as to the parameters that can be modified to increase the signal detected as well as other variations of ELISAs known in the art. For further discussion regarding ELISAs see, e.g., Ausubel et al, eds, 1994, Current Protocols in Molecular Biology, Vol. 1, John Wiley & Sons, Inc., New York at 11.2.1.

The binding affinity of an antibody to an antigen and the off-rate of an antibody-antigen interaction can be determined by competitive binding assays. One example of a competitive binding assay is a radioimmunoassay comprising the incubation of labeled antigen (e.g., ³H or 1251 with the antibody of interest in the presence of increasing amounts of unlabeled antigen, and the detection of the antibody bound to the labeled antigen. The affinity of the antibody of the present invention or a fragment thereof for a RSV antigen and the binding off-rates can be determined from the data by scatchard plot analysis. Competition with a second antibody can also be determined using radioimmunoassays. In a specific embodiment, a first antibody or an antigen-binding fragment thereof is conjugated to a labeled compound (e.g., ³H or ¹²⁵I) in the presence of increasing amounts of an unlabeled second antibody.

In a preferred embodiment, BIAcore kinetic analysis is used to determine the binding on and off rates of antibodies or antigen-binding fragments thereof to a RSV, PUV and/or hMPV antigen. BIAcore kinetic analysis comprises analyzing the binding and dissociation of a RSV antigen from chips with immobilized antibodies or antigen-binding fragments thereof on their surface (see the Example section infra).

The antibodies of the invention or fragments thereof can also be assayed for their ability to inhibit the binding of RSV, PIV and/or hMPV to its host cell receptor using techniques known to those of skill in the art. For example, cells expressing the receptor for RSV, PIV and/or hMPV, respectively, can be contacted with RSV, PIV and/or hMPV, respectively, in the presence or absence of an antibody or fragment thereof and the ability of the antibody or fragment thereof to inhibit RSV, PIV and/or hMPV's binding can measured by, for example, flow cytometry or a scintillation assay. RSV, PIV and/or hMPV (e.g., a RSV, PIV and/or hMPV antigen such as F glycoprotein or G glycoprotein) or the antibody or antibody fragment can be labeled with a detectable compound such as a radioactive label (e.g., ³²P, ³⁵S, and ¹²⁵I) or a fluorescent label (e.g., fluorescein isothiocyanate, rhodamine, phycoerythrin, phycocyanin, allophycocyanin, α-phthaldehyde and fluorescamine) to enable detection of an interaction between RSV, PIV and/or hMPV and its respective host cell receptor. Alternatively, the ability of antibodies or antigen-binding fragments thereof to inhibit RSV, PIV and/or hMPV from binding to its receptor can be determined in cell-free assays. For example, RSV, PIV and/or hMPV or a RSV, PIV and/or hMPV antigen such as G glycoprotein can be contacted with an antibody or fragment thereof and the ability of the antibody or antibody fragment to inhibit RSV, PIV and/or hMPV or the RSV, PIV and/or hMPV antigen from binding to its host cell receptor can be determined. Preferably, the antibody or the antibody fragment is immobilized on a solid support and RSV, PIV and/or hMPV, or a RSV, PIV and/or hMPV antigen is labeled with a detectable compound. Alternatively, RSV, PIV and/or hMPV, or a RSV, PIV and/or hMPV antigen is immobilized on a solid support and the antibody or fragment thereof is labeled with a detectable compound. RSV, PIV and/or hMPV, or a RSV, PIV and/or hMPV antigen may be partially or completely purified (e.g., partially or completely free of other polypeptides) or part of a cell lysate. Further, a RSV, PIV and/or hMPV antigen may be a fusion protein comprising the RSV, PIV and/or hMPV antigen and a domain such as glutathionine-S-transferase. Alternatively, a RSV, PIV and/or hMPV antigen can be biotinylated using techniques well known to those of skill in the art (e.g., biotinylation kit, Pierce Chemicals; Rockford, Ill.).

The antibodies of the invention or fragments thereof can also be assayed for their ability to inhibit or downregulate RSV, PIV and/or hMPV replication using techniques known to those of skill in the art. For example, RSV, PIV and/or hMPV replication can be assayed by a plaque assay such as described, e.g., by Johnson et al., 1997, Journal of Infectious Diseases 176:1215-1224. The antibodies of the invention or fragments thereof can also be assayed for their ability to inhibit or downregulate the expression of RSV, PIV and/or hMPV polypeptides. Techniques known to those of skill in the art, including, but not limited to, Western blot analysis, Northern blot analysis, and RT-PCR can be used to measure the expression of RSV, PIV and/or hMPV polypeptides. Further, the antibodies of the invention or fragments thereof can be assayed for their ability to prevent the formation of syncytia.

The antibodies of the invention or fragments thereof are preferably tested in vitro, and then in vivo for the desired therapeutic or prophylactic activity, prior to use in humans. For example, in vitro assays which can be used to determine whether administration of a specific antibody or composition of the present invention is indicated, include in vitro cell culture assays in which a subject tissue sample is grown in culture, and exposed to or otherwise administered an antibody or composition of the present invention, and the effect of such an antibody or composition of the present invention upon the tissue sample is observed. In various specific embodiments, in vitro assays can be carried out with representative cells of cell types involved in a RSV, PIV and/or hMPV infection (e.g., respiratory epithelial cells), to determine if an antibody or composition of the present invention has a desired effect upon such cell types. Preferably, the antibodies or compositions comprising the antibodies are also tested in in vitro assays and animal model systems prior to administration to humans. In a specific embodiment, cotton rats are administered an antibody or fragment thereof, or a composition of the invention, challenged with 10⁵ pfu of RSV, PIV and/or hMPV, and four or more days later the rats are sacrificed and RSV, PIV and/or hMPV titer and anti-RSV, anti-PIV and/or anti-hMPV antibody serum level is determined. Further, in accordance with this embodiment, the tissues (e.g., the lung tissues) from the sacrificed rats can be examined for histological changes.

In accordance with the invention, clinical trials with human subjects need not be performed in order to demonstrate the prophylactic and/or therapeutic efficacy of antibodies of the invention or fragments thereof. In vitro and animal model studies using the antibodies or antigen-binding fragments thereof can be extrapolated to humans and are sufficient for demonstrating the prophylactic and/or therapeutic utility of said antibodies or antibody fragments.

Antibodies or compositions that can be used with the methods of the present invention can be tested for their toxicity in suitable animal model systems, including but not limited to rats, mice, cows, monkeys, and rabbits. For in vivo testing of an antibody or composition's toxicity any animal model system known in the art may be used.

The treatment is considered therapeutic if there is, for example, a reduction is viral load, amelioration of one or more symptoms, a reduction in the duration of a respiratory viral infection, or a decrease in mortality and/or morbidity following administration of an antibody or composition of the invention. Further, the treatment is considered therapeutic if there is an increase in the immune response following the administration of one or more antibodies or antigen-binding fragments thereof which immunospecifically bind to one or more RSV, PIV, and/or hMPV antigens.

Antibodies can be tested in vitro and in vivo for the ability to affect the expression levels of cytokines such as, but not limited to, IFN-α, IFN-β, IFN-γ, IL-2, IL-3, IL-4, IL-5, IL-6, IL-7, IL-8, IL-9, IL-10, IL-12 and IL-15. In a more specific embodiment, an antibody or composition of the invention is tested for its ability to affect the expression level of one or more cytokines, the expression of which have been induced by a respiratory viral infection. In an even more specific embodiment, an antibody or composition of the invention is tested for its ability to reduce the expression level of one or more virus-induced cytokines. Techniques known to those of skill in the art can be used to measure the level of expression of cytokines. For example, the level of expression of cytokines can be measured by analyzing the level of RNA of cytokines by, for example, RT-PCR and Northern blot analysis, and by analyzing the level of cytokines by, for example, immunoprecipitation followed by western blot analysis and ELISA. In a preferred embodiment, an antibody or composition of the invention is tested for its ability to affect the expression of IFN-γ. In a more specific embodiment, an antibody or composition of the invention is tested for its ability to affect the expression level of IFN-γ the expression of which has been induced by a respiratory viral infection. In an even more specific embodiment, an antibody or composition of the invention is tested for its ability to reduce the expression level of virus-induced IFN-γ.

Antibodies can be tested in vitro and in vivo for their ability to modulate the biological activity of immune cells, preferably human immune cells (e.g., but not limited to, T-cells, B-cells, and Natural Killer cells). In more specific embodiments, antibodies can be tested in vitro and in vivo for their ability to modulate the biological activity of immune cells that has been induced by a respiratory viral infection. In even more specific embodiments, antibodies can be tested for their ability to reduce the one or more biological activities of immune cells that have been induced by a respiratory viral infection. The ability of antibodies or antigen-binding fragments thereof to modulate the biological activity of immune cells can be assessed by detecting the expression of antigens, detecting the proliferation of immune cells, detecting the activation of signaling molecules, detecting the effector function of immune cells, or detecting the differentiation of immune cells. Techniques known to those of skill in the art can be used for measuring these activities. For example, cellular proliferation can be assayed by 3H-thymidine incorporation assays and trypan blue cell counts. Antigen expression can be assayed, for example, by immunoassays including, but are not limited to, competitive and non-competitive assay systems using techniques such as western blots, immunohistochemistry radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, protein A immunoassays and FACS analysis. The activation of signaling molecules can be assayed, for example, by kinase assays and electrophoretic shift assays (EMSAs).

Antibodies can also be tested for their ability to inhibit viral replication or reduce viral load in in vitro, ex vivo and in vivo assays. Antibodies can also be tested for their ability to decrease the time course of a respiratory viral infection. Antibodies can also be tested for their ability to increase the survival period of humans suffering from RSV infection by at least 25%, preferably at least 50%, at least 60%, at least 75%, at least 85%, at least 95%, or at least 99%. Further, antibodies can be tested for their ability reduce the hospitalization period of humans suffering from respiratory viral infection by at least 60%, preferably at least 75%, at least 85%, at least 95%, or at least 99%. Techniques known to those of skill in the art can be used to analyze the function of the antibodies or compositions of the invention in vivo.

4.7 Kits

The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

The present invention provides kits that can be used in the above methods. In one embodiment, a kit comprises (i) one or more anti-RSV-antigen antibodies or antigen-binding fragments thereof and one or more anti-PIV-antigen antibodies or antigen-binding fragments thereof; (ii) one or more anti-PIV-antigen antibodies or antigen-binding fragments thereof and one or more anti-hMPV-antigen antibodies or antigen-binding fragments thereof; or (iii) one or more anti-RSV-antigen antibodies or antigen-binding fragments thereof, one or more anti-hMPV-antigen antibodies or antigen-binding fragments thereof. In certain embodiments, a kit comprises one or more anti-PIV-antigen antibodies, one or more anti-hMPV-antigen antibodies, and one or more anti-RSV-antigen antibodies.

In certain embodiments, the kits of the present invention further comprise a control antibody which does not react with a RSV antigen, a PIV antigen, and a hMPV antigen. In another specific embodiments, the kits of the present invention contain a means for detecting the binding of an antibody to a RSV antigen, a PIV antigen, and/or a hMPV antigen (e.g., the antibody may be conjugated to a detectable substrate such as a fluorescent compound, an enzymatic substrate, a radioactive compound or a luminescent compound, or a second antibody which recognizes the first antibody may be conjugated to a detectable substrate). In specific embodiments, the kit may include a recombinantly produced or chemically synthesized RSV antigen, a PIV antigen, and/or a hMPV antigen. The RSV antigen, a PIV antigen, and/or a hMPV antigen provided in the kit may also be attached to a solid support. In a more specific embodiment the detecting means of the above-described kit includes a solid support to which RSV antigen, a PIV antigen, and/or a hMPV antigen is attached. Such a kit may also include a non-attached reporter-labeled anti-human antibody. In this embodiment, binding of the antibody to the RSV antigen, a PIV antigen, and/or a hMPV antigen can be detected by binding of the said reporter-labeled antibody.

4.8. Assays for Use with the Invention

4.8.1 Measurement of Incidence of Infection Rate

The incidence of infection can be determined by any method well-known in the art, for example, but not limited to, clinical samples (e.g., nasal swabs) can be tested for the presence of RSV, PIV, and/or hMPV by immunofluorescence assay (IFA) using an anti-RSV-antigen antibody, an anti-PIV-antigen antibody, and/or an anti-hMPV-antigen antibody, respectively. Samples containing intact cells can be directly processed, whereas isolates without intact cells should first be cultured on a permissive cell line (e.g. HEp-2 cells). Cultured cell suspensions should be cleared by centrifugation at, e.g., 300×g for 5 minutes at room temperature, followed by a PBS, pH 7.4 (Ca++ and Mg++ free) wash under the same conditions. Cell pellets are resuspended in a small volume of PBS for analysis. Primary clinical isolates containing intact cells are mixed with PBS and centrifuged at 300×g for 5 minutes at room temperature. Mucus is removed from the interface with a sterile pipette tip and cell pellets are washed once more with PBS under the same conditions. Pellets are then resuspended in a small volume of PBS for analysis. Five to ten microliters of each cell suspension are spotted per 5 mm well on acetone washed 12-well HTC supercured glass slides and allowed to air dry. Slides are fixed in cold (−20° C.) acetone for 10 minutes. Reactions are blocked by adding PBS −1% BSA to each well followed by a 10 minute incubation at room temperature. Slides are washed three times in PBS −0.1% Tween-20 and air dried. Ten microliters of each primary antibody reagent diluted to 250 ng/ml in blocking buffer is spotted per well and reactions are incubated in a humidified 37° C. environment for 30 minutes. Slides are then washed extensively in three changes of PBS −0.1% Tween-20 and air dried. Ten microliters of appropriate secondary conjugated antibody reagent diluted to 250 ng/ml in blocking buffer are spotted per respective well and reactions are incubated in a humidified 37° C. environment for an additional 30 minutes. Slides are then washed in three changes of PBS −0.1% Tween-20. Five microliters of PBS-50% glycerol-10 mM Tris pH 8.0-1 mM EDTA are spotted per reaction well, and slides are mounted with cover slips. Each reaction well is subsequently analyzed by fluorescence microscopy at 200× power using a B2A filter (EX 450-490 nm). Positive reactions are scored against an autofluorescent background obtained from unstained cells or cells stained with secondary reagent alone. RSV positive reactions are characterized by bright fluorescence punctuated with small inclusions in the cytoplasm of infected cells.

4.8.2 Measurement of Serum Titer

The antibody serum titer can be determined by any method well-known in the art, for example, but not limited to, the amount of antibody or antibody fragment in serum samples can be quantitated by a sandwich ELISA. Briefly, the ELISA consists of coating microtiter plates overnight at 4° C. with an antibody that recognizes the antibody or antibody fragment in the serum. The plates are then blocked for approximately 30 minutes at room temperature with PBS-Tween-0.5% BSA. Standard curves are constructed using purified antibody or antibody fragment diluted in PBS-TWEEN-BSA, and samples are diluted in PBS-BSA-BSA. The samples and standards are added to duplicate wells of the assay plate and are incubated for approximately 1 hour at room temperature. Next, the non-bound antibody is washed away with PBS-TWEEN and the bound antibody is treated with a labeled secondary antibody (e.g., horseradish peroxidase conjugated goat-anti-human IgG) for approximately 1 hour at room temperature. Binding of the labeled antibody is detected by adding a chromogenic substrate specific for the label and measuring the rate of substrate turnover, e.g., by a spectrophotometer. The concentration of antibody or antibody fragment levels in the serum is determined by comparison of the rate of substrate turnover for the samples to the rate of substrate turnover for the standard curve.

4.8.3 BIAcore Assay

Determination of the kinetic parameters of antibody binding can be determined for example by the injection of 250 μL of monoclonal antibody (“mAb”) at varying concentration in HBS buffer containing 0.05% Tween-20 over a sensor chip surface, onto which has been immobilized the antigen. The flow rate is maintained constant at 75 uL/min. Dissociation data is collected for 15 min, or longer as necessary. Following each injection/dissociation cycle, the bound mAb is removed from the antigen surface using brief, 1 min pulses of dilute acid, typically 10-100 mM HCl, though other regenerants are employed as the circumstances warrant.

More specifically, for measurement of the rates of association, kₒₙ, and dissociation, k_{off}, the antigen is directly immobilized onto the sensor chip surface through the use of standard amine coupling chemistries, namely the EDC/NHS method (EDC=N-diethylaminopropyl)-carbodiimide). Briefly, a 5-100 nM solution of the antigen in 10 mM NaOAc, pH4 or pH5 is prepared and passed over the EDC/NHS-activated surface until approximately 30-50 RU's worth of antigen are immobilized. Following this, the unreacted active esters are “capped” off with an injection of 1M Et-NH2. A blank surface, containing no antigen, is prepared under identical immobilization conditions for reference purposes. Once a suitable surface has been prepared, an appropriate dilution series of each one of the antibody reagents is prepared in HBS/Tween-20, and passed over both the antigen and reference cell surfaces, which are connected in series. The range of antibody concentrations that are prepared varies depending on what the equilibrium binding constant, K_{D}, is estimated to be. As described above, the bound antibody is removed after each injection/dissociation cycle using an appropriate regenerant.

Once an entire data set is collected, the resulting binding curves are globally fitted using algorithms supplied by the instrument manufacturer, BIAcore, Inc. (Piscataway, N.J.). All data are fitted to a 1:1 Langmuir binding model. These algorithm calculate both the kₒₙ and the k_{off}, from which the apparent equilibrium binding constant, K_{D}, is deduced as the ratio of the two rate constants (i.e. k_{off}/kₒₙ). More detailed treatments of how the individual rate constants are derived can be found in the BIAevaluation Software Handbook (BIAcore, Inc., Piscataway, N.J.).

4.8.4 Microneutralization Assay

The ability of antibodies or antigen-binding fragments thereof to neutralize virus infectivity is determined by a microneutralization assay. This microneutralization assay is a modification of the procedures described by Anderson et al. (1985, J. Clin. Microbiol. 22:1050-1052, the disclosure of which is hereby incorporated by reference in its entirety). The procedure is also described in Johnson et al., 1999, J. Infectious Diseases 180:35-40, the disclosure of which is hereby incorporated by reference in its entirety.

Antibody dilutions are made in triplicate using a 96-well plate. Ten TCID₅₀ of RSV, PIV, APV, and/or hMPV are incubated with serial dilutions of the antibody or antigen-binding fragments thereof to be tested for 2 hours at 37_C in the wells of a 96-well plate.

RSV susceptible cultured liver cells, such as, but not limited to HEp-2 cells (2.5×10⁴) are then added to each well and cultured for 5 days at 37_C. in 5% CO₂. After 5 days, the medium is aspirated and cells are washed and fixed to the plates with 80% methanol and 20% PBS. Virus replication is then determined by viral antigen, such as F protein expression. Fixed cells are incubated with a biotin-conjugated anti-viral antigen, such as anti-F protein monoclonal antibody (e.g., pan F protein, C-site-specific MAb 133-1H) washed and horseradish peroxidase conjugated avidin is added to the wells. The wells are washed again and turnover of substrate TMB (thionitrobenzoic acid) is measured at 450 nm. The neutralizing titer is expressed as the antibody concentration that causes at least 50% reduction in absorbency at 450 nm (the OD₄₅₀) from virus-only control cells.

4.8.5 Viral Fusion Inhibition Assay

The ability of anti-RSV-antigen antibodies, anti-PIV-antigen antibodies, and/or anti-hMPV-antigen antibodies or antigen-binding fragments thereof to block RSV, PIV, and hMPV, respectively, induced fusion after viral attachment to the cells is determined in a fusion inhibition assay. This assay is identical to the microneutralization assay, except that the cells are infected with the respective virus for four hours prior to addition of antibody (Taylor et al, 1992, J. Gen. Virol. 73:2217-2223).

4.8.6 Isothermal Titration Calorimetry

Thermodynamic binding affinities and enthalpies are determined from isothermal titration calorimetry (ITC) measurements on the interaction of antibodies with their respective antigen.

Antibodies are diluted in dialysate and the concentrations were determined by UV spectroscopic absorption measurements with a Perkin-Elmer Lambda 4B Spectrophotometer using an extinction coefficient of 217,000 M⁻¹ cm⁻¹ at the peak maximum at 280 nm. The diluted RSV-antigen, PIV-antigen, and/or hMPV-antigen concentrations are calculated from the ratio of the mass of the original sample to that of the diluted sample since its extinction coefficient is too low to determine an accurate concentration without employing and losing a large amount of sample.

ITC Measurements

The binding thermodynamics of the antibodies are determined from ITC measurements using a Microcal, Inc. VP Titration Calorimeter. The VP titration calorimeter consists of a matched pair of sample and reference vessels (1.409 ml) enclosed in an adiabatic enclosure and a rotating stirrer-syringe for titrating ligand solutions into the sample vessel. The ITC measurements are performed at 25° C. and 35° C. The sample vessel contained the antibody in the phosphate buffer while the reference vessel contains just the buffer solution. The phosphate buffer solution is saline 67 mM PO₄ at pH 7.4 from HyClone, Inc. Five or ten μl aliquots of the 0.05 to 0.1 mM RSV-antigen, PIV-antigen, and/or hMPV-antigen solution are titrated 3 to 4 minutes apart into the antibody sample solution until the binding is saturated as evident by the lack of a heat exchange signal.

A non-linear, least square minimization software program from Microcal, Inc., Origin 5.0, is used to fit the incremental heat of the ith titration (ΔQ (i)) of the total heat, Qₜ, to the total titrant concentration, Xₜ, according to the following equations (I),

Qₜ=nCₜΔH_{b°}V{1+Xₜ/nCₜ+1/nK_{b}Cₜ−[(1+Xₜ/nCₜ+1/nK_{b}Cₜ)²−4Xₜ/nCₜ]^{1/2}}/2 (1a)

ΔQ(i)=Q(i)+dVi/2V {Q(i)+Q(i−1)}−Q(i−1) (1b)

where Cₜ is the initial antibody concentration in the sample vessel, V is the volume of the sample vessel, and n is the stoichiometry of the binding reaction, to yield values of K_{b}, ΔH_{b}°, and n. The optimum range of sample concentrations for the determination of K_{b} depends on the value of K_{b} and is defined by the following relationship.

CₜK_{b}n≦100 (2)

so that at 1 μM the maximum K_{b} that can be determined is less than 2.5×10⁸ M⁻¹. If the first titrant addition does not fit the binding isotherm, it was neglected in the final analysis since it may reflect release of an air bubble at the syringe opening-solution interface.

4.8.7 Cotton Rat Prophylaxis

This assay is used to determine the ability of anti-RSV-antigen antibodies, anti-PIV-antigen antibodies, and/or anti-hMPV-antigen antibodies or fragments thereof to prevent lower respiratory tract viral infection in cotton rats when administered by intravenous (IV) route. In certain other embodiments, the antibodies are administered by intramuscular (IM) route or by intranasal route (IN). The antibodies can be administered by any technique well-known to the skilled artisan. This assay is also used to correlate the serum concentration of anti-RSV-antigen antibodies, anti-PIV-antigen antibodies, and/or anti-hMPV-antigen antibodies with a reduction in lung RSV, PIV, and/or hMPV, respectively, titer.

Bovine serum albumin (BSA; fraction V) can be obtained from Sigma Chemicals. RSV-Long (A subtype), RSV B subtype, PIV, or hMPV is propagated in cultured liver cells, such as, but not limited to Hep-2 cells. On day 0, groups of cotton rats (Sigmodon hispidis, average weight 100 g) are administered the antibody of interest or BSA by intramuscular injection, by intravenous injection, or by intranasal route. Four days after the infection, animals are sacrificed, and their lung tissue is harvested and pulmonary virus titers are determined by plaque titration. In certain embodiments, 0.31, 0.63, 1.25, 2.5, 5.5 and 10 mg/kg (body weight) of antibody are administered. Bovine serum albumin (BSA) 10 mg/kg is used as a negative control. Antibody concentrations in the serum at the time of challenge are determined using a sandwich ELISA.

4.8.8 Bioavailability

The percent of dose entering the systemic circulation after administration of a given dosage of antibodies (drug) is referred to as bioavailability. More explicitly, bioavailability is defined as the ratio of the amount of antibodies “absorbed” from a test formulation to the amount “absorbed” after administration of a standard formulation. Frequently, the “standard formulation” used in assessing bioavailability is the aqueous solution of the drug, given intravenously.

The amount of antibodies absorbed is taken as a measure of the ability of the formulation to deliver the antibodies to the sites of drug action; this will depend on such factors as, e.g., disintegration and dissolution properties of the dosage form, and the rate of biotransformation relative to rate of absorption—dosage forms containing identical amounts of active drug may differ markedly in their abilities to make drug available, and therefore, in their abilities to permit the drug to manifest its expected pharmacodynamic and therapeutic properties.

“Amount absorbed” is conventionally measured by one of two criteria, either the area under the time-plasma concentration curve (A UC) or the total (cumulative) amount of drug excreted in the urine following drug administration. A linear relationship exists between “area under the curve” and dose when the fraction of drug absorbed is independent of dose, and elimination rate (half-life) and volume of distribution are independent of dose and dosage form. A linearity of the relationship between area under the curve and dose may occur if, for example, the absorption process is a saturable one, or if drug fails to reach the systemic circulation because of, e.g., binding of drug in the intestine or biotransformation in the liver during the drug's first transit through the portal system.

4.8.9 Clinical Trials

Antibodies of the invention or fragments thereof tested in in vitro assays and animal models may be further evaluated for safety, tolerance and pharmacokinetics in groups of normal healthy adult volunteers. The volunteers are administered intramuscularly, intravenously or by a pulmonary delivery system a single dose of 0.5 mg/kg, 3 mg/kg, 5 mg/kg, 10 mg/kg or 15 mg/kg of an antibody or fragment thereof which immunospecifically binds to a RSV, PIV, and/or hMPV antigen. Each volunteer is monitored at least 24 hours prior to receiving the single dose of the antibody or fragment thereof and each volunteer will be monitored for at least 48 hours after receiving the dose at a clinical site. Then volunteers are monitored as outpatients on days 3, 7, 14, 21, 28, 35, 42, 49, and 56 postdose.

Blood samples are collected via an indwelling catheter or direct venipuncture using 10 ml red-top Vacutainer tubes at the following intervals: (1) prior to administering the dose of the antibody or antibody fragment; (2) during the administration of the dose of the antibody or antibody fragment; (3) 5 minutes, 10 minutes, 15 minutes, 20 minutes, 30 minutes, 1 hour, 2 hours, 4 hours, 8 hours, 12 hours, 24 hours, and 48 hours after administering the dose of the is antibody or antibody fragment; and (4) 3 days, 7 days 14 days, 21 days, 28 days, 35 days, 42 days, 49 days, and 56 days after administering the dose of the antibody or antibody fragment.

Samples are allowed to clot at room temperature and serum will be collected after centrifugation.

The antibody or antibody fragment is partially purified from the serum samples and the amount of antibody or antibody fragment in the samples will be quantitated by ELISA. Briefly, the ELISA consists of coating microtiter plates overnight at 4° C. with an antibody that recognizes the antibody or antibody fragment administered to the volunteer. The plates are then blocked for approximately 30 minutes at room temperate with PBS-Tween-0.5% BSA. Standard curves are constructed using purified antibody or antibody fragment, not administered to a volunteer. Samples are diluted in PBS-Tween-BSA. The samples and standards are incubated for approximately 1 hour at room temperature. Next, the bound antibody is treated with a labeled antibody (e.g., horseradish peroxidase conjugated goat-anti-human IgG) for approximately 1 hour at room temperature. Binding of the labeled antibody is detected, e.g., by a spectrophotometer.

The concentration of antibody or antibody fragment levels in the serum of volunteers are corrected by subtracting the predose serum level (background level) from the serum levels at each collection interval after administration of the dose. For each volunteer the pharmacokinetic parameters are computed according to the model-independent approach (Gibaldi et al., eds., 1982, Pharmacokinetics, 2^{nd} edition, Marcel Dekker, New York) from the corrected serum antibody or antibody fragment concentrations.

4.8.10 Methods to Identify MPV

The invention encompasses treatment of any isolates of MPV, including those which are characterized as belonging to the subgroups and variants described in section 4.1.7.1, or belonging to a yet to be characterized subgroup or variant.

Immunoassays can be used in order to characterize the protein components that are present in a given sample. Immunoassays are an effective way to compare viral isolates using peptides components of the viruses for identification. For example, a method for identifying an isolates of MPV comprises inoculating an essentially MPV-uninfected or specific-pathogen-free guinea pig or ferret (in the detailed description the animal is inoculated intranasally but other was of inoculation such as intramuscular or intradermal inoculation, and using an other experimental animal, is also feasible) with the prototype isolate I-2614 or related isolates. Sera are collected from the animal at day zero, two weeks and three weeks post inoculation. The animal specifically seroconverted as measured in virus neutralization (VN) assay and indirect immunofluorescence assay against the respective isolate I-2614 and the sera from the seroconverted animal are used in the immunological detection of said further isolates. As an example, the invention provides the characterization of a new member in the family of Paramyxoviridae, a human metapneumovirus or metapneumovirus-like virus (since its final taxonomy awaits discussion by a viral taxonomy committee the MPV is herein for example described as taxonomically corresponding to APV) (MPV) which may cause severe respiratory tract infection in humans. The clinical signs of the disease caused by MPV are essentially similar to those caused by hRSV, such as cough, myalgia, vomiting, fever broncheolitis or pneumonia, possible conjunctivitis, or combinations thereof. As is seen with hRSV infected children, specifically very young children may require hospitalization. As an example an MPV which was deposited Jan. 19, 2001 as I-2614 with CNCM, Institute Pasteur, Paris or a virus isolate phylogenetically corresponding therewith can be used

4.8.10.1 Phylogenetic Analysis

Phylogenetic relationships between isolates of mammalian MPV can be evaluated by the methods set forth below or any other technique known to the skilled artisan. Many methods or approaches are available to analyze phylogenetic relationship; these include distance, maximum likelihood, and maximum parsimony methods (Swofford, D L., et. al., Phylogenetic Inference. In Molecular Systematics. Eds. Hillis, D M, Mortiz, C, and Mable, B K. 1996. Sinauer Associates: Massachusetts, USA. pp. 407-514; Felsenstein, J., 1981, J. Mol. Evol. 17:368-376). In addition, bootstrapping techniques are an effective means of preparing and examining confidence intervals of resultant phylogenetic trees (Felsenstein, J., 1985, Evolution. 29:783-791). Any method or approach using nucleotide or peptide sequence information to compare mammalian MPV isolates can be used to establish phylogenetic relationships, including, but not limited to, distance, maximum likelihood, and maximum parsimony methods or approaches. Any method known in the art can be used to analyze the quality of phylogenetic data, including but not limited to bootstrapping. Alignment of nucleotide or peptide sequence data for use in phylogenetic approaches, include but are not limited to, manual alignment, computer pairwise alignment, and computer multiple alignment. One skilled in the art would be familiar with the preferable alignment method or phylogenetic approach to be used based upon the information required and the time allowed.

In one embodiment, a DNA maximum likehood method is used to infer relationships between hMPV isolates. In another embodiment, bootstrapping techniques are used to determine the certainty of phylogenetic data created using one of said phylogenetic approaches. In another embodiment, jumbling techniques are applied to the phylogenetic approach before the input of data in order to minimize the effect of sequence order entry on the phylogenetic analyses. In one specific embodiment, a DNA maximum likelihood method is used with bootstrapping. In another specific embodiment, a DNA maximum likelihood method is used with bootstrapping and jumbling. In another more specific embodiment, a DNA maximum likelihood method is used with 50 bootstraps. In another specific embodiment, a DNA maximum likelihood method is used with 50 bootstraps and 3 jumbles. In another specific embodiment, a DNA maximum likelihood method is used with 100 bootstraps and 3 jumbles.

In one embodiment, nucleic acid or peptide sequence information from an isolate of hMPV is compared or aligned with sequences of other hMPV isolates. The amino acid sequence can be the amino acid sequence of the L protein, the M protein, the N protein, the P protein, or the F protein. In another embodiment, nucleic acid or peptide sequence information from an hMPV isolate or a number of hMPV isolates is compared or aligned with sequences of other viruses. In another embodiment, phylogenetic approaches are applied to sequence alignment data so that phylogenetic relationships can be inferred and/or phylogenetic trees constructed. Any method or approach that uses nucleotide or peptide sequence information to compare hMPV isolates can be used to infer said phylogenetic relationships, including, but not limited to, distance, maximum likelihood, and maximum parsimony methods or approaches.

Other methods for the phylogenetic analysis are disclosed in International Patent Application PCT/NL02/00040, published as WO 02/057302, which is incorporated in its entirety herein. In particular, PCT/NL02/00040 discloses nucleic acid sequences that are suitable for phylogenetic analysis at page 12, line 27 to page 19, line 29, which is incorporated herein by reference.

For the phylogenetic analyses it is most useful to obtain the nucleic acid sequence of a non-MPV as outgroup with which the virus is to be compared, a very useful outgroup isolate can be obtained from avian pneumovirus serotype C (APV-C), see, e.g., FIG. 16.

Many methods and programs are known in the art and can be used in the inference of phylogenetic relationships, including, but not limited to BioEdit, ClustalW, TreeView, and NJPlot. Methods that would be used to align sequences and to generate phylogenetic trees or relationships would require the input of sequence information to be compared. Many methods or formats are known in the art and can be used to input sequence information, including, but not limited to, FASTA, NBRF, EMBL/SWISS, GDE protein, GDE nucleotide, CLUSTAL, and GCG/MSF. Methods that would be used to align sequences and to generate phylogenetic trees or relationships would require the output of results. Many methods or formats can be used in the output of information or results, including, but not limited to, CLUSTAL, NBRF/PIR, MSF, PHYLIP, and GDE. In one embodiment, ClustalW is used in conjunction with DNA maximum likelihood methods with 100 bootstraps and 3 jumbles in order to generate phylogenetic relationships.

4.8.10.2 Alignment of Sequences

Two or more amino acid sequences can be compared by BLAST (Altschul, S. F. et al., 1990, J. Mol. Biol. 215:403-410) to determine their sequence homology and sequence identities to each other. Two or more nucleotide sequences can be compared by BLAST (Altschul, S. F. et al., 1990, J. Mol. Biol. 215:403-410) to determine their sequence homology and sequence identities to each other. BLAST comparisons can be performed using the Clustal W method (MacVector™). In certain specific embodiments, the alignment of two or more sequences by a computer program can be followed by manual re-adjustment.

The determination of percent identity between two sequences can be accomplished using a mathematical algorithm. A preferred, non-limiting example of a mathematical algorithm utilized for the comparison of two sequences is the algorithm of Karlin and Altschul, 1990, Proc. Natl. Acad. Sci. USA 87:2264-2268, modified as in Karlin and Altschul, 1993, Proc. Natl. Acad. Sci. USA 90:5873-5877. Such an algorithm is incorporated into the NBLAST and XBLAST programs of Altschul et al., 1990, J. Mol. Biol. 215:403-410. BLAST nucleotide comparisons can be performed with the NBLAST program. BLAST amino acid sequence comparisons can be performed with the XBLAST program. To obtain gapped alignments for comparison purposes, Gapped BLAST can be utilized as described in Altschul et al., 1997, Nucleic Acids Res.25:3389-3402. Alternatively, PSI-Blast can be used to perform an iterated search which detects distant relationships between molecules (Altschul et al., 1997, supra). When utilizing BLAST, Gapped BLAST, and PSI-Blast programs, the default parameters of the respective programs (e.g., XBLAST and NBLAST) can be used (see http://www.ncbi.nlm.nih.gov). Another preferred, non-limiting example of a mathematical algorithm utilized for the comparison of sequences is the algorithm of Myers and Miller, 1988, CABIOS 4:11-17. Such an algorithm is incorporated into the ALIGN program (version 2.0) which is part of the GCG sequence alignment software package. When utilizing the ALIGN program for comparing amino acid sequences, a PAM120 weight residue table can be used. The gap length penalty can be set by the skilled artisan. The percent identity between two sequences can be determined using techniques similar to those described above, with or without allowing gaps. In calculating percent identity, typically only exact matches are counted.

4.8.10.3 Hybridization Conditions

A nucleic acid which is hybridizable to a nucleic acid of a mammalian MPV, or to its reverse complement, or to its complement can be used in the methods of the invention to determine their sequence homology and identities to each other. In certain embodiments, the nucleic acids are hybridized under conditions of high stringency. By way of example and not limitation, procedures using such conditions of high stringency are as follows. Prehybridization of filters containing DNA is carried out for 8 h to overnight at 65 C in buffer composed of 6×SSC, 50 mM Tris-HCl (pH 7.5), 1 mM EDTA, 0.02% PVP, 0.02% Ficoll, 0.02% BSA, and 500 μg/ml denatured salmon sperm DNA. Filters are hybridized for 48 h at 65 C in prehybridization mixture containing 100 μg/ml denatured salmon sperm DNA and 5-20×106 cpm of ³²P-labeled probe. Washing of filters is done at 37 C for 1 h in a solution containing 2×SSC, 0.01% PVP, 0.01% Ficoll, and 0.01% BSA. This is followed by a wash in 0.1×SSC at 50 C for 45 min before autoradiography. Other conditions of high stringency which may be used are well known in the art. In other embodiments of the invention, hybridization is performed under moderate of low stringency conditions, such conditions are well-known to the skilled artisan (see e.g., Sambrook et al., 1989, Molecular Cloning, A Laboratory Manual, 2d Ed., Cold Spring Harbor Laboratory Press, Cold Spring Harbor, N.Y.; see also, Ausubel et al., eds., in the Current Protocols in Molecular Biology series of laboratory technique manuals, 1987-1997 Current Protocols,© 1994-1997 John Wiley and Sons, Inc.).

TABLE 5
LEGEND FOR SEQUENCE LISTING

| | |
|---|---|
| SEQ ID NO: 1 | Human metapneumovirus isolate 00-1 matrix protein |
| | (M) and fusion protein (F) genes |
| SEQ ID NO: 2 | Avian pneumovirus fusion protein gene, partial cds |
| SEQ ID NO: 3 | Avian pneumovirus isolate 1b fusion protein |
| | mRNA, complete cds |
| SEQ ID NO: 4 | Turkey rhinotracheitis virus gene for fusion |
| | protein (F1 and F2 subunits), complete cds |
| SEQ ID NO: 5 | Avian pneumovirus matrix protein (M) gene, partial |
| | cds and Avian pneumovirus fusion glycoprotein (F) |
| | gene, complete cds |
| SEQ ID NO: 6 | paramyxovirus F protein hRSV B |
| SEQ ID NO: 7 | paramyxovirus F protein hRSV A2 |
| SEQ ID NO: 8 | human metapneumovirus01-71 (partial sequence) |
| SEQ ID NO: 9 | Human metapneumovirus isolate 00-1 matrix |
| | protein(M) and fusion protein (F) genes |
| SEQ ID NO: 10 | Avian pneumovirus fusion protein gene, partial cds |
| SEQ ID NO: 11 | Avian pneumovirus isolate 1b fusion protein |
| | mRNA, complete cds |
| SEQ ID NO: 12 | Turkey rhinotracheitis virus gene for fusion |
| | protein (F1 and F2 subunits), complete cds |
| SEQ ID NO: 13 | Avian pneumovirus fusion glycoprotein (F) gene, |
| | complete cds |
| SEQ ID NO: 14 | Turkey rhinotracheitis virus (strain |
| | CVL14/1)attachment protien (G) mRNA, complete cds |
| SEQ ID NO: 15 | Turkey rhinotracheitis virus (strain |
| | 6574) attachment protein (G), complete cds |
| SEQ ID NO: 16 | Turkey rhinotracheitis virus (strain |
| | CVL14/1)attachment protein (G) mRNA, complete cds |
| SEQ ID NO: 17 | Turkey rhinotracheitis virus (strain |
| | 6574)attachment protein (G), complete cds |
| SEQ ID NO: 18 | isolate NL/1/99 (99-1) HMPV (Human |
| | Metapneumovirus) cDNA sequence |
| SEQ ID NO: 19 | isolate NL/1/00 (00-1) HMPV cDNA sequence |
| SEQ ID NO: 20 | isolate NL/17/00 HMPV cDNA sequence |
| SEQ ID NO: 21 | isolate NL/1/94 HMPV cDNA sequence |
| SEQ ID NO: 22 | RT-PCR primer TR1 |
| SEQ ID NO: 23 | RT-PCR primer N1 |
| SEQ ID NO: 24 | RT-PCR primer N2 |
| SEQ ID NO: 25 | RT-PCR primer M1 |
| SEQ ID NO: 26 | RT-PCR primer M2 |
| SEQ ID NO: 27 | RT-PCR primer F1 |
| SEQ ID NO: 28 | RT-PCR primer N3 |
| SEQ ID NO: 29 | RT-PCR primer N4 |
| SEQ ID NO: 30 | RT-PCR primer M3 |
| SEQ ID NO: 31 | RT-PCR primer M4 |
| SEQ ID NO: 32 | RT-PCR primer F7 |
| SEQ ID NO: 33 | RT-PCR primer F8 |
| SEQ ID NO: 34 | RT-PCR primer L6 |
| SEQ ID NO: 35 | RT-PCR primer L7 |
| SEQ ID NO: 36 | Oligonucleotide probe M |
| SEQ ID NO: 37 | Oligonucleotide probe N |
| SEQ ID NO: 38 | Oligonucleotide probe L |
| SEQ ID NO: 39 | TaqMan primer and probe sequences for isolates |
| | NL/1/00, BI/1/01, FI/4/01, NL/8/01, FI/2/01 |
| SEQ ID NO: 40 | TaqMan primer and probe sequences for isolates |
| | NL/30/01 |
| SEQ ID NO: 41 | TaqMan primer and probe sequences for isolates |
| | NL/22/01 and NL/23/01 |
| SEQ ID NO: 42 | TaqMan primer and probe sequences for isolate |
| | NL/17/01 |
| SEQ ID NO: 43 | TaqMan primer and probe sequences for isolate |
| | NL/17/00 |
| SEQ ID NO: 44 | TaqMan primer and probe sequences for isolates |
| | NL/9/01, NL/21/01, and NL/5/01 |
| SEQ ID NO: 45 | TaqMan primer and probe sequences for isolates |
| | FI/1/01 and FI/10/01 |
| SEQ ID NO: 46 | Primer ZF1 |
| SEQ ID NO: 47 | Primer ZF4 |
| SEQ ID NO: 48 | Primer ZF7 |
| SEQ ID NO: 49 | Primer ZF10 |
| SEQ ID NO: 50 | Primer ZF13 |
| SEQ ID NO: 51 | Primer ZF16 |
| SEQ ID NO: 52 | Primer CS1 |
| SEQ ID NO: 53 | Primer CS4 |
| SEQ ID NO: 54 | Primer CS7 |
| SEQ ID NO: 55 | Primer CS10 |
| SEQ ID NO: 56 | Primer CS13 |
| SEQ ID NO: 57 | Primer CS16 |
| SEQ ID NO: 58 | Forward primer for amplification of HPIV-1 |
| SEQ ID NO: 59 | Reverse primer for amplification of HPIV-1 |
| SEQ ID NO: 60 | Forward primer for amplification of HPIV-2 |
| SEQ ID NO: 61 | Reverse primer for amplification of HPIV-2 |
| SEQ ID NO: 62 | Forward primer for amplification of HPIV-3 |
| SEQ ID NO: 63 | Reverse primer for amplification of HPIV-3 |
| SEQ ID NO: 64 | Forward primer for amplification of HPIV-4 |
| SEQ ID NO: 65 | Reverse primer for amplification of HPIV-4 |
| SEQ ID NO: 66 | Forward primer for amplification of Mumps |
| SEQ ID NO: 67 | Reverse primer for amplification of Mumps |
| SEQ ID NO: 68 | Forward primer for amplification of NDV |
| SEQ ID NO: 69 | Reverse primer for amplification of NDV |
| SEQ ID NO: 70 | Forward primer for amplification of Tupaia |
| SEQ ID NO: 71 | Reverse primer for amplification of Tupaia |
| SEQ ID NO: 72 | Forward primer for amplification of Mapuera |
| SEQ ID NO: 73 | Reverse primer for amplification of Mapuera |
| SEQ ID NO: 74 | Forward primer for amplification of Hendra |
| SEQ ID NO: 75 | Reverse primer for amplification of Hendra |
| SEQ ID NO: 76 | Forward primer for amplification of Nipah |
| SEQ ID NO: 77 | Reverse primer for amplification of Nipah |
| SEQ ID NO: 78 | Forward primer for amplification of HRSV |
| SEQ ID NO: 79 | Reverse primer for amplification of HRSV |
| SEQ ID NO: 80 | Forward primer for amplification of Measles |
| SEQ ID NO: 81 | Reverse primer for amplification of Measles |
| SEQ ID NO: 82 | Forward primer to amplify general |
| | paramyxoviridae viruses |
| SEQ ID NO: 83 | Reverse primer to amplify general paramyxoviridae |
| | viruses |
| SEQ ID NO: 84 | G-gene coding sequence for isolate NL/1/00 (A1) |
| SEQ ID NO: 85 | G-gene coding sequence for isolate BR/2/01 (A1) |
| SEQ ID NO: 86 | G-gene coding sequence for isolate FL/4/01 (A1) |
| SEQ ID NO: 87 | G-gene coding sequence for isolate FL/3/01 (A1) |
| SEQ ID NO: 88 | G-gene coding sequence for isolate FL/8/01 (A1) |
| SEQ ID NO: 89 | G-gene coding sequence for isolate FL/10/01 (A1) |
| SEQ ID NO: 90 | G-gene coding sequence for isolate NL/10/01 (A1) |
| SEQ ID NO: 91 | G-gene coding sequence for isolate NL/2/02 (A1) |
| SEQ ID NO: 92 | G-gene coding sequence for isolate NL/17/00 (A2) |
| SEQ ID NO: 93 | G-gene coding sequence for isolate NL/1/81 (A2) |
| SEQ ID NO: 94 | G-gene coding sequence for isolate NL/1/93 (A2) |
| SEQ ID NO: 95 | G-gene coding sequence for isolate NL/2/93 (A2) |
| SEQ ID NO: 96 | G-gene coding sequence for isolate NL/3/93 (A2) |
| SEQ ID NO: 97 | G-gene coding sequence for isolate NL/1/95 (A2) |
| SEQ ID NO: 98 | G-gene coding sequence for isolate NL/2/96 (A2) |
| SEQ ID NO: 99 | G-gene coding sequence for isolate NL/3/96 (A2) |
| SEQ ID NO: 100 | G-gene coding sequence for isolate NL/22/01 |
| | (A2) |
| SEQ ID NO: 101 | G-gene coding sequence for isolate NL/24/01 |
| | (A2) |
| SEQ ID NO: 102 | G-gene coding sequence for isolate NL/23/01 |
| | (A2) |
| SEQ ID NO: 103 | G-gene coding sequence for isolate NL/29/01 |
| | (A2) |
| SEQ ID NO: 104 | G-gene coding sequence for isolate NL/3/02 (A2) |
| SEQ ID NO: 105 | G-gene coding sequence for isolate NL/1/99 (B1) |
| SEQ ID NO: 106 | G-gene coding sequence for isolate NL/11/00 |
| | (B1) |
| SEQ ID NO: 107 | G-gene coding sequence for isolate NL/12/00 |
| | (B1) |
| SEQ ID NO: 108 | G-gene coding sequence for isolate NL/5/01 (B1) |
| SEQ ID NO: 109 | G-gene coding sequence for isolate NL/9/01 (B1) |
| SEQ ID NO: 110 | G-gene coding sequence for isolate NL/21/01 |
| | (B1) |
| SEQ ID NO: 111 | G-gene coding sequence for isolate NL/1/94 (B2) |
| SEQ ID NO: 112 | G-gene coding sequence for isolate NL/1/82 (B2) |
| SEQ ID NO: 113 | G-gene coding sequence for isolate NL/1/96 (B2) |
| SEQ ID NO: 114 | G-gene coding sequence for isolate NL/6/97 (B2) |
| SEQ ID NO: 115 | G-gene coding sequence for isolate NL/9/00 (B2) |
| SEQ ID NO: 116 | G-gene coding sequence for isolate NL/3/01 (B2) |
| SEQ ID NO: 117 | G-gene coding sequence for isolate NL/4/01 (B2) |
| SEQ ID NO: 118 | G-gene coding sequence for isolate UK/5/01 (B2) |
| SEQ ID NO: 119 | G-protein sequence for isolate NL/1/00 (A1) |
| SEQ ID NO: 120 | G-protein sequence for isolate BR/2/01 (A1) |
| SEQ ID NO: 121 | G-protein sequence for isolate FL/4/01 (A1) |
| SEQ ID NO: 122 | G-protein sequence for isolate FL/3/01 (A1) |
| SEQ ID NO: 123 | G-protein sequence for isolate FL/8/01 (A1) |
| SEQ ID NO: 124 | G-protein sequence for isolate FL/10/01 (A1) |
| SEQ ID NO: 125 | G-protein sequence for isolate NL/10/01 (A1) |
| SEQ ID NO: 126 | G-protein sequence for isolate NL/2/02 (A1) |
| SEQ ID NO: 127 | G-protein sequence for isolate NL/17/00 (A2) |
| SEQ ID NO: 128 | G-protein sequence for isolate NL/1/81 (A2) |
| SEQ ID NO: 129 | G-protein sequence for isolate NL/1/93 (A2) |
| SEQ ID NO: 130 | G-protein sequence for isolate NL/2/93 (A2) |
| SEQ ID NO: 131 | G-protein sequence for isolate NL/3/93 (A2) |
| SEQ ID NO: 132 | G-protein sequence for isolate NL/1/95 (A2) |
| SEQ ID NO: 133 | G-protein sequence for isolate NL/2/96 (A2) |
| SEQ ID NO: 134 | G-protein sequence for isolate NL/3/96 (A2) |
| SEQ ID NO: 135 | G-protein sequence for isolate NL/22/01 (A2) |
| SEQ ID NO: 136 | G-protein sequence for isolate NL/24/01 (A2) |
| SEQ ID NO: 137 | G-protein sequence for isolate NL/23/01 (A2) |
| SEQ ID NO: 138 | G-protein sequence for isolate NL/29/01 (A2) |
| SEQ ID NO: 139 | G-protein sequence for isolate NL/3/02 (A2) |
| SEQ ID NO: 140 | G-protein sequence for isolate NL/1/99 (B1) |
| SEQ ID NO: 141 | G-protein sequence for isolate NL/11/00 (B1) |
| SEQ ID NO: 142 | G-protein sequence for isolate NL/12/00 (B1) |
| SEQ ID NO: 143 | G-protein sequence for isolate NL/5/01 (B1) |
| SEQ ID NO: 144 | G-protein sequence for isolate NL/9/01 (B1) |
| SEQ ID NO: 145 | G-protein sequence for isolate NL/21/01 (B1) |
| SEQ ID NO: 146 | G-protein sequence for isolate NL/1/94 (B2) |
| SEQ ID NO: 147 | G-protein sequence for isolate NL/1/82 (B2) |
| SEQ ID NO: 148 | G-protein sequence for isolate NL/1/96 (B2) |
| SEQ ID NO: 149 | G-protein sequence for isolate NL/6/97 (B2) |
| SEQ ID NO: 150 | G-protein sequence for isolate NL/9/00 (B2) |
| SEQ ID NO: 151 | G-protein sequence for isolate NL/3/01 (B2) |
| SEQ ID NO: 152 | G-protein sequence for isolate NL/4/01 (B2) |
| SEQ ID NO: 153 | G-protein sequence for isolate NL/5/01 (B2) |
| SEQ ID NO: 154 | F-gene coding sequence for isolate NL/1/00 |
| SEQ ID NO: 155 | F-gene coding sequence for isolate UK/1/00 |
| SEQ ID NO: 156 | F-gene coding sequence for isolate NL/2/00 |
| SEQ ID NO: 157 | F-gene coding sequence for isolate NL/13/00 |
| SEQ ID NO: 158 | F-gene coding sequence for isolate NL/14/00 |
| SEQ ID NO: 159 | F-gene coding sequence for isolate FL/3/01 |
| SEQ ID NO: 160 | F-gene coding sequence for isolate FL/4/01 |
| SEQ ID NO: 161 | F-gene coding sequence for isolate FL/8/01 |
| SEQ ID NO: 162 | F-gene coding sequence for isolate UK/1/01 |
| SEQ ID NO: 163 | F-gene coding sequence for isolate UK/7/01 |
| SEQ ID NO: 164 | F-gene coding sequence for isolate FL/10/01 |
| SEQ ID NO: 165 | F-gene coding sequence for isolate NL/6/01 |
| SEQ ID NO: 166 | F-gene coding sequence for isolate NL/8/01 |
| SEQ ID NO: 167 | F-gene coding sequence for isolate NL/10/01 |
| SEQ ID NO: 168 | F-gene coding sequence for isolate NL/14/01 |
| SEQ ID NO: 169 | F-gene coding sequence for isolate NL/20/01 |
| SEQ ID NO: 170 | F-gene coding sequence for isolate NL/25/01 |
| SEQ ID NO: 171 | F-gene coding sequence for isolate NL/26/01 |
| SEQ ID NO: 172 | F-gene coding sequence for isolate NL/28/01 |
| SEQ ID NO: 173 | F-gene coding sequence for isolate NL/30/01 |
| SEQ ID NO: 174 | F-gene coding sequence for isolate BR/2/01 |
| SEQ ID NO: 175 | F-gene coding sequence for isolate BR/3/01 |
| SEQ ID NO: 176 | F-gene coding sequence for isolate NL/2/02 |
| SEQ ID NO: 177 | F-gene coding sequence for isolate NL/4/02 |
| SEQ ID NO: 178 | F-gene coding sequence for isolate NL/5/02 |
| SEQ ID NO: 179 | F-gene coding sequence for isolate NL/6/02 |
| SEQ ID NO: 180 | F-gene coding sequence for isolate NL/7/02 |
| SEQ ID NO: 181 | F-gene coding sequence for isolate NL/9/02 |
| SEQ ID NO: 182 | F-gene coding sequence for isolate FL/1/02 |
| SEQ ID NO: 183 | F-gene coding sequence for isolate NL/1/81 |
| SEQ ID NO: 184 | F-gene coding sequence for isolate NL/1/93 |
| SEQ ID NO: 185 | F-gene coding sequence for isolate NL/2/93 |
| SEQ ID NO: 186 | F-gene coding sequence for isolate NL/4/93 |
| SEQ ID NO: 187 | F-gene coding sequence for isolate NL/1/95 |
| SEQ ID NO: 188 | F-gene coding sequence for isolate NL/2/96 |
| SEQ ID NO: 189 | F-gene coding sequence for isolate NL/3/96 |
| SEQ ID NO: 190 | F-gene coding sequence for isolate NL/1/98 |
| SEQ ID NO: 191 | F-gene coding sequence for isolate NL/17/00 |
| SEQ ID NO: 192 | F-gene coding sequence for isolate NL/22/01 |
| SEQ ID NO: 193 | F-gene coding sequence for isolate NL/29/01 |
| SEQ ID NO: 194 | F-gene coding sequence for isolate NL/23/01 |
| SEQ ID NO: 195 | F-gene coding sequence for isolate NL/17/01 |
| SEQ ID NO: 196 | F-gene coding sequence for isolate NL/24/01 |
| SEQ ID NO: 197 | F-gene coding sequence for isolate NL/3/02 |
| SEQ ID NO: 198 | F-gene coding sequence for isolate NL/3/98 |
| SEQ ID NO: 199 | F-gene coding sequence for isolate NL/1/99 |
| SEQ ID NO: 200 | F-gene coding sequence for isolate NL/2/99 |
| SEQ ID NO: 201 | F-gene coding sequence for isolate NL/3/99 |
| SEQ ID NO: 202 | F-gene coding sequence for isolate NL/11/00 |
| SEQ ID NO: 203 | F-gene coding sequence for isolate NL/12/00 |
| SEQ ID NO: 204 | F-gene coding sequence for isolate NL/1/01 |
| SEQ ID NO: 205 | F-gene coding sequence for isolate NL/5/01 |
| SEQ ID NO: 206 | F-gene coding sequence for isolate NL/9/01 |
| SEQ ID NO: 207 | F-gene coding sequence for isolate NL/19/01 |
| SEQ ID NO: 208 | F-gene coding sequence for isolate NL/21/01 |
| SEQ ID NO: 209 | F-gene coding sequence for isolate |
| | UK/11/01 |
| SEQ ID NO: 210 | F-gene coding sequence for isolate FL/1/01 |
| SEQ ID NO: 211 | F-gene coding sequence for isolate FL/2/01 |
| SEQ ID NO: 212 | F-gene coding sequence for isolate FL/5/01 |
| SEQ ID NO: 213 | F-gene coding sequence for isolate FL/7/01 |
| SEQ ID NO: 214 | F-gene coding sequence for isolate FL/9/01 |
| SEQ ID NO: 215 | F-gene coding sequence for isolate UK/10/01 |
| SEQ ID NO: 216 | F-gene coding sequence for isolate NL/1/02 |
| SEQ ID NO: 217 | F-gene coding sequence for isolate NL/1/94 |
| SEQ ID NO: 218 | F-gene coding sequence for isolate NL/1/96 |
| SEQ ID NO: 219 | F-gene coding sequence for isolate NL/6/97 |
| SEQ ID NO: 220 | F-gene coding sequence for isolate NL/7/00 |
| SEQ ID NO: 221 | F-gene coding sequence for isolate NL/9/00 |
| SEQ ID NO: 222 | F-gene coding sequence for isolate NL/19/00 |
| SEQ ID NO: 223 | F-gene coding sequence for isolate NL/28/00 |
| SEQ ID NO: 224 | F-gene coding sequence for isolate NL/3/01 |
| SEQ ID NO: 225 | F-gene coding sequence for isolate NL/4/01 |
| SEQ ID NO: 226 | F-gene coding sequence for isolate NL/11/01 |
| SEQ ID NO: 227 | F-gene coding sequence for isolate NL/15/01 |
| SEQ ID NO: 228 | F-gene coding sequence for isolate NL/18/01 |
| SEQ ID NO: 229 | F-gene coding sequence for isolate FL/6/01 |
| SEQ ID NO: 230 | F-gene coding sequence for isolate UK/5/01 |
| SEQ ID NO: 231 | F-gene coding sequence for isolate UK/8/01 |
| SEQ ID NO: 232 | F-gene coding sequence for isolate NL/12/02 |
| SEQ ID NO: 233 | F-gene coding sequence for isolate HK/1/02 |
| SEQ ID NO: 234 | F-protein sequence for isolate NL/1/00 |
| SEQ ID NO: 235 | F-protein sequence for isolate UK/1/00 |
| SEQ ID NO: 236 | F-protein sequence for isolate NL/2/00 |
| SEQ ID NO: 237 | F-protein sequence for isolate NL/13/00 |
| SEQ ID NO: 238 | F-protein sequence for isolate NL/14/00 |
| SEQ ID NO: 239 | F-protein sequence for isolate FL/3/01 |
| SEQ ID NO: 240 | F-protein sequence for isolate FL/4/01 |
| SEQ ID NO: 241 | F-protein sequence for isolate FL/8/01 |
| SEQ ID NO: 242 | F-protein sequence for isolate UK/1/01 |
| SEQ ID NO: 243 | F-protein sequence for isolate UK/7/01 |
| SEQ ID NO: 244 | F-protein sequence for isolate FL/10/01 |
| SEQ ID NO: 245 | F-protein sequence for isolate NL/6/01 |
| SEQ ID NO: 246 | F-protein sequence for isolate NL/8/01 |
| SEQ ID NO: 247 | F-protein sequence for isolate NL/10/01 |
| SEQ ID NO: 248 | F-protein sequence for isolate NL/14/01 |
| SEQ ID NO: 249 | F-protein sequence for isolate NL/20/01 |
| SEQ ID NO: 250 | F-protein sequence for isolate NL/25/01 |
| SEQ ID NO: 251 | F-protein sequence for isolate NL/26/01 |
| SEQ ID NO: 252 | F-protein sequence for isolate NL/28/01 |
| SEQ ID NO: 253 | F-protein sequence for isolate NL/30/01 |
| SEQ ID NO: 254 | F-protein sequence for isolate BR/2/01 |
| SEQ ID NO: 255 | F-protein sequence for isolate BR/3/01 |
| SEQ ID NO: 256 | F-protein sequence for isolate NL/2/02 |
| SEQ ID NO: 257 | F-protein sequence for isolate NL/4/02 |
| SEQ ID NO: 258 | F-protein sequence for isolate NL/5/02 |
| SEQ ID NO: 259 | F-protein sequence for isolate NL/6/02 |
| SEQ ID NO: 260 | F-protein sequence for isolate NL/7/02 |
| SEQ ID NO: 261 | F-protein sequence for isolate NL/9/02 |
| SEQ ID NO: 262 | F-protein sequence for isolate FL/1/02 |
| SEQ ID NO: 263 | F-protein sequence for isolate NL/1/81 |
| SEQ ID NO: 264 | F-protein sequence for isolate NL/1/93 |
| SEQ ID NO: 265 | F-protein sequence for isolate NL/2/93 |
| SEQ ID NO: 266 | F-protein sequence for isolate NL/4/93 |
| SEQ ID NO: 267 | F-protein sequence for isolate NL/1/95 |
| SEQ ID NO: 268 | F-protein sequence for isolate NL/2/96 |
| SEQ ID NO: 269 | F-protein sequence for isolate NL/3/96 |
| SEQ ID NO: 270 | F-protein sequence for isolate NL/1/98 |
| SEQ ID NO: 271 | F-protein sequence for isolate NL/17/00 |
| SEQ ID NO: 272 | F-protein sequence for isolate NL/22/01 |
| SEQ ID NO: 273 | F-protein sequence for isolate NL/29/01 |
| SEQ ID NO: 274 | F-protein sequence for isolate NL/23/01 |
| SEQ ID NO: 275 | F-protein sequence for isolate NL/17/01 |
| SEQ ID NO: 276 | F-protein sequence for isolate NL/24/01 |
| SEQ ID NO: 277 | F-protein sequence for isolate NL/3/02 |
| SEQ ID NO: 278 | F-protein sequence for isolate NL/3/98 |
| SEQ ID NO: 279 | F-protein sequence for isolate NL/1/99 |
| SEQ ID NQ: 280 | F-protein sequence for isolate NL/2/99 |
| SEQ ID NO: 281 | F-protein sequence for isolate NL/3/99 |
| SEQ ID NO: 282 | F-protein sequence for isolate NL/11/00 |
| SEQ ID NO: 283 | F-protein sequence for isolate NL/12/00 |
| SEQ ID NO: 284 | F-protein sequence for isolate NL/1/01 |
| SEQ ID NO: 285 | F-protein sequence for isolate NL/5/01 |
| SEQ ID NO: 286 | F-protein sequence for isolate NL/9/01 |
| SEQ ID NO: 287 | F-protein sequence for isolate NL/19/01 |
| SEQ ID NO: 288 | F-protein sequence for isolate NL/21/01 |
| SEQ ID NO: 289 | F-protein sequence for isolate UK/11/01 |
| SEQ ID NO: 290 | F-protein sequence for isolate FL/1/01 |
| SEQ ID NO: 291 | F-protein sequence for isolate FL/2/01 |
| SEQ ID NO: 292 | F-protein sequence for isolate FL/5/01 |
| SEQ ID NO: 293 | F-protein sequence for isolate FL/7/01 |
| SEQ ID NO: 294 | F-protein sequence for isolate FL/9/01 |
| SEQ ID NO: 295 | F-protein sequence for isolate UK/10/01 |
| SEQ ID NO: 296 | F-protein sequence for isolate NL/1/02 |
| SEQ ID NO: 297 | F-protein sequence for isolate NL/1/94 |
| SEQ ID NO: 298 | F-protein sequence for isolate NL/1/96 |
| SEQ ID NO: 299 | F-protein sequence for isolate NL/6/97 |
| SEQ ID NO: 300 | F-protein sequence for isolate NL/7/00 |
| SEQ ID NO: 301 | F-protein sequence for isolate NL/9/00 |
| SEQ ID NO: 302 | F-protein sequence for isolate NL/19/00 |
| SEQ ID NO: 303 | F-protein sequence for isolate NL/28/00 |
| SEQ ID NO: 304 | F-protein sequence for isolate NL/3/01 |
| SEQ ID NO: 305 | F-protein sequence for isolate NL/4/01 |
| SEQ ID NO: 306 | F-protein sequence for isolate NL/11/01 |
| SEQ ID NO: 307 | F-protein sequence for isolate NL/15/01 |
| SEQ ID NO: 308 | F-protein sequence for isolate NL/18/01 |
| SEQ ID NO: 309 | F-protein sequence for isolate FL/6/01 |
| SEQ ID NO: 310 | F-protein sequence for isolate UK/5/01 |
| SEQ ID NO: 311 | F-protein sequence for isolate UK/8/01 |
| SEQ ID NO: 312 | F-protein sequence for isolate NL/12/02 |
| SEQ ID NO: 313 | F-protein sequence for isolate HK/1/02 |
| SEQ ID NO: 314 | F protein sequence for HMPV isolate NL/1/00 |
| SEQ ID NO: 315 | F protein sequence for HMPV isolate NL/17/00 |
| SEQ ID NO: 316 | F protein sequence for HMPV isolate NL/1/99 |
| SEQ ID NO: 317 | F protein sequence for HMPV isolate NL/1/94 |
| SEQ ID NO: 318 | F-gene sequence for HMPV isolate NL/1/00 |
| SEQ ID NO: 319 | F-gene sequence for HMPV isolate NL/17/00 |
| SEQ ID NO: 320 | F-gene sequence for HMPV isolate NL/1/99 |
| SEQ ID NO: 321 | F-gene sequence for HMPV isolate NL/1/94 |
| SEQ ID NO: 322 | G protein sequence for HMPV isolate NL/1/00 |
| SEQ ID NO: 323 | G protein sequence for HMPV isolate NL/17/00 |
| SEQ ID NO: 324 | G protein sequence for HMPV isolate NL/1/99 |
| SEQ ID NO: 325 | G protein sequence for HMPV isolate NL/1/94 |
| SEQ ID NO: 326 | G-gene sequence for HMPV isolate NL/1/00 |
| SEQ ID NO: 327 | G-gene sequence for HMPV isolate NL/17/00 |
| SEQ ID NO: 328 | G-gene sequence for HMPV isolate NL/1/99 |
| SEQ ID NO: 329 | G-gene sequence for HMPV isolate NL/1/94 |
| SEQ ID NO: 330 | L protein sequence for HMPV isolate NL/1/00 |
| SEQ ID NO: 331 | L protein sequence for HMPV isolate NL/17/00 |
| SEQ ID NO: 332 | L protein sequence for HMPV isolate NL/1/99 |
| SEQ ID NO: 333 | L protein sequence for HMPV isolate NL/1/94 |
| SEQ ID NO: 334 | L-gene sequence for HMPV isolate NL/1/00 |
| SEQ ID NO: 335 | L-gene sequence for HMPV isolate NL/17/00 |
| SEQ ID NO: 336 | L-gene sequence for HMPV isolate NL/1/99 |
| SEQ ID NO: 337 | L-gene sequence for HMPV isolate NL/1/94 |
| SEQ ID NO: 338 | M2-1 protein sequence for HMPV isolate NL/1/00 |
| SEQ ID NO: 339 | M2-1 protein sequence for HMPV isolate NL/17/00 |
| SEQ ID NO: 340 | M2-1 protein sequence for HMPV isolate NL/1/99 |
| SEQ ID NO: 341 | M2-1 protein sequence for HMPV isolate NL/1/94 |
| SEQ ID NO: 342 | M2-1 gene sequence for HMPV isolate NL/1/00 |
| SEQ ID NO: 343 | M2-1 gene sequence for HMPV isolate NL/17/00 |
| SEQ ID NO: 344 | M2-1 gene sequence for HMPV isolate NL/1/99 |
| SEQ ID NO: 345 | M2-1 gene sequence for HMPV isolate NL/1/94 |
| SEQ ID NO: 346 | M2-2 protein sequence for HMPV isolate NL/1/00 |
| SEQ ID NO: 347 | M2-2 protein sequence for HMPV isolate NL/17/00 |
| SEQ ID NO: 348 | M2-2 protein sequence for HMPV isolate NL/1/99 |
| SEQ ID NO: 349 | M2-2 protein sequence for HMPV isolate NL/1/94 |
| SEQ ID NO: 350 | M2-2 gene sequence for HMPV isolate NL/1/00 |
| SEQ ID NO: 351 | M2-2 gene sequence for HMPV isolate NL/17/00 |
| SEQ ID NO: 352 | M2-2 gene sequence for HMPV isolate NL/1/99 |
| SEQ ID NO: 353 | M2-2 gene sequence for HMPV isolate |
| | NL/1/94 |
| SEQ ID NO: 354 | M2 gene sequence for HMPV isolate NL/1/00 |
| SEQ ID NO: 355 | M2 gene sequence for HMPV isolate NL/17/00 |
| SEQ ID NO: 356 | M2 gene sequence for HMPV isolate NL/1/99 |
| SEQ ID NO: 357 | M2 gene sequence for HMPV isolate NL/1/94 |
| SEQ ID NO: 358 | M protein sequence for HMPV isolate NL/1/00 |
| SEQ ID NO: 359 | M protein sequence for HMPV isolate NL/17/00 |
| SEQ ID NO: 360 | M protein sequence for HMPV isolate NL/1/99 |
| SEQ ID NO: 361 | M protein sequence for HMPV isolate NL/1/94 |
| SEQ ID NO: 362 | M gene sequence for HMPV isolate NL/1/00 |
| SEQ ID NO: 363 | M gene sequence for HMPV isolate NL/17/00 |
| SEQ ID NO: 364 | M gene sequence for HMPV isolate NL/1/99 |
| SEQ ID NO: 365 | M gene sequence for HMPV isolate NL/1/94 |
| SEQ ID NO: 366 | N protein sequence for HMPV isolate NL/1/00 |
| SEQ ID NO: 367 | N protein sequence for HMPV isolate NL/17/00 |
| SEQ ID NO: 368 | N protein sequence for HMPV isolate NL/1/99 |
| SEQ ID NO: 369 | N protein sequence for HMPV isolate NL/1/94 |
| SEQ ID NO: 370 | N gene sequence for HMPV isolate NL/1/00 |
| SEQ ID NO: 371 | N gene sequence for HMPV isolate NL/17/00 |
| SEQ ID NO: 372 | N gene sequence for HMPV isolate NL/1/99 |
| SEQ ID NO: 373 | N gene sequence for HMPV isolate NL/1/94 |
| SEQ ID NO: 374 | P protein sequence for HMPV isolate NL/1/00 |
| SEQ ID NO: 375 | P protein sequence for HMPV isolate NL/17/00 |
| SEQ ID NO: 376 | P protein sequence for HMPV isolate NL/1/99 |
| SEQ ID NO: 377 | P protein sequence for HMPV isolate NL/1/94 |
| SEQ ID NO: 378 | P gene sequence for HMPV isolate NL/1/00 |
| SEQ ID NO: 379 | P gene sequence for HMPV isolate NL/17/00 |
| SEQ ID NO: 380 | P gene sequence for HMPV isolate NL/1/99 |
| SEQ ID NO: 381 | P gene sequence for HMPV isolate NL/1/94 |
| SEQ ID NO: 382 | SH protein sequence for HMPV isolate NL/1/00 |
| SEQ ID NO: 383 | SH protein sequence for HMPV isolate NL/17/00 |
| SEQ ID NO: 384 | SH protein sequence for HMPV isolate NL/1/99 |
| SEQ ID NO: 385 | SH protein sequence for HMPV isolate NL/1/94 |
| SEQ ID NO: 386 | SH gene sequence for HMPV isolate NL/1/00 |
| SEQ ID NO: 387 | SH gene sequence for HMPV isolate NL/17/00 |
| SEQ ID NO: 388 | SH gene sequence for HMPV isolate NL/1/99 |
| SEQ ID NO: 389 | SH gene sequence for HMPV isolate NL/1/94 |
| SEQ ID NO: 390 | attachment glycoprotein of Human respiratory |
| | syncytial virus |
| SEQ ID NO: 391 | fusion glycoprotein of Human respiratory |
| | syncytial virus |
| SEQ ID NO: 392 | small hydrophobic protein of Human respiratory |
| | syncytial virus |
| SEQ ID NO: 393 | RNA polymerase beta subunit (Large structural |
| | protein) (L protein) of Human respiratory |
| | syncytial virus |
| SEQ ID NO: 394 | phosphoprotein P of Human respiratory syncytial |
| | virus |
| SEQ ID NO: 395 | attachment glycoprotein G of Human respiratory |
| | syncytial virus |
| SEQ ID NO: 396 | nucleocapsid protein of Human respiratory |
| | syncytial virus |
| SEQ ID NO: 397 | nucleoprotein (N) of Human respiratory |
| | syncytial virus |
| SEQ ID NO: 398 | matrix protein of Human respiratory syncytial |
| | virus |
| SEQ ID NO: 399 | Nucleoprotein (N) |
| SEQ ID NO: 400 | Phosphoprotein (P) |
| SEQ ID NO: 401 | Matrix Protein (M) |
| SEQ ID NO: 402 | Matrix Protein 2-1 (M2) |
| SEQ ID NO: 403 | Matrix Protein 2-2 (M2) |
| SEQ ID NO: 404 | Small Hydrophobic Protein (SH) |
| SEQ ID NO: 405 | RNA-dependent RNA polymerase (L) of Human |
| | metapneumovirus |
| SEQ ID NO: 406 | RNA-dependent RNA polymerase (L) of Human |
| | metapneumovirus |
| SEQ ID NO: 407 | RNA polymerase alpha subunit (Nucleocapsid |
| | phosphoprotein) of Human parainfluenza 1 virus |
| SEQ ID NO: 408 | L polymerase protein of Human parainfluenza 1 |
| | virus |
| SEQ ID NO: 409 | HN glycoprotein of Human parainfluenza 1 virus |
| SEQ ID NO: 410 | matrix protein of Human parainfluenza 1 virus |
| SEQ ID NO: 411 | Y1 protein of Human parainfluenza 1 virus |
| SEQ ID NO: 412 | C protein of Human parainfluenza 1 virus |
| SEQ ID NO: 413 | phosphoprotein of Human parainfluenza 1 virus |
| SEQ ID NO: 414 | nucleoprotein of Human parainfluenza 1 virus |
| SEQ ID NO: 415 | F glycoprotein of Human parainfluenza 1 virus |
| SEQ ID NO: 416 | D protein of Human parainfluenza virus 3 |
| SEQ ID NO: 417 | hemagglutinin-neuraminidase of Human |
| | parainfluenza virus 3 |
| SEQ ID NO: 418 | nucleocapsid protein of Human parainfluenza |
| | virus 3 |
| SEQ ID NO: 419 | P protein of Human parainfluenza virus 2 |
| SEQ ID NO: 420 | F protein of Human parainfluenza virus |
| SEQ ID NO: 421 | G protein of Human parainfluenza virus |
| SEQ ID NO: 422 | Homo sapiens |
| SEQ ID NO: 423 | Homo sapiens |
| SEQ ID NO: 424 | Avian pneumovirus fusion protein gene |
| SEQ ID NO: 425 | Avian pneumovirus isolate 1b fusion protein |
| | mRNA |
| SEQ ID NO: 426 | Turkey rhinotracheitis virus gene for fusion |
| | protein (F1 and F2 subunits), complete cds |
| SEQ ID NO: 427 | Avian pneumovirus fusion glycoprotein (F) gene, |
| | complete cds |
| SEQ ID NO: 428 | Turkey rhinotracheitis virus (strain CVL14/1) |
| | attachment protien (G) mRNA, complete cds |
| SEQ ID NO: 429 | Turkey rhinotracheitis virus (strain 6574) |
| | attachment protein (G) |
| SEQ ID NO: 430 | Postulated HRA sequence of strain NL1/00 |
| SEQ ID NO: 431 | Postulated HRA sequence of strain NL17/00 |
| SEQ ID NO: 432 | Postulated HRA sequence of strain NL1/99 |
| SEQ ID NO: 433 | Postulated HRA sequence of strain NL1/94 |
| SEQ ID NO: 434 | Postulated HRB sequence of strain NL1/00 |
| SEQ ID NO: 435 | Postulated HRB sequence of strain NL17/00 |
| SEQ ID NO: 436 | Postulated HRB sequence of strain NL1/99 |
| SEQ ID NO: 437 | Postulated HRB sequence of strain NL1/94 |

Equivalents

Those skilled in the art will recognize, or be able to ascertain using no more than routine experimentation, many equivalents to the specific embodiments of the invention described herein. Such equivalents are intended to be encompassed by the following claims.

All publications, patents and patent applications mentioned in this specification are herein incorporated by reference into the specification to the same extent as if each individual publication, patent or patent application was specifically and individually indicated to be incorporated herein by reference.

What is claimed is:

1. A method of preventing a viral infection in a subject, said method comprising administering to the subject:
(i) a prophylactically effective amount of one or more first antibodies or antigen-binding fragments thereof, wherein one or more of said first antibodies or antigen-binding fragments thereof bind immunospecifically to a RSV antigen; and
(ii) a prophylactically effective amount of one or more second antibodies or antigen-binding fragments thereof, wherein one or more of said second antibodies or antigen-binding fragments thereof bind immunospecifically to a hMPV antigen.

2. The method of claim 1, wherein one or more of said first antibodies or antigen-binding fragments thereof neutralize RSV.

3. The method of claim 1, wherein one or more of said second antibodies or antigen-binding fragments thereof neutralize hMPV.

4. The method of claim 1, wherein one or more of said first antibodies or antigen-binding fragments thereof block RSV infection of cells of the subject.

5. The method of claim 1, wherein one or more of said second antibodies or antigen-binding fragments thereof block hMPV infection of cells of the subject.

6. A method of treating one or more symptoms of a respiratory viral infection in a subject, said method comprising administering to the subject:
(i) a therapeutically effective amount of one or more first antibodies or antigen-binding fragments thereof, wherein one or more of said first antibodies or antigen-binding fragments thereof bind immunospecifically to a RSV antigen; and
(ii) a therapeutically effective amount of one or more second antibodies or antigen-binding fragments thereof, wherein one or more of said second antibodies or antigen-binding fragments thereof bind immunospecifically to a hMPV antigen.

7. A method of passive immunotherapy, said method comprising administering to a subject:
(i) a first dose of one or more first antibodies or antigen-binding fragments thereof, wherein one or more of said first antibodies or a fragments thereof bind immunospecifically to a RSV antigen; and
(ii) a second dose of one or more second antibodies or antigen-binding fragments thereof, wherein one or more of said second antibodies or a fragments thereof bind immunospecifically to a hMPV antigen,
wherein the first dose reduces the incidence of RSV infection by at least 25% and wherein the second dose reduces the incidence of hMPV infection by at least 25%.

8. A method of passive immunotherapy, said method comprising administering to a subject:
(i) a first dose of one or more first antibodies or antigen-binding fragments thereof, wherein one or more of said first antibodies or antigen-binding fragments thereof bind immunospecifically to a RSV antigen; and
(ii) a second dose of one or more second antibodies or antigen-binding fragments thereof, wherein one or more of said second antibodies or antigen-binding fragments thereof bind immunospecifically to a hMPV antigen,
wherein the serum titer of one or more of said first antibodies or antigen-binding fragments thereof in the subject is at least 10 μg/ml after 15 days of administering one or more of said first antibodies or antigen-binding fragments thereof and wherein the serum titer of one or more of said second antibodies or antigen-binding fragments thereof in the subject is at least 10 μg/ml after 15 days of administering one or more of said second antibodies or antigen-binding fragments thereof.

9. The method of claim 1, 6, 7, or 8, wherein the amino acid sequence of the RSV antigen is that of SEQ ID NO:390 to 398, respectively.

10. The method of claim 1, 6, 7, or 8, wherein the amino acid sequence of the RSV antigen is 90% identical to the amino acid sequence of RSV nucleoprotein, RSV phosphoprotein, RSV matrix protein, RSV small hydrophobic protein, RSV RNA-dependent RNA polymerase, RSV F protein, or RSV G protein.

11. The method of claim 1, 6, 7, or 8, wherein the RSV antigen is selected from the group consisting of RSV nucleoprotein, RSV phosphoprotein, RSV matrix protein, RSV small hydrophobic protein, RSV RNA-dependent RNA polymerase, RSV F protein, and RSV G protein.

12. The method of claim 1, 6, 7, or 8, wherein one or more of said first antibodies immunospecifically bind to an antigen of Group A or Group B RSV.

13. The method of claim 1, 6, 7, or 8, wherein the RSV antigen is RSV F protein.

14. The method of claim 1, 6, 7, or 8, wherein one or more of said second antibodies cross-react with a turkey APV antigen.

15. The method of claim 1, 6, 7, or 8, wherein one or more of said second antibodies are (i) human or humanized antibodies and (ii) cross-react with a turkey APV antigen.

16. The method of claim 15, wherein said turkey APV antigen is selected from the group consisting of turkey APV nucleoprotein, turkey APV phosphoprotein, turkey APV matrix protein, turkey APV small hydrophobic protein, turkey APV RNA-dependent RNA polymerase, turkey APV F protein, and turkey APV G protein.

17. The method of claim 15, wherein said turkey APV antigen is an antigen of avian pneumovirus type A, avian pneumovirus type B, or avian pneumovirus type C.

18. The method of claim 15, wherein the amino acid sequence of said turkey APV antigen is that of SEQ ID NO:424 to 429, respectively.

19. The method of claim 1, 6, 7, or 8, wherein the amino acid sequence of the hMPV antigen is that of SEQ ID NO: 399-406, 420, or 421, respectively.

20. The method of claim 1, 6, 7, or 8, wherein the hMPV antigen is selected from the group consisting of hMPV nucleoprotein, hMPV phosphoprotein, hMPV matrix protein, hMPV small hydrophobic protein, hMPV RNA-dependent RNA polymerase, hMPV F protein, and hMPV G protein.

21. The method of claim 1, 6, 7, or 8, wherein the hMPV antigen is hMPV F protein.

22. The method of claim 1, 6, 7, or 8, wherein the first antibody is Palivizumab; AFFF; P12f2 P12f4; P11d4; Ale9; A12a6; A13c4; A17d4; A4B4; 1X-493L1; FR H3-3F4; M3H9; Y10H6; DG; AFFF(1); 6H8; L1-7E5; L2-15B10; A13a11; A1h5; A4B4(1);A4B4-F52S; or A4B4L1FR-S28R.

23. The method of claim 1, 6, 7, or 8, wherein one or more of said first antibodies or antigen-binding fragments thereof are administered at a time period prior to administering of one or more of said second antibodies or antigen-binding fragments thereof.

24. The method of claim 1, 6, 7, or 8, wherein one or more of said second antibodies or antigen-binding fragments thereof are administered at a time period prior to administering of one or more of said first antibodies or antigen-binding fragments thereof.

25. The method of claim 1, 6, 7, or 8, wherein one or more of said first antibodies or antigen-binding fragments thereof and one or more of said second antibodies or antigen-binding fragments thereof are administered concurrently.

26. The method of claim 1, 6, 7, or 8, wherein one or more of said first antibodies or antigen-binding fragments thereof are administered in a sequence of two or more administrations, wherein the administrations of one or more of said first antibodies or antigen-binding fragments thereof are separated by a time period from each other, and wherein one or more of said second antibodies or antigen-binding fragments thereof are administered before, during, or after the sequence.

27. The method of claim 1, 6, 7, or 8, wherein one or more of said first antibodies or antigen-binding fragments thereof are administered in a sequence of two or more administrations, wherein the administrations of one or more of said second antibodies or antigen-binding fragments thereof are separated by a time period from each other, and wherein one or more of said first antibodies or antigen-binding fragments thereof are administered before, during, or after the sequence.

28. The method of claim 1, 6, 7, or 8, wherein one or more of said first antibodies or antigen-binding fragments thereof and one or more of said second antibodies or antigen-binding fragments thereof are administered in a sequence of two or more administrations, wherein the administrations are separated by a time period from each other.

29. The method of claim 1 or 6, wherein the viral infection is an infection with RSV and hMPV or an infection with RSV and APV.

30. A method of preventing a viral infection in a subject, said method comprising administering to the subject:
(i) a dose of one or more antibodies or antigen-binding fragments thereof, wherein one or more of said antibodies or antigen-binding fragments thereof (i) are human or humanized, (ii) cross-react with a turkey APV antigen, and (iii) bind immunospecifically to a hMPV antigen.

31. A method of treating one or more symptoms of a respiratory viral infection in a subject, said method comprising administering to the subject:
(i) a dose of one or more antibodies or antigen-binding fragments thereof, wherein one or more of said antibodies or antigen-binding fragments thereof (i) are human or humanized, (ii) cross-react with a turkey APV antigen, and (iii) bind immunospecifically to a hMPV antigen.

32. A method of passive immunotherapy, said method comprising administering to a subject:
(i) a dose of one or more antibodies or antigen-binding fragments thereof, wherein one or more of said antibodies or antigen-binding fragments thereof (i) are human or humanized, (ii) cross-react with a turkey APV antigen, and (iii) bind immunospecifically to a hMPV antigen,
wherein the dose reduces the incidence of hMPV infection by at least 25%.

33. A method of passive immunotherapy, said method comprising administering to a subject:
(i) a dose of one or more antibodies or antigen-binding fragments thereof, wherein one or more of said antibodies or antigen-binding fragments thereof
(i) are human or humanized, (ii) cross-react with a turkey APV antigen, and (iii) bind immunospecifically to a hMPV antigen,
wherein the serum titer of one or more of said antibodies or antigen-binding fragments thereof in the subject is at least 10 μg/ml after 15 days of administering one or more of said antibodies or antigen-binding fragments thereof.

34. A pharmaceutical composition, said composition comprising:
(i) one or more first antibodies or antigen-binding fragments thereof, wherein one or more of said first antibodies or antigen-binding fragments thereof bind immunospecifically to a RSV antigen; and
(ii) one or more second antibodies or antigen-binding fragments thereof, wherein one or more of said second antibodies or antigen-binding fragments thereof bind immunospecifically to a hMPV antigen.

35. The pharmaceutical composition of claim 34, wherein the amino acid sequence of the RSV antigen is that of SEQ ID NO:390 to 398, respectively.

36. The pharmaceutical composition of claim 34, wherein the amino acid sequence of the RSV antigen is 90% identical to the amino acid sequence of RSV nucleoprotein, RSV phosphoprotein, RSV matrix protein, RSV small hydrophobic protein, RSV RNA-dependent RNA polymerase, RSV F protein, or RSV G protein.

37. The pharmaceutical composition of claim 34, wherein the RSV antigen is selected from the group consisting of RSV nucleoprotein, RSV phosphoprotein, RSV matrix protein, RSV small hydrophobic protein, RSV RNA-dependent RNA polymerase, RSV F protein, and RSV G protein.

38. The pharmaceutical composition of claim 34, wherein one or more of said first antibodies or antigen-binding fragments thereof immunospecifically bind to an antigen of Group A or Group B RSV.

39. The pharmaceutical composition of claim 34, wherein the RSV antigen is RSV F protein.

40. The pharmaceutical composition of claim 34, wherein one or more of said second antibodies cross-react with a turkey APV antigen.

41. The pharmaceutical composition of claim 34, wherein one or more of said second antibodies are (i) human or humanized antibodies and (ii) cross-react with a turkey APV antigen.

42. The pharmaceutical composition of claim 40, wherein said turkey APV antigen is selected from the group consisting of turkey APV nucleoprotein, turkey APV phosphoprotein, turkey APV matrix protein, turkey APV small hydrophobic protein, turkey APV RNA-dependent RNA polymerase, turkey APV F protein, and turkey APV G protein.

43. The pharmaceutical composition of claim 40, wherein said turkey APV antigen is an antigen of avian pneumovirus type A, avian pneumovirus type B, or avian pneumovirus type C.

44. The pharmaceutical composition of claim 40, wherein the amino acid sequence of said turkey APV antigen is that of SEQ ID NO:424 to 429, respectively.

45. The pharmaceutical composition of claim 34, wherein the amino acid sequence of the hMPV antigen is that of SEQ ID NO: 399-406, 420, or 421, respectively.

46. The pharmaceutical composition of claim 34, wherein the hMPV antigen is selected from the group consisting of hMPV nucleoprotein, hMPV phosphoprotein, hMPV matrix protein, hMPV small hydrophobic protein, hMPV RNA-dependent RNA polymerase, hMPV F protein, and hMPV G protein.

47. The pharmaceutical composition of claim 34, wherein the hMPV antigen is hMPV F protein.

48. The pharmaceutical composition of claim 34, wherein the first antibody is Palivizumab; AFFF; P12f2 P12f4; P11d4; Ale9; A12a6; A13c4; A17d4; A4B4; 1X-493L1; FR H3-3F4; M3H9; Y10H6; DG; AFFF(1); 6H8; L1-7E5; L2-15B10; A13a11; A1h5; A4B4(1);A4B4-F52S; or A4B4L1FR-S28R.

49. A pharmaceutical composition, said composition comprising: one or more antibodies or antigen-binding fragments thereof, wherein one or more of said antibodies or antigen-binding fragments thereof (i) are human or humanized, (ii) cross-react with a turkey APV antigen, and (iii) bind immunospecifically to a hMPV antigen.

50. A method of preventing a viral infection in a subject, said method comprising administering to the subject:
(i) a prophylactically effective amount of one or more first antibodies or antigen-binding fragments thereof, wherein one or more of said first antibodies or antigen-binding fragments thereof bind immunospecifically to a PIV antigen; and
(ii) a prophylactically effective amount of one or more second antibodies or antigen-binding fragments thereof, wherein one or more of said second antibodies or antigen-binding fragments thereof bind immunospecifically to a hMPV antigen.

51. The method of claim 50, wherein one or more of said first antibodies or antigen-binding fragments thereof neutralize PIV.

52. The method of claim 50, wherein one or more of said second antibodies or antigen-binding fragments thereof neutralize hMPV.

53. The method of claim 50, wherein one or more of said first antibodies or antigen-binding fragments thereof block PIV infection of cells of the subject.

54. The method of claim 50, wherein one or more of said second antibodies or antigen-binding fragments thereof block hMPV infection of cells of the subject.

55. A method of treating one or more symptoms of a respiratory viral infection in a subject, said method comprising administering to the subject:
(i) a therapeutically effective amount of one or more first antibodies or antigen-binding fragments thereof, wherein one or more of said first antibodies or antigen-binding fragments thereof bind immunospecifically to a PIV antigen; and
(ii) a therapeutically effective amount of one or more second antibodies or antigen-binding fragments thereof, wherein one or more of said second antibodies or antigen-binding fragments thereof bind immunospecifically to a hMPV antigen.

56. A method of passive immunotherapy, said method comprising administering to a subject:
(i) a first dose of one or more first antibodies or antigen-binding fragments thereof, wherein one or more of said first antibodies or a fragments thereof bind immunospecifically to a PIV antigen; and
(ii) a second dose of one or more second antibodies or antigen-binding fragments thereof, wherein one or more of said second antibodies or a fragments thereof bind immunospecifically to a hMPV antigen,
wherein the first dose reduces the incidence of PIV infection by at least 25% and wherein the second dose reduces the incidence of hMPV infection by at least 25%.

57. A method of passive immunotherapy, said method comprising administering to a subject:
(i) a first dose of one or more first antibodies or antigen-binding fragments thereof, wherein one or more of said first antibodies or antigen-binding fragments thereof bind immunospecifically to a PIV antigen; and
(ii) a second dose of one or more second antibodies or antigen-binding fragments thereof, wherein one or more of said second antibodies or antigen-binding fragments thereof bind immunospecifically to a hMPV antigen,
wherein the serum titer of one or more of said first antibodies or antigen-binding fragments thereof in the subject is at least 10 μg/ml after 15 days of administering one or more of said first antibodies or antigen-binding fragments thereof and wherein the serum titer of one or more of said second antibodies or antigen-binding fragments thereof in the subject is at least 10 μg/ml after 15 days of administering one or more of said second antibodies or antigen-binding fragments thereof.

58. The method of claim 50, 55, 56, or 57, wherein the amino acid sequence of the PIV antigen is that of SEQ ID NO:407 to 419, respectively.

59. The method of claim 50, 55, 56, or 57, wherein the amino acid sequence of the PIV antigen is 90% identical to the amino acid sequence of PIV nucleocapsid phosphoprotein, PIV L protein, PIV matrix protein, PIV HN glycoprotein, PIV RNA-dependent RNA polymerase, PIV Y1 protein, PIV D protein, or PIV C protein.

60. The method of claim 50, 55, 56, or 57, wherein the PIV antigen is selected from the group consisting of PIV nucleocapsid phosphoprotein, PIV L protein, PIV matrix protein, PIV HN glycoprotein, PIV RNA-dependent RNA polymerase, PIV Y1 protein, PIV D protein, or PIV C protein.

61. The method of claim 50, 55, 56, or 57, wherein one or more of said first antibodies immunospecifically bind to an antigen of human PIV type 1, human PIV type 2, human PIV type 3, or human PIV type 4.

62. The method of claim 50, 55, 56, or 57, wherein the PUV antigen is PIV F protein.

63. The method of claim 50, 55, 56, or 57, wherein one or more of said second antibodies cross-react with a turkey APV antigen.

64. The method of claim 50, 55, 56, or 57, wherein one or more of said second antibodies are (i) human or humanized antibodies and (ii) cross-react with a turkey APV antigen.

65. The method of claim 63, or 64, wherein said turkey APV antigen is selected from the group consisting of turkey APV nucleoprotein, turkey APV phosphoprotein, turkey APV matrix protein, turkey APV small hydrophobic protein, turkey APV RNA-dependent RNA polymerase, turkey APV F protein, and turkey APV G protein.

66. The method of claim 63, 64, wherein said turkey APV antigen is an antigen of avian pneumovirus type A, avian pneumovirus type B, or avian pneumovirus type C.

67. The method of claim 63, or 64, wherein the amino acid sequence of said turkey APV antigen is that of SEQ ID NO:424 to 429, respectively.

68. The method of claim 50, 55, 56, or 57, wherein the amino acid sequence of the hMPV antigen is that of SEQ ID NO: 399-406, 420, or 421, respectively.

69. The method of claim 50, 55, 56, or 57, wherein the hMPV antigen is selected from the group consisting of hMPV nucleoprotein, hMPV phosphoprotein, hMPV matrix protein, hMPV small hydrophobic protein, hMPV RNA-dependent RNA polymerase, hMPV F protein, and hMPV G protein.

70. The method of claim 50, 55, 56, or 57, wherein the hMPV antigen is hMPV F protein.

71. The method of claim 50 or 107, wherein the viral infection is an infection with PIV and hMPV or an infection with PIV and APV.

72. A method of preventing a viral infection in a subject, said method comprising administering to the subject:
(i) a prophylactically effective amount of one or more first antibodies or antigen-binding fragments thereof, wherein one or more of said first antibodies or antigen-binding fragments thereof bind immunospecifically to a RSV antigen;
(ii) a prophylactically effective amount of one or more second antibodies or antigen-binding fragments thereof, wherein one or more of said second antibodies or antigen-binding fragments thereof bind immunospecifically to a hMPV antigen; and
(iii) a prophylactically effective amount of one or more third antibodies or antigen-binding fragments thereof, wherein one or more of said third antibodies or antigen-binding fragments thereof bind immunospecifically to a PIV antigen.

73. The method of claim 72, wherein one or more of said first antibodies or antigen-binding fragments thereof neutralize RSV.

74. The method of claim 72, wherein one or more of said second antibodies or antigen-binding fragments thereof neutralize hMPV.

75. The method of claim 72, wherein one or more of said third antibodies or antigen-binding fragments thereof neutralize PIV.

76. The method of claim 72, wherein one or more of said first antibodies or antigen-binding fragments thereof block RSV infection of cells of the subject.

77. The method of claim 72, wherein one or more of said second antibodies or antigen-binding fragments thereof block hMPV infection of cells of the subject.

78. The method of claim 72, wherein one or more of said third antibodies or antigen-binding fragments thereof block PIV infection of cells of the subject.

79. A method of treating one or more symptoms of a respiratory viral infection in a subject, said method comprising administering to the subject:
(i) a therapeutically effective amount of one or more first antibodies or antigen-binding fragments thereof, wherein one or more of said first antibodies or antigen-binding fragments thereof bind immunospecifically to a RSV antigen;
(ii) a therapeutically effective amount of one or more second antibodies or antigen-binding fragments thereof, wherein one or more of said second antibodies or antigen-binding fragments thereof bind immunospecifically to a hMPV antigen; and
(iii) a therapeutically effective amount of one or more third antibodies or antigen-binding fragments thereof, wherein one or more of said third antibodies or antigen-binding fragments thereof bind immunospecifically to a PIV antigen.

80. A method of passive immunotherapy, said method comprising administering to a subject:
(i) a first dose of one or more first antibodies or antigen-binding fragments thereof, wherein one or more of said first antibodies or a fragments thereof bind immunospecifically to a RSV antigen;
(ii) a second dose of one or more second antibodies or antigen-binding fragments thereof, wherein one or more of said second antibodies or a fragments thereof bind immunospecifically to a hMPV antigen; and
(iii) a third dose of one or more third antibodies or antigen-binding fragments thereof, wherein one or more of said third antibodies or antigen-binding fragments thereof bind immunospecifically to a PIV antigen.
wherein the first dose reduces the incidence of RSV infection by at least 25%, wherein the second dose reduces the incidence of hMPV infection by at least 25%, and wherein the third dose reduces the incidence of PIV infection by at least 25%.

81. A method of passive immunotherapy, said method comprising administering to a subject:
(i) a first dose of one or more first antibodies or antigen-binding fragments thereof, wherein one or more of said first antibodies or antigen-binding fragments thereof bind immunospecifically to a RSV antigen;
(ii) a second dose of one or more second antibodies or antigen-binding fragments thereof, wherein one or more of said second antibodies or antigen-binding fragments thereof bind immunospecifically to a hMPV antigen; and
(iii) a third dose of one or more third antibodies or antigen-binding fragments thereof, wherein one or more of said third antibodies or antigen-binding fragments thereof bind immunospecifically to a PW antigen,
wherein the serum titer of one or more of said first antibodies or antigen-binding fragments thereof in the subject is at least 10 μg/ml after 15 days of administering one or more of said first antibodies or antigen-binding fragments thereof, wherein the serum titer of one or more of said second antibodies or antigen-binding fragments thereof in the subject is at least 10 μg/ml after 15 days of administering one or more of said second antibodies or antigen-binding fragments thereof, and wherein the serum titer of one or more of said third antibodies or antigen-binding fragments thereof in the subject is at least 10 μg/ml after 15 days of administering one or more of said third antibodies or antigen-binding fragments thereof.

82. The method of claim 79, 80, or 81, wherein the amino acid sequence of the PIV antigen is that of SEQ ID NO:407 to 419, respectively.

83. The method of claim 79, 80, or 81, wherein the amino acid sequence of the PIV antigen is 90% identical to the amino acid sequence of PIV nucleoprotein, PIV phosphoprotein, PIV matrix protein, PIV small hydrophobic protein, PIV RNA-dependent RNA polymerase, PIV F protein, or PIV G protein.

84. The method of claim 79, 80, or 81, wherein the PIV antigen is selected from the group consisting of PIV nucleoprotein, PIV phosphoprotein, PIV matrix protein, PIV small hydrophobic protein, PIV RNA polymerase, PIV F protein, and PIV G protein.
